(19) United States
(12) Patent Application Publication
(10) Pub. No.: US 2024/0252616 A1
(43) Pub. Date: Aug. 1, 2024

(54) LIVE-ATTENUATED VIRUS VACCINE

(71) Applicants: GRIFFITH UNIVERSITY, Nathan, Queensland4111 (AU); INDIAN IMMUNOLOGICALS LIMITED, Hyderabad, Andhra Pradesh (IN)

(72) Inventors: Surendran Mahalingam, Queensland (AU); Andres Merits, Tartu (EE); Xiang Liu, Queensland (AU)

(73) Assignees: GRIFFITH UNIVERSITY, Nathan, Queensland (AU); INDIAN IMMUNOLOGICALS LIMITED, Hyderabad, Andhra Pradesh (IN)

(21) Appl. No.: 18/016,137

(22) PCT Filed: Jul. 16, 2021
(86) PCT No.: PCT/AU2021/050763

§ 371 Date: Jan. 13, 2023

(30) Foreign Application Priority Data
Jul. 16, 2020 (IN) 202041030397
Dec. 23, 2020 (IN) 202041056151

(51) Int. Cl. A61K 39/215; A61K 39/00; A61P 37/04; C12N 7/00

(52) U.S. Cl. CPC A61K 39/215; A61P 37/04; C12N 7/00; A61K 2039/5254; A61K 2039/53; A61K 2039/543; C12N 2770/20034; C12N 2770/20062

(57) ABSTRACT

This invention relates to a codon deoptimized severe acute respiratory syndrome coronavirus 2 (SARS-COV-2) genome. In particular, embodiments of the invention concern a vaccine comprising live attenuated SARS-COV-2 comprising a partly codon deoptimized viral genome, SARS-COV-2 comprising a partly codon deoptimized viral genome, as well as their use in methods of treatment and prevention of viral infection. The ORF1a region of the viral genome has been codon deoptimized.

### RELATED APPLICATIONS

This application claims priority of Indian Provisional Patent Application No. 202041030397, filed 16 Jul. 2020, and Indian Provisional Patent Application No. 202041056151, filed 23 Dec. 2020, as well as the associated sequence listings filed on those same dates, the entire contents of which are incorporated herein by reference.

### TECHNICAL FIELD

This invention generally relates to a codon deoptimized severe acute respiratory syndrome coronavirus 2 (SARS-COV-2) genome. In particular, embodiments of the invention concern a vaccine comprising live attenuated SARS-COV-2 comprising a partly codon deoptimized viral genome, SARS-COV-2 comprising a partly codon deoptimized viral genome, as well as their use in methods of treatment and prevention of viral infection.

### BACKGROUND ART

Severe acute respiratory syndrome coronavirus 2 (SARS-COV-2) is a strain of beta-coronavirus that causes respiratory illness and is responsible for the COVID-19 pandemic. Multiple other vaccine formulations are currently under development around the world (e.g. RNA and DNA vaccine, subunit vaccine, inactivated whole virus vaccine, and recombinant virus vaccine). Codon deoptimization technology, applicable for construction of live attenuated vaccine candidates, at the time of filing has not been used to develop a commercially available live attenuated SARS-COV-2 vaccine.

Codon usage bias refers to the redundancy of the genetic code, where amino acids are determined by synonymous codons that occur in different organisms at different frequencies. The process of codon optimization, where each amino acid is encoded by the most abundant codon, is frequently exploited to improve gene expression in heterologous systems, a strategy that is used to increase immune responses to antigens. In contrast, codon deoptimization (CD), where all or a selected number of amino acid residues are encoded by a less or the least abundant codon(s), is used to decrease gene expression leading to reduced viral protein production and consequently reduced replication while the composition of viral antigens remains the same. The approach can also result in additional virus attenuation by removing/altering of RNA secondary structures of functional importance (Song Y, Gorbatsevych O, Liu Y, Mugavero J, Shen S H, Ward C B, Asare E, Jiang P, Paul A V, Mueller S. Wimmer E. Limits of variation, specific infectivity, and genome packaging of massively recoded poliovirus genomes. Proc Natl Acad Sci USA. 2017 Oct. 10; 114(41): E8731-E8740. doi: 10.1073/pnas.1714385114. Epub 2017 Sep. 25).

### SUMMARY OF THE INVENTION

Described herein, amongst other things, is a vaccine comprising live attenuated severe acute respiratory syndrome coronavirus 2 (SARS-COV-2) comprising a partly codon deoptimized viral genome, SARS-COV-2 comprising a partly codon deoptimized viral genome, as well as their use in methods of treatment and prevention of viral infection.

According to a first embodiment of the present invention, there is provided a live attenuated severe acute respiratory syndrome coronavirus 2 (SARS-COV-2), SARS-COV-2, SARS-COV-2 particle or SARS-COV-2 nucleic acid comprising a partly codon deoptimized SARS-COV-2 genome.

According to a second embodiment of the present invention, there is provided a recombinant, isolated or substantially purified nucleic acid comprising a partly codon deoptimized SARS-COV-2 genome or partly codon deoptimized region thereof.

According to a third embodiment of the present invention, there is provided a vector, plasmid or genetic construct comprising the nucleic acid of the second embodiment.

According to a fourth embodiment of the present invention, there is provided a cell or isolate containing the live attenuated SARS-COV-2, SARS-COV-2, SARS-COV-2 particle or SARS-COV-2 nucleic acid of the first embodiment, the nucleic acid of the second embodiment, or the vector, plasmid or genetic construct of the third embodiment.

According to a fifth embodiment of the present invention, there is provided a vaccine comprising the live attenuated SARS-COV-2, SARS-COV-2, SARS-COV-2 particle or SARS-COV-2 nucleic acid of the first embodiment, the recombinant, isolated or substantially purified nucleic acid of the second embodiment, the vector, plasmid or genetic construct of the third embodiment, or the cell or isolate of the fourth embodiment.

According to a sixth embodiment of the present invention, there is provided a pharmaceutical preparation comprising the live attenuated SARS-COV-2, SARS-COV-2, SARS-COV-2 particle or SARS-COV-2 nucleic acid of the first embodiment, the recombinant, isolated or substantially purified nucleic acid of the second embodiment, the vector, plasmid or genetic construct of the third embodiment, or the cell or isolate of the fourth embodiment.

According to a seventh embodiment of the present invention, there is provided an immunogenic composition comprising the live attenuated SARS-COV-2, SARS-COV-2, SARS-COV-2 particle or SARS-COV-2 nucleic acid of the first embodiment, the recombinant, isolated or substantially purified nucleic acid of the second embodiment, the vector, plasmid or genetic construct of the third embodiment, or the cell or isolate of the fourth embodiment.

According to an eighth embodiment of the present invention, there is provided a method of: (1) vaccinating a subject; (2) prophylactically immunizing a subject against SARS-CoV-2 or SARS-COV-2-like virus; (3) preventing a subject from contracting a SARS-COV-2 infection naturally or a SARS-COV-2-like infection naturally; (4) reducing the severity of a natural SARS-COV-2 disease or natural SARS-COV-2-like disease in a subject; or (5) treating a subject having a natural SARS-COV-2 infection or natural SARS-COV-2-like infection, said method comprising the step of administering to the subject: the live attenuated SARS-COV-2, SARS-COV-2, SARS-COV-2 particle or SARS-COV-2 nucleic acid of the first embodiment; the recombinant, isolated or substantially purified nucleic acid of the second embodiment; the vector, plasmid or genetic construct of the third embodiment; the cell or isolate of the fourth embodiment; the vaccine of the fifth embodiment; the pharmaceutical preparation of the sixth embodiment; or the immunogenic composition of the seventh embodiment.

According to a ninth embodiment of the present invention, there is provided the use of: the live attenuated SARS-COV-2, SARS-COV-2, SARS-COV-2 particle or SARS-COV-2 nucleic acid of the first embodiment; the recombinant, isolated or substantially purified nucleic acid of the second embodiment; the vector, plasmid or genetic construct of the third embodiment; the cell or isolate of the fourth embodiment; the vaccine of the fifth embodiment; the pharmaceutical preparation of the sixth embodiment; or the immunogenic composition of the seventh embodiment, in the preparation of a medicament for: (1) vaccinating a subject; (2) prophylactically immunizing a subject against SARS-COV-2 or SARS-COV-2-like virus; (3) preventing a subject from contracting a SARS-COV-2 infection naturally or a SARS-COV-2-like infection naturally; (4) reducing the severity of a natural SARS-COV-2 disease or natural SARS-COV-2-like disease in a subject; or (5) treating a subject having a natural SARS-COV-2 infection or natural SARS-COV-2-like infection.

According to a tenth embodiment of the present invention, there is provided: a live attenuated SARS-COV-2, SARS-COV-2, SARS-COV-2 particle or SARS-COV-2 nucleic acid of the first embodiment; a recombinant, isolated or substantially purified nucleic acid of the second embodiment; a vector, plasmid or genetic construct of the third embodiment; a cell or isolate of the fourth embodiment; a vaccine of the fifth embodiment; a pharmaceutical preparation of the sixth embodiment; or an immunogenic composition of the seventh embodiment, for use in: (1) vaccinating a subject; (2) prophylactically immunizing a subject against SARS-COV-2 or SARS-COV-2-like virus; (3) preventing a subject from contracting a SARS-COV-2 infection naturally or a SARS-COV-2-like infection naturally; (4) reducing the severity of a natural SARS-COV-2 disease or natural SARS-COV-2-like disease in a subject; or (5) treating a subject having a natural SARS-COV-2 infection or natural SARS-COV-2-like infection.

According to an eleventh embodiment of the present invention, there is provided a method of generating a live attenuated SARS-COV-2 vaccine, SARS-COV-2, SARS-COV-2 particle or SARS-COV-2 nucleic acid, or recombinant, isolated or substantially purified nucleic acid comprising a partly codon deoptimized SARS-COV-2 genome or partly codon deoptimized region thereof, comprising the step of partly codon deoptimizing a SARS-COV-2 genome.

According to a twelfth embodiment of the present invention, there is provided a method of preparing a vaccine comprising live attenuated SARS-COV-2, said method comprising the steps of: (1) codon deoptimizing a SARS-COV-2 genome to produce a partly codon deoptimized live attenuated SARS-COV-2; and (2) enabling the partly codon deoptimized live attenuated SARS-COV-2 to replicate.

According to a thirteenth embodiment of the present invention, there is provided a method of preparing a vaccine comprising codon deoptimized SARS-COV-2, said method comprising the steps of: optionally. (1) codon deoptimizing a SARS-COV-2 genome to produce a partly codon deoptimized live attenuated SARS-COV-2; (2) enabling the partly codon deoptimized live attenuated SARS-COV-2 to replicate; and (3) preparing a vaccine dose containing the replicated SARS-COV-2 of step (2).

According to a fourteenth embodiment of the present invention, there is provided a method of eliciting an immune response in a subject, said method comprising the step of administering a live attenuated SARS-COV-2, SARS-COV-2, SARS-COV-2 particle or SARS-CoV-2 nucleic acid of the first embodiment; the recombinant, isolated or substantially purified nucleic acid of the second embodiment; the vector, plasmid or genetic construct of the third embodiment; the cell or isolate of the fourth embodiment; the vaccine of the fifth embodiment; the pharmaceutical preparation of the sixth embodiment; or the immunogenic composition of the seventh embodiment to the subject to thereby elicit an immune response.

### BRIEF DESCRIPTION OF THE FIGURES

FIG. 1. Schematic representation of the SARS-COV-2 genome, showing the open reading frame ORF1a, and expressed polypeptides.

FIG. 2. Schematic representation of a bacterial artificial chromosome (BAC) genetic construct comprising the cDNA of SARS-COV-2 genome, for use in a transfection strategy for obtaining a first generation of infectious virus or vaccine candidates.

FIG. 3. Schematic representation showing how ORF1a fragments 2 and 3 can be cleaved using restriction enzymes to produce sub-fragments 2A, 2B, 2C, 3A and 3B, wherein these sub-fragments can be used in the generation of SARS-COV-2 vaccine candidates (first generation).

FIG. 4. Table characterising deoptimized sub-fragments 2A, 2B, 2C, 3A and 3B. All fragments are within the ORF1a region. Genomic positions within the Wuhan virus strain are shown in brackets. Regions presumed or known for cis-activities such as frame-shift signal at the junction of ORF1a and ORF1b were excluded.

FIG. 5. Schematic representation of an immunisation trial using SARS-COV-2 vaccine candidates in a non-human primate model.

FIG. 6. Schematic representation of an immunisation trial using SARS-COV-2 vaccine candidates in a mouse model.

FIG. 7. Flowchart shows steps from first generation SARS-COV-2 vaccine candidate construction, testing, to vaccine production.

FIG. 8. Codon deoptimized nucleotide sequence of sub-fragment 2A (SEQ ID NO:33). Codon deoptimized nucleotides have been underlined.

FIG. 9. Codon deoptimized nucleotide sequence of sub-fragment 2B (SEQ ID NO:34). Codon deoptimized nucleotides have been underlined.

FIG. 10. Codon deoptimized nucleotide sequence of sub-fragment 2C (SEQ ID NO:35). Codon deoptimized nucleotides have been underlined.

FIG. 11. Codon deoptimized nucleotide sequence of sub-fragment 3A (SEQ ID NO:36). Codon deoptimized nucleotides have been underlined.

FIG. 12. Codon deoptimized nucleotide sequence of sub-fragment 3B (SEQ ID NO:37). Codon deoptimized nucleotides have been underlined.

FIG. 13. Schematic representation of a bacterial artificial chromosome (BAC) construct comprising the cDNA of SARS-COV-2 genome, for use in a transfection strategy for obtaining a second generation of infectious virus or vaccine candidates.

FIG. 14. Schematic representation showing how the ORF1a can be cleaved using restriction enzymes to produce fragments 1, 2 and 3, wherein these fragments can be used in the generation of SARS-COV-2 vaccine candidates (second generation). Nucleotide positions within the Wuhan viral genome are indicated.

FIG. 15. Table characterising deoptimized fragments 1, 2 and 3. All fragments are within the ORF1a region. Nucleotide positions within the Wuhan viral genome are indicated. Regions presumed or known for cis-activities such as frame-shift signal at the junction of ORF1a and ORF1b were excluded.

FIG. 16. Flowchart showing steps from second generation SARS-COV-2 vaccine candidate construction, testing, to vaccine production.

FIG. 17. Growth curve of second generation clones/candidates SARS-COV-2 (circle symbol), SARS-COV-2-160-7 (square symbol), SARS-COV-2-4N-1 (triangle symbol) and SARS-COV-2-7N-1 (inverted triangle symbol) in Vero E6 cells at MOI 0.1 infection.

FIG. 18. Day 1 post infection CPE development in Vero E6 cells at MOI 0.1 infection A) Mock, B) SARS-COV-2, C) SARS-COV-2-160-7, D) SARS-COV-2-4N-1 and E) SARS-COV-2-7N-1.

FIG. 19. Day 2 post infection CPE development in Vero E6 cells at MOI 0.1 infection A) Mock, B) SARS-COV-2, C) SARS-COV-2-160-7, D) SARS-COV-2-4N-1 and E) SARS-COV-2-7N-1.

FIG. 20. Day 3 post infection CPE development in Vero E6 cells at MOI 0.1 infection A) Mock, B) SARS-COV-2, C) SARS-COV-2-160-7, D) SARS-COV-2-4N-1 and E) SARS-COV-2-7N-1.

FIG. 21. Plaque morphology in Vero E6 cells at MOI 0.1 infection A) SARS-CoV-2, B) SARS-COV-2-160-7, C) SARS-COV-2-4N-1 and D) SARS-COV-2-7N-1.

FIG. 22. Codon deoptimized nucleotide sequence between SanDI to PacI (SEQ ID NO:45) for clone SARS-COV-2-77. Codon deoptimized nucleotides are shown in red and boxed.

FIG. 23. Codon deoptimized nucleotide sequence between SanDI to PacI (SEQ ID NO:52) for clone SARS-COV-2-160. The sequence of SARS-COV-2 wildtype is shown above the clone. Codon deoptimized nucleotides are shown in red and boxed.

FIG. 24. Codon deoptimized nucleotide sequence between SanDI to PacI (SEQ ID NO:59) for clone SARS-COV-2-4N. The sequence of SARS-COV-2 wildtype is shown above the clone. Codon deoptimized nucleotides are shown in red and boxed.

FIG. 25. Codon deoptimized nucleotide sequence between SanDI to PacI (SEQ ID NO:66) for clone SARS-COV-2-7N. The sequence of SARS-COV-2 wildtype is shown above the clone. Codon deoptimized nucleotides are shown in red and boxed.

FIG. 26. Histopathological evaluation of hamster lungs, for cell and tissue damage and reactive inflammation, following infection with wild-type SARS-COV-2 or vaccine candidates 4N-1, 7N-1, 77-7, 160-4 and 160-7. The star symbol shows a bronchiole. The arrow shows tissue damage with reactive inflammatory cell infiltration. 26A—at day 3. 26B—at day 5. 26C—at day 7. 26D—at day 14.

FIG. 27. Histopathological evaluation of hamster lungs, for distribution of lesions, bronchial and peribronchial distribution of inflammatory cells, following infection with wild-type SARS-COV-2 or vaccine candidates 4N-1, 7N-1, 77-7, 160-4 and 160-7. The star symbol shows a bronchiole. The arrow shows bronchial and peribronchial distribution of inflammatory cells. 27A—at day 3. 27B—at day 5. 27C—at day 7. 27D—at day 14.

FIG. 28. Histopathological evaluation of hamster lungs, for circulatory and vascular lesions, including perivascular edema, desquamation of endothelial cells and endothelialitis, following infection with wild-type SARS-COV-2 or vaccine candidates 4N-1, 7N-1, 77-7, 160-4 and 160-7. The star symbol shows a bronchiole. The arrow shows perivascular edema and desquamation of endothelial cell with endothelialitis. 28A—at day 3. 28B—at day 5. 28C—at day 7. 28D—at day 14.

FIG. 29. Histopathological evaluation of hamster lungs, for regeneration and repair, following infection with wild-type SARS-COV-2 or vaccine candidates 4N-1, 7N-1, 77-7. 160-4 and 160-7. The star symbol shows a bronchiole. The arrow shows hyperplasia bronchial epithelial cells. 29A—at day 3. 29B—at day 5. 29C—at day 7. 29D—at day 14.

FIG. 30. Plotted results of a challenge experiment, showing the efficacy of vaccine candidate 7N-1. ‘7N-1 SC’ means subcutaneous administration. ‘7N-1 IN’ means intranasal administration. ‘WT nCOV’ means wild-type mouse-adapted SARS-COV. 7N-1 provided full protection from rechallenge mortality when given via an intranasal route in HFH4-hACE2 mice.

FIG. 31. Preclinical immunogenicity data in animal models. Hamsters were given a single dose of 10⁴ PFU of live attenuated virus (‘LAV’) candidate 160-7 or 7N-1 subcutaneously. The graphs show neutralizing antibody titres on day 14 after immunisation with LAV, wherein: PRNT₁₀₀ is the end point serum dilution where 100% neutralization was observed; PRNT₉₀ is the end point serum dilution where 90% neutralization was observed; and, PRNT₅₀ is the end point serum dilution where 50% neutralization was observed.

FIG. 32. Vaccine plaque size after multiple (2 and 4) in vitro passage. 32A—wildtype SARS-COV-2. 32B—vaccine candidate 160-7. 32C—vaccine candidates 77-7. 32D—vaccine candidate 160-4. 32E—vaccine candidate 4N-1. 32F—vaccine candidate 7N-1. Vaccine candidates 7N-1, 77-7, 4N-1, 160-4 and 160-7 were passaged up to 4 times in Vero GMP cells at multiplicity of infection of 0.01 PFU/cell. Each dot represents one plaque.

FIG. 33. Live attenuated COVID-19 vaccine showing attenuation in vitro. Multistep growth kinetics in Vero cells were obtained by infecting cells with WT SARS-COV-2 (Wildtype COVID-19) or LAV (vaccine candidate 7N-1) at an MOI of 0.01 PFU/cell.

FIG. 34. Survival plot. hACE-2 Tg mice were inoculated via the intranasal route with 10⁵ PFU wildtype SARS-COV-2 or LAV (candidate 7N-1).

FIG. 35. Survival plot. hACE-2 Tg mice were immunised via the intranasal route with 10³ PFU of vaccine 7N-1. Three weeks later, the mice were challenged with 10⁵ PFU wild-type SARS-COV-2 intranasally and monitored over a 12-day period.

FIG. 36. Histopathological evaluation of hamster lungs following infection with wild-type SARS-COV-2 or vaccine candidate 7N-1. Day 7: Distribution of lesions—bronchial and peribronchial distribution of inflammatory cells.

FIG. 37. Vaccine candidate 7N-1 provided full protection from rechallenge mortality when given via intranasal route in HFH4-hACE2 mice. A. Survival plot. Plotted results of a challenge experiment, showing the efficacy of vaccine candidate 7N-1. ‘7N-1 SC’ means subcutaneous administration. ‘7N-1 IN’ means intranasal administration. ‘WT nCOV’ means wild-type mouse-adapted SARS-COV. B. Graphed results for PBS, being unimmunized mice. C. Graphed results for WT nCOV (nCOV WT). D. Graphed results for mouse body weights post infection. E. Graphed results for 7N-1 SC. F. Graphed results for 7N-1 IN. Disease scores are as follows: 1=no disease; 2=mild fur ruffling; 3=moderate fur ruffling/disease signs; 4=moribund; 5=dead/euthanized.

### DESCRIPTION OF SEQUENCES

### First Generation of Clones.

For the description of clones below, ‘D’ denotes deoptimized and ‘W’ denotes wildtype and therefore not deoptimized.

SEQ ID NO:1. Clone pCCI-4K-SARS-COV-2-DDDDD. All five sub-fragments were deoptimized.

SEQ ID NO:2. Clone pCCI-4K-SARS-COV-2-DDDDW. The first four sub-fragments were deoptimized.

SEQ ID NO:3. Clone pCCI-4K-SARS-COV-2-DDDWD. Sub-fragments one, two, three, and five were deoptimized.

SEQ ID NO:4. Clone pCCI-4K-SARS-COV-2-DDDWW. The first three sub-fragments were deoptimized.

SEQ ID NO:5. Clone pCCI-4K-SARS-COV-2-DDWDD. Sub-fragments one, two, four, and five were deoptimized.

SEQ ID NO:6. Clone pCCI-4K-SARS-COV-2-DDWDW. Sub-fragments one, two, and four were deoptimized.

SEQ ID NO:7. Clone pCCI-4K-SARS-COV-2-DDWWD. Sub-fragments one, two, and five were deoptimized.

SEQ ID NO:8. Clone pCCI-4K-SARS-COV-2-DDWWW. The first two sub-fragments were deoptimized.

SEQ ID NO:9. Clone pCCI-4K-SARS-COV-2-DWDDD. First, third, fourth, and fifth sub-fragments were deoptimized.

SEQ ID NO:10. Clone pCCI-4K-SARS-COV-2-DWDDW. First, third, and fourth sub-fragments were deoptimized.

SEQ ID NO:11. Clone pCCI-4K-SARS-COV-2-DWDWD. First, third, and fifth sub-fragments were deoptimized.

SEQ ID NO:12. Clone pCCI-4K-SARS-COV-2-DWDWW. First and third sub-fragments were deoptimized.

SEQ ID NO:13. Clone pCCI-4K-SARS-COV-2-DWWDD. The first, fourth, and fifth sub-fragments were deoptimized.

SEQ ID NO:14. Clone pCCI-4K-SARS-COV-2-DWWDW. The first and fourth sub-fragments were deoptimized.

SEQ ID NO:15. Clone pCCI-4K-SARS-COV-2-DWWWD. The first and fifth sub-fragments were deoptimized.

SEQ ID NO:16. Clone pCCI-4K-SARS-COV-2-DWWWW. The first sub-fragment was deoptimized.

SEQ ID NO:17. Clone pCCI-4K-SARS-COV-2-WDDDW. The second, third, and fourth sub-fragments were deoptimized.

SEQ ID NO:18. Clone pCCI-4K-SARS-COV-2-WDDWD. The second, third, and fifth sub-fragments were deoptimized.

SEQ ID NO:19. Clone pCCI-4K-SARS-COV-2-WDDWW. The second and third sub-fragments were deoptimized.

SEQ ID NO:20. Clone pCCI-4K-SARS-COV-2-WDWDD. The second, fourth, and fifth sub-fragments were deoptimized.

SEQ ID NO:21. Clone pCCI-4K-SARS-COV-2-WDWDW. The second and fourth sub-fragments were deoptimized.

SEQ ID NO:22. Clone pCCI-4K-SARS-COV-2-WDWWD. The second and fifth sub-fragments were deoptimized.

SEQ ID NO:23. Clone pCCI-4K-SARS-COV-2-WDWWW. The second sub-fragment was deoptimized.

SEQ ID NO:24. Clone pCCI-4K-SARS-COV-2-WWDDD. The last three sub-fragments were deoptimized.

SEQ ID NO:25. Clone pCCI-4K-SARS-COV-2-WWDDW. The third and fourth sub-fragments were deoptimized.

SEQ ID NO:26. Clone pCCI-4K-SARS-COV-2-WWDWD. The third and fifth sub-fragments were deoptimized.

SEQ ID NO:27. Clone pCCI-4K-SARS-COV-2-WWDWW. The third sub-fragment was deoptimized.

SEQ ID NO:28. Clone pCCI-4K-SARS-COV-2-WWWDD. The last two sub-fragments were deoptimized.

SEQ ID NO:29. Clone pCCI-4K-SARS-COV-2-WWWDW. The fourth sub-fragment was deoptimized.

SEQ ID NO:30. Clone pCCI-4K-SARS-COV-2-WWWWD. The last sub-fragment was deoptimized.

SEQ ID NO:31. Clone pCCI-4K-SARS-COV-2-WDDDD. The last four sub-fragments were deoptimized.

SEQ ID NO:32. Clone pCCI-4K-SARS-COV-2. No sub-fragment was deoptimized (wild-type).

SEQ ID NO:33. Codon deoptimized nucleotide sequence of sub-fragment 2A.

SEQ ID NO:34. Codon deoptimized nucleotide sequence of sub-fragment 2B.

SEQ ID NO:35. Codon deoptimized nucleotide sequence of sub-fragment 2C.

SEQ ID NO:36. Codon deoptimized nucleotide sequence of sub-fragment 3A.

SEQ ID NO:37. Codon deoptimized nucleotide sequence of sub-fragment 3B.

SEQ ID NO:38. Wild-type nucleotide sequence encoding the E protein of the SARS-COV-2 genome.

### Second Generation of Clones.

SEQ ID NO: 39 - Clone pCC1-4K-SARS-CoV-2-77-1. Only fragment 3 has been
deoptimized:
GGGTCCCACGTGCTAGCGCTAACATAGGTTGTAACCATACAGGTGTTG
TTGGAGAAGGTTCCGAAGGTCTTAATGACAACCTTCTTGAAATACTCCAAAAAG
AGAAAGTCAACATCAATATTGTTGGTGACTTTAAACTTAATGAAGAGATCGCCAT
TATTTTGGCATCTTTTTCTGCTTCCACAAGTGCTTTTGTGGAAACTGTGAAAGGTT
TGGATTATAAAGCATTCAAACAAATTGTTGAATCCTGTGGTAATTTTAAAGTTAC
AAAAGGAAAAGCTAAAAAAGGTGCCTGGAATATTGGTGAACAGAAATCAATACT
GAGTCCTCTTTATGCATTTGCATCAGAGGCTGCTCGTGTTGTACGATCAATTTTCT
CCCGCACTCTTGAAACTGCTCAAAATTCTGTGCGTGTTTTACAGAAGGCCGCTAT
AACAATACTAGATGGAATTTCACAGTATTCACTGAGACTCATTGATGCTATGATG
TTCACATCTGATTTGGCTACTAACAATCTAGTTGTAATGGCCTACATTACAGGTG
GTGTTGTTCAGTTGACTTCGCAGTGGCTAACTAACATCTTTGGCACTGTTTATGAA
AAACTCAAACCCGTCCTTGATTGGCTTGAAGAGAAGTTTAAGGAAGGTGTAGAG
TTTCTTAGAGACGGTTGGGAAATTGTTAAATTTATCTCAACCTGTGCTTGTGAAAT
TGTCGGTGGACAAATTGTCACCTGTGCAAAGGAAATTAAGGAGAGTGTTCAGAC
ATTCTTTAAGCTTGTAAATAAATTTTTGGCTTTGTGTGCTGACTCTATCATTATTG
GTGGAGCTAAACTTAAAGCCTTGAATTTAGGTGAAACATTTGTCACGCACTCAAA
GGGATTGTACAGAAAGTGTGTTAAATCCAGAGAAGAAACTGGCCTACTCATGCC
TCTAAAAGCCCCAAAAGAAATTATCTTCTTAGAGGGAGAAACACTTCCCACAGA
AGTGTTAACAGAGGAAGTTGTCTTGAAAACTGGTGATTTACAACCATTAGAACA
ACCTACTAGTGAAGCTGTTGAAGCTCCATTGGTTGGTACACCAGTTTGTATTAAC
GGGCTTATGTTGCTCGAAATCAAAGACACAGAAAAGTACTGTGCCCTTGCACCTA
ATATGATGGTAACAAACAATACCTTCACACTCAAAGGCGGTGCACCAACAAAGG
TTACTTTTGGTGATGACACTGTGATAGAAGTGCAAGGTTACAAGAGTGTGAATAT
CACTTTTGAACTTGATGAAAGGATTGATAAAGTACTTAATGAGAAGTGCTCTGCC
TATACAGTTGAACTCGGTACAGAAGTAAATGAGTTCGCCTGTGTTGTGGCAGATG
CTGTCATAAAAACTTTGCAACCAGTATCTGAATTACTTACACCACTGGGCATTGA
TTTAGATGAGTGGAGTATGGCTACATACTACTTATTTGATGAGTCTGGTGAGTTT
AAATTGGCTTCACATATGTATTGTTCTTTCTACCCTCCAGATGAGGATGAAGAAG
AAGGTGATTGTGAAGAAGAAGAGTTTGAGCCATCAACTCAATATGAGTATGGTA
CTGAAGATGATTACCAAGGTAAACCTTTGGAATTTGGTGCCACTTCTGCTGCTCT
TCAACCTGAAGAAGAGCAAGAAGAAGATTGGTTAGATGATGATAGTCAACAAAC
TGTTGGTCAACAAGACGGCAGTGAGGACAATCAGACAACTACTATTCAAACAAT
TGTTGAGGTTCAACCTCAATTAGAGATGGAACTTACACCAGTTGTTCAGACTATT
GAAGTGAATAGTTTTAGTGGTTATTTAAAACTTACTGACAATGTATACATTAAAA
ATGCAGACATTGTGGAAGAAGCTAAAAAGGTAAAACCAACAGTGGTTGTTAATG
CAGCCAATGTTTACCTTAAACATGGAGGAGGTGTTGCAGGAGCCTTAAATAAGG
CTACTAACAATGCCATGCAAGTTGAATCTGATGATTACATAGCTACTAATGGACC
ACTTAAAGTGGGTGGTAGTTGTGTTTTAAGCGGACACAATCTTGCTAAACACTGT
CTTCATGTTGTCGGCCCAAATGTTAACAAAGGTGAAGACATTCAACTTCTTAAGA
GTGCTTATGAAAATTTTAATCAGCACGAAGTTCTACTTGCACCATTATTATCAGCT
GGTATTTTTGGTGCTGACCCTATACATTCTTTAAGAGTTTGTGTAGATACTGTTCG
CACAAATGTCTACTTAGCTGTCTTTGATAAAAATCTCTATGACAAACTTGTTTCAA
GCTTTTTGGAAATGAAGAGTGAAAAGCAAGTTGAACAAAAGATCGCTGAGATTC
CTAAAGAGGAAGTTAAGCCATTTATAACTGAAAGTAAACCTTCAGTTGAACAGA
GAAAACAAGATGATAAGAAAATCAAAGCTTGTGTTGAAGAAGTTACAACAACTC
TGGAAGAAACTAAGTTCCTCACAGAAAACTTGTTACTTTATATTGACATTAATGG
CAATCTTCATCCAGATTCTGCCACTCTTGTTAGTGACATTGACATCACTTTCTTAA
AGAAAGATGCTCCATATATAGTGGGTGATGTTGTTCAAGAGGGTGTTTTAACTGC
TGTGGTTATACCTACTAAAAAGGCTGGTGGCACTACTGAAATGCTAGCGAAAGC
TTTGAGAAAAGTGCCAACAGACAATTATATAACCACTTACCCGGGTCAGGGTTTA
AATGGTTACACTGTAGAGGAGGCAAAGACAGTGCTTAAAAAGTGTAAAAGTGCC
TTTTACATTCTACCATCTATTATCTCTAATGAGAAGCAAGAAATTCTTGGAACTGT
TTCTTGGAATTTGCGAGAAATGCTTGCACATGCAGAAGAAACACGCAAATTAAT
GCCTGTCTGTGTGGAAACTAAAGCCATAGTTTCAACTATACAGCGTAAATATAAG
GGTATTAAAATACAAGAGGGTGTGGTTGATTATGGTGCTAGATTTTACTTTTACA
CCAGTAAAACAACTGTAGCGTCACTTATCAACACACTTAACGATCTAAATGAAA
CTCTTGTTACAATGCCACTTGGCTATGTAACACATGGCTTAAATTTGGAAGAAGC
TGCTCGGTATATGAGATCTCTCAAAGTGCCAGCTACAGTTTCTGTTTCTTCACCTG
ATGCTGTTACAGCGTATAATGGTTATCTTACTTCTTCTTCTAAAACACCTGAAGAA
CATTTTATTGAAACCATCTCACTTGCTGGTTCCTATAAAGATTGGTCCTATTCTGG
ACAATCTACACAACTAGGTATAGAATTTCTTAAGAGAGGTGATAAAAGTGTATAT
TACACTAGTAATCCTACCACATTCCACCTAGATGGTGAAGTTATCACCTTTGACA
ATCTTAAGACACTTCTTTCTTTGAGAGAAGTGAGGACTATTAAGGTGTTTACAAC
AGTAGACAACATTAACCTCCACACGCAAGTTGTGGACATGTCAATGACATATGG
ACAACAGTTTGGTCCAACTTATTTGGATGGAGCTGATGTTACTAAAATAAAACCT
CATAATTCACATGAAGGTAAAACATTTTATGTTTTACCTAATGATGACACTCTAC
GTGTTGAGGCTTTTGAGTACTACCACACAACTGATCCTAGTTTTCTGGGTAGGTA
CATGTCAGCATTAAATCACACTAAAAAGTGGAAATACCCACAAGTTAATGGTTT
AACTTCTATTAAATGGGCAGATAACAACTGTTATCTTGCCACTGCATTGTTAACA
CTCCAACAAATAGAGTTGAAGTTTAATCCACCTGCTCTACAAGATGCTTATTACA
GAGCAAGGGCTGGTGAAGCTGCTAACTTTTGTGCACTTATCTTAGCCTACTGTAA
TAAGACAGTAGGTGAGTTAGGTGATGTTAGAGAAACAATGAGTTACTTGTTTCAA
CATGCCAATTTAGATTCTTGCAAAAGAGTCTTGAACGTGGTGTGTAAAACTTGTG
GACAACAGCAGACAACCCTTAAGGGTGTAGAAGCTGTTATGTACATGGGCACAC
TTTCTTATGAACAATTTAAGAAAGGTGTTCAGATACCTTGTACGTGTGGTAAACA
AGCTACAAAATATCTAGTACAACAGGAGTCACCTTTTGTTATGATGTCAGCACCA
CCTGCTCAGTATGAACTTAAGCATGGTACATTTACTTGTGCTAGTGAGTACACTG
GTAATTACCAGTGTGGTCACTATAAACATATAACTTCTAAAGAAACTTTGTATTG
CATAGACGGTGCTTTACTTACAAAGTCCTCAGAATACAAAGGTCCTATTACGGAT
GTTTTCTACAAAGAAAACAGTTACACAACAACCATAAAACCAGTTACTTATAAAT
TGGATGGTGTTGTTTGTACAGAAATTGACCCTAAGTTGGACAATTATTATAAGAA
AGACAATTCTTATTTCACAGAGCAACCAATTGATCTTGTACCAAACCAACCATAT
CCAAACGCAAGCTTCGATAATTTTAAGTTTGTATGTGATAATATCAAATTTGCTG
ATGATTTAAACCAGTTAACTGGTTATAAGAAACCTGCTTCAAGAGAGCTTAAAGT
TACATTTTTCCCTGACTTAAATGGTGATGTGGTGGCTATTGATTATAAACACTACA
CACCCTCTTTTAAGAAAGGAGCTAAATTGTTACATAAACCTATTGTTTGGCATGT
TAACAATGCAACTAATAAAGCCACGTATAAACCAAATACCTGGTGTATACGTTGT
CTTTGGAGCACAAAACCAGTTGAAACATCAAATTCGTTTGATGTACTGAAGTCAG
AGGACGCGCAGGGAATGGATAATCTTGCCTGCGAAGATCTAAAACCAGTCTCTG
AAGAAGTAGTGGAAAATCCTACCATACAGAAAGACGTTCTTGAGTGTAATGTGA
AAACTACCGAAGTTGTAGGAGACATTATACTTAAACCAGCAAATAATAGTTTAA
AAATTACAGAAGAGGTTGGCCACACAGATCTAATGGCTGCTTATGTAGACAATT
CTAGTCTTACTATTAAGAAACCTAATGAATTATCTAGAGTATTAGGTTTGAAAAC
CCTTGCTACTCATGGTTTAGCTGCTGTTAATAGTGTCCCTTGGGATACTATAGCTA
ATTATGCTAAGCCTTTTCTTAACAAAGTTGTTAGTACAACTACTAACATAGTTAC
ACGGTGTTTAAACCGTGTTTGTACTAATTATATGCCTTATTTCTTTACTTTATTGCT
ACAATTGTGTACTTTTACTAGAAGTACAAATTCTAGAATTAAAGCATCTATGCCG
ACTACTATAGCAAAGAATACTGTTAAGAGTGTCGGTAAATTTTGTCTAGAGGCTT
CATTTAATTATTTGAAGTCACCTAATTTTTCTAAACTGATAAATATTATAATTTGG
TTTTTACTATTAAGTGTTTGCCTAGGTTCGTTAATCTACTCAACCGCTGCTTTAGG
TGTTTTAATGTCGAATTTAGGCATGCCTTCTTACTGTACTGGTTACAGAGAAGGCT
ATTTGAACTCGACTAATGTCACTATTGCAACCTACTGTACTGGTTCTATACCTTGT
TCGGTTTGTCTTAGTGGTTTAGATTCTTTAGACACCTATCCTTCGTTAGAAACTAT
ACAAATTACCATTTCATCTTTTAAATGGGATTTAACTGCTTTTGGCTTAGTTGCAG
AGTGGTTTTTGGCATATATTCTTTTCACTAGGTTTTTCTATGTACTTGGATTGGCT
GCAATCATGCAATTGTTTTTCTCGTATTTTGCAGTACATTTTATTAGTAATTCGTG
GCTTATGTGGTTAATAATTAATCTTGTACAAATGGCCCCGATTTCAGCTATGGTTA
GAATGTACATCTTCTTTGCATCGTTTTATTATGTATGGAAAAGTTATGTGCATGTT
GTAGACGGTTGTAATTCGTCAACTTGTATGATGTGTTACAAACGTAATAGAGCAA
CAAGAGTCGAATGTACAACTATTGTTAATGGTGTTAGAAGGTCGTTTTATGTCTA
TGCTAATGGAGGTAAAGGCTTTTGCAAACTACACAATTGGAATTGTGTTAATTGT
GATACATTCTGTGCTGGTAGTACATTTATTTCGGATGAAGTTGCGAGAGACTTGT
CACTACAGTTTAAAAGACCAATAAATCCTACTGACCAGTCGTCTTACATCGTTGA
TAGTGTTACAGTGAAGAATGGTTCGATCCATCTTTACTTTGATAAAGCTGGTCAA
AAGACTTATGAAAGACATTCTCTCTCGCATTTTGTTAACTTAGACAACCTGAGAG
CTAATAACACTAAAGGTTCATTGCCTATTAATGTTATAGTTTTTGATGGTAAATCG
AAATGTGAAGAATCATCGGCAAAATCAGCGTCGGTTTACTACAGTCAGCTTATGT
GTCAACCTATACTGTTACTAGATCAGGCATTAGTGTCGGATGTTGGTGATAGTGC
GGAAGTTGCAGTTAAAATGTTTGATGCTTACGTTAATACGTTTTCGTCAACTTTTA
ACGTACCAATGGAAAAACTCAAAACACTAGTTGCAACTGCAGAAGCTGAACTTG
CAAAGAATGTGTCCTTAGACAATGTCTTATCGACTTTTATTTCAGCAGCTCGGCA
AGGGTTTGTTGATTCGGATGTAGAAACTAAAGATGTTGTTGAATGTCTTAAATTG
TCACATCAATCGGACATAGAAGTTACTGGCGATAGTTGTAATAACTATATGCTCA
CCTATAACAAAGTTGAAAACATGACACCCCGTGACCTTGGTGCTTGTATTGACTG
TTCGGCGCGTCATATTAATGCGCAGGTAGCAAAAAGTCACAACATTGCTTTGATA
TGGAACGTTAAAGATTTCATGTCGTTGTCTGAACAACTACGAAAACAAATACGTT
CGGCTGCTAAAAAGAATAACTTACCTTTTAAGTTGACATGTGCAACTACTAGACA
AGTTGTTAATGTTGTAACAACAAAGATAGCACTTAAGGGTGGTAAAATTGTTAAT
AATTGGTTGAAGCAGTTAATTAAA
SEQ ID NO: 40 - Clone pCC1-4K-SARS-COV-2-77-2. Only fragment 2 has been
deoptimized:
GGGTCCCACGTGCTAGCGCTAACATAGGTTGTAACCATACAGGTGTTG
TTGGAGAAGGTTCCGAAGGTCTTAATGACAACCTTCTTGAAATACTCCAAAAAG
AGAAAGTCAACATCAATATTGTTGGTGACTTTAAACTTAATGAAGAGATCGCCAT
TATTTTGGCATCTTTTTCTGCTTCCACAAGTGCTTTTGTGGAAACTGTGAAAGGTT
TGGATTATAAAGCATTCAAACAAATTGTTGAATCCTGTGGTAATTTTAAAGTTAC
AAAAGGAAAAGCTAAAAAAGGTGCCTGGAATATTGGTGAACAGAAATCAATACT
GAGTCCTCTTTATGCATTTGCATCAGAGGCTGCTCGTGTTGTACGATCAATTTTCT
CCCGCACTCTTGAAACTGCTCAAAATTCTGTGCGTGTTTTACAGAAGGCCGCTAT
AACAATACTAGATGGAATTTCACAGTATTCACTGAGACTCATTGATGCTATGATG
TTCACATCTGATTTGGCTACTAACAATCTAGTTGTAATGGCCTACATTACAGGTG
GTGTTGTTCAGTTGACTTCGCAGTGGCTAACTAACATCTTTGGCACTGTTTATGAA
AAACTCAAACCCGTCCTTGATTGGCTTGAAGAGAAGTTTAAGGAAGGTGTAGAG
TTTCTTAGAGACGGTTGGGAAATTGTTAAATTTATCTCAACCTGTGCTTGTGAAAT
TGTCGGTGGACAAATTGTCACCTGTGCAAAGGAAATTAAGGAGAGTGTTCAGAC
ATTCTTTAAGCTTGTAAATAAATTTTTGGCTTTGTGTGCTGACTCTATCATTATTG
GTGGAGCTAAACTTAAAGCCTTGAATTTAGGTGAAACATTTGTCACGCACTCAAA
GGGATTGTACAGAAAGTGTGTTAAATCCAGAGAAGAAACTGGCCTACTCATGCC
TCTAAAAGCCCCAAAAGAAATTATCTTCTTAGAGGGAGAAACACTTCCCACAGA
AGTGTTAACAGAGGAAGTTGTCTTGAAAACTGGTGATTTACAACCATTAGAACA
ACCTACTAGTGAAGCTGTTGAAGCTCCATTGGTTGGTACACCAGTTTGTATTAAC
GGGCTTATGTTGCTCGAAATCAAAGACACAGAAAAGTACTGTGCCCTTGCACCTA
ATATGATGGTAACAAACAATACCTTCACACTCAAAGGCGGTGCACCAACAAAGG
TTACTTTTGGTGATGACACTGTGATAGAAGTGCAAGGTTACAAGAGTGTGAATAT
CACTTTTGAACTTGATGAAAGGATTGATAAAGTACTTAATGAGAAGTGCTCTGCC
TATACAGTTGAACTCGGTACAGAAGTAAATGAGTTCGCCTGTGTTGTGGCAGATG
CTGTCATAAAAACTTTGCAACCAGTATCTGAATTACTTACACCACTGGGCATTGA
TTTAGATGAGTGGAGTATGGCTACATACTACTTATTTGATGAGTCTGGTGAGTTT
AAATTGGCTTCACATATGTATTGTTCTTTCTACCCTCCAGATGAGGATGAAGAAG
AAGGTGATTGTGAAGAAGAAGAGTTTGAGCCATCAACTCAATATGAGTATGGTA
CTGAAGATGATTACCAAGGTAAACCTTTGGAATTTGGTGCCACTTCTGCTGCTCT
TCAACCTGAAGAAGAGCAAGAAGAAGATTGGTTAGATGATGATAGTCAACAAAC
TGTTGGTCAACAAGACGGCAGTGAGGACAATCAGACAACTACTATTCAAACAAT
TGTTGAGGTTCAACCTCAATTAGAGATGGAACTTACACCAGTTGTTCAGACTATT
GAAGTGAATAGTTTTAGTGGTTATTTAAAACTTACTGACAATGTATACATTAAAA
ATGCAGACATTGTGGAAGAAGCTAAAAAGGTAAAACCAACAGTGGTTGTTAATG
CAGCCAATGTTTACCTTAAACATGGAGGAGGTGTTGCAGGAGCCTTAAATAAGG
CTACTAACAATGCCATGCAAGTTGAATCTGATGATTACATAGCTACTAATGGACC
ACTTAAAGTGGGTGGTAGTTGTGTTTTAAGCGGACACAATCTTGCTAAACACTGT
CTTCATGTTGTCGGCCCAAATGTTAACAAAGGTGAAGACATTCAACTTCTTAAGA
GTGCTTATGAAAATTTTAATCAGCACGAAGTTCTACTTGCACCATTATTATCAGCT
GGTATTTTTGGTGCTGACCCTATACATTCTTTAAGAGTTTGTGTAGATACTGTTCG
CACAAATGTCTACTTAGCTGTCTTTGATAAAAATCTCTATGACAAACTTGTTTCAA
GCTTTTTGGAAATGAAGAGTGAAAAGCAAGTTGAACAAAAGATCGCTGAGATTC
CTAAAGAGGAAGTTAAGCCATTTATAACTGAAAGTAAACCTTCAGTTGAACAGA
GAAAACAAGATGATAAGAAAATCAAAGCTTGTGTTGAAGAAGTTACAACAACTC
TGGAAGAAACTAAGTTCCTCACAGAAAACTTGTTACTTTATATTGACATTAATGG
CAATCTTCATCCAGATTCTGCCACTCTTGTTAGTGACATTGACATCACTTTCTTAA
AGAAAGATGCTCCATATATAGTGGGTGATGTTGTTCAAGAGGGTGTTTTAACTGC
TGTGGTTATACCTACTAAAAAGGCTGGTGGCACTACTGAAATGCTAGCGAAAGC
TTTGAGAAAAGTGCCAACAGACAATTATATAACCACTTACCCGGGTCAGGGTTTA
AATGGTTACACTGTAGAGGAGGCAAAGACAGTGCTTAAAAAGTGTAAATCGGCC
TTTTACATTCTACCATCTATTATCTCGAATGAGAAGCAAGAAATTCTTGGAACTG
TTTCTTGGAATTTGCGAGAAATGCTTGCACATGCAGAAGAAACACGCAAATTAAT
GCCTGTCTGTGTGGAAACTAAAGCCATAGTTTCGACTATACAGCGTAAATATAAG
GGTATTAAAATACAAGAGGGTGTGGTTGATTATGGTGCTAGATTTTACTTTTACA
CCAGTAAAACAACTGTAGCGTCGCTTATCAACACACTTAACGATCTAAATGAAA
CTCTTGTTACAATGCCACTTGGCTATGTAACACATGGCTTAAATTTGGAAGAAGC
TGCTCGGTATATGAGATCTCTCAAAGTGCCAGCTACAGTTTCGGTTTCTTCGCCTG
ATGCTGTTACAGCGTATAATGGTTATCTTACTTCTTCGTCTAAAACACCTGAAGA
ACATTTTATTGAAACCATCTCACTTGCTGGTTCGTATAAAGATTGGTCCTATTCGG
GACAATCTACACAACTAGGTATAGAATTTCTTAAGAGAGGTGATAAATCGGTAT
ATTACACTAGTAATCCTACCACATTCCACCTAGATGGTGAAGTTATCACCTTTGA
CAATCTTAAGACACTTCTTTCGTTGAGAGAAGTGAGGACTATTAAGGTGTTTACA
ACAGTAGACAACATTAACCTCCACACGCAAGTTGTGGACATGTCAATGACATAT
GGACAACAGTTTGGTCCAACTTATTTGGATGGAGCTGATGTTACTAAAATAAAAC
CTCATAATTCGCATGAAGGTAAAACATTTTATGTTTTACCTAATGATGACACTCT
ACGTGTTGAGGCTTTTGAGTACTACCACACAACTGATCCTAGTTTTCTGGGTAGG
TACATGTCGGCATTAAATCACACTAAAAAGTGGAAATACCCACAAGTTAATGGT
TTAACTTCTATTAAATGGGCAGATAACAACTGTTATCTTGCCACTGCATTGTTAAC
ACTCCAACAAATAGAGTTGAAGTTTAATCCACCTGCTCTACAAGATGCTTATTAC
AGAGCAAGGGCTGGTGAAGCTGCTAACTTTTGTGCACTTATCTTAGCCTACTGTA
ATAAGACAGTAGGTGAGTTAGGTGATGTTAGAGAAACAATGTCGTACTTGTTTCA
ACATGCCAATTTAGATTCTTGCAAAAGAGTCTTGAACGTGGTGTGTAAAACTTGT
GGACAACAGCAGACAACCCTTAAGGGTGTAGAAGCTGTTATGTACATGGGCACA
CTTTCGTATGAACAATTTAAGAAAGGTGTTCAGATACCTTGTACGTGTGGTAAAC
AAGCTACAAAATATCTAGTACAACAGGAGTCACCTTTTGTTATGATGTCGGCACC
ACCTGCTCAGTATGAACTTAAGCATGGTACATTTACTTGTGCTAGTGAGTACACT
GGTAATTACCAGTGTGGTCACTATAAACATATAACTTCGAAAGAAACTTTGTATT
GCATAGACGGTGCTTTACTTACAAAGTCCTCGGAATACAAAGGTCCTATTACGGA
TGTTTTCTACAAAGAAAACAGTTACACAACAACCATAAAACCAGTTACTTATAAA
TTGGATGGTGTTGTTTGTACAGAAATTGACCCTAAGTTGGACAATTATTATAAGA
AAGACAATTCGTATTTCACAGAGCAACCAATTGATCTTGTACCAAACCAACCATA
TCCAAACGCAAGCTTCGATAATTTTAAGTTTGTATGTGATAATATCAAATTTGCT
GATGATTTAAACCAGTTAACTGGTTATAAGAAACCTGCTTCGAGAGAGCTTAAAG
TTACATTTTTCCCTGACTTAAATGGTGATGTGGTGGCTATTGATTATAAACACTAC
ACACCCTCTTTTAAGAAAGGAGCTAAATTGTTACATAAACCTATTGTTTGGCATG
TTAACAATGCAACTAATAAAGCCACGTATAAACCAAATACCTGGTGTATACGTTG
TCTTTGGTCGACAAAACCAGTTGAAACATCAAATTCGTTTGATGTACTGAAGTCA
GAGGACGCGCAGGGAATGGATAATCTTGCCTGCGAAGATCTAAAACCAGTCTCG
GAAGAAGTAGTGGAAAATCCTACCATACAGAAAGACGTTCTTGAGTGTAATGTG
AAAACTACCGAAGTTGTAGGAGACATTATACTTAAACCAGCAAATAATAGTTTA
AAAATTACAGAAGAGGTTGGCCACACAGATCTAATGGCTGCTTATGTAGACAAT
TCGAGTCTTACTATTAAGAAACCTAATGAATTATCTAGAGTATTAGGTTTGAAAA
CCCTTGCTACTCATGGTTTAGCTGCTGTTAATTCGGTCCCTTGGGATACTATAGCT
AATTATGCTAAGCCTTTTCTTAACAAAGTTGTTAGTACAACTACTAACATAGTTA
CACGGTGTTTAAACCGTGTTTGTACTAATTATATGCCTTATTTCTTTACTTTATTGC
TACAATTGTGTACTTTTACTAGATCGACAAATTCTAGAATTAAAGCATCGATGCC
GACTACTATAGCAAAGAATACTGTTAAGAGTGTCGGTAAATTTTGTCTAGAGGCT
TCGTTTAATTATTTGAAGTCACCTAATTTTTCGAAACTGATAAATATTATAATTTG
GTTTTTACTATTAAGTGTTTGCCTAGGTTCTTTAATCTACTCAACCGCTGCTTTAG
GTGTTTTAATGTCTAATTTAGGCATGCCTTCTTACTGTACTGGTTACAGAGAAGGC
TATTTGAACTCTACTAATGTCACTATTGCAACCTACTGTACTGGTTCTATACCTTG
TAGTGTTTGTCTTAGTGGTTTAGATTCTTTAGACACCTATCCTTCTTTAGAAACTA
TACAAATTACCATTTCATCTTTTAAATGGGATTTAACTGCTTTTGGCTTAGTTGCA
GAGTGGTTTTTGGCATATATTCTTTTCACTAGGTTTTTCTATGTACTTGGATTGGC
TGCAATCATGCAATTGTTTTTCAGCTATTTTGCAGTACATTTTATTAGTAATTCTT
GGCTTATGTGGTTAATAATTAATCTTGTACAAATGGCCCCGATTTCAGCTATGGTT
AGAATGTACATCTTCTTTGCATCATTTTATTATGTATGGAAAAGTTATGTGCATGT
TGTAGACGGTTGTAATTCATCAACTTGTATGATGTGTTACAAACGTAATAGAGCA
ACAAGAGTCGAATGTACAACTATTGTTAATGGTGTTAGAAGGTCCTTTTATGTCT
ATGCTAATGGAGGTAAAGGCTTTTGCAAACTACACAATTGGAATTGTGTTAATTG
TGATACATTCTGTGCTGGTAGTACATTTATTAGTGATGAAGTTGCGAGAGACTTG
TCACTACAGTTTAAAAGACCAATAAATCCTACTGACCAGTCTTCTTACATCGTTG
ATAGTGTTACAGTGAAGAATGGTTCCATCCATCTTTACTTTGATAAAGCTGGTCA
AAAGACTTATGAAAGACATTCTCTCTCTCATTTTGTTAACTTAGACAACCTGAGA
GCTAATAACACTAAAGGTTCATTGCCTATTAATGTTATAGTTTTTGATGGTAAATC
AAAATGTGAAGAATCATCTGCAAAATCAGCGTCTGTTTACTACAGTCAGCTTATG
TGTCAACCTATACTGTTACTAGATCAGGCATTAGTGTCTGATGTTGGTGATAGTG
CGGAAGTTGCAGTTAAAATGTTTGATGCTTACGTTAATACGTTTTCATCAACTTTT
AACGTACCAATGGAAAAACTCAAAACACTAGTTGCAACTGCAGAAGCTGAACTT
GCAAAGAATGTGTCCTTAGACAATGTCTTATCTACTTTTATTTCAGCAGCTCGGC
AAGGGTTTGTTGATTCAGATGTAGAAACTAAAGATGTTGTTGAATGTCTTAAATT
GTCACATCAATCTGACATAGAAGTTACTGGCGATAGTTGTAATAACTATATGCTC
ACCTATAACAAAGTTGAAAACATGACACCCCGTGACCTTGGTGCTTGTATTGACT
GTAGTGCGCGTCATATTAATGCGCAGGTAGCAAAAAGTCACAACATTGCTTTGAT
ATGGAACGTTAAAGATTTCATGTCATTGTCTGAACAACTACGAAAACAAATACGT
AGTGCTGCTAAAAAGAATAACTTACCTTTTAAGTTGACATGTGCAACTACTAGAC
AAGTTGTTAATGTTGTAACAACAAAGATAGCACTTAAGGGTGGTAAAATTGTTAA
TAATTGGTTGAAGCAGTTAATTAAA
SEQ ID NO: 41 - Clone pCC1-4K-SARS-COV-2-77-3. Only fragment 1 has been
deoptimized:
GGGTCCCACGTGCTAGCGCTAACATAGGTTGTAACCATACAGGTGTTG
TTGGAGAAGGTTCGGAAGGTCTTAATGACAACCTTCTTGAAATACTCCAAAAAG
AGAAAGTCAACATCAATATTGTTGGTGACTTTAAACTTAATGAAGAGATCGCCAT
TATTTTGGCATCTTTTTCGGCTTCCACATCGGCTTTTGTGGAAACTGTGAAAGGTT
TGGATTATAAAGCATTCAAACAAATTGTTGAATCCTGTGGTAATTTTAAAGTTAC
AAAAGGAAAAGCTAAAAAAGGTGCCTGGAATATTGGTGAACAGAAATCGATACT
GAGTCCTCTTTATGCATTTGCATCGGAGGCTGCTCGTGTTGTACGATCAATTTTCT
CGCGCACTCTTGAAACTGCTCAAAATTCTGTGCGTGTTTTACAGAAGGCCGCTAT
AACAATACTAGATGGAATTTCGCAGTATTCACTGAGACTCATTGATGCTATGATG
TTCACATCGGATTTGGCTACTAACAATCTAGTTGTAATGGCCTACATTACAGGTG
GTGTTGTTCAGTTGACTTCGCAGTGGCTAACTAACATCTTTGGCACTGTTTATGAA
AAACTCAAACCCGTCCTTGATTGGCTTGAAGAGAAGTTTAAGGAAGGTGTAGAG
TTTCTTAGAGACGGTTGGGAAATTGTTAAATTTATCTCAACCTGTGCTTGTGAAAT
TGTCGGTGGACAAATTGTCACCTGTGCAAAGGAAATTAAGGAGTCGGTTCAGAC
ATTCTTTAAGCTTGTAAATAAATTTTTGGCTTTGTGTGCTGACTCTATCATTATTG
GTGGAGCTAAACTTAAAGCCTTGAATTTAGGTGAAACATTTGTCACGCACTCGAA
GGGATTGTACAGAAAGTGTGTTAAATCCAGAGAAGAAACTGGCCTACTCATGCC
TCTAAAAGCCCCAAAAGAAATTATCTTCTTAGAGGGAGAAACACTTCCCACAGA
AGTGTTAACAGAGGAAGTTGTCTTGAAAACTGGTGATTTACAACCATTAGAACA
ACCTACTTCGGAAGCTGTTGAAGCTCCATTGGTTGGTACACCAGTTTGTATTAAC
GGGCTTATGTTGCTCGAAATCAAAGACACAGAAAAGTACTGTGCCCTTGCACCTA
ATATGATGGTAACAAACAATACCTTCACACTCAAAGGCGGTGCACCAACAAAGG
TTACTTTTGGTGATGACACTGTGATAGAAGTGCAAGGTTACAAGAGTGTGAATAT
CACTTTTGAACTTGATGAAAGGATTGATAAAGTACTTAATGAGAAGTGCTCGGCC
TATACAGTTGAACTCGGTACAGAAGTAAATGAGTTCGCCTGTGTTGTGGCAGATG
CTGTCATAAAAACTTTGCAACCAGTATCTGAATTACTTACACCACTGGGCATTGA
TTTAGATGAGTGGTCGATGGCTACATACTACTTATTTGATGAGTCTGGTGAGTTT
AAATTGGCTTCGCATATGTATTGTTCTTTCTACCCTCCAGATGAGGATGAAGAAG
AAGGTGATTGTGAAGAAGAAGAGTTTGAGCCATCGACTCAATATGAGTATGGTA
CTGAAGATGATTACCAAGGTAAACCTTTGGAATTTGGTGCCACTTCTGCTGCTCT
TCAACCTGAAGAAGAGCAAGAAGAAGATTGGTTAGATGATGATTCGCAACAAAC
TGTTGGTCAACAAGACGGCAGTGAGGACAATCAGACAACTACTATTCAAACAAT
TGTTGAGGTTCAACCTCAATTAGAGATGGAACTTACACCAGTTGTTCAGACTATT
GAAGTGAATTCGTTTAGTGGTTATTTAAAACTTACTGACAATGTATACATTAAAA
ATGCAGACATTGTGGAAGAAGCTAAAAAGGTAAAACCAACAGTGGTTGTTAATG
CAGCCAATGTTTACCTTAAACATGGAGGAGGTGTTGCAGGAGCCTTAAATAAGG
CTACTAACAATGCCATGCAAGTTGAATCGGATGATTACATAGCTACTAATGGACC
ACTTAAAGTGGGTGGTTCGTGTGTTTTAAGCGGACACAATCTTGCTAAACACTGT
CTTCATGTTGTCGGCCCAAATGTTAACAAAGGTGAAGACATTCAACTTCTTAAGT
CGGCTTATGAAAATTTTAATCAGCACGAAGTTCTACTTGCACCATTATTATCAGC
TGGTATTTTTGGTGCTGACCCTATACATTCGTTAAGAGTTTGTGTAGATACTGTTC
GCACAAATGTCTACTTAGCTGTCTTTGATAAAAATCTCTATGACAAACTTGTTTCA
TCGTTTTTGGAAATGAAGAGTGAAAAGCAAGTTGAACAAAAGATCGCTGAGATT
CCTAAAGAGGAAGTTAAGCCATTTATAACTGAATCGAAACCTTCAGTTGAACAG
AGAAAACAAGATGATAAGAAAATCAAAGCTTGTGTTGAAGAAGTTACAACAACT
CTGGAAGAAACTAAGTTCCTCACAGAAAACTTGTTACTTTATATTGACATTAATG
GCAATCTTCATCCAGATTCGGCCACTCTTGTTAGTGACATTGACATCACTTTCTTA
AAGAAAGATGCTCCATATATAGTGGGTGATGTTGTTCAAGAGGGTGTTTTAACTG
CTGTGGTTATACCTACTAAAAAGGCTGGTGGCACTACTGAAATGCTAGCGAAAG
CTTTGAGAAAAGTGCCAACAGACAATTATATAACCACTTACCCGGGTCAGGGTTT
AAATGGTTACACTGTAGAGGAGGCAAAGACAGTGCTTAAAAAGTGTAAAAGTGC
CTTTTACATTCTACCATCTATTATCTCTAATGAGAAGCAAGAAATTCTTGGAACTG
TTTCTTGGAATTTGCGAGAAATGCTTGCACATGCAGAAGAAACACGCAAATTAAT
GCCTGTCTGTGTGGAAACTAAAGCCATAGTTTCAACTATACAGCGTAAATATAAG
GGTATTAAAATACAAGAGGGTGTGGTTGATTATGGTGCTAGATTTTACTTTTACA
CCAGTAAAACAACTGTAGCGTCACTTATCAACACACTTAACGATCTAAATGAAA
CTCTTGTTACAATGCCACTTGGCTATGTAACACATGGCTTAAATTTGGAAGAAGC
TGCTCGGTATATGAGATCTCTCAAAGTGCCAGCTACAGTTTCTGTTTCTTCACCTG
ATGCTGTTACAGCGTATAATGGTTATCTTACTTCTTCTTCTAAAACACCTGAAGAA
CATTTTATTGAAACCATCTCACTTGCTGGTTCCTATAAAGATTGGTCCTATTCTGG
ACAATCTACACAACTAGGTATAGAATTTCTTAAGAGAGGTGATAAAAGTGTATAT
TACACTAGTAATCCTACCACATTCCACCTAGATGGTGAAGTTATCACCTTTGACA
ATCTTAAGACACTTCTTTCTTTGAGAGAAGTGAGGACTATTAAGGTGTTTACAAC
AGTAGACAACATTAACCTCCACACGCAAGTTGTGGACATGTCAATGACATATGG
ACAACAGTTTGGTCCAACTTATTTGGATGGAGCTGATGTTACTAAAATAAAACCT
CATAATTCACATGAAGGTAAAACATTTTATGTTTTACCTAATGATGACACTCTAC
GTGTTGAGGCTTTTGAGTACTACCACACAACTGATCCTAGTTTTCTGGGTAGGTA
CATGTCAGCATTAAATCACACTAAAAAGTGGAAATACCCACAAGTTAATGGTTT
AACTTCTATTAAATGGGCAGATAACAACTGTTATCTTGCCACTGCATTGTTAACA
CTCCAACAAATAGAGTTGAAGTTTAATCCACCTGCTCTACAAGATGCTTATTACA
GAGCAAGGGCTGGTGAAGCTGCTAACTTTTGTGCACTTATCTTAGCCTACTGTAA
TAAGACAGTAGGTGAGTTAGGTGATGTTAGAGAAACAATGAGTTACTTGTTTCAA
CATGCCAATTTAGATTCTTGCAAAAGAGTCTTGAACGTGGTGTGTAAAACTTGTG
GACAACAGCAGACAACCCTTAAGGGTGTAGAAGCTGTTATGTACATGGGCACAC
TTTCTTATGAACAATTTAAGAAAGGTGTTCAGATACCTTGTACGTGTGGTAAACA
AGCTACAAAATATCTAGTACAACAGGAGTCACCTTTTGTTATGATGTCAGCACCA
CCTGCTCAGTATGAACTTAAGCATGGTACATTTACTTGTGCTAGTGAGTACACTG
GTAATTACCAGTGTGGTCACTATAAACATATAACTTCTAAAGAAACTTTGTATTG
CATAGACGGTGCTTTACTTACAAAGTCCTCAGAATACAAAGGTCCTATTACGGAT
GTTTTCTACAAAGAAAACAGTTACACAACAACCATAAAACCAGTTACTTATAAAT
TGGATGGTGTTGTTTGTACAGAAATTGACCCTAAGTTGGACAATTATTATAAGAA
AGACAATTCTTATTTCACAGAGCAACCAATTGATCTTGTACCAAACCAACCATAT
CCAAACGCAAGCTTCGATAATTTTAAGTTTGTATGTGATAATATCAAATTTGCTG
ATGATTTAAACCAGTTAACTGGTTATAAGAAACCTGCTTCAAGAGAGCTTAAAGT
TACATTTTTCCCTGACTTAAATGGTGATGTGGTGGCTATTGATTATAAACACTACA
CACCCTCTTTTAAGAAAGGAGCTAAATTGTTACATAAACCTATTGTTTGGCATGT
TAACAATGCAACTAATAAAGCCACGTATAAACCAAATACCTGGTGTATACGTTGT
CTTTGGAGCACAAAACCAGTTGAAACATCAAATTCGTTTGATGTACTGAAGTCAG
AGGACGCGCAGGGAATGGATAATCTTGCCTGCGAAGATCTAAAACCAGTCTCTG
AAGAAGTAGTGGAAAATCCTACCATACAGAAAGACGTTCTTGAGTGTAATGTGA
AAACTACCGAAGTTGTAGGAGACATTATACTTAAACCAGCAAATAATAGTTTAA
AAATTACAGAAGAGGTTGGCCACACAGATCTAATGGCTGCTTATGTAGACAATT
CTAGTCTTACTATTAAGAAACCTAATGAATTATCTAGAGTATTAGGTTTGAAAAC
CCTTGCTACTCATGGTTTAGCTGCTGTTAATAGTGTCCCTTGGGATACTATAGCTA
ATTATGCTAAGCCTTTTCTTAACAAAGTTGTTAGTACAACTACTAACATAGTTAC
ACGGTGTTTAAACCGTGTTTGTACTAATTATATGCCTTATTTCTTTACTTTATTGCT
ACAATTGTGTACTTTTACTAGAAGTACAAATTCTAGAATTAAAGCATCTATGCCG
ACTACTATAGCAAAGAATACTGTTAAGAGTGTCGGTAAATTTTGTCTAGAGGCTT
CATTTAATTATTTGAAGTCACCTAATTTTTCTAAACTGATAAATATTATAATTTGG
TTTTTACTATTAAGTGTTTGCCTAGGTTCTTTAATCTACTCAACCGCTGCTTTAGG
TGTTTTAATGTCTAATTTAGGCATGCCTTCTTACTGTACTGGTTACAGAGAAGGCT
ATTTGAACTCTACTAATGTCACTATTGCAACCTACTGTACTGGTTCTATACCTTGT
AGTGTTTGTCTTAGTGGTTTAGATTCTTTAGACACCTATCCTTCTTTAGAAACTAT
ACAAATTACCATTTCATCTTTTAAATGGGATTTAACTGCTTTTGGCTTAGTTGCAG
AGTGGTTTTTGGCATATATTCTTTTCACTAGGTTTTTCTATGTACTTGGATTGGCT
GCAATCATGCAATTGTTTTTCAGCTATTTTGCAGTACATTTTATTAGTAATTCTTG
GCTTATGTGGTTAATAATTAATCTTGTACAAATGGCCCCGATTTCAGCTATGGTTA
GAATGTACATCTTCTTTGCATCATTTTATTATGTATGGAAAAGTTATGTGCATGTT
GTAGACGGTTGTAATTCATCAACTTGTATGATGTGTTACAAACGTAATAGAGCAA
CAAGAGTCGAATGTACAACTATTGTTAATGGTGTTAGAAGGTCCTTTTATGTCTA
TGCTAATGGAGGTAAAGGCTTTTGCAAACTACACAATTGGAATTGTGTTAATTGT
GATACATTCTGTGCTGGTAGTACATTTATTAGTGATGAAGTTGCGAGAGACTTGT
CACTACAGTTTAAAAGACCAATAAATCCTACTGACCAGTCTTCTTACATCGTTGA
TAGTGTTACAGTGAAGAATGGTTCCATCCATCTTTACTTTGATAAAGCTGGTCAA
AAGACTTATGAAAGACATTCTCTCTCTCATTTTGTTAACTTAGACAACCTGAGAG
CTAATAACACTAAAGGTTCATTGCCTATTAATGTTATAGTTTTTGATGGTAAATCA
AAATGTGAAGAATCATCTGCAAAATCAGCGTCTGTTTACTACAGTCAGCTTATGT
GTCAACCTATACTGTTACTAGATCAGGCATTAGTGTCTGATGTTGGTGATAGTGC
GGAAGTTGCAGTTAAAATGTTTGATGCTTACGTTAATACGTTTTCATCAACTTTTA
ACGTACCAATGGAAAAACTCAAAACACTAGTTGCAACTGCAGAAGCTGAACTTG
CAAAGAATGTGTCCTTAGACAATGTCTTATCTACTTTTATTTCAGCAGCTCGGCA
AGGGTTTGTTGATTCAGATGTAGAAACTAAAGATGTTGTTGAATGTCTTAAATTG
TCACATCAATCTGACATAGAAGTTACTGGCGATAGTTGTAATAACTATATGCTCA
CCTATAACAAAGTTGAAAACATGACACCCCGTGACCTTGGTGCTTGTATTGACTG
TAGTGCGCGTCATATTAATGCGCAGGTAGCAAAAAGTCACAACATTGCTTTGATA
TGGAACGTTAAAGATTTCATGTCATTGTCTGAACAACTACGAAAACAAATACGTA
GTGCTGCTAAAAAGAATAACTTACCTTTTAAGTTGACATGTGCAACTACTAGACA
AGTTGTTAATGTTGTAACAACAAAGATAGCACTTAAGGGTGGTAAAATTGTTAAT
AATTGGTTGAAGCAGTTAATTAAA
SEQ ID NO: 42 - Clone pCC1-4K-SARS-COV-2-77-4. Only fragments 1 and 2
have been deoptimized:
GGGTCCCACGTGCTAGCGCTAACATAGGTTGTAACCATACAGGTGTTG
TTGGAGAAGGTTCGGAAGGTCTTAATGACAACCTTCTTGAAATACTCCAAAAAG
AGAAAGTCAACATCAATATTGTTGGTGACTTTAAACTTAATGAAGAGATCGCCAT
TATTTTGGCATCTTTTTCGGCTTCCACATCGGCTTTTGTGGAAACTGTGAAAGGTT
TGGATTATAAAGCATTCAAACAAATTGTTGAATCCTGTGGTAATTTTAAAGTTAC
AAAAGGAAAAGCTAAAAAAGGTGCCTGGAATATTGGTGAACAGAAATCGATACT
GAGTCCTCTTTATGCATTTGCATCGGAGGCTGCTCGTGTTGTACGATCAATTTTCT
CGCGCACTCTTGAAACTGCTCAAAATTCTGTGCGTGTTTTACAGAAGGCCGCTAT
AACAATACTAGATGGAATTTCGCAGTATTCACTGAGACTCATTGATGCTATGATG
TTCACATCGGATTTGGCTACTAACAATCTAGTTGTAATGGCCTACATTACAGGTG
GTGTTGTTCAGTTGACTTCGCAGTGGCTAACTAACATCTTTGGCACTGTTTATGAA
AAACTCAAACCCGTCCTTGATTGGCTTGAAGAGAAGTTTAAGGAAGGTGTAGAG
TTTCTTAGAGACGGTTGGGAAATTGTTAAATTTATCTCAACCTGTGCTTGTGAAAT
TGTCGGTGGACAAATTGTCACCTGTGCAAAGGAAATTAAGGAGTCGGTTCAGAC
ATTCTTTAAGCTTGTAAATAAATTTTTGGCTTTGTGTGCTGACTCTATCATTATTG
GTGGAGCTAAACTTAAAGCCTTGAATTTAGGTGAAACATTTGTCACGCACTCGAA
GGGATTGTACAGAAAGTGTGTTAAATCCAGAGAAGAAACTGGCCTACTCATGCC
TCTAAAAGCCCCAAAAGAAATTATCTTCTTAGAGGGAGAAACACTTCCCACAGA
AGTGTTAACAGAGGAAGTTGTCTTGAAAACTGGTGATTTACAACCATTAGAACA
ACCTACTTCGGAAGCTGTTGAAGCTCCATTGGTTGGTACACCAGTTTGTATTAAC
GGGCTTATGTTGCTCGAAATCAAAGACACAGAAAAGTACTGTGCCCTTGCACCTA
ATATGATGGTAACAAACAATACCTTCACACTCAAAGGCGGTGCACCAACAAAGG
TTACTTTTGGTGATGACACTGTGATAGAAGTGCAAGGTTACAAGAGTGTGAATAT
CACTTTTGAACTTGATGAAAGGATTGATAAAGTACTTAATGAGAAGTGCTCGGCC
TATACAGTTGAACTCGGTACAGAAGTAAATGAGTTCGCCTGTGTTGTGGCAGATG
CTGTCATAAAAACTTTGCAACCAGTATCTGAATTACTTACACCACTGGGCATTGA
TTTAGATGAGTGGTCGATGGCTACATACTACTTATTTGATGAGTCTGGTGAGTTT
AAATTGGCTTCGCATATGTATTGTTCTTTCTACCCTCCAGATGAGGATGAAGAAG
AAGGTGATTGTGAAGAAGAAGAGTTTGAGCCATCGACTCAATATGAGTATGGTA
CTGAAGATGATTACCAAGGTAAACCTTTGGAATTTGGTGCCACTTCTGCTGCTCT
TCAACCTGAAGAAGAGCAAGAAGAAGATTGGTTAGATGATGATTCGCAACAAAC
TGTTGGTCAACAAGACGGCAGTGAGGACAATCAGACAACTACTATTCAAACAAT
TGTTGAGGTTCAACCTCAATTAGAGATGGAACTTACACCAGTTGTTCAGACTATT
GAAGTGAATTCGTTTAGTGGTTATTTAAAACTTACTGACAATGTATACATTAAAA
ATGCAGACATTGTGGAAGAAGCTAAAAAGGTAAAACCAACAGTGGTTGTTAATG
CAGCCAATGTTTACCTTAAACATGGAGGAGGTGTTGCAGGAGCCTTAAATAAGG
CTACTAACAATGCCATGCAAGTTGAATCGGATGATTACATAGCTACTAATGGACC
ACTTAAAGTGGGTGGTTCGTGTGTTTTAAGCGGACACAATCTTGCTAAACACTGT
CTTCATGTTGTCGGCCCAAATGTTAACAAAGGTGAAGACATTCAACTTCTTAAGT
CGGCTTATGAAAATTTTAATCAGCACGAAGTTCTACTTGCACCATTATTATCAGC
TGGTATTTTTGGTGCTGACCCTATACATTCGTTAAGAGTTTGTGTAGATACTGTTC
GCACAAATGTCTACTTAGCTGTCTTTGATAAAAATCTCTATGACAAACTTGTTTCA
TCGTTTTTGGAAATGAAGAGTGAAAAGCAAGTTGAACAAAAGATCGCTGAGATT
CCTAAAGAGGAAGTTAAGCCATTTATAACTGAATCGAAACCTTCAGTTGAACAG
AGAAAACAAGATGATAAGAAAATCAAAGCTTGTGTTGAAGAAGTTACAACAACT
CTGGAAGAAACTAAGTTCCTCACAGAAAACTTGTTACTTTATATTGACATTAATG
GCAATCTTCATCCAGATTCGGCCACTCTTGTTAGTGACATTGACATCACTTTCTTA
AAGAAAGATGCTCCATATATAGTGGGTGATGTTGTTCAAGAGGGTGTTTTAACTG
CTGTGGTTATACCTACTAAAAAGGCTGGTGGCACTACTGAAATGCTAGCGAAAG
CTTTGAGAAAAGTGCCAACAGACAATTATATAACCACTTACCCGGGTCAGGGTTT
AAATGGTTACACTGTAGAGGAGGCAAAGACAGTGCTTAAAAAGTGTAAATCGGC
CTTTTACATTCTACCATCTATTATCTCGAATGAGAAGCAAGAAATTCTTGGAACT
GTTTCTTGGAATTTGCGAGAAATGCTTGCACATGCAGAAGAAACACGCAAATTA
ATGCCTGTCTGTGTGGAAACTAAAGCCATAGTTTCGACTATACAGCGTAAATATA
AGGGTATTAAAATACAAGAGGGTGTGGTTGATTATGGTGCTAGATTTTACTTTTA
CACCAGTAAAACAACTGTAGCGTCGCTTATCAACACACTTAACGATCTAAATGA
AACTCTTGTTACAATGCCACTTGGCTATGTAACACATGGCTTAAATTTGGAAGAA
GCTGCTCGGTATATGAGATCTCTCAAAGTGCCAGCTACAGTTTCGGTTTCTTCGC
CTGATGCTGTTACAGCGTATAATGGTTATCTTACTTCTTCGTCTAAAACACCTGAA
GAACATTTTATTGAAACCATCTCACTTGCTGGTTCGTATAAAGATTGGTCCTATTC
GGGACAATCTACACAACTAGGTATAGAATTTCTTAAGAGAGGTGATAAATCGGT
ATATTACACTAGTAATCCTACCACATTCCACCTAGATGGTGAAGTTATCACCTTT
GACAATCTTAAGACACTTCTTTCGTTGAGAGAAGTGAGGACTATTAAGGTGTTTA
CAACAGTAGACAACATTAACCTCCACACGCAAGTTGTGGACATGTCAATGACAT
ATGGACAACAGTTTGGTCCAACTTATTTGGATGGAGCTGATGTTACTAAAATAAA
ACCTCATAATTCGCATGAAGGTAAAACATTTTATGTTTTACCTAATGATGACACT
CTACGTGTTGAGGCTTTTGAGTACTACCACACAACTGATCCTAGTTTTCTGGGTA
GGTACATGTCGGCATTAAATCACACTAAAAAGTGGAAATACCCACAAGTTAATG
GTTTAACTTCTATTAAATGGGCAGATAACAACTGTTATCTTGCCACTGCATTGTTA
ACACTCCAACAAATAGAGTTGAAGTTTAATCCACCTGCTCTACAAGATGCTTATT
ACAGAGCAAGGGCTGGTGAAGCTGCTAACTTTTGTGCACTTATCTTAGCCTACTG
TAATAAGACAGTAGGTGAGTTAGGTGATGTTAGAGAAACAATGTCGTACTTGTTT
CAACATGCCAATTTAGATTCTTGCAAAAGAGTCTTGAACGTGGTGTGTAAAACTT
GTGGACAACAGCAGACAACCCTTAAGGGTGTAGAAGCTGTTATGTACATGGGCA
CACTTTCGTATGAACAATTTAAGAAAGGTGTTCAGATACCTTGTACGTGTGGTAA
ACAAGCTACAAAATATCTAGTACAACAGGAGTCACCTTTTGTTATGATGTCGGCA
CCACCTGCTCAGTATGAACTTAAGCATGGTACATTTACTTGTGCTAGTGAGTACA
CTGGTAATTACCAGTGTGGTCACTATAAACATATAACTTCGAAAGAAACTTTGTA
TTGCATAGACGGTGCTTTACTTACAAAGTCCTCGGAATACAAAGGTCCTATTACG
GATGTTTTCTACAAAGAAAACAGTTACACAACAACCATAAAACCAGTTACTTATA
AATTGGATGGTGTTGTTTGTACAGAAATTGACCCTAAGTTGGACAATTATTATAA
GAAAGACAATTCGTATTTCACAGAGCAACCAATTGATCTTGTACCAAACCAACC
ATATCCAAACGCAAGCTTCGATAATTTTAAGTTTGTATGTGATAATATCAAATTT
GCTGATGATTTAAACCAGTTAACTGGTTATAAGAAACCTGCTTCGAGAGAGCTTA
AAGTTACATTTTTCCCTGACTTAAATGGTGATGTGGTGGCTATTGATTATAAACA
CTACACACCCTCTTTTAAGAAAGGAGCTAAATTGTTACATAAACCTATTGTTTGG
CATGTTAACAATGCAACTAATAAAGCCACGTATAAACCAAATACCTGGTGTATA
CGTTGTCTTTGGTCGACAAAACCAGTTGAAACATCAAATTCGTTTGATGTACTGA
AGTCAGAGGACGCGCAGGGAATGGATAATCTTGCCTGCGAAGATCTAAAACCAG
TCTCGGAAGAAGTAGTGGAAAATCCTACCATACAGAAAGACGTTCTTGAGTGTA
ATGTGAAAACTACCGAAGTTGTAGGAGACATTATACTTAAACCAGCAAATAATA
GTTTAAAAATTACAGAAGAGGTTGGCCACACAGATCTAATGGCTGCTTATGTAGA
CAATTCGAGTCTTACTATTAAGAAACCTAATGAATTATCTAGAGTATTAGGTTTG
AAAACCCTTGCTACTCATGGTTTAGCTGCTGTTAATTCGGTCCCTTGGGATACTAT
AGCTAATTATGCTAAGCCTTTTCTTAACAAAGTTGTTAGTACAACTACTAACATA
GTTACACGGTGTTTAAACCGTGTTTGTACTAATTATATGCCTTATTTCTTTACTTT
ATTGCTACAATTGTGTACTTTTACTAGATCGACAAATTCTAGAATTAAAGCATCG
ATGCCGACTACTATAGCAAAGAATACTGTTAAGAGTGTCGGTAAATTTTGTCTAG
AGGCTTCGTTTAATTATTTGAAGTCACCTAATTTTTCGAAACTGATAAATATTATA
ATTTGGTTTTTACTATTAAGTGTTTGCCTAGGTTCTTTAATCTACTCAACCGCTGC
TTTAGGTGTTTTAATGTCTAATTTAGGCATGCCTTCTTACTGTACTGGTTACAGAG
AAGGCTATTTGAACTCTACTAATGTCACTATTGCAACCTACTGTACTGGTTCTATA
CCTTGTAGTGTTTGTCTTAGTGGTTTAGATTCTTTAGACACCTATCCTTCTTTAGA
AACTATACAAATTACCATTTCATCTTTTAAATGGGATTTAACTGCTTTTGGCTTAG
TTGCAGAGTGGTTTTTGGCATATATTCTTTTCACTAGGTTTTTCTATGTACTTGGA
TTGGCTGCAATCATGCAATTGTTTTTCAGCTATTTTGCAGTACATTTTATTAGTAA
TTCTTGGCTTATGTGGTTAATAATTAATCTTGTACAAATGGCCCCGATTTCAGCTA
TGGTTAGAATGTACATCTTCTTTGCATCATTTTATTATGTATGGAAAAGTTATGTG
CATGTTGTAGACGGTTGTAATTCATCAACTTGTATGATGTGTTACAAACGTAATA
GAGCAACAAGAGTCGAATGTACAACTATTGTTAATGGTGTTAGAAGGTCCTTTTA
TGTCTATGCTAATGGAGGTAAAGGCTTTTGCAAACTACACAATTGGAATTGTGTT
AATTGTGATACATTCTGTGCTGGTAGTACATTTATTAGTGATGAAGTTGCGAGAG
ACTTGTCACTACAGTTTAAAAGACCAATAAATCCTACTGACCAGTCTTCTTACAT
CGTTGATAGTGTTACAGTGAAGAATGGTTCCATCCATCTTTACTTTGATAAAGCT
GGTCAAAAGACTTATGAAAGACATTCTCTCTCTCATTTTGTTAACTTAGACAACC
TGAGAGCTAATAACACTAAAGGTTCATTGCCTATTAATGTTATAGTTTTTGATGG
TAAATCAAAATGTGAAGAATCATCTGCAAAATCAGCGTCTGTTTACTACAGTCAG
CTTATGTGTCAACCTATACTGTTACTAGATCAGGCATTAGTGTCTGATGTTGGTGA
TAGTGCGGAAGTTGCAGTTAAAATGTTTGATGCTTACGTTAATACGTTTTCATCA
ACTTTTAACGTACCAATGGAAAAACTCAAAACACTAGTTGCAACTGCAGAAGCT
GAACTTGCAAAGAATGTGTCCTTAGACAATGTCTTATCTACTTTTATTTCAGCAGC
TCGGCAAGGGTTTGTTGATTCAGATGTAGAAACTAAAGATGTTGTTGAATGTCTT
AAATTGTCACATCAATCTGACATAGAAGTTACTGGCGATAGTTGTAATAACTATA
TGCTCACCTATAACAAAGTTGAAAACATGACACCCCGTGACCTTGGTGCTTGTAT
TGACTGTAGTGCGCGTCATATTAATGCGCAGGTAGCAAAAAGTCACAACATTGCT
TTGATATGGAACGTTAAAGATTTCATGTCATTGTCTGAACAACTACGAAAACAAA
TACGTAGTGCTGCTAAAAAGAATAACTTACCTTTTAAGTTGACATGTGCAACTAC
TAGACAAGTTGTTAATGTTGTAACAACAAAGATAGCACTTAAGGGTGGTAAAAT
TGTTAATAATTGGTTGAAGCAGTTAATTAAA
SEQ ID NO: 43 - Clone pCC1-4K-SARS-COV-2-77-5. Only fragments 1 and 3
have been deoptimized:
GGGTCCCACGTGCTAGCGCTAACATAGGTTGTAACCATACAGGTGTTG
TTGGAGAAGGTTCGGAAGGTCTTAATGACAACCTTCTTGAAATACTCCAAAAAG
AGAAAGTCAACATCAATATTGTTGGTGACTTTAAACTTAATGAAGAGATCGCCAT
TATTTTGGCATCTTTTTCGGCTTCCACATCGGCTTTTGTGGAAACTGTGAAAGGTT
TGGATTATAAAGCATTCAAACAAATTGTTGAATCCTGTGGTAATTTTAAAGTTAC
AAAAGGAAAAGCTAAAAAAGGTGCCTGGAATATTGGTGAACAGAAATCGATACT
GAGTCCTCTTTATGCATTTGCATCGGAGGCTGCTCGTGTTGTACGATCAATTTTCT
CGCGCACTCTTGAAACTGCTCAAAATTCTGTGCGTGTTTTACAGAAGGCCGCTAT
AACAATACTAGATGGAATTTCGCAGTATTCACTGAGACTCATTGATGCTATGATG
TTCACATCGGATTTGGCTACTAACAATCTAGTTGTAATGGCCTACATTACAGGTG
GTGTTGTTCAGTTGACTTCGCAGTGGCTAACTAACATCTTTGGCACTGTTTATGAA
AAACTCAAACCCGTCCTTGATTGGCTTGAAGAGAAGTTTAAGGAAGGTGTAGAG
TTTCTTAGAGACGGTTGGGAAATTGTTAAATTTATCTCAACCTGTGCTTGTGAAAT
TGTCGGTGGACAAATTGTCACCTGTGCAAAGGAAATTAAGGAGTCGGTTCAGAC
ATTCTTTAAGCTTGTAAATAAATTTTTGGCTTTGTGTGCTGACTCTATCATTATTG
GTGGAGCTAAACTTAAAGCCTTGAATTTAGGTGAAACATTTGTCACGCACTCGAA
GGGATTGTACAGAAAGTGTGTTAAATCCAGAGAAGAAACTGGCCTACTCATGCC
TCTAAAAGCCCCAAAAGAAATTATCTTCTTAGAGGGAGAAACACTTCCCACAGA
AGTGTTAACAGAGGAAGTTGTCTTGAAAACTGGTGATTTACAACCATTAGAACA
ACCTACTTCGGAAGCTGTTGAAGCTCCATTGGTTGGTACACCAGTTTGTATTAAC
GGGCTTATGTTGCTCGAAATCAAAGACACAGAAAAGTACTGTGCCCTTGCACCTA
ATATGATGGTAACAAACAATACCTTCACACTCAAAGGCGGTGCACCAACAAAGG
TTACTTTTGGTGATGACACTGTGATAGAAGTGCAAGGTTACAAGAGTGTGAATAT
CACTTTTGAACTTGATGAAAGGATTGATAAAGTACTTAATGAGAAGTGCTCGGCC
TATACAGTTGAACTCGGTACAGAAGTAAATGAGTTCGCCTGTGTTGTGGCAGATG
CTGTCATAAAAACTTTGCAACCAGTATCTGAATTACTTACACCACTGGGCATTGA
TTTAGATGAGTGGTCGATGGCTACATACTACTTATTTGATGAGTCTGGTGAGTTT
AAATTGGCTTCGCATATGTATTGTTCTTTCTACCCTCCAGATGAGGATGAAGAAG
AAGGTGATTGTGAAGAAGAAGAGTTTGAGCCATCGACTCAATATGAGTATGGTA
CTGAAGATGATTACCAAGGTAAACCTTTGGAATTTGGTGCCACTTCTGCTGCTCT
TCAACCTGAAGAAGAGCAAGAAGAAGATTGGTTAGATGATGATTCGCAACAAAC
TGTTGGTCAACAAGACGGCAGTGAGGACAATCAGACAACTACTATTCAAACAAT
TGTTGAGGTTCAACCTCAATTAGAGATGGAACTTACACCAGTTGTTCAGACTATT
GAAGTGAATTCGTTTAGTGGTTATTTAAAACTTACTGACAATGTATACATTAAAA
ATGCAGACATTGTGGAAGAAGCTAAAAAGGTAAAACCAACAGTGGTTGTTAATG
CAGCCAATGTTTACCTTAAACATGGAGGAGGTGTTGCAGGAGCCTTAAATAAGG
CTACTAACAATGCCATGCAAGTTGAATCGGATGATTACATAGCTACTAATGGACC
ACTTAAAGTGGGTGGTTCGTGTGTTTTAAGCGGACACAATCTTGCTAAACACTGT
CTTCATGTTGTCGGCCCAAATGTTAACAAAGGTGAAGACATTCAACTTCTTAAGT
CGGCTTATGAAAATTTTAATCAGCACGAAGTTCTACTTGCACCATTATTATCAGC
TGGTATTTTTGGTGCTGACCCTATACATTCGTTAAGAGTTTGTGTAGATACTGTTC
GCACAAATGTCTACTTAGCTGTCTTTGATAAAAATCTCTATGACAAACTTGTTTCA
TCGTTTTTGGAAATGAAGAGTGAAAAGCAAGTTGAACAAAAGATCGCTGAGATT
CCTAAAGAGGAAGTTAAGCCATTTATAACTGAATCGAAACCTTCAGTTGAACAG
AGAAAACAAGATGATAAGAAAATCAAAGCTTGTGTTGAAGAAGTTACAACAACT
CTGGAAGAAACTAAGTTCCTCACAGAAAACTTGTTACTTTATATTGACATTAATG
GCAATCTTCATCCAGATTCGGCCACTCTTGTTAGTGACATTGACATCACTTTCTTA
AAGAAAGATGCTCCATATATAGTGGGTGATGTTGTTCAAGAGGGTGTTTTAACTG
CTGTGGTTATACCTACTAAAAAGGCTGGTGGCACTACTGAAATGCTAGCGAAAG
CTTTGAGAAAAGTGCCAACAGACAATTATATAACCACTTACCCGGGTCAGGGTTT
AAATGGTTACACTGTAGAGGAGGCAAAGACAGTGCTTAAAAAGTGTAAAAGTGC
CTTTTACATTCTACCATCTATTATCTCTAATGAGAAGCAAGAAATTCTTGGAACTG
TTTCTTGGAATTTGCGAGAAATGCTTGCACATGCAGAAGAAACACGCAAATTAAT
GCCTGTCTGTGTGGAAACTAAAGCCATAGTTTCAACTATACAGCGTAAATATAAG
GGTATTAAAATACAAGAGGGTGTGGTTGATTATGGTGCTAGATTTTACTTTTACA
CCAGTAAAACAACTGTAGCGTCACTTATCAACACACTTAACGATCTAAATGAAA
CTCTTGTTACAATGCCACTTGGCTATGTAACACATGGCTTAAATTTGGAAGAAGC
TGCTCGGTATATGAGATCTCTCAAAGTGCCAGCTACAGTTTCTGTTTCTTCACCTG
ATGCTGTTACAGCGTATAATGGTTATCTTACTTCTTCTTCTAAAACACCTGAAGAA
CATTTTATTGAAACCATCTCACTTGCTGGTTCCTATAAAGATTGGTCCTATTCTGG
ACAATCTACACAACTAGGTATAGAATTTCTTAAGAGAGGTGATAAAAGTGTATAT
TACACTAGTAATCCTACCACATTCCACCTAGATGGTGAAGTTATCACCTTTGACA
ATCTTAAGACACTTCTTTCTTTGAGAGAAGTGAGGACTATTAAGGTGTTTACAAC
AGTAGACAACATTAACCTCCACACGCAAGTTGTGGACATGTCAATGACATATGG
ACAACAGTTTGGTCCAACTTATTTGGATGGAGCTGATGTTACTAAAATAAAACCT
CATAATTCACATGAAGGTAAAACATTTTATGTTTTACCTAATGATGACACTCTAC
GTGTTGAGGCTTTTGAGTACTACCACACAACTGATCCTAGTTTTCTGGGTAGGTA
CATGTCAGCATTAAATCACACTAAAAAGTGGAAATACCCACAAGTTAATGGTTT
AACTTCTATTAAATGGGCAGATAACAACTGTTATCTTGCCACTGCATTGTTAACA
CTCCAACAAATAGAGTTGAAGTTTAATCCACCTGCTCTACAAGATGCTTATTACA
GAGCAAGGGCTGGTGAAGCTGCTAACTTTTGTGCACTTATCTTAGCCTACTGTAA
TAAGACAGTAGGTGAGTTAGGTGATGTTAGAGAAACAATGAGTTACTTGTTTCAA
CATGCCAATTTAGATTCTTGCAAAAGAGTCTTGAACGTGGTGTGTAAAACTTGTG
GACAACAGCAGACAACCCTTAAGGGTGTAGAAGCTGTTATGTACATGGGCACAC
TTTCTTATGAACAATTTAAGAAAGGTGTTCAGATACCTTGTACGTGTGGTAAACA
AGCTACAAAATATCTAGTACAACAGGAGTCACCTTTTGTTATGATGTCAGCACCA
CCTGCTCAGTATGAACTTAAGCATGGTACATTTACTTGTGCTAGTGAGTACACTG
GTAATTACCAGTGTGGTCACTATAAACATATAACTTCTAAAGAAACTTTGTATTG
CATAGACGGTGCTTTACTTACAAAGTCCTCAGAATACAAAGGTCCTATTACGGAT
GTTTTCTACAAAGAAAACAGTTACACAACAACCATAAAACCAGTTACTTATAAAT
TGGATGGTGTTGTTTGTACAGAAATTGACCCTAAGTTGGACAATTATTATAAGAA
AGACAATTCTTATTTCACAGAGCAACCAATTGATCTTGTACCAAACCAACCATAT
CCAAACGCAAGCTTCGATAATTTTAAGTTTGTATGTGATAATATCAAATTTGCTG
ATGATTTAAACCAGTTAACTGGTTATAAGAAACCTGCTTCAAGAGAGCTTAAAGT
TACATTTTTCCCTGACTTAAATGGTGATGTGGTGGCTATTGATTATAAACACTACA
CACCCTCTTTTAAGAAAGGAGCTAAATTGTTACATAAACCTATTGTTTGGCATGT
TAACAATGCAACTAATAAAGCCACGTATAAACCAAATACCTGGTGTATACGTTGT
CTTTGGAGCACAAAACCAGTTGAAACATCAAATTCGTTTGATGTACTGAAGTCAG
AGGACGCGCAGGGAATGGATAATCTTGCCTGCGAAGATCTAAAACCAGTCTCTG
AAGAAGTAGTGGAAAATCCTACCATACAGAAAGACGTTCTTGAGTGTAATGTGA
AAACTACCGAAGTTGTAGGAGACATTATACTTAAACCAGCAAATAATAGTTTAA
AAATTACAGAAGAGGTTGGCCACACAGATCTAATGGCTGCTTATGTAGACAATT
CTAGTCTTACTATTAAGAAACCTAATGAATTATCTAGAGTATTAGGTTTGAAAAC
CCTTGCTACTCATGGTTTAGCTGCTGTTAATAGTGTCCCTTGGGATACTATAGCTA
ATTATGCTAAGCCTTTTCTTAACAAAGTTGTTAGTACAACTACTAACATAGTTAC
ACGGTGTTTAAACCGTGTTTGTACTAATTATATGCCTTATTTCTTTACTTTATTGCT
ACAATTGTGTACTTTTACTAGAAGTACAAATTCTAGAATTAAAGCATCTATGCCG
ACTACTATAGCAAAGAATACTGTTAAGAGTGTCGGTAAATTTTGTCTAGAGGCTT
CATTTAATTATTTGAAGTCACCTAATTTTTCTAAACTGATAAATATTATAATTTGG
TTTTTACTATTAAGTGTTTGCCTAGGTTCGTTAATCTACTCAACCGCTGCTTTAGG
TGTTTTAATGTCGAATTTAGGCATGCCTTCTTACTGTACTGGTTACAGAGAAGGCT
ATTTGAACTCGACTAATGTCACTATTGCAACCTACTGTACTGGTTCTATACCTTGT
TCGGTTTGTCTTAGTGGTTTAGATTCTTTAGACACCTATCCTTCGTTAGAAACTAT
ACAAATTACCATTTCATCTTTTAAATGGGATTTAACTGCTTTTGGCTTAGTTGCAG
AGTGGTTTTTGGCATATATTCTTTTCACTAGGTTTTTCTATGTACTTGGATTGGCT
GCAATCATGCAATTGTTTTTCTCGTATTTTGCAGTACATTTTATTAGTAATTCGTG
GCTTATGTGGTTAATAATTAATCTTGTACAAATGGCCCCGATTTCAGCTATGGTTA
GAATGTACATCTTCTTTGCATCGTTTTATTATGTATGGAAAAGTTATGTGCATGTT
GTAGACGGTTGTAATTCGTCAACTTGTATGATGTGTTACAAACGTAATAGAGCAA
CAAGAGTCGAATGTACAACTATTGTTAATGGTGTTAGAAGGTCGTTTTATGTCTA
TGCTAATGGAGGTAAAGGCTTTTGCAAACTACACAATTGGAATTGTGTTAATTGT
GATACATTCTGTGCTGGTAGTACATTTATTTCGGATGAAGTTGCGAGAGACTTGT
CACTACAGTTTAAAAGACCAATAAATCCTACTGACCAGTCGTCTTACATCGTTGA
TAGTGTTACAGTGAAGAATGGTTCGATCCATCTTTACTTTGATAAAGCTGGTCAA
AAGACTTATGAAAGACATTCTCTCTCGCATTTTGTTAACTTAGACAACCTGAGAG
CTAATAACACTAAAGGTTCATTGCCTATTAATGTTATAGTTTTTGATGGTAAATCG
AAATGTGAAGAATCATCGGCAAAATCAGCGTCGGTTTACTACAGTCAGCTTATGT
GTCAACCTATACTGTTACTAGATCAGGCATTAGTGTCGGATGTTGGTGATAGTGC
GGAAGTTGCAGTTAAAATGTTTGATGCTTACGTTAATACGTTTTCGTCAACTTTTA
ACGTACCAATGGAAAAACTCAAAACACTAGTTGCAACTGCAGAAGCTGAACTTG
CAAAGAATGTGTCCTTAGACAATGTCTTATCGACTTTTATTTCAGCAGCTCGGCA
AGGGTTTGTTGATTCGGATGTAGAAACTAAAGATGTTGTTGAATGTCTTAAATTG
TCACATCAATCGGACATAGAAGTTACTGGCGATAGTTGTAATAACTATATGCTCA
CCTATAACAAAGTTGAAAACATGACACCCCGTGACCTTGGTGCTTGTATTGACTG
TTCGGCGCGTCATATTAATGCGCAGGTAGCAAAAAGTCACAACATTGCTTTGATA
TGGAACGTTAAAGATTTCATGTCGTTGTCTGAACAACTACGAAAACAAATACGTT
CGGCTGCTAAAAAGAATAACTTACCTTTTAAGTTGACATGTGCAACTACTAGACA
AGTTGTTAATGTTGTAACAACAAAGATAGCACTTAAGGGTGGTAAAATTGTTAAT
AATTGGTTGAAGCAGTTAATTAAA
SEQ ID NO: 44 - Clone pCC1-4K-SARS-COV-2-77-6. Only fragments 2 and 3
have been deoptimized:
GGGTCCCACGTGCTAGCGCTAACATAGGTTGTAACCATACAGGTGTTG
TTGGAGAAGGTTCCGAAGGTCTTAATGACAACCTTCTTGAAATACTCCAAAAAG
AGAAAGTCAACATCAATATTGTTGGTGACTTTAAACTTAATGAAGAGATCGCCAT
TATTTTGGCATCTTTTTCTGCTTCCACAAGTGCTTTTGTGGAAACTGTGAAAGGTT
TGGATTATAAAGCATTCAAACAAATTGTTGAATCCTGTGGTAATTTTAAAGTTAC
AAAAGGAAAAGCTAAAAAAGGTGCCTGGAATATTGGTGAACAGAAATCAATACT
GAGTCCTCTTTATGCATTTGCATCAGAGGCTGCTCGTGTTGTACGATCAATTTTCT
CCCGCACTCTTGAAACTGCTCAAAATTCTGTGCGTGTTTTACAGAAGGCCGCTAT
AACAATACTAGATGGAATTTCACAGTATTCACTGAGACTCATTGATGCTATGATG
TTCACATCTGATTTGGCTACTAACAATCTAGTTGTAATGGCCTACATTACAGGTG
GTGTTGTTCAGTTGACTTCGCAGTGGCTAACTAACATCTTTGGCACTGTTTATGAA
AAACTCAAACCCGTCCTTGATTGGCTTGAAGAGAAGTTTAAGGAAGGTGTAGAG
TTTCTTAGAGACGGTTGGGAAATTGTTAAATTTATCTCAACCTGTGCTTGTGAAAT
TGTCGGTGGACAAATTGTCACCTGTGCAAAGGAAATTAAGGAGAGTGTTCAGAC
ATTCTTTAAGCTTGTAAATAAATTTTTGGCTTTGTGTGCTGACTCTATCATTATTG
GTGGAGCTAAACTTAAAGCCTTGAATTTAGGTGAAACATTTGTCACGCACTCAAA
GGGATTGTACAGAAAGTGTGTTAAATCCAGAGAAGAAACTGGCCTACTCATGCC
TCTAAAAGCCCCAAAAGAAATTATCTTCTTAGAGGGAGAAACACTTCCCACAGA
AGTGTTAACAGAGGAAGTTGTCTTGAAAACTGGTGATTTACAACCATTAGAACA
ACCTACTAGTGAAGCTGTTGAAGCTCCATTGGTTGGTACACCAGTTTGTATTAAC
GGGCTTATGTTGCTCGAAATCAAAGACACAGAAAAGTACTGTGCCCTTGCACCTA
ATATGATGGTAACAAACAATACCTTCACACTCAAAGGCGGTGCACCAACAAAGG
TTACTTTTGGTGATGACACTGTGATAGAAGTGCAAGGTTACAAGAGTGTGAATAT
CACTTTTGAACTTGATGAAAGGATTGATAAAGTACTTAATGAGAAGTGCTCTGCC
TATACAGTTGAACTCGGTACAGAAGTAAATGAGTTCGCCTGTGTTGTGGCAGATG
CTGTCATAAAAACTTTGCAACCAGTATCTGAATTACTTACACCACTGGGCATTGA
TTTAGATGAGTGGAGTATGGCTACATACTACTTATTTGATGAGTCTGGTGAGTTT
AAATTGGCTTCACATATGTATTGTTCTTTCTACCCTCCAGATGAGGATGAAGAAG
AAGGTGATTGTGAAGAAGAAGAGTTTGAGCCATCAACTCAATATGAGTATGGTA
CTGAAGATGATTACCAAGGTAAACCTTTGGAATTTGGTGCCACTTCTGCTGCTCT
TCAACCTGAAGAAGAGCAAGAAGAAGATTGGTTAGATGATGATAGTCAACAAAC
TGTTGGTCAACAAGACGGCAGTGAGGACAATCAGACAACTACTATTCAAACAAT
TGTTGAGGTTCAACCTCAATTAGAGATGGAACTTACACCAGTTGTTCAGACTATT
GAAGTGAATAGTTTTAGTGGTTATTTAAAACTTACTGACAATGTATACATTAAAA
ATGCAGACATTGTGGAAGAAGCTAAAAAGGTAAAACCAACAGTGGTTGTTAATG
CAGCCAATGTTTACCTTAAACATGGAGGAGGTGTTGCAGGAGCCTTAAATAAGG
CTACTAACAATGCCATGCAAGTTGAATCTGATGATTACATAGCTACTAATGGACC
ACTTAAAGTGGGTGGTAGTTGTGTTTTAAGCGGACACAATCTTGCTAAACACTGT
CTTCATGTTGTCGGCCCAAATGTTAACAAAGGTGAAGACATTCAACTTCTTAAGA
GTGCTTATGAAAATTTTAATCAGCACGAAGTTCTACTTGCACCATTATTATCAGCT
GGTATTTTTGGTGCTGACCCTATACATTCTTTAAGAGTTTGTGTAGATACTGTTCG
CACAAATGTCTACTTAGCTGTCTTTGATAAAAATCTCTATGACAAACTTGTTTCAA
GCTTTTTGGAAATGAAGAGTGAAAAGCAAGTTGAACAAAAGATCGCTGAGATTC
CTAAAGAGGAAGTTAAGCCATTTATAACTGAAAGTAAACCTTCAGTTGAACAGA
GAAAACAAGATGATAAGAAAATCAAAGCTTGTGTTGAAGAAGTTACAACAACTC
TGGAAGAAACTAAGTTCCTCACAGAAAACTTGTTACTTTATATTGACATTAATGG
CAATCTTCATCCAGATTCTGCCACTCTTGTTAGTGACATTGACATCACTTTCTTAA
AGAAAGATGCTCCATATATAGTGGGTGATGTTGTTCAAGAGGGTGTTTTAACTGC
TGTGGTTATACCTACTAAAAAGGCTGGTGGCACTACTGAAATGCTAGCGAAAGC
TTTGAGAAAAGTGCCAACAGACAATTATATAACCACTTACCCGGGTCAGGGTTTA
AATGGTTACACTGTAGAGGAGGCAAAGACAGTGCTTAAAAAGTGTAAATCGGCC
TTTTACATTCTACCATCTATTATCTCGAATGAGAAGCAAGAAATTCTTGGAACTG
TTTCTTGGAATTTGCGAGAAATGCTTGCACATGCAGAAGAAACACGCAAATTAAT
GCCTGTCTGTGTGGAAACTAAAGCCATAGTTTCGACTATACAGCGTAAATATAAG
GGTATTAAAATACAAGAGGGTGTGGTTGATTATGGTGCTAGATTTTACTTTTACA
CCAGTAAAACAACTGTAGCGTCGCTTATCAACACACTTAACGATCTAAATGAAA
CTCTTGTTACAATGCCACTTGGCTATGTAACACATGGCTTAAATTTGGAAGAAGC
TGCTCGGTATATGAGATCTCTCAAAGTGCCAGCTACAGTTTCGGTTTCTTCGCCTG
ATGCTGTTACAGCGTATAATGGTTATCTTACTTCTTCGTCTAAAACACCTGAAGA
ACATTTTATTGAAACCATCTCACTTGCTGGTTCGTATAAAGATTGGTCCTATTCGG
GACAATCTACACAACTAGGTATAGAATTTCTTAAGAGAGGTGATAAATCGGTAT
ATTACACTAGTAATCCTACCACATTCCACCTAGATGGTGAAGTTATCACCTTTGA
CAATCTTAAGACACTTCTTTCGTTGAGAGAAGTGAGGACTATTAAGGTGTTTACA
ACAGTAGACAACATTAACCTCCACACGCAAGTTGTGGACATGTCAATGACATAT
GGACAACAGTTTGGTCCAACTTATTTGGATGGAGCTGATGTTACTAAAATAAAAC
CTCATAATTCGCATGAAGGTAAAACATTTTATGTTTTACCTAATGATGACACTCT
ACGTGTTGAGGCTTTTGAGTACTACCACACAACTGATCCTAGTTTTCTGGGTAGG
TACATGTCGGCATTAAATCACACTAAAAAGTGGAAATACCCACAAGTTAATGGT
TTAACTTCTATTAAATGGGCAGATAACAACTGTTATCTTGCCACTGCATTGTTAAC
ACTCCAACAAATAGAGTTGAAGTTTAATCCACCTGCTCTACAAGATGCTTATTAC
AGAGCAAGGGCTGGTGAAGCTGCTAACTTTTGTGCACTTATCTTAGCCTACTGTA
ATAAGACAGTAGGTGAGTTAGGTGATGTTAGAGAAACAATGTCGTACTTGTTTCA
ACATGCCAATTTAGATTCTTGCAAAAGAGTCTTGAACGTGGTGTGTAAAACTTGT
GGACAACAGCAGACAACCCTTAAGGGTGTAGAAGCTGTTATGTACATGGGCACA
CTTTCGTATGAACAATTTAAGAAAGGTGTTCAGATACCTTGTACGTGTGGTAAAC
AAGCTACAAAATATCTAGTACAACAGGAGTCACCTTTTGTTATGATGTCGGCACC
ACCTGCTCAGTATGAACTTAAGCATGGTACATTTACTTGTGCTAGTGAGTACACT
GGTAATTACCAGTGTGGTCACTATAAACATATAACTTCGAAAGAAACTTTGTATT
GCATAGACGGTGCTTTACTTACAAAGTCCTCGGAATACAAAGGTCCTATTACGGA
TGTTTTCTACAAAGAAAACAGTTACACAACAACCATAAAACCAGTTACTTATAAA
TTGGATGGTGTTGTTTGTACAGAAATTGACCCTAAGTTGGACAATTATTATAAGA
AAGACAATTCGTATTTCACAGAGCAACCAATTGATCTTGTACCAAACCAACCATA
TCCAAACGCAAGCTTCGATAATTTTAAGTTTGTATGTGATAATATCAAATTTGCT
GATGATTTAAACCAGTTAACTGGTTATAAGAAACCTGCTTCGAGAGAGCTTAAAG
TTACATTTTTCCCTGACTTAAATGGTGATGTGGTGGCTATTGATTATAAACACTAC
ACACCCTCTTTTAAGAAAGGAGCTAAATTGTTACATAAACCTATTGTTTGGCATG
TTAACAATGCAACTAATAAAGCCACGTATAAACCAAATACCTGGTGTATACGTTG
TCTTTGGTCGACAAAACCAGTTGAAACATCAAATTCGTTTGATGTACTGAAGTCA
GAGGACGCGCAGGGAATGGATAATCTTGCCTGCGAAGATCTAAAACCAGTCTCG
GAAGAAGTAGTGGAAAATCCTACCATACAGAAAGACGTTCTTGAGTGTAATGTG
AAAACTACCGAAGTTGTAGGAGACATTATACTTAAACCAGCAAATAATAGTTTA
AAAATTACAGAAGAGGTTGGCCACACAGATCTAATGGCTGCTTATGTAGACAAT
TCGAGTCTTACTATTAAGAAACCTAATGAATTATCTAGAGTATTAGGTTTGAAAA
CCCTTGCTACTCATGGTTTAGCTGCTGTTAATTCGGTCCCTTGGGATACTATAGCT
AATTATGCTAAGCCTTTTCTTAACAAAGTTGTTAGTACAACTACTAACATAGTTA
CACGGTGTTTAAACCGTGTTTGTACTAATTATATGCCTTATTTCTTTACTTTATTGC
TACAATTGTGTACTTTTACTAGATCGACAAATTCTAGAATTAAAGCATCGATGCC
GACTACTATAGCAAAGAATACTGTTAAGAGTGTCGGTAAATTTTGTCTAGAGGCT
TCGTTTAATTATTTGAAGTCACCTAATTTTTCGAAACTGATAAATATTATAATTTG
GTTTTTACTATTAAGTGTTTGCCTAGGTTCGTTAATCTACTCAACCGCTGCTTTAG
GTGTTTTAATGTCGAATTTAGGCATGCCTTCTTACTGTACTGGTTACAGAGAAGG
CTATTTGAACTCGACTAATGTCACTATTGCAACCTACTGTACTGGTTCTATACCTT
GTTCGGTTTGTCTTAGTGGTTTAGATTCTTTAGACACCTATCCTTCGTTAGAAACT
ATACAAATTACCATTTCATCTTTTAAATGGGATTTAACTGCTTTTGGCTTAGTTGC
AGAGTGGTTTTTGGCATATATTCTTTTCACTAGGTTTTTCTATGTACTTGGATTGG
CTGCAATCATGCAATTGTTTTTCTCGTATTTTGCAGTACATTTTATTAGTAATTCG
TGGCTTATGTGGTTAATAATTAATCTTGTACAAATGGCCCCGATTTCAGCTATGGT
TAGAATGTACATCTTCTTTGCATCGTTTTATTATGTATGGAAAAGTTATGTGCATG
TTGTAGACGGTTGTAATTCGTCAACTTGTATGATGTGTTACAAACGTAATAGAGC
AACAAGAGTCGAATGTACAACTATTGTTAATGGTGTTAGAAGGTCGTTTTATGTC
TATGCTAATGGAGGTAAAGGCTTTTGCAAACTACACAATTGGAATTGTGTTAATT
GTGATACATTCTGTGCTGGTAGTACATTTATTTCGGATGAAGTTGCGAGAGACTT
GTCACTACAGTTTAAAAGACCAATAAATCCTACTGACCAGTCGTCTTACATCGTT
GATAGTGTTACAGTGAAGAATGGTTCGATCCATCTTTACTTTGATAAAGCTGGTC
AAAAGACTTATGAAAGACATTCTCTCTCGCATTTTGTTAACTTAGACAACCTGAG
AGCTAATAACACTAAAGGTTCATTGCCTATTAATGTTATAGTTTTTGATGGTAAA
TCGAAATGTGAAGAATCATCGGCAAAATCAGCGTCGGTTTACTACAGTCAGCTTA
TGTGTCAACCTATACTGTTACTAGATCAGGCATTAGTGTCGGATGTTGGTGATAG
TGCGGAAGTTGCAGTTAAAATGTTTGATGCTTACGTTAATACGTTTTCGTCAACTT
TTAACGTACCAATGGAAAAACTCAAAACACTAGTTGCAACTGCAGAAGCTGAAC
TTGCAAAGAATGTGTCCTTAGACAATGTCTTATCGACTTTTATTTCAGCAGCTCGG
CAAGGGTTTGTTGATTCGGATGTAGAAACTAAAGATGTTGTTGAATGTCTTAAAT
TGTCACATCAATCGGACATAGAAGTTACTGGCGATAGTTGTAATAACTATATGCT
CACCTATAACAAAGTTGAAAACATGACACCCCGTGACCTTGGTGCTTGTATTGAC
TGTTCGGCGCGTCATATTAATGCGCAGGTAGCAAAAAGTCACAACATTGCTTTGA
TATGGAACGTTAAAGATTTCATGTCGTTGTCTGAACAACTACGAAAACAAATACG
TTCGGCTGCTAAAAAGAATAACTTACCTTTTAAGTTGACATGTGCAACTACTAGA
CAAGTTGTTAATGTTGTAACAACAAAGATAGCACTTAAGGGTGGTAAAATTGTTA
ATAATTGGTTGAAGCAGTTAATTAAA
SEQ ID NO: 45 - Clone pCC1-4K-SARS-COV-2-77-7. All 3 fragments were
deoptimized. Deoptimized sequence between SanDI to PacI in ORF1a:
GGGTCCCACGTGCTAGCGCTAACATAGGTTGTAACCATACAGGTGTTG
TTGGAGAAGGTTCGGAAGGTCTTAATGACAACCTTCTTGAAATACTCCAAAAAG
AGAAAGTCAACATCAATATTGTTGGTGACTTTAAACTTAATGAAGAGATCGCCAT
TATTTTGGCATCTTTTTCGGCTTCCACATCGGCTTTTGTGGAAACTGTGAAAGGTT
TGGATTATAAAGCATTCAAACAAATTGTTGAATCCTGTGGTAATTTTAAAGTTAC
AAAAGGAAAAGCTAAAAAAGGTGCCTGGAATATTGGTGAACAGAAATCGATACT
GAGTCCTCTTTATGCATTTGCATCGGAGGCTGCTCGTGTTGTACGATCAATTTTCT
CGCGCACTCTTGAAACTGCTCAAAATTCTGTGCGTGTTTTACAGAAGGCCGCTAT
AACAATACTAGATGGAATTTCGCAGTATTCACTGAGACTCATTGATGCTATGATG
TTCACATCGGATTTGGCTACTAACAATCTAGTTGTAATGGCCTACATTACAGGTG
GTGTTGTTCAGTTGACTTCGCAGTGGCTAACTAACATCTTTGGCACTGTTTATGAA
AAACTCAAACCCGTCCTTGATTGGCTTGAAGAGAAGTTTAAGGAAGGTGTAGAG
TTTCTTAGAGACGGTTGGGAAATTGTTAAATTTATCTCAACCTGTGCTTGTGAAAT
TGTCGGTGGACAAATTGTCACCTGTGCAAAGGAAATTAAGGAGTCGGTTCAGAC
ATTCTTTAAGCTTGTAAATAAATTTTTGGCTTTGTGTGCTGACTCTATCATTATTG
GTGGAGCTAAACTTAAAGCCTTGAATTTAGGTGAAACATTTGTCACGCACTCGAA
GGGATTGTACAGAAAGTGTGTTAAATCCAGAGAAGAAACTGGCCTACTCATGCC
TCTAAAAGCCCCAAAAGAAATTATCTTCTTAGAGGGAGAAACACTTCCCACAGA
AGTGTTAACAGAGGAAGTTGTCTTGAAAACTGGTGATTTACAACCATTAGAACA
ACCTACTTCGGAAGCTGTTGAAGCTCCATTGGTTGGTACACCAGTTTGTATTAAC
GGGCTTATGTTGCTCGAAATCAAAGACACAGAAAAGTACTGTGCCCTTGCACCTA
ATATGATGGTAACAAACAATACCTTCACACTCAAAGGCGGTGCACCAACAAAGG
TTACTTTTGGTGATGACACTGTGATAGAAGTGCAAGGTTACAAGAGTGTGAATAT
CACTTTTGAACTTGATGAAAGGATTGATAAAGTACTTAATGAGAAGTGCTCGGCC
TATACAGTTGAACTCGGTACAGAAGTAAATGAGTTCGCCTGTGTTGTGGCAGATG
CTGTCATAAAAACTTTGCAACCAGTATCTGAATTACTTACACCACTGGGCATTGA
TTTAGATGAGTGGTCGATGGCTACATACTACTTATTTGATGAGTCTGGTGAGTTT
AAATTGGCTTCGCATATGTATTGTTCTTTCTACCCTCCAGATGAGGATGAAGAAG
AAGGTGATTGTGAAGAAGAAGAGTTTGAGCCATCGACTCAATATGAGTATGGTA
CTGAAGATGATTACCAAGGTAAACCTTTGGAATTTGGTGCCACTTCTGCTGCTCT
TCAACCTGAAGAAGAGCAAGAAGAAGATTGGTTAGATGATGATTCGCAACAAAC
TGTTGGTCAACAAGACGGCAGTGAGGACAATCAGACAACTACTATTCAAACAAT
TGTTGAGGTTCAACCTCAATTAGAGATGGAACTTACACCAGTTGTTCAGACTATT
GAAGTGAATTCGTTTAGTGGTTATTTAAAACTTACTGACAATGTATACATTAAAA
ATGCAGACATTGTGGAAGAAGCTAAAAAGGTAAAACCAACAGTGGTTGTTAATG
CAGCCAATGTTTACCTTAAACATGGAGGAGGTGTTGCAGGAGCCTTAAATAAGG
CTACTAACAATGCCATGCAAGTTGAATCGGATGATTACATAGCTACTAATGGACC
ACTTAAAGTGGGTGGTTCGTGTGTTTTAAGCGGACACAATCTTGCTAAACACTGT
CTTCATGTTGTCGGCCCAAATGTTAACAAAGGTGAAGACATTCAACTTCTTAAGT
CGGCTTATGAAAATTTTAATCAGCACGAAGTTCTACTTGCACCATTATTATCAGC
TGGTATTTTTGGTGCTGACCCTATACATTCGTTAAGAGTTTGTGTAGATACTGTTC
GCACAAATGTCTACTTAGCTGTCTTTGATAAAAATCTCTATGACAAACTTGTTTCA
TCGTTTTTGGAAATGAAGAGTGAAAAGCAAGTTGAACAAAAGATCGCTGAGATT
CCTAAAGAGGAAGTTAAGCCATTTATAACTGAATCGAAACCTTCAGTTGAACAG
AGAAAACAAGATGATAAGAAAATCAAAGCTTGTGTTGAAGAAGTTACAACAACT
CTGGAAGAAACTAAGTTCCTCACAGAAAACTTGTTACTTTATATTGACATTAATG
GCAATCTTCATCCAGATTCGGCCACTCTTGTTAGTGACATTGACATCACTTTCTTA
AAGAAAGATGCTCCATATATAGTGGGTGATGTTGTTCAAGAGGGTGTTTTAACTG
CTGTGGTTATACCTACTAAAAAGGCTGGTGGCACTACTGAAATGCTAGCGAAAG
CTTTGAGAAAAGTGCCAACAGACAATTATATAACCACTTACCCGGGTCAGGGTTT
AAATGGTTACACTGTAGAGGAGGCAAAGACAGTGCTTAAAAAGTGTAAATCGGC
CTTTTACATTCTACCATCTATTATCTCGAATGAGAAGCAAGAAATTCTTGGAACT
GTTTCTTGGAATTTGCGAGAAATGCTTGCACATGCAGAAGAAACACGCAAATTA
ATGCCTGTCTGTGTGGAAACTAAAGCCATAGTTTCGACTATACAGCGTAAATATA
AGGGTATTAAAATACAAGAGGGTGTGGTTGATTATGGTGCTAGATTTTACTTTTA
CACCAGTAAAACAACTGTAGCGTCGCTTATCAACACACTTAACGATCTAAATGA
AACTCTTGTTACAATGCCACTTGGCTATGTAACACATGGCTTAAATTTGGAAGAA
GCTGCTCGGTATATGAGATCTCTCAAAGTGCCAGCTACAGTTTCGGTTTCTTCGC
CTGATGCTGTTACAGCGTATAATGGTTATCTTACTTCTTCGTCTAAAACACCTGAA
GAACATTTTATTGAAACCATCTCACTTGCTGGTTCGTATAAAGATTGGTCCTATTC
GGGACAATCTACACAACTAGGTATAGAATTTCTTAAGAGAGGTGATAAATCGGT
ATATTACACTAGTAATCCTACCACATTCCACCTAGATGGTGAAGTTATCACCTTT
GACAATCTTAAGACACTTCTTTCGTTGAGAGAAGTGAGGACTATTAAGGTGTTTA
CAACAGTAGACAACATTAACCTCCACACGCAAGTTGTGGACATGTCAATGACAT
ATGGACAACAGTTTGGTCCAACTTATTTGGATGGAGCTGATGTTACTAAAATAAA
ACCTCATAATTCGCATGAAGGTAAAACATTTTATGTTTTACCTAATGATGACACT
CTACGTGTTGAGGCTTTTGAGTACTACCACACAACTGATCCTAGTTTTCTGGGTA
GGTACATGTCGGCATTAAATCACACTAAAAAGTGGAAATACCCACAAGTTAATG
GTTTAACTTCTATTAAATGGGCAGATAACAACTGTTATCTTGCCACTGCATTGTTA
ACACTCCAACAAATAGAGTTGAAGTTTAATCCACCTGCTCTACAAGATGCTTATT
ACAGAGCAAGGGCTGGTGAAGCTGCTAACTTTTGTGCACTTATCTTAGCCTACTG
TAATAAGACAGTAGGTGAGTTAGGTGATGTTAGAGAAACAATGTCGTACTTGTTT
CAACATGCCAATTTAGATTCTTGCAAAAGAGTCTTGAACGTGGTGTGTAAAACTT
GTGGACAACAGCAGACAACCCTTAAGGGTGTAGAAGCTGTTATGTACATGGGCA
CACTTTCGTATGAACAATTTAAGAAAGGTGTTCAGATACCTTGTACGTGTGGTAA
ACAAGCTACAAAATATCTAGTACAACAGGAGTCACCTTTTGTTATGATGTCGGCA
CCACCTGCTCAGTATGAACTTAAGCATGGTACATTTACTTGTGCTAGTGAGTACA
CTGGTAATTACCAGTGTGGTCACTATAAACATATAACTTCGAAAGAAACTTTGTA
TTGCATAGACGGTGCTTTACTTACAAAGTCCTCGGAATACAAAGGTCCTATTACG
GATGTTTTCTACAAAGAAAACAGTTACACAACAACCATAAAACCAGTTACTTATA
AATTGGATGGTGTTGTTTGTACAGAAATTGACCCTAAGTTGGACAATTATTATAA
GAAAGACAATTCGTATTTCACAGAGCAACCAATTGATCTTGTACCAAACCAACC
ATATCCAAACGCAAGCTTCGATAATTTTAAGTTTGTATGTGATAATATCAAATTT
GCTGATGATTTAAACCAGTTAACTGGTTATAAGAAACCTGCTTCGAGAGAGCTTA
AAGTTACATTTTTCCCTGACTTAAATGGTGATGTGGTGGCTATTGATTATAAACA
CTACACACCCTCTTTTAAGAAAGGAGCTAAATTGTTACATAAACCTATTGTTTGG
CATGTTAACAATGCAACTAATAAAGCCACGTATAAACCAAATACCTGGTGTATA
CGTTGTCTTTGGTCGACAAAACCAGTTGAAACATCAAATTCGTTTGATGTACTGA
AGTCAGAGGACGCGCAGGGAATGGATAATCTTGCCTGCGAAGATCTAAAACCAG
TCTCGGAAGAAGTAGTGGAAAATCCTACCATACAGAAAGACGTTCTTGAGTGTA
ATGTGAAAACTACCGAAGTTGTAGGAGACATTATACTTAAACCAGCAAATAATA
GTTTAAAAATTACAGAAGAGGTTGGCCACACAGATCTAATGGCTGCTTATGTAGA
CAATTCGAGTCTTACTATTAAGAAACCTAATGAATTATCTAGAGTATTAGGTTTG
AAAACCCTTGCTACTCATGGTTTAGCTGCTGTTAATTCGGTCCCTTGGGATACTAT
AGCTAATTATGCTAAGCCTTTTCTTAACAAAGTTGTTAGTACAACTACTAACATA
GTTACACGGTGTTTAAACCGTGTTTGTACTAATTATATGCCTTATTTCTTTACTTT
ATTGCTACAATTGTGTACTTTTACTAGATCGACAAATTCTAGAATTAAAGCATCG
ATGCCGACTACTATAGCAAAGAATACTGTTAAGAGTGTCGGTAAATTTTGTCTAG
AGGCTTCGTTTAATTATTTGAAGTCACCTAATTTTTCGAAACTGATAAATATTATA
ATTTGGTTTTTACTATTAAGTGTTTGCCTAGGTTCGTTAATCTACTCAACCGCTGC
TTTAGGTGTTTTAATGTCGAATTTAGGCATGCCTTCTTACTGTACTGGTTACAGAG
AAGGCTATTTGAACTCGACTAATGTCACTATTGCAACCTACTGTACTGGTTCTAT
ACCTTGTTCGGTTTGTCTTAGTGGTTTAGATTCTTTAGACACCTATCCTTCGTTAG
AAACTATACAAATTACCATTTCATCTTTTAAATGGGATTTAACTGCTTTTGGCTTA
GTTGCAGAGTGGTTTTTGGCATATATTCTTTTCACTAGGTTTTTCTATGTACTTGG
ATTGGCTGCAATCATGCAATTGTTTTTCTCGTATTTTGCAGTACATTTTATTAGTA
ATTCGTGGCTTATGTGGTTAATAATTAATCTTGTACAAATGGCCCCGATTTCAGCT
ATGGTTAGAATGTACATCTTCTTTGCATCGTTTTATTATGTATGGAAAAGTTATGT
GCATGTTGTAGACGGTTGTAATTCGTCAACTTGTATGATGTGTTACAAACGTAAT
AGAGCAACAAGAGTCGAATGTACAACTATTGTTAATGGTGTTAGAAGGTCGTTTT
ATGTCTATGCTAATGGAGGTAAAGGCTTTTGCAAACTACACAATTGGAATTGTGT
TAATTGTGATACATTCTGTGCTGGTAGTACATTTATTTCGGATGAAGTTGCGAGA
GACTTGTCACTACAGTTTAAAAGACCAATAAATCCTACTGACCAGTCGTCTTACA
TCGTTGATAGTGTTACAGTGAAGAATGGTTCGATCCATCTTTACTTTGATAAAGC
TGGTCAAAAGACTTATGAAAGACATTCTCTCTCGCATTTTGTTAACTTAGACAAC
CTGAGAGCTAATAACACTAAAGGTTCATTGCCTATTAATGTTATAGTTTTTGATG
GTAAATCGAAATGTGAAGAATCATCGGCAAAATCAGCGTCGGTTTACTACAGTC
AGCTTATGTGTCAACCTATACTGTTACTAGATCAGGCATTAGTGTCGGATGTTGG
TGATAGTGCGGAAGTTGCAGTTAAAATGTTTGATGCTTACGTTAATACGTTTTCG
TCAACTTTTAACGTACCAATGGAAAAACTCAAAACACTAGTTGCAACTGCAGAA
GCTGAACTTGCAAAGAATGTGTCCTTAGACAATGTCTTATCGACTTTTATTTCAGC
AGCTCGGCAAGGGTTTGTTGATTCGGATGTAGAAACTAAAGATGTTGTTGAATGT
CTTAAATTGTCACATCAATCGGACATAGAAGTTACTGGCGATAGTTGTAATAACT
ATATGCTCACCTATAACAAAGTTGAAAACATGACACCCCGTGACCTTGGTGCTTG
TATTGACTGTTCGGCGCGTCATATTAATGCGCAGGTAGCAAAAAGTCACAACATT
GCTTTGATATGGAACGTTAAAGATTTCATGTCGTTGTCTGAACAACTACGAAAAC
AAATACGTTCGGCTGCTAAAAAGAATAACTTACCTTTTAAGTTGACATGTGCAAC
TACTAGACAAGTTGTTAATGTTGTAACAACAAAGATAGCACTTAAGGGTGGTAA
AATTGTTAATAATTGGTTGAAGCAGTTAATTAAA
SEQ ID NO: 46 - Clone pCC1-4K-SARS-COV-2-160-1. Only fragment 3 has been
deoptimized:
GGGTCCCACGTGCTAGCGCTAACATAGGTTGTAACCATACAGGTGTTG
TTGGAGAAGGTTCCGAAGGTCTTAATGACAACCTTCTTGAAATACTCCAAAAAG
AGAAAGTCAACATCAATATTGTTGGTGACTTTAAACTTAATGAAGAGATCGCCAT
TATTTTGGCATCTTTTTCTGCTTCCACAAGTGCTTTTGTGGAAACTGTGAAAGGTT
TGGATTATAAAGCATTCAAACAAATTGTTGAATCCTGTGGTAATTTTAAAGTTAC
AAAAGGAAAAGCTAAAAAAGGTGCCTGGAATATTGGTGAACAGAAATCAATACT
GAGTCCTCTTTATGCATTTGCATCAGAGGCTGCTCGTGTTGTACGATCAATTTTCT
CCCGCACTCTTGAAACTGCTCAAAATTCTGTGCGTGTTTTACAGAAGGCCGCTAT
AACAATACTAGATGGAATTTCACAGTATTCACTGAGACTCATTGATGCTATGATG
TTCACATCTGATTTGGCTACTAACAATCTAGTTGTAATGGCCTACATTACAGGTG
GTGTTGTTCAGTTGACTTCGCAGTGGCTAACTAACATCTTTGGCACTGTTTATGAA
AAACTCAAACCCGTCCTTGATTGGCTTGAAGAGAAGTTTAAGGAAGGTGTAGAG
TTTCTTAGAGACGGTTGGGAAATTGTTAAATTTATCTCAACCTGTGCTTGTGAAAT
TGTCGGTGGACAAATTGTCACCTGTGCAAAGGAAATTAAGGAGAGTGTTCAGAC
ATTCTTTAAGCTTGTAAATAAATTTTTGGCTTTGTGTGCTGACTCTATCATTATTG
GTGGAGCTAAACTTAAAGCCTTGAATTTAGGTGAAACATTTGTCACGCACTCAAA
GGGATTGTACAGAAAGTGTGTTAAATCCAGAGAAGAAACTGGCCTACTCATGCC
TCTAAAAGCCCCAAAAGAAATTATCTTCTTAGAGGGAGAAACACTTCCCACAGA
AGTGTTAACAGAGGAAGTTGTCTTGAAAACTGGTGATTTACAACCATTAGAACA
ACCTACTAGTGAAGCTGTTGAAGCTCCATTGGTTGGTACACCAGTTTGTATTAAC
GGGCTTATGTTGCTCGAAATCAAAGACACAGAAAAGTACTGTGCCCTTGCACCTA
ATATGATGGTAACAAACAATACCTTCACACTCAAAGGCGGTGCACCAACAAAGG
TTACTTTTGGTGATGACACTGTGATAGAAGTGCAAGGTTACAAGAGTGTGAATAT
CACTTTTGAACTTGATGAAAGGATTGATAAAGTACTTAATGAGAAGTGCTCTGCC
TATACAGTTGAACTCGGTACAGAAGTAAATGAGTTCGCCTGTGTTGTGGCAGATG
CTGTCATAAAAACTTTGCAACCAGTATCTGAATTACTTACACCACTGGGCATTGA
TTTAGATGAGTGGAGTATGGCTACATACTACTTATTTGATGAGTCTGGTGAGTTT
AAATTGGCTTCACATATGTATTGTTCTTTCTACCCTCCAGATGAGGATGAAGAAG
AAGGTGATTGTGAAGAAGAAGAGTTTGAGCCATCAACTCAATATGAGTATGGTA
CTGAAGATGATTACCAAGGTAAACCTTTGGAATTTGGTGCCACTTCTGCTGCTCT
TCAACCTGAAGAAGAGCAAGAAGAAGATTGGTTAGATGATGATAGTCAACAAAC
TGTTGGTCAACAAGACGGCAGTGAGGACAATCAGACAACTACTATTCAAACAAT
TGTTGAGGTTCAACCTCAATTAGAGATGGAACTTACACCAGTTGTTCAGACTATT
GAAGTGAATAGTTTTAGTGGTTATTTAAAACTTACTGACAATGTATACATTAAAA
ATGCAGACATTGTGGAAGAAGCTAAAAAGGTAAAACCAACAGTGGTTGTTAATG
CAGCCAATGTTTACCTTAAACATGGAGGAGGTGTTGCAGGAGCCTTAAATAAGG
CTACTAACAATGCCATGCAAGTTGAATCTGATGATTACATAGCTACTAATGGACC
ACTTAAAGTGGGTGGTAGTTGTGTTTTAAGCGGACACAATCTTGCTAAACACTGT
CTTCATGTTGTCGGCCCAAATGTTAACAAAGGTGAAGACATTCAACTTCTTAAGA
GTGCTTATGAAAATTTTAATCAGCACGAAGTTCTACTTGCACCATTATTATCAGCT
GGTATTTTTGGTGCTGACCCTATACATTCTTTAAGAGTTTGTGTAGATACTGTTCG
CACAAATGTCTACTTAGCTGTCTTTGATAAAAATCTCTATGACAAACTTGTTTCAA
GCTTTTTGGAAATGAAGAGTGAAAAGCAAGTTGAACAAAAGATCGCTGAGATTC
CTAAAGAGGAAGTTAAGCCATTTATAACTGAAAGTAAACCTTCAGTTGAACAGA
GAAAACAAGATGATAAGAAAATCAAAGCTTGTGTTGAAGAAGTTACAACAACTC
TGGAAGAAACTAAGTTCCTCACAGAAAACTTGTTACTTTATATTGACATTAATGG
CAATCTTCATCCAGATTCTGCCACTCTTGTTAGTGACATTGACATCACTTTCTTAA
AGAAAGATGCTCCATATATAGTGGGTGATGTTGTTCAAGAGGGTGTTTTAACTGC
TGTGGTTATACCTACTAAAAAGGCTGGTGGCACTACTGAAATGCTAGCGAAAGC
TTTGAGAAAAGTGCCAACAGACAATTATATAACCACTTACCCGGGTCAGGGTTTA
AATGGTTACACTGTAGAGGAGGCAAAGACAGTGCTTAAAAAGTGTAAAAGTGCC
TTTTACATTCTACCATCTATTATCTCTAATGAGAAGCAAGAAATTCTTGGAACTGT
TTCTTGGAATTTGCGAGAAATGCTTGCACATGCAGAAGAAACACGCAAATTAAT
GCCTGTCTGTGTGGAAACTAAAGCCATAGTTTCAACTATACAGCGTAAATATAAG
GGTATTAAAATACAAGAGGGTGTGGTTGATTATGGTGCTAGATTTTACTTTTACA
CCAGTAAAACAACTGTAGCGTCACTTATCAACACACTTAACGATCTAAATGAAA
CTCTTGTTACAATGCCACTTGGCTATGTAACACATGGCTTAAATTTGGAAGAAGC
TGCTCGGTATATGAGATCTCTCAAAGTGCCAGCTACAGTTTCTGTTTCTTCACCTG
ATGCTGTTACAGCGTATAATGGTTATCTTACTTCTTCTTCTAAAACACCTGAAGAA
CATTTTATTGAAACCATCTCACTTGCTGGTTCCTATAAAGATTGGTCCTATTCTGG
ACAATCTACACAACTAGGTATAGAATTTCTTAAGAGAGGTGATAAAAGTGTATAT
TACACTAGTAATCCTACCACATTCCACCTAGATGGTGAAGTTATCACCTTTGACA
ATCTTAAGACACTTCTTTCTTTGAGAGAAGTGAGGACTATTAAGGTGTTTACAAC
AGTAGACAACATTAACCTCCACACGCAAGTTGTGGACATGTCAATGACATATGG
ACAACAGTTTGGTCCAACTTATTTGGATGGAGCTGATGTTACTAAAATAAAACCT
CATAATTCACATGAAGGTAAAACATTTTATGTTTTACCTAATGATGACACTCTAC
GTGTTGAGGCTTTTGAGTACTACCACACAACTGATCCTAGTTTTCTGGGTAGGTA
CATGTCAGCATTAAATCACACTAAAAAGTGGAAATACCCACAAGTTAATGGTTT
AACTTCTATTAAATGGGCAGATAACAACTGTTATCTTGCCACTGCATTGTTAACA
CTCCAACAAATAGAGTTGAAGTTTAATCCACCTGCTCTACAAGATGCTTATTACA
GAGCAAGGGCTGGTGAAGCTGCTAACTTTTGTGCACTTATCTTAGCCTACTGTAA
TAAGACAGTAGGTGAGTTAGGTGATGTTAGAGAAACAATGAGTTACTTGTTTCAA
CATGCCAATTTAGATTCTTGCAAAAGAGTCTTGAACGTGGTGTGTAAAACTTGTG
GACAACAGCAGACAACCCTTAAGGGTGTAGAAGCTGTTATGTACATGGGCACAC
TTTCTTATGAACAATTTAAGAAAGGTGTTCAGATACCTTGTACGTGTGGTAAACA
AGCTACAAAATATCTAGTACAACAGGAGTCACCTTTTGTTATGATGTCAGCACCA
CCTGCTCAGTATGAACTTAAGCATGGTACATTTACTTGTGCTAGTGAGTACACTG
GTAATTACCAGTGTGGTCACTATAAACATATAACTTCTAAAGAAACTTTGTATTG
CATAGACGGTGCTTTACTTACAAAGTCCTCAGAATACAAAGGTCCTATTACGGAT
GTTTTCTACAAAGAAAACAGTTACACAACAACCATAAAACCAGTTACTTATAAAT
TGGATGGTGTTGTTTGTACAGAAATTGACCCTAAGTTGGACAATTATTATAAGAA
AGACAATTCTTATTTCACAGAGCAACCAATTGATCTTGTACCAAACCAACCATAT
CCAAACGCAAGCTTCGATAATTTTAAGTTTGTATGTGATAATATCAAATTTGCTG
ATGATTTAAACCAGTTAACTGGTTATAAGAAACCTGCTTCAAGAGAGCTTAAAGT
TACATTTTTCCCTGACTTAAATGGTGATGTGGTGGCTATTGATTATAAACACTACA
CACCCTCTTTTAAGAAAGGAGCTAAATTGTTACATAAACCTATTGTTTGGCATGT
TAACAATGCAACTAATAAAGCCACGTATAAACCAAATACCTGGTGTATACGTTGT
CTTTGGAGCACAAAACCAGTTGAAACATCAAATTCGTTTGATGTACTGAAGTCAG
AGGACGCGCAGGGAATGGATAATCTTGCCTGCGAAGATCTAAAACCAGTCTCTG
AAGAAGTAGTGGAAAATCCTACCATACAGAAAGACGTTCTTGAGTGTAATGTGA
AAACTACCGAAGTTGTAGGAGACATTATACTTAAACCAGCAAATAATAGTTTAA
AAATTACAGAAGAGGTTGGCCACACAGATCTAATGGCTGCTTATGTAGACAATT
CTAGTCTTACTATTAAGAAACCTAATGAATTATCTAGAGTATTAGGTTTGAAAAC
CCTTGCTACTCATGGTTTAGCTGCTGTTAATAGTGTCCCTTGGGATACTATAGCTA
ATTATGCTAAGCCTTTTCTTAACAAAGTTGTTAGTACAACTACTAACATAGTTAC
ACGGTGTTTAAACCGTGTTTGTACTAATTATATGCCTTATTTCTTTACTTTATTGCT
ACAATTGTGTACTTTTACTAGAAGTACAAATTCTAGAATTAAAGCATCTATGCCG
ACTACTATAGCAAAGAATACTGTTAAGAGTGTCGGTAAATTTTGTCTAGAGGCTT
CATTTAATTATTTGAAGTCACCTAATTTTTCTAAACTGATAAATATTATAATTTGG
TTTTTACTATTAAGTGTTTGCCTAGGTTCGTTAATCTACTCGACCGCTGCTTTAGG
TGTTTTAATGTCGAATTTAGGCATGCCTTCGTACTGTACTGGTTACAGAGAAGGC
TATTTGAACTCGACTAATGTCACTATTGCAACCTACTGTACTGGTTCGATACCTTG
TTCGGTTTGTCTTTCGGGTTTAGATTCGTTAGACACCTATCCTTCGTTAGAAACTA
TACAAATTACCATTTCGTCGTTTAAATGGGATTTAACTGCTTTTGGCTTAGTTGCA
GAGTGGTTTTTGGCATATATTCTTTTCACTAGGTTTTTCTATGTACTTGGATTGGC
TGCAATCATGCAATTGTTTTTCTCGTATTTTGCAGTACATTTTATTTCGAATTCGT
GGCTTATGTGGTTAATAATTAATCTTGTACAAATGGCCCCGATTTCGGCTATGGTT
AGAATGTACATCTTCTTTGCATCGTTTTATTATGTATGGAAATCGTATGTGCATGT
TGTAGACGGTTGTAATTCGTCGACTTGTATGATGTGTTACAAACGTAATAGAGCA
ACAAGAGTCGAATGTACAACTATTGTTAATGGTGTTAGAAGGTCGTTTTATGTCT
ATGCTAATGGAGGTAAAGGCTTTTGCAAACTACACAATTGGAATTGTGTTAATTG
TGATACATTCTGTGCTGGTTCGACATTTATTTCGGATGAAGTTGCGAGAGACTTG
TCGCTACAGTTTAAAAGACCAATAAATCCTACTGACCAGTCGTCGTACATCGTTG
ATTCGGTTACAGTGAAGAATGGTTCGATCCATCTTTACTTTGATAAAGCTGGTCA
AAAGACTTATGAAAGACATTCGCTCTCGCATTTTGTTAACTTAGACAACCTGAGA
GCTAATAACACTAAAGGTTCGTTGCCTATTAATGTTATAGTTTTTGATGGTAAATC
GAAATGTGAAGAATCGTCGGCAAAATCGGCGTCGGTTTACTACTCGCAGCTTATG
TGTCAACCTATACTGTTACTAGATCAGGCATTAGTGTCGGATGTTGGTGATTCGG
CGGAAGTTGCAGTTAAAATGTTTGATGCTTACGTTAATACGTTTTCGTCGACTTTT
AACGTACCAATGGAAAAACTCAAAACACTAGTTGCAACTGCAGAAGCTGAACTT
GCAAAGAATGTGTCGTTAGACAATGTCTTATCGACTTTTATTTCGGCAGCTCGGC
AAGGGTTTGTTGATTCGGATGTAGAAACTAAAGATGTTGTTGAATGTCTTAAATT
GTCGCATCAATCGGACATAGAAGTTACTGGCGATTCGTGTAATAACTATATGCTC
ACCTATAACAAAGTTGAAAACATGACACCCCGTGACCTTGGTGCTTGTATTGACT
GTTCGGCGCGTCATATTAATGCGCAGGTAGCAAAATCGCACAACATTGCTTTGAT
ATGGAACGTTAAAGATTTCATGTCGTTGTCGGAACAACTACGAAAACAAATACG
TTCGGCTGCTAAAAAGAATAACTTACCTTTTAAGTTGACATGTGCAACTACTAGA
CAAGTTGTTAATGTTGTAACAACAAAGATAGCACTTAAGGGTGGTAAAATTGTTA
ATAATTGGTTGAAGCAGTTAATTAAA
SEQ ID NO: 47 - Clone pCC1-4K-SARS-COV-2-160-2. Only fragment 2 has been
deoptimized:
GGGTCCCACGTGCTAGCGCTAACATAGGTTGTAACCATACAGGTGTTG
TTGGAGAAGGTTCCGAAGGTCTTAATGACAACCTTCTTGAAATACTCCAAAAAG
AGAAAGTCAACATCAATATTGTTGGTGACTTTAAACTTAATGAAGAGATCGCCAT
TATTTTGGCATCTTTTTCTGCTTCCACAAGTGCTTTTGTGGAAACTGTGAAAGGTT
TGGATTATAAAGCATTCAAACAAATTGTTGAATCCTGTGGTAATTTTAAAGTTAC
AAAAGGAAAAGCTAAAAAAGGTGCCTGGAATATTGGTGAACAGAAATCAATACT
GAGTCCTCTTTATGCATTTGCATCAGAGGCTGCTCGTGTTGTACGATCAATTTTCT
CCCGCACTCTTGAAACTGCTCAAAATTCTGTGCGTGTTTTACAGAAGGCCGCTAT
AACAATACTAGATGGAATTTCACAGTATTCACTGAGACTCATTGATGCTATGATG
TTCACATCTGATTTGGCTACTAACAATCTAGTTGTAATGGCCTACATTACAGGTG
GTGTTGTTCAGTTGACTTCGCAGTGGCTAACTAACATCTTTGGCACTGTTTATGAA
AAACTCAAACCCGTCCTTGATTGGCTTGAAGAGAAGTTTAAGGAAGGTGTAGAG
TTTCTTAGAGACGGTTGGGAAATTGTTAAATTTATCTCAACCTGTGCTTGTGAAAT
TGTCGGTGGACAAATTGTCACCTGTGCAAAGGAAATTAAGGAGAGTGTTCAGAC
ATTCTTTAAGCTTGTAAATAAATTTTTGGCTTTGTGTGCTGACTCTATCATTATTG
GTGGAGCTAAACTTAAAGCCTTGAATTTAGGTGAAACATTTGTCACGCACTCAAA
GGGATTGTACAGAAAGTGTGTTAAATCCAGAGAAGAAACTGGCCTACTCATGCC
TCTAAAAGCCCCAAAAGAAATTATCTTCTTAGAGGGAGAAACACTTCCCACAGA
AGTGTTAACAGAGGAAGTTGTCTTGAAAACTGGTGATTTACAACCATTAGAACA
ACCTACTAGTGAAGCTGTTGAAGCTCCATTGGTTGGTACACCAGTTTGTATTAAC
GGGCTTATGTTGCTCGAAATCAAAGACACAGAAAAGTACTGTGCCCTTGCACCTA
ATATGATGGTAACAAACAATACCTTCACACTCAAAGGCGGTGCACCAACAAAGG
TTACTTTTGGTGATGACACTGTGATAGAAGTGCAAGGTTACAAGAGTGTGAATAT
CACTTTTGAACTTGATGAAAGGATTGATAAAGTACTTAATGAGAAGTGCTCTGCC
TATACAGTTGAACTCGGTACAGAAGTAAATGAGTTCGCCTGTGTTGTGGCAGATG
CTGTCATAAAAACTTTGCAACCAGTATCTGAATTACTTACACCACTGGGCATTGA
TTTAGATGAGTGGAGTATGGCTACATACTACTTATTTGATGAGTCTGGTGAGTTT
AAATTGGCTTCACATATGTATTGTTCTTTCTACCCTCCAGATGAGGATGAAGAAG
AAGGTGATTGTGAAGAAGAAGAGTTTGAGCCATCAACTCAATATGAGTATGGTA
CTGAAGATGATTACCAAGGTAAACCTTTGGAATTTGGTGCCACTTCTGCTGCTCT
TCAACCTGAAGAAGAGCAAGAAGAAGATTGGTTAGATGATGATAGTCAACAAAC
TGTTGGTCAACAAGACGGCAGTGAGGACAATCAGACAACTACTATTCAAACAAT
TGTTGAGGTTCAACCTCAATTAGAGATGGAACTTACACCAGTTGTTCAGACTATT
GAAGTGAATAGTTTTAGTGGTTATTTAAAACTTACTGACAATGTATACATTAAAA
ATGCAGACATTGTGGAAGAAGCTAAAAAGGTAAAACCAACAGTGGTTGTTAATG
CAGCCAATGTTTACCTTAAACATGGAGGAGGTGTTGCAGGAGCCTTAAATAAGG
CTACTAACAATGCCATGCAAGTTGAATCTGATGATTACATAGCTACTAATGGACC
ACTTAAAGTGGGTGGTAGTTGTGTTTTAAGCGGACACAATCTTGCTAAACACTGT
CTTCATGTTGTCGGCCCAAATGTTAACAAAGGTGAAGACATTCAACTTCTTAAGA
GTGCTTATGAAAATTTTAATCAGCACGAAGTTCTACTTGCACCATTATTATCAGCT
GGTATTTTTGGTGCTGACCCTATACATTCTTTAAGAGTTTGTGTAGATACTGTTCG
CACAAATGTCTACTTAGCTGTCTTTGATAAAAATCTCTATGACAAACTTGTTTCAA
GCTTTTTGGAAATGAAGAGTGAAAAGCAAGTTGAACAAAAGATCGCTGAGATTC
CTAAAGAGGAAGTTAAGCCATTTATAACTGAAAGTAAACCTTCAGTTGAACAGA
GAAAACAAGATGATAAGAAAATCAAAGCTTGTGTTGAAGAAGTTACAACAACTC
TGGAAGAAACTAAGTTCCTCACAGAAAACTTGTTACTTTATATTGACATTAATGG
CAATCTTCATCCAGATTCTGCCACTCTTGTTAGTGACATTGACATCACTTTCTTAA
AGAAAGATGCTCCATATATAGTGGGTGATGTTGTTCAAGAGGGTGTTTTAACTGC
TGTGGTTATACCTACTAAAAAGGCTGGTGGCACTACTGAAATGCTAGCGAAAGC
TTTGAGAAAAGTGCCAACAGACAATTATATAACCACTTACCCGGGTCAGGGTTTA
AATGGTTACACTGTAGAGGAGGCAAAGACAGTGCTTAAAAAGTGTAAATCGGCC
TTTTACATTCTACCATCGATTATCTCGAATGAGAAGCAAGAAATTCTTGGAACTG
TTTCGTGGAATTTGCGAGAAATGCTTGCACATGCAGAAGAAACACGCAAATTAA
TGCCTGTCTGTGTGGAAACTAAAGCCATAGTTTCGACTATACAGCGTAAATATAA
GGGTATTAAAATACAAGAGGGTGTGGTTGATTATGGTGCTAGATTTTACTTTTAC
ACCTCGAAAACAACTGTAGCGTCGCTTATCAACACACTTAACGATCTAAATGAA
ACTCTTGTTACAATGCCACTTGGCTATGTAACACATGGCTTAAATTTGGAAGAAG
CTGCTCGGTATATGAGATCGCTCAAAGTGCCAGCTACAGTTTCGGTTTCGTCGCC
TGATGCTGTTACAGCGTATAATGGTTATCTTACTTCGTCGTCGAAAACACCTGAA
GAACATTTTATTGAAACCATCTCGCTTGCTGGTTCGTATAAAGATTGGTCGTATTC
GGGACAATCGACACAACTAGGTATAGAATTTCTTAAGAGAGGTGATAAATCGGT
ATATTACACTTCGAATCCTACCACATTCCACCTAGATGGTGAAGTTATCACCTTT
GACAATCTTAAGACACTTCTTTCGTTGAGAGAAGTGAGGACTATTAAGGTGTTTA
CAACAGTAGACAACATTAACCTCCACACGCAAGTTGTGGACATGTCGATGACAT
ATGGACAACAGTTTGGTCCAACTTATTTGGATGGAGCTGATGTTACTAAAATAAA
ACCTCATAATTCGCATGAAGGTAAAACATTTTATGTTTTACCTAATGATGACACT
CTACGTGTTGAGGCTTTTGAGTACTACCACACAACTGATCCTTCGTTTCTGGGTA
GGTACATGTCGGCATTAAATCACACTAAAAAGTGGAAATACCCACAAGTTAATG
GTTTAACTTCGATTAAATGGGCAGATAACAACTGTTATCTTGCCACTGCATTGTT
AACACTCCAACAAATAGAGTTGAAGTTTAATCCACCTGCTCTACAAGATGCTTAT
TACAGAGCAAGGGCTGGTGAAGCTGCTAACTTTTGTGCACTTATCTTAGCCTACT
GTAATAAGACAGTAGGTGAGTTAGGTGATGTTAGAGAAACAATGTCGTACTTGTT
TCAACATGCCAATTTAGATTCGTGCAAAAGAGTCTTGAACGTGGTGTGTAAAACT
TGTGGACAACAGCAGACAACCCTTAAGGGTGTAGAAGCTGTTATGTACATGGGC
ACACTTTCGTATGAACAATTTAAGAAAGGTGTTCAGATACCTTGTACGTGTGGTA
AACAAGCTACAAAATATCTAGTACAACAGGAGTCGCCTTTTGTTATGATGTCGGC
ACCACCTGCTCAGTATGAACTTAAGCATGGTACATTTACTTGTGCTTCGGAGTAC
ACTGGTAATTACCAGTGTGGTCACTATAAACATATAACTTCGAAAGAAACTTTGT
ATTGCATAGACGGTGCTTTACTTACAAAGTCGTCGGAATACAAAGGTCCTATTAC
GGATGTTTTCTACAAAGAAAACTCGTACACAACAACCATAAAACCAGTTACTTAT
AAATTGGATGGTGTTGTTTGTACAGAAATTGACCCTAAGTTGGACAATTATTATA
AGAAAGACAATTCGTATTTCACAGAGCAACCAATTGATCTTGTACCAAACCAAC
CATATCCAAACGCATCGTTCGATAATTTTAAGTTTGTATGTGATAATATCAAATTT
GCTGATGATTTAAACCAGTTAACTGGTTATAAGAAACCTGCTTCGAGAGAGCTTA
AAGTTACATTTTTCCCTGACTTAAATGGTGATGTGGTGGCTATTGATTATAAACA
CTACACACCCTCGTTTAAGAAAGGAGCTAAATTGTTACATAAACCTATTGTTTGG
CATGTTAACAATGCAACTAATAAAGCCACGTATAAACCAAATACCTGGTGTATA
CGTTGTCTTTGGTCGACAAAACCAGTTGAAACATCGAATTCGTTTGATGTACTGA
AGTCGGAGGACGCGCAGGGAATGGATAATCTTGCCTGCGAAGATCTAAAACCAG
TCTCGGAAGAAGTAGTGGAAAATCCTACCATACAGAAAGACGTTCTTGAGTGTA
ATGTGAAAACTACCGAAGTTGTAGGAGACATTATACTTAAACCAGCAAATAATT
CGTTAAAAATTACAGAAGAGGTTGGCCACACAGATCTAATGGCTGCTTATGTAG
ACAATTCGTCGCTTACTATTAAGAAACCTAATGAATTATCGAGAGTATTAGGTTT
GAAAACCCTTGCTACTCATGGTTTAGCTGCTGTTAATTCGGTCCCTTGGGATACT
ATAGCTAATTATGCTAAGCCTTTTCTTAACAAAGTTGTTTCGACAACTACTAACAT
AGTTACACGGTGTTTAAACCGTGTTTGTACTAATTATATGCCTTATTTCTTTACTT
TATTGCTACAATTGTGTACTTTTACTAGATCGACAAATTCGAGAATTAAAGCATC
GATGCCGACTACTATAGCAAAGAATACTGTTAAGTCGGTCGGTAAATTTTGTCTA
GAGGCTTCGTTTAATTATTTGAAGTCGCCTAATTTTTCGAAACTGATAAATATTAT
AATTTGGTTTTTACTATTATCGGTTTGCCTAGGTTCTTTAATCTACTCAACCGCTG
CTTTAGGTGTTTTAATGTCTAATTTAGGCATGCCTTCTTACTGTACTGGTTACAGA
GAAGGCTATTTGAACTCTACTAATGTCACTATTGCAACCTACTGTACTGGTTCTAT
ACCTTGTAGTGTTTGTCTTAGTGGTTTAGATTCTTTAGACACCTATCCTTCTTTAG
AAACTATACAAATTACCATTTCATCTTTTAAATGGGATTTAACTGCTTTTGGCTTA
GTTGCAGAGTGGTTTTTGGCATATATTCTTTTCACTAGGTTTTTCTATGTACTTGG
ATTGGCTGCAATCATGCAATTGTTTTTCAGCTATTTTGCAGTACATTTTATTAGTA
ATTCTTGGCTTATGTGGTTAATAATTAATCTTGTACAAATGGCCCCGATTTCAGCT
ATGGTTAGAATGTACATCTTCTTTGCATCATTTTATTATGTATGGAAAAGTTATGT
GCATGTTGTAGACGGTTGTAATTCATCAACTTGTATGATGTGTTACAAACGTAAT
AGAGCAACAAGAGTCGAATGTACAACTATTGTTAATGGTGTTAGAAGGTCCTTTT
ATGTCTATGCTAATGGAGGTAAAGGCTTTTGCAAACTACACAATTGGAATTGTGT
TAATTGTGATACATTCTGTGCTGGTAGTACATTTATTAGTGATGAAGTTGCGAGA
GACTTGTCACTACAGTTTAAAAGACCAATAAATCCTACTGACCAGTCTTCTTACA
TCGTTGATAGTGTTACAGTGAAGAATGGTTCCATCCATCTTTACTTTGATAAAGCT
GGTCAAAAGACTTATGAAAGACATTCTCTCTCTCATTTTGTTAACTTAGACAACC
TGAGAGCTAATAACACTAAAGGTTCATTGCCTATTAATGTTATAGTTTTTGATGG
TAAATCAAAATGTGAAGAATCATCTGCAAAATCAGCGTCTGTTTACTACAGTCAG
CTTATGTGTCAACCTATACTGTTACTAGATCAGGCATTAGTGTCTGATGTTGGTGA
TAGTGCGGAAGTTGCAGTTAAAATGTTTGATGCTTACGTTAATACGTTTTCATCA
ACTTTTAACGTACCAATGGAAAAACTCAAAACACTAGTTGCAACTGCAGAAGCT
GAACTTGCAAAGAATGTGTCCTTAGACAATGTCTTATCTACTTTTATTTCAGCAGC
TCGGCAAGGGTTTGTTGATTCAGATGTAGAAACTAAAGATGTTGTTGAATGTCTT
AAATTGTCACATCAATCTGACATAGAAGTTACTGGCGATAGTTGTAATAACTATA
TGCTCACCTATAACAAAGTTGAAAACATGACACCCCGTGACCTTGGTGCTTGTAT
TGACTGTAGTGCGCGTCATATTAATGCGCAGGTAGCAAAAAGTCACAACATTGCT
TTGATATGGAACGTTAAAGATTTCATGTCATTGTCTGAACAACTACGAAAACAAA
TACGTAGTGCTGCTAAAAAGAATAACTTACCTTTTAAGTTGACATGTGCAACTAC
TAGACAAGTTGTTAATGTTGTAACAACAAAGATAGCACTTAAGGGTGGTAAAAT
TGTTAATAATTGGTTGAAGCAGTTAATTAAA
SEQ ID NO: 48 - Clone pCC1-4K-SARS-COV-2-160-3. Only fragment 1 has been
deoptimized:
GGGTCCCACGTGCTTCGGCTAACATAGGTTGTAACCATACAGGTGTTG
TTGGAGAAGGTTCGGAAGGTCTTAATGACAACCTTCTTGAAATACTCCAAAAAG
AGAAAGTCAACATCAATATTGTTGGTGACTTTAAACTTAATGAAGAGATCGCCAT
TATTTTGGCATCGTTTTCGGCTTCGACATCGGCTTTTGTGGAAACTGTGAAAGGTT
TGGATTATAAAGCATTCAAACAAATTGTTGAATCGTGTGGTAATTTTAAAGTTAC
AAAAGGAAAAGCTAAAAAAGGTGCCTGGAATATTGGTGAACAGAAATCGATACT
GTCGCCTCTTTATGCATTTGCATCGGAGGCTGCTCGTGTTGTACGATCGATTTTCT
CGCGCACTCTTGAAACTGCTCAAAATTCGGTGCGTGTTTTACAGAAGGCCGCTAT
AACAATACTAGATGGAATTTCGCAGTATTCGCTGAGACTCATTGATGCTATGATG
TTCACATCGGATTTGGCTACTAACAATCTAGTTGTAATGGCCTACATTACAGGTG
GTGTTGTTCAGTTGACTTCGCAGTGGCTAACTAACATCTTTGGCACTGTTTATGAA
AAACTCAAACCCGTCCTTGATTGGCTTGAAGAGAAGTTTAAGGAAGGTGTAGAG
TTTCTTAGAGACGGTTGGGAAATTGTTAAATTTATCTCGACCTGTGCTTGTGAAAT
TGTCGGTGGACAAATTGTCACCTGTGCAAAGGAAATTAAGGAGTCGGTTCAGAC
ATTCTTTAAGCTTGTAAATAAATTTTTGGCTTTGTGTGCTGACTCGATCATTATTG
GTGGAGCTAAACTTAAAGCCTTGAATTTAGGTGAAACATTTGTCACGCACTCGAA
GGGATTGTACAGAAAGTGTGTTAAATCGAGAGAAGAAACTGGCCTACTCATGCC
TCTAAAAGCCCCAAAAGAAATTATCTTCTTAGAGGGAGAAACACTTCCCACAGA
AGTGTTAACAGAGGAAGTTGTCTTGAAAACTGGTGATTTACAACCATTAGAACA
ACCTACTTCGGAAGCTGTTGAAGCTCCATTGGTTGGTACACCAGTTTGTATTAAC
GGGCTTATGTTGCTCGAAATCAAAGACACAGAAAAGTACTGTGCCCTTGCACCTA
ATATGATGGTAACAAACAATACCTTCACACTCAAAGGCGGTGCACCAACAAAGG
TTACTTTTGGTGATGACACTGTGATAGAAGTGCAAGGTTACAAGTCGGTGAATAT
CACTTTTGAACTTGATGAAAGGATTGATAAAGTACTTAATGAGAAGTGCTCGGCC
TATACAGTTGAACTCGGTACAGAAGTAAATGAGTTCGCCTGTGTTGTGGCAGATG
CTGTCATAAAAACTTTGCAACCAGTATCGGAATTACTTACACCACTGGGCATTGA
TTTAGATGAGTGGTCGATGGCTACATACTACTTATTTGATGAGTCGGGTGAGTTT
AAATTGGCTTCGCATATGTATTGTTCGTTCTACCCTCCAGATGAGGATGAAGAAG
AAGGTGATTGTGAAGAAGAAGAGTTTGAGCCATCGACTCAATATGAGTATGGTA
CTGAAGATGATTACCAAGGTAAACCTTTGGAATTTGGTGCCACTTCGGCTGCTCT
TCAACCTGAAGAAGAGCAAGAAGAAGATTGGTTAGATGATGATTCGCAACAAAC
TGTTGGTCAACAAGACGGCTCGGAGGACAATCAGACAACTACTATTCAAACAAT
TGTTGAGGTTCAACCTCAATTAGAGATGGAACTTACACCAGTTGTTCAGACTATT
GAAGTGAATTCGTTTTCGGGTTATTTAAAACTTACTGACAATGTATACATTAAAA
ATGCAGACATTGTGGAAGAAGCTAAAAAGGTAAAACCAACAGTGGTTGTTAATG
CAGCCAATGTTTACCTTAAACATGGAGGAGGTGTTGCAGGAGCCTTAAATAAGG
CTACTAACAATGCCATGCAAGTTGAATCGGATGATTACATAGCTACTAATGGACC
ACTTAAAGTGGGTGGTTCGTGTGTTTTATCGGGACACAATCTTGCTAAACACTGT
CTTCATGTTGTCGGCCCAAATGTTAACAAAGGTGAAGACATTCAACTTCTTAAGT
CGGCTTATGAAAATTTTAATCAGCACGAAGTTCTACTTGCACCATTATTATCGGC
TGGTATTTTTGGTGCTGACCCTATACATTCGTTAAGAGTTTGTGTAGATACTGTTC
GCACAAATGTCTACTTAGCTGTCTTTGATAAAAATCTCTATGACAAACTTGTTTCG
TCGTTTTTGGAAATGAAGTCGGAAAAGCAAGTTGAACAAAAGATCGCTGAGATT
CCTAAAGAGGAAGTTAAGCCATTTATAACTGAATCGAAACCTTCGGTTGAACAG
AGAAAACAAGATGATAAGAAAATCAAAGCTTGTGTTGAAGAAGTTACAACAACT
CTGGAAGAAACTAAGTTCCTCACAGAAAACTTGTTACTTTATATTGACATTAATG
GCAATCTTCATCCAGATTCGGCCACTCTTGTTTCGGACATTGACATCACTTTCTTA
AAGAAAGATGCTCCATATATAGTGGGTGATGTTGTTCAAGAGGGTGTTTTAACTG
CTGTGGTTATACCTACTAAAAAGGCTGGTGGCACTACTGAAATGCTAGCGAAAG
CTTTGAGAAAAGTGCCAACAGACAATTATATAACCACTTACCCGGGTCAGGGTTT
AAATGGTTACACTGTAGAGGAGGCAAAGACAGTGCTTAAAAAGTGTAAAAGTGC
CTTTTACATTCTACCATCTATTATCTCTAATGAGAAGCAAGAAATTCTTGGAACTG
TTTCTTGGAATTTGCGAGAAATGCTTGCACATGCAGAAGAAACACGCAAATTAAT
GCCTGTCTGTGTGGAAACTAAAGCCATAGTTTCAACTATACAGCGTAAATATAAG
GGTATTAAAATACAAGAGGGTGTGGTTGATTATGGTGCTAGATTTTACTTTTACA
CCAGTAAAACAACTGTAGCGTCACTTATCAACACACTTAACGATCTAAATGAAA
CTCTTGTTACAATGCCACTTGGCTATGTAACACATGGCTTAAATTTGGAAGAAGC
TGCTCGGTATATGAGATCTCTCAAAGTGCCAGCTACAGTTTCTGTTTCTTCACCTG
ATGCTGTTACAGCGTATAATGGTTATCTTACTTCTTCTTCTAAAACACCTGAAGAA
CATTTTATTGAAACCATCTCACTTGCTGGTTCCTATAAAGATTGGTCCTATTCTGG
ACAATCTACACAACTAGGTATAGAATTTCTTAAGAGAGGTGATAAAAGTGTATAT
TACACTAGTAATCCTACCACATTCCACCTAGATGGTGAAGTTATCACCTTTGACA
ATCTTAAGACACTTCTTTCTTTGAGAGAAGTGAGGACTATTAAGGTGTTTACAAC
AGTAGACAACATTAACCTCCACACGCAAGTTGTGGACATGTCAATGACATATGG
ACAACAGTTTGGTCCAACTTATTTGGATGGAGCTGATGTTACTAAAATAAAACCT
CATAATTCACATGAAGGTAAAACATTTTATGTTTTACCTAATGATGACACTCTAC
GTGTTGAGGCTTTTGAGTACTACCACACAACTGATCCTAGTTTTCTGGGTAGGTA
CATGTCAGCATTAAATCACACTAAAAAGTGGAAATACCCACAAGTTAATGGTTT
AACTTCTATTAAATGGGCAGATAACAACTGTTATCTTGCCACTGCATTGTTAACA
CTCCAACAAATAGAGTTGAAGTTTAATCCACCTGCTCTACAAGATGCTTATTACA
GAGCAAGGGCTGGTGAAGCTGCTAACTTTTGTGCACTTATCTTAGCCTACTGTAA
TAAGACAGTAGGTGAGTTAGGTGATGTTAGAGAAACAATGAGTTACTTGTTTCAA
CATGCCAATTTAGATTCTTGCAAAAGAGTCTTGAACGTGGTGTGTAAAACTTGTG
GACAACAGCAGACAACCCTTAAGGGTGTAGAAGCTGTTATGTACATGGGCACAC
TTTCTTATGAACAATTTAAGAAAGGTGTTCAGATACCTTGTACGTGTGGTAAACA
AGCTACAAAATATCTAGTACAACAGGAGTCACCTTTTGTTATGATGTCAGCACCA
CCTGCTCAGTATGAACTTAAGCATGGTACATTTACTTGTGCTAGTGAGTACACTG
GTAATTACCAGTGTGGTCACTATAAACATATAACTTCTAAAGAAACTTTGTATTG
CATAGACGGTGCTTTACTTACAAAGTCCTCAGAATACAAAGGTCCTATTACGGAT
GTTTTCTACAAAGAAAACAGTTACACAACAACCATAAAACCAGTTACTTATAAAT
TGGATGGTGTTGTTTGTACAGAAATTGACCCTAAGTTGGACAATTATTATAAGAA
AGACAATTCTTATTTCACAGAGCAACCAATTGATCTTGTACCAAACCAACCATAT
CCAAACGCAAGCTTCGATAATTTTAAGTTTGTATGTGATAATATCAAATTTGCTG
ATGATTTAAACCAGTTAACTGGTTATAAGAAACCTGCTTCAAGAGAGCTTAAAGT
TACATTTTTCCCTGACTTAAATGGTGATGTGGTGGCTATTGATTATAAACACTACA
CACCCTCTTTTAAGAAAGGAGCTAAATTGTTACATAAACCTATTGTTTGGCATGT
TAACAATGCAACTAATAAAGCCACGTATAAACCAAATACCTGGTGTATACGTTGT
CTTTGGAGCACAAAACCAGTTGAAACATCAAATTCGTTTGATGTACTGAAGTCAG
AGGACGCGCAGGGAATGGATAATCTTGCCTGCGAAGATCTAAAACCAGTCTCTG
AAGAAGTAGTGGAAAATCCTACCATACAGAAAGACGTTCTTGAGTGTAATGTGA
AAACTACCGAAGTTGTAGGAGACATTATACTTAAACCAGCAAATAATAGTTTAA
AAATTACAGAAGAGGTTGGCCACACAGATCTAATGGCTGCTTATGTAGACAATT
CTAGTCTTACTATTAAGAAACCTAATGAATTATCTAGAGTATTAGGTTTGAAAAC
CCTTGCTACTCATGGTTTAGCTGCTGTTAATAGTGTCCCTTGGGATACTATAGCTA
ATTATGCTAAGCCTTTTCTTAACAAAGTTGTTAGTACAACTACTAACATAGTTAC
ACGGTGTTTAAACCGTGTTTGTACTAATTATATGCCTTATTTCTTTACTTTATTGCT
ACAATTGTGTACTTTTACTAGAAGTACAAATTCTAGAATTAAAGCATCTATGCCG
ACTACTATAGCAAAGAATACTGTTAAGAGTGTCGGTAAATTTTGTCTAGAGGCTT
CATTTAATTATTTGAAGTCACCTAATTTTTCTAAACTGATAAATATTATAATTTGG
TTTTTACTATTAAGTGTTTGCCTAGGTTCTTTAATCTACTCAACCGCTGCTTTAGG
TGTTTTAATGTCTAATTTAGGCATGCCTTCTTACTGTACTGGTTACAGAGAAGGCT
ATTTGAACTCTACTAATGTCACTATTGCAACCTACTGTACTGGTTCTATACCTTGT
AGTGTTTGTCTTAGTGGTTTAGATTCTTTAGACACCTATCCTTCTTTAGAAACTAT
ACAAATTACCATTTCATCTTTTAAATGGGATTTAACTGCTTTTGGCTTAGTTGCAG
AGTGGTTTTTGGCATATATTCTTTTCACTAGGTTTTTCTATGTACTTGGATTGGCT
GCAATCATGCAATTGTTTTTCAGCTATTTTGCAGTACATTTTATTAGTAATTCTTG
GCTTATGTGGTTAATAATTAATCTTGTACAAATGGCCCCGATTTCAGCTATGGTTA
GAATGTACATCTTCTTTGCATCATTTTATTATGTATGGAAAAGTTATGTGCATGTT
GTAGACGGTTGTAATTCATCAACTTGTATGATGTGTTACAAACGTAATAGAGCAA
CAAGAGTCGAATGTACAACTATTGTTAATGGTGTTAGAAGGTCCTTTTATGTCTA
TGCTAATGGAGGTAAAGGCTTTTGCAAACTACACAATTGGAATTGTGTTAATTGT
GATACATTCTGTGCTGGTAGTACATTTATTAGTGATGAAGTTGCGAGAGACTTGT
CACTACAGTTTAAAAGACCAATAAATCCTACTGACCAGTCTTCTTACATCGTTGA
TAGTGTTACAGTGAAGAATGGTTCCATCCATCTTTACTTTGATAAAGCTGGTCAA
AAGACTTATGAAAGACATTCTCTCTCTCATTTTGTTAACTTAGACAACCTGAGAG
CTAATAACACTAAAGGTTCATTGCCTATTAATGTTATAGTTTTTGATGGTAAATCA
AAATGTGAAGAATCATCTGCAAAATCAGCGTCTGTTTACTACAGTCAGCTTATGT
GTCAACCTATACTGTTACTAGATCAGGCATTAGTGTCTGATGTTGGTGATAGTGC
GGAAGTTGCAGTTAAAATGTTTGATGCTTACGTTAATACGTTTTCATCAACTTTTA
ACGTACCAATGGAAAAACTCAAAACACTAGTTGCAACTGCAGAAGCTGAACTTG
CAAAGAATGTGTCCTTAGACAATGTCTTATCTACTTTTATTTCAGCAGCTCGGCA
AGGGTTTGTTGATTCAGATGTAGAAACTAAAGATGTTGTTGAATGTCTTAAATTG
TCACATCAATCTGACATAGAAGTTACTGGCGATAGTTGTAATAACTATATGCTCA
CCTATAACAAAGTTGAAAACATGACACCCCGTGACCTTGGTGCTTGTATTGACTG
TAGTGCGCGTCATATTAATGCGCAGGTAGCAAAAAGTCACAACATTGCTTTGATA
TGGAACGTTAAAGATTTCATGTCATTGTCTGAACAACTACGAAAACAAATACGTA
GTGCTGCTAAAAAGAATAACTTACCTTTTAAGTTGACATGTGCAACTACTAGACA
AGTTGTTAATGTTGTAACAACAAAGATAGCACTTAAGGGTGGTAAAATTGTTAAT
AATTGGTTGAAGCAGTTAATTAAA
SEQ ID NO: 49 - Clone pCC1-4K-SARS-COV-2-160-4. Only fragments 1 and 2
have been deoptimized:
GGGTCCCACGTGCTTCGGCTAACATAGGTTGTAACCATACAGGTGTTG
TTGGAGAAGGTTCGGAAGGTCTTAATGACAACCTTCTTGAAATACTCCAAAAAG
AGAAAGTCAACATCAATATTGTTGGTGACTTTAAACTTAATGAAGAGATCGCCAT
TATTTTGGCATCGTTTTCGGCTTCGACATCGGCTTTTGTGGAAACTGTGAAAGGTT
TGGATTATAAAGCATTCAAACAAATTGTTGAATCGTGTGGTAATTTTAAAGTTAC
AAAAGGAAAAGCTAAAAAAGGTGCCTGGAATATTGGTGAACAGAAATCGATACT
GTCGCCTCTTTATGCATTTGCATCGGAGGCTGCTCGTGTTGTACGATCGATTTTCT
CGCGCACTCTTGAAACTGCTCAAAATTCGGTGCGTGTTTTACAGAAGGCCGCTAT
AACAATACTAGATGGAATTTCGCAGTATTCGCTGAGACTCATTGATGCTATGATG
TTCACATCGGATTTGGCTACTAACAATCTAGTTGTAATGGCCTACATTACAGGTG
GTGTTGTTCAGTTGACTTCGCAGTGGCTAACTAACATCTTTGGCACTGTTTATGAA
AAACTCAAACCCGTCCTTGATTGGCTTGAAGAGAAGTTTAAGGAAGGTGTAGAG
TTTCTTAGAGACGGTTGGGAAATTGTTAAATTTATCTCGACCTGTGCTTGTGAAAT
TGTCGGTGGACAAATTGTCACCTGTGCAAAGGAAATTAAGGAGTCGGTTCAGAC
ATTCTTTAAGCTTGTAAATAAATTTTTGGCTTTGTGTGCTGACTCGATCATTATTG
GTGGAGCTAAACTTAAAGCCTTGAATTTAGGTGAAACATTTGTCACGCACTCGAA
GGGATTGTACAGAAAGTGTGTTAAATCGAGAGAAGAAACTGGCCTACTCATGCC
TCTAAAAGCCCCAAAAGAAATTATCTTCTTAGAGGGAGAAACACTTCCCACAGA
AGTGTTAACAGAGGAAGTTGTCTTGAAAACTGGTGATTTACAACCATTAGAACA
ACCTACTTCGGAAGCTGTTGAAGCTCCATTGGTTGGTACACCAGTTTGTATTAAC
GGGCTTATGTTGCTCGAAATCAAAGACACAGAAAAGTACTGTGCCCTTGCACCTA
ATATGATGGTAACAAACAATACCTTCACACTCAAAGGCGGTGCACCAACAAAGG
TTACTTTTGGTGATGACACTGTGATAGAAGTGCAAGGTTACAAGTCGGTGAATAT
CACTTTTGAACTTGATGAAAGGATTGATAAAGTACTTAATGAGAAGTGCTCGGCC
TATACAGTTGAACTCGGTACAGAAGTAAATGAGTTCGCCTGTGTTGTGGCAGATG
CTGTCATAAAAACTTTGCAACCAGTATCGGAATTACTTACACCACTGGGCATTGA
TTTAGATGAGTGGTCGATGGCTACATACTACTTATTTGATGAGTCGGGTGAGTTT
AAATTGGCTTCGCATATGTATTGTTCGTTCTACCCTCCAGATGAGGATGAAGAAG
AAGGTGATTGTGAAGAAGAAGAGTTTGAGCCATCGACTCAATATGAGTATGGTA
CTGAAGATGATTACCAAGGTAAACCTTTGGAATTTGGTGCCACTTCGGCTGCTCT
TCAACCTGAAGAAGAGCAAGAAGAAGATTGGTTAGATGATGATTCGCAACAAAC
TGTTGGTCAACAAGACGGCTCGGAGGACAATCAGACAACTACTATTCAAACAAT
TGTTGAGGTTCAACCTCAATTAGAGATGGAACTTACACCAGTTGTTCAGACTATT
GAAGTGAATTCGTTTTCGGGTTATTTAAAACTTACTGACAATGTATACATTAAAA
ATGCAGACATTGTGGAAGAAGCTAAAAAGGTAAAACCAACAGTGGTTGTTAATG
CAGCCAATGTTTACCTTAAACATGGAGGAGGTGTTGCAGGAGCCTTAAATAAGG
CTACTAACAATGCCATGCAAGTTGAATCGGATGATTACATAGCTACTAATGGACC
ACTTAAAGTGGGTGGTTCGTGTGTTTTATCGGGACACAATCTTGCTAAACACTGT
CTTCATGTTGTCGGCCCAAATGTTAACAAAGGTGAAGACATTCAACTTCTTAAGT
CGGCTTATGAAAATTTTAATCAGCACGAAGTTCTACTTGCACCATTATTATCGGC
TGGTATTTTTGGTGCTGACCCTATACATTCGTTAAGAGTTTGTGTAGATACTGTTC
GCACAAATGTCTACTTAGCTGTCTTTGATAAAAATCTCTATGACAAACTTGTTTCG
TCGTTTTTGGAAATGAAGTCGGAAAAGCAAGTTGAACAAAAGATCGCTGAGATT
CCTAAAGAGGAAGTTAAGCCATTTATAACTGAATCGAAACCTTCGGTTGAACAG
AGAAAACAAGATGATAAGAAAATCAAAGCTTGTGTTGAAGAAGTTACAACAACT
CTGGAAGAAACTAAGTTCCTCACAGAAAACTTGTTACTTTATATTGACATTAATG
GCAATCTTCATCCAGATTCGGCCACTCTTGTTTCGGACATTGACATCACTTTCTTA
AAGAAAGATGCTCCATATATAGTGGGTGATGTTGTTCAAGAGGGTGTTTTAACTG
CTGTGGTTATACCTACTAAAAAGGCTGGTGGCACTACTGAAATGCTAGCGAAAG
CTTTGAGAAAAGTGCCAACAGACAATTATATAACCACTTACCCGGGTCAGGGTTT
AAATGGTTACACTGTAGAGGAGGCAAAGACAGTGCTTAAAAAGTGTAAATCGGC
CTTTTACATTCTACCATCGATTATCTCGAATGAGAAGCAAGAAATTCTTGGAACT
GTTTCGTGGAATTTGCGAGAAATGCTTGCACATGCAGAAGAAACACGCAAATTA
ATGCCTGTCTGTGTGGAAACTAAAGCCATAGTTTCGACTATACAGCGTAAATATA
AGGGTATTAAAATACAAGAGGGTGTGGTTGATTATGGTGCTAGATTTTACTTTTA
CACCTCGAAAACAACTGTAGCGTCGCTTATCAACACACTTAACGATCTAAATGAA
ACTCTTGTTACAATGCCACTTGGCTATGTAACACATGGCTTAAATTTGGAAGAAG
CTGCTCGGTATATGAGATCGCTCAAAGTGCCAGCTACAGTTTCGGTTTCGTCGCC
TGATGCTGTTACAGCGTATAATGGTTATCTTACTTCGTCGTCGAAAACACCTGAA
GAACATTTTATTGAAACCATCTCGCTTGCTGGTTCGTATAAAGATTGGTCGTATTC
GGGACAATCGACACAACTAGGTATAGAATTTCTTAAGAGAGGTGATAAATCGGT
ATATTACACTTCGAATCCTACCACATTCCACCTAGATGGTGAAGTTATCACCTTT
GACAATCTTAAGACACTTCTTTCGTTGAGAGAAGTGAGGACTATTAAGGTGTTTA
CAACAGTAGACAACATTAACCTCCACACGCAAGTTGTGGACATGTCGATGACAT
ATGGACAACAGTTTGGTCCAACTTATTTGGATGGAGCTGATGTTACTAAAATAAA
ACCTCATAATTCGCATGAAGGTAAAACATTTTATGTTTTACCTAATGATGACACT
CTACGTGTTGAGGCTTTTGAGTACTACCACACAACTGATCCTTCGTTTCTGGGTA
GGTACATGTCGGCATTAAATCACACTAAAAAGTGGAAATACCCACAAGTTAATG
GTTTAACTTCGATTAAATGGGCAGATAACAACTGTTATCTTGCCACTGCATTGTT
AACACTCCAACAAATAGAGTTGAAGTTTAATCCACCTGCTCTACAAGATGCTTAT
TACAGAGCAAGGGCTGGTGAAGCTGCTAACTTTTGTGCACTTATCTTAGCCTACT
GTAATAAGACAGTAGGTGAGTTAGGTGATGTTAGAGAAACAATGTCGTACTTGTT
TCAACATGCCAATTTAGATTCGTGCAAAAGAGTCTTGAACGTGGTGTGTAAAACT
TGTGGACAACAGCAGACAACCCTTAAGGGTGTAGAAGCTGTTATGTACATGGGC
ACACTTTCGTATGAACAATTTAAGAAAGGTGTTCAGATACCTTGTACGTGTGGTA
AACAAGCTACAAAATATCTAGTACAACAGGAGTCGCCTTTTGTTATGATGTCGGC
ACCACCTGCTCAGTATGAACTTAAGCATGGTACATTTACTTGTGCTTCGGAGTAC
ACTGGTAATTACCAGTGTGGTCACTATAAACATATAACTTCGAAAGAAACTTTGT
ATTGCATAGACGGTGCTTTACTTACAAAGTCGTCGGAATACAAAGGTCCTATTAC
GGATGTTTTCTACAAAGAAAACTCGTACACAACAACCATAAAACCAGTTACTTAT
AAATTGGATGGTGTTGTTTGTACAGAAATTGACCCTAAGTTGGACAATTATTATA
AGAAAGACAATTCGTATTTCACAGAGCAACCAATTGATCTTGTACCAAACCAAC
CATATCCAAACGCATCGTTCGATAATTTTAAGTTTGTATGTGATAATATCAAATTT
GCTGATGATTTAAACCAGTTAACTGGTTATAAGAAACCTGCTTCGAGAGAGCTTA
AAGTTACATTTTTCCCTGACTTAAATGGTGATGTGGTGGCTATTGATTATAAACA
CTACACACCCTCGTTTAAGAAAGGAGCTAAATTGTTACATAAACCTATTGTTTGG
CATGTTAACAATGCAACTAATAAAGCCACGTATAAACCAAATACCTGGTGTATA
CGTTGTCTTTGGTCGACAAAACCAGTTGAAACATCGAATTCGTTTGATGTACTGA
AGTCGGAGGACGCGCAGGGAATGGATAATCTTGCCTGCGAAGATCTAAAACCAG
TCTCGGAAGAAGTAGTGGAAAATCCTACCATACAGAAAGACGTTCTTGAGTGTA
ATGTGAAAACTACCGAAGTTGTAGGAGACATTATACTTAAACCAGCAAATAATT
CGTTAAAAATTACAGAAGAGGTTGGCCACACAGATCTAATGGCTGCTTATGTAG
ACAATTCGTCGCTTACTATTAAGAAACCTAATGAATTATCGAGAGTATTAGGTTT
GAAAACCCTTGCTACTCATGGTTTAGCTGCTGTTAATTCGGTCCCTTGGGATACT
ATAGCTAATTATGCTAAGCCTTTTCTTAACAAAGTTGTTTCGACAACTACTAACAT
AGTTACACGGTGTTTAAACCGTGTTTGTACTAATTATATGCCTTATTTCTTTACTT
TATTGCTACAATTGTGTACTTTTACTAGATCGACAAATTCGAGAATTAAAGCATC
GATGCCGACTACTATAGCAAAGAATACTGTTAAGTCGGTCGGTAAATTTTGTCTA
GAGGCTTCGTTTAATTATTTGAAGTCGCCTAATTTTTCGAAACTGATAAATATTAT
AATTTGGTTTTTACTATTATCGGTTTGCCTAGGTTCTTTAATCTACTCAACCGCTG
CTTTAGGTGTTTTAATGTCTAATTTAGGCATGCCTTCTTACTGTACTGGTTACAGA
GAAGGCTATTTGAACTCTACTAATGTCACTATTGCAACCTACTGTACTGGTTCTAT
ACCTTGTAGTGTTTGTCTTAGTGGTTTAGATTCTTTAGACACCTATCCTTCTTTAG
AAACTATACAAATTACCATTTCATCTTTTAAATGGGATTTAACTGCTTTTGGCTTA
GTTGCAGAGTGGTTTTTGGCATATATTCTTTTCACTAGGTTTTTCTATGTACTTGG
ATTGGCTGCAATCATGCAATTGTTTTTCAGCTATTTTGCAGTACATTTTATTAGTA
ATTCTTGGCTTATGTGGTTAATAATTAATCTTGTACAAATGGCCCCGATTTCAGCT
ATGGTTAGAATGTACATCTTCTTTGCATCATTTTATTATGTATGGAAAAGTTATGT
GCATGTTGTAGACGGTTGTAATTCATCAACTTGTATGATGTGTTACAAACGTAAT
AGAGCAACAAGAGTCGAATGTACAACTATTGTTAATGGTGTTAGAAGGTCCTTTT
ATGTCTATGCTAATGGAGGTAAAGGCTTTTGCAAACTACACAATTGGAATTGTGT
TAATTGTGATACATTCTGTGCTGGTAGTACATTTATTAGTGATGAAGTTGCGAGA
GACTTGTCACTACAGTTTAAAAGACCAATAAATCCTACTGACCAGTCTTCTTACA
TCGTTGATAGTGTTACAGTGAAGAATGGTTCCATCCATCTTTACTTTGATAAAGCT
GGTCAAAAGACTTATGAAAGACATTCTCTCTCTCATTTTGTTAACTTAGACAACC
TGAGAGCTAATAACACTAAAGGTTCATTGCCTATTAATGTTATAGTTTTTGATGG
TAAATCAAAATGTGAAGAATCATCTGCAAAATCAGCGTCTGTTTACTACAGTCAG
CTTATGTGTCAACCTATACTGTTACTAGATCAGGCATTAGTGTCTGATGTTGGTGA
TAGTGCGGAAGTTGCAGTTAAAATGTTTGATGCTTACGTTAATACGTTTTCATCA
ACTTTTAACGTACCAATGGAAAAACTCAAAACACTAGTTGCAACTGCAGAAGCT
GAACTTGCAAAGAATGTGTCCTTAGACAATGTCTTATCTACTTTTATTTCAGCAGC
TCGGCAAGGGTTTGTTGATTCAGATGTAGAAACTAAAGATGTTGTTGAATGTCTT
AAATTGTCACATCAATCTGACATAGAAGTTACTGGCGATAGTTGTAATAACTATA
TGCTCACCTATAACAAAGTTGAAAACATGACACCCCGTGACCTTGGTGCTTGTAT
TGACTGTAGTGCGCGTCATATTAATGCGCAGGTAGCAAAAAGTCACAACATTGCT
TTGATATGGAACGTTAAAGATTTCATGTCATTGTCTGAACAACTACGAAAACAAA
TACGTAGTGCTGCTAAAAAGAATAACTTACCTTTTAAGTTGACATGTGCAACTAC
TAGACAAGTTGTTAATGTTGTAACAACAAAGATAGCACTTAAGGGTGGTAAAAT
TGTTAATAATTGGTTGAAGCAGTTAATTAAA
SEQ ID NO: 50 - Clone pCC1-4K-SARS-COV-2-160-5. Only fragments 1 and 3
have been deoptimized:
GGGTCCCACGTGCTTCGGCTAACATAGGTTGTAACCATACAGGTGTTG
TTGGAGAAGGTTCGGAAGGTCTTAATGACAACCTTCTTGAAATACTCCAAAAAG
AGAAAGTCAACATCAATATTGTTGGTGACTTTAAACTTAATGAAGAGATCGCCAT
TATTTTGGCATCGTTTTCGGCTTCGACATCGGCTTTTGTGGAAACTGTGAAAGGTT
TGGATTATAAAGCATTCAAACAAATTGTTGAATCGTGTGGTAATTTTAAAGTTAC
AAAAGGAAAAGCTAAAAAAGGTGCCTGGAATATTGGTGAACAGAAATCGATACT
GTCGCCTCTTTATGCATTTGCATCGGAGGCTGCTCGTGTTGTACGATCGATTTTCT
CGCGCACTCTTGAAACTGCTCAAAATTCGGTGCGTGTTTTACAGAAGGCCGCTAT
AACAATACTAGATGGAATTTCGCAGTATTCGCTGAGACTCATTGATGCTATGATG
TTCACATCGGATTTGGCTACTAACAATCTAGTTGTAATGGCCTACATTACAGGTG
GTGTTGTTCAGTTGACTTCGCAGTGGCTAACTAACATCTTTGGCACTGTTTATGAA
AAACTCAAACCCGTCCTTGATTGGCTTGAAGAGAAGTTTAAGGAAGGTGTAGAG
TTTCTTAGAGACGGTTGGGAAATTGTTAAATTTATCTCGACCTGTGCTTGTGAAAT
TGTCGGTGGACAAATTGTCACCTGTGCAAAGGAAATTAAGGAGTCGGTTCAGAC
ATTCTTTAAGCTTGTAAATAAATTTTTGGCTTTGTGTGCTGACTCGATCATTATTG
GTGGAGCTAAACTTAAAGCCTTGAATTTAGGTGAAACATTTGTCACGCACTCGAA
GGGATTGTACAGAAAGTGTGTTAAATCGAGAGAAGAAACTGGCCTACTCATGCC
TCTAAAAGCCCCAAAAGAAATTATCTTCTTAGAGGGAGAAACACTTCCCACAGA
AGTGTTAACAGAGGAAGTTGTCTTGAAAACTGGTGATTTACAACCATTAGAACA
ACCTACTTCGGAAGCTGTTGAAGCTCCATTGGTTGGTACACCAGTTTGTATTAAC
GGGCTTATGTTGCTCGAAATCAAAGACACAGAAAAGTACTGTGCCCTTGCACCTA
ATATGATGGTAACAAACAATACCTTCACACTCAAAGGCGGTGCACCAACAAAGG
TTACTTTTGGTGATGACACTGTGATAGAAGTGCAAGGTTACAAGTCGGTGAATAT
CACTTTTGAACTTGATGAAAGGATTGATAAAGTACTTAATGAGAAGTGCTCGGCC
TATACAGTTGAACTCGGTACAGAAGTAAATGAGTTCGCCTGTGTTGTGGCAGATG
CTGTCATAAAAACTTTGCAACCAGTATCGGAATTACTTACACCACTGGGCATTGA
TTTAGATGAGTGGTCGATGGCTACATACTACTTATTTGATGAGTCGGGTGAGTTT
AAATTGGCTTCGCATATGTATTGTTCGTTCTACCCTCCAGATGAGGATGAAGAAG
AAGGTGATTGTGAAGAAGAAGAGTTTGAGCCATCGACTCAATATGAGTATGGTA
CTGAAGATGATTACCAAGGTAAACCTTTGGAATTTGGTGCCACTTCGGCTGCTCT
TCAACCTGAAGAAGAGCAAGAAGAAGATTGGTTAGATGATGATTCGCAACAAAC
TGTTGGTCAACAAGACGGCTCGGAGGACAATCAGACAACTACTATTCAAACAAT
TGTTGAGGTTCAACCTCAATTAGAGATGGAACTTACACCAGTTGTTCAGACTATT
GAAGTGAATTCGTTTTCGGGTTATTTAAAACTTACTGACAATGTATACATTAAAA
ATGCAGACATTGTGGAAGAAGCTAAAAAGGTAAAACCAACAGTGGTTGTTAATG
CAGCCAATGTTTACCTTAAACATGGAGGAGGTGTTGCAGGAGCCTTAAATAAGG
CTACTAACAATGCCATGCAAGTTGAATCGGATGATTACATAGCTACTAATGGACC
ACTTAAAGTGGGTGGTTCGTGTGTTTTATCGGGACACAATCTTGCTAAACACTGT
CTTCATGTTGTCGGCCCAAATGTTAACAAAGGTGAAGACATTCAACTTCTTAAGT
CGGCTTATGAAAATTTTAATCAGCACGAAGTTCTACTTGCACCATTATTATCGGC
TGGTATTTTTGGTGCTGACCCTATACATTCGTTAAGAGTTTGTGTAGATACTGTTC
GCACAAATGTCTACTTAGCTGTCTTTGATAAAAATCTCTATGACAAACTTGTTTCG
TCGTTTTTGGAAATGAAGTCGGAAAAGCAAGTTGAACAAAAGATCGCTGAGATT
CCTAAAGAGGAAGTTAAGCCATTTATAACTGAATCGAAACCTTCGGTTGAACAG
AGAAAACAAGATGATAAGAAAATCAAAGCTTGTGTTGAAGAAGTTACAACAACT
CTGGAAGAAACTAAGTTCCTCACAGAAAACTTGTTACTTTATATTGACATTAATG
GCAATCTTCATCCAGATTCGGCCACTCTTGTTTCGGACATTGACATCACTTTCTTA
AAGAAAGATGCTCCATATATAGTGGGTGATGTTGTTCAAGAGGGTGTTTTAACTG
CTGTGGTTATACCTACTAAAAAGGCTGGTGGCACTACTGAAATGCTAGCGAAAG
CTTTGAGAAAAGTGCCAACAGACAATTATATAACCACTTACCCGGGTCAGGGTTT
AAATGGTTACACTGTAGAGGAGGCAAAGACAGTGCTTAAAAAGTGTAAAAGTGC
CTTTTACATTCTACCATCTATTATCTCTAATGAGAAGCAAGAAATTCTTGGAACTG
TTTCTTGGAATTTGCGAGAAATGCTTGCACATGCAGAAGAAACACGCAAATTAAT
GCCTGTCTGTGTGGAAACTAAAGCCATAGTTTCAACTATACAGCGTAAATATAAG
GGTATTAAAATACAAGAGGGTGTGGTTGATTATGGTGCTAGATTTTACTTTTACA
CCAGTAAAACAACTGTAGCGTCACTTATCAACACACTTAACGATCTAAATGAAA
CTCTTGTTACAATGCCACTTGGCTATGTAACACATGGCTTAAATTTGGAAGAAGC
TGCTCGGTATATGAGATCTCTCAAAGTGCCAGCTACAGTTTCTGTTTCTTCACCTG
ATGCTGTTACAGCGTATAATGGTTATCTTACTTCTTCTTCTAAAACACCTGAAGAA
CATTTTATTGAAACCATCTCACTTGCTGGTTCCTATAAAGATTGGTCCTATTCTGG
ACAATCTACACAACTAGGTATAGAATTTCTTAAGAGAGGTGATAAAAGTGTATAT
TACACTAGTAATCCTACCACATTCCACCTAGATGGTGAAGTTATCACCTTTGACA
ATCTTAAGACACTTCTTTCTTTGAGAGAAGTGAGGACTATTAAGGTGTTTACAAC
AGTAGACAACATTAACCTCCACACGCAAGTTGTGGACATGTCAATGACATATGG
ACAACAGTTTGGTCCAACTTATTTGGATGGAGCTGATGTTACTAAAATAAAACCT
CATAATTCACATGAAGGTAAAACATTTTATGTTTTACCTAATGATGACACTCTAC
GTGTTGAGGCTTTTGAGTACTACCACACAACTGATCCTAGTTTTCTGGGTAGGTA
CATGTCAGCATTAAATCACACTAAAAAGTGGAAATACCCACAAGTTAATGGTTT
AACTTCTATTAAATGGGCAGATAACAACTGTTATCTTGCCACTGCATTGTTAACA
CTCCAACAAATAGAGTTGAAGTTTAATCCACCTGCTCTACAAGATGCTTATTACA
GAGCAAGGGCTGGTGAAGCTGCTAACTTTTGTGCACTTATCTTAGCCTACTGTAA
TAAGACAGTAGGTGAGTTAGGTGATGTTAGAGAAACAATGAGTTACTTGTTTCAA
CATGCCAATTTAGATTCTTGCAAAAGAGTCTTGAACGTGGTGTGTAAAACTTGTG
GACAACAGCAGACAACCCTTAAGGGTGTAGAAGCTGTTATGTACATGGGCACAC
TTTCTTATGAACAATTTAAGAAAGGTGTTCAGATACCTTGTACGTGTGGTAAACA
AGCTACAAAATATCTAGTACAACAGGAGTCACCTTTTGTTATGATGTCAGCACCA
CCTGCTCAGTATGAACTTAAGCATGGTACATTTACTTGTGCTAGTGAGTACACTG
GTAATTACCAGTGTGGTCACTATAAACATATAACTTCTAAAGAAACTTTGTATTG
CATAGACGGTGCTTTACTTACAAAGTCCTCAGAATACAAAGGTCCTATTACGGAT
GTTTTCTACAAAGAAAACAGTTACACAACAACCATAAAACCAGTTACTTATAAAT
TGGATGGTGTTGTTTGTACAGAAATTGACCCTAAGTTGGACAATTATTATAAGAA
AGACAATTCTTATTTCACAGAGCAACCAATTGATCTTGTACCAAACCAACCATAT
CCAAACGCAAGCTTCGATAATTTTAAGTTTGTATGTGATAATATCAAATTTGCTG
ATGATTTAAACCAGTTAACTGGTTATAAGAAACCTGCTTCAAGAGAGCTTAAAGT
TACATTTTTCCCTGACTTAAATGGTGATGTGGTGGCTATTGATTATAAACACTACA
CACCCTCTTTTAAGAAAGGAGCTAAATTGTTACATAAACCTATTGTTTGGCATGT
TAACAATGCAACTAATAAAGCCACGTATAAACCAAATACCTGGTGTATACGTTGT
CTTTGGAGCACAAAACCAGTTGAAACATCAAATTCGTTTGATGTACTGAAGTCAG
AGGACGCGCAGGGAATGGATAATCTTGCCTGCGAAGATCTAAAACCAGTCTCTG
AAGAAGTAGTGGAAAATCCTACCATACAGAAAGACGTTCTTGAGTGTAATGTGA
AAACTACCGAAGTTGTAGGAGACATTATACTTAAACCAGCAAATAATAGTTTAA
AAATTACAGAAGAGGTTGGCCACACAGATCTAATGGCTGCTTATGTAGACAATT
CTAGTCTTACTATTAAGAAACCTAATGAATTATCTAGAGTATTAGGTTTGAAAAC
CCTTGCTACTCATGGTTTAGCTGCTGTTAATAGTGTCCCTTGGGATACTATAGCTA
ATTATGCTAAGCCTTTTCTTAACAAAGTTGTTAGTACAACTACTAACATAGTTAC
ACGGTGTTTAAACCGTGTTTGTACTAATTATATGCCTTATTTCTTTACTTTATTGCT
ACAATTGTGTACTTTTACTAGAAGTACAAATTCTAGAATTAAAGCATCTATGCCG
ACTACTATAGCAAAGAATACTGTTAAGAGTGTCGGTAAATTTTGTCTAGAGGCTT
CATTTAATTATTTGAAGTCACCTAATTTTTCTAAACTGATAAATATTATAATTTGG
TTTTTACTATTAAGTGTTTGCCTAGGTTCGTTAATCTACTCGACCGCTGCTTTAGG
TGTTTTAATGTCGAATTTAGGCATGCCTTCGTACTGTACTGGTTACAGAGAAGGC
TATTTGAACTCGACTAATGTCACTATTGCAACCTACTGTACTGGTTCGATACCTTG
TTCGGTTTGTCTTTCGGGTTTAGATTCGTTAGACACCTATCCTTCGTTAGAAACTA
TACAAATTACCATTTCGTCGTTTAAATGGGATTTAACTGCTTTTGGCTTAGTTGCA
GAGTGGTTTTTGGCATATATTCTTTTCACTAGGTTTTTCTATGTACTTGGATTGGC
TGCAATCATGCAATTGTTTTTCTCGTATTTTGCAGTACATTTTATTTCGAATTCGT
GGCTTATGTGGTTAATAATTAATCTTGTACAAATGGCCCCGATTTCGGCTATGGTT
AGAATGTACATCTTCTTTGCATCGTTTTATTATGTATGGAAATCGTATGTGCATGT
TGTAGACGGTTGTAATTCGTCGACTTGTATGATGTGTTACAAACGTAATAGAGCA
ACAAGAGTCGAATGTACAACTATTGTTAATGGTGTTAGAAGGTCGTTTTATGTCT
ATGCTAATGGAGGTAAAGGCTTTTGCAAACTACACAATTGGAATTGTGTTAATTG
TGATACATTCTGTGCTGGTTCGACATTTATTTCGGATGAAGTTGCGAGAGACTTG
TCGCTACAGTTTAAAAGACCAATAAATCCTACTGACCAGTCGTCGTACATCGTTG
ATTCGGTTACAGTGAAGAATGGTTCGATCCATCTTTACTTTGATAAAGCTGGTCA
AAAGACTTATGAAAGACATTCGCTCTCGCATTTTGTTAACTTAGACAACCTGAGA
GCTAATAACACTAAAGGTTCGTTGCCTATTAATGTTATAGTTTTTGATGGTAAATC
GAAATGTGAAGAATCGTCGGCAAAATCGGCGTCGGTTTACTACTCGCAGCTTATG
TGTCAACCTATACTGTTACTAGATCAGGCATTAGTGTCGGATGTTGGTGATTCGG
CGGAAGTTGCAGTTAAAATGTTTGATGCTTACGTTAATACGTTTTCGTCGACTTTT
AACGTACCAATGGAAAAACTCAAAACACTAGTTGCAACTGCAGAAGCTGAACTT
GCAAAGAATGTGTCGTTAGACAATGTCTTATCGACTTTTATTTCGGCAGCTCGGC
AAGGGTTTGTTGATTCGGATGTAGAAACTAAAGATGTTGTTGAATGTCTTAAATT
GTCGCATCAATCGGACATAGAAGTTACTGGCGATTCGTGTAATAACTATATGCTC
ACCTATAACAAAGTTGAAAACATGACACCCCGTGACCTTGGTGCTTGTATTGACT
GTTCGGCGCGTCATATTAATGCGCAGGTAGCAAAATCGCACAACATTGCTTTGAT
ATGGAACGTTAAAGATTTCATGTCGTTGTCGGAACAACTACGAAAACAAATACG
TTCGGCTGCTAAAAAGAATAACTTACCTTTTAAGTTGACATGTGCAACTACTAGA
CAAGTTGTTAATGTTGTAACAACAAAGATAGCACTTAAGGGTGGTAAAATTGTTA
ATAATTGGTTGAAGCAGTTAATTAAA
SEQ ID NO: 51 - Clone pCC1-4K-SARS-COV-2-160-6. Only fragments 2 and 3
have been deoptimized:
GGGTCCCACGTGCTAGCGCTAACATAGGTTGTAACCATACAGGTGTTG
TTGGAGAAGGTTCCGAAGGTCTTAATGACAACCTTCTTGAAATACTCCAAAAAG
AGAAAGTCAACATCAATATTGTTGGTGACTTTAAACTTAATGAAGAGATCGCCAT
TATTTTGGCATCTTTTTCTGCTTCCACAAGTGCTTTTGTGGAAACTGTGAAAGGTT
TGGATTATAAAGCATTCAAACAAATTGTTGAATCCTGTGGTAATTTTAAAGTTAC
AAAAGGAAAAGCTAAAAAAGGTGCCTGGAATATTGGTGAACAGAAATCAATACT
GAGTCCTCTTTATGCATTTGCATCAGAGGCTGCTCGTGTTGTACGATCAATTTTCT
CCCGCACTCTTGAAACTGCTCAAAATTCTGTGCGTGTTTTACAGAAGGCCGCTAT
AACAATACTAGATGGAATTTCACAGTATTCACTGAGACTCATTGATGCTATGATG
TTCACATCTGATTTGGCTACTAACAATCTAGTTGTAATGGCCTACATTACAGGTG
GTGTTGTTCAGTTGACTTCGCAGTGGCTAACTAACATCTTTGGCACTGTTTATGAA
AAACTCAAACCCGTCCTTGATTGGCTTGAAGAGAAGTTTAAGGAAGGTGTAGAG
TTTCTTAGAGACGGTTGGGAAATTGTTAAATTTATCTCAACCTGTGCTTGTGAAAT
TGTCGGTGGACAAATTGTCACCTGTGCAAAGGAAATTAAGGAGAGTGTTCAGAC
ATTCTTTAAGCTTGTAAATAAATTTTTGGCTTTGTGTGCTGACTCTATCATTATTG
GTGGAGCTAAACTTAAAGCCTTGAATTTAGGTGAAACATTTGTCACGCACTCAAA
GGGATTGTACAGAAAGTGTGTTAAATCCAGAGAAGAAACTGGCCTACTCATGCC
TCTAAAAGCCCCAAAAGAAATTATCTTCTTAGAGGGAGAAACACTTCCCACAGA
AGTGTTAACAGAGGAAGTTGTCTTGAAAACTGGTGATTTACAACCATTAGAACA
ACCTACTAGTGAAGCTGTTGAAGCTCCATTGGTTGGTACACCAGTTTGTATTAAC
GGGCTTATGTTGCTCGAAATCAAAGACACAGAAAAGTACTGTGCCCTTGCACCTA
ATATGATGGTAACAAACAATACCTTCACACTCAAAGGCGGTGCACCAACAAAGG
TTACTTTTGGTGATGACACTGTGATAGAAGTGCAAGGTTACAAGAGTGTGAATAT
CACTTTTGAACTTGATGAAAGGATTGATAAAGTACTTAATGAGAAGTGCTCTGCC
TATACAGTTGAACTCGGTACAGAAGTAAATGAGTTCGCCTGTGTTGTGGCAGATG
CTGTCATAAAAACTTTGCAACCAGTATCTGAATTACTTACACCACTGGGCATTGA
TTTAGATGAGTGGAGTATGGCTACATACTACTTATTTGATGAGTCTGGTGAGTTT
AAATTGGCTTCACATATGTATTGTTCTTTCTACCCTCCAGATGAGGATGAAGAAG
AAGGTGATTGTGAAGAAGAAGAGTTTGAGCCATCAACTCAATATGAGTATGGTA
CTGAAGATGATTACCAAGGTAAACCTTTGGAATTTGGTGCCACTTCTGCTGCTCT
TCAACCTGAAGAAGAGCAAGAAGAAGATTGGTTAGATGATGATAGTCAACAAAC
TGTTGGTCAACAAGACGGCAGTGAGGACAATCAGACAACTACTATTCAAACAAT
TGTTGAGGTTCAACCTCAATTAGAGATGGAACTTACACCAGTTGTTCAGACTATT
GAAGTGAATAGTTTTAGTGGTTATTTAAAACTTACTGACAATGTATACATTAAAA
ATGCAGACATTGTGGAAGAAGCTAAAAAGGTAAAACCAACAGTGGTTGTTAATG
CAGCCAATGTTTACCTTAAACATGGAGGAGGTGTTGCAGGAGCCTTAAATAAGG
CTACTAACAATGCCATGCAAGTTGAATCTGATGATTACATAGCTACTAATGGACC
ACTTAAAGTGGGTGGTAGTTGTGTTTTAAGCGGACACAATCTTGCTAAACACTGT
CTTCATGTTGTCGGCCCAAATGTTAACAAAGGTGAAGACATTCAACTTCTTAAGA
GTGCTTATGAAAATTTTAATCAGCACGAAGTTCTACTTGCACCATTATTATCAGCT
GGTATTTTTGGTGCTGACCCTATACATTCTTTAAGAGTTTGTGTAGATACTGTTCG
CACAAATGTCTACTTAGCTGTCTTTGATAAAAATCTCTATGACAAACTTGTTTCAA
GCTTTTTGGAAATGAAGAGTGAAAAGCAAGTTGAACAAAAGATCGCTGAGATTC
CTAAAGAGGAAGTTAAGCCATTTATAACTGAAAGTAAACCTTCAGTTGAACAGA
GAAAACAAGATGATAAGAAAATCAAAGCTTGTGTTGAAGAAGTTACAACAACTC
TGGAAGAAACTAAGTTCCTCACAGAAAACTTGTTACTTTATATTGACATTAATGG
CAATCTTCATCCAGATTCTGCCACTCTTGTTAGTGACATTGACATCACTTTCTTAA
AGAAAGATGCTCCATATATAGTGGGTGATGTTGTTCAAGAGGGTGTTTTAACTGC
TGTGGTTATACCTACTAAAAAGGCTGGTGGCACTACTGAAATGCTAGCGAAAGC
TTTGAGAAAAGTGCCAACAGACAATTATATAACCACTTACCCGGGTCAGGGTTTA
AATGGTTACACTGTAGAGGAGGCAAAGACAGTGCTTAAAAAGTGTAAATCGGCC
TTTTACATTCTACCATCGATTATCTCGAATGAGAAGCAAGAAATTCTTGGAACTG
TTTCGTGGAATTTGCGAGAAATGCTTGCACATGCAGAAGAAACACGCAAATTAA
TGCCTGTCTGTGTGGAAACTAAAGCCATAGTTTCGACTATACAGCGTAAATATAA
GGGTATTAAAATACAAGAGGGTGTGGTTGATTATGGTGCTAGATTTTACTTTTAC
ACCTCGAAAACAACTGTAGCGTCGCTTATCAACACACTTAACGATCTAAATGAA
ACTCTTGTTACAATGCCACTTGGCTATGTAACACATGGCTTAAATTTGGAAGAAG
CTGCTCGGTATATGAGATCGCTCAAAGTGCCAGCTACAGTTTCGGTTTCGTCGCC
TGATGCTGTTACAGCGTATAATGGTTATCTTACTTCGTCGTCGAAAACACCTGAA
GAACATTTTATTGAAACCATCTCGCTTGCTGGTTCGTATAAAGATTGGTCGTATTC
GGGACAATCGACACAACTAGGTATAGAATTTCTTAAGAGAGGTGATAAATCGGT
ATATTACACTTCGAATCCTACCACATTCCACCTAGATGGTGAAGTTATCACCTTT
GACAATCTTAAGACACTTCTTTCGTTGAGAGAAGTGAGGACTATTAAGGTGTTTA
CAACAGTAGACAACATTAACCTCCACACGCAAGTTGTGGACATGTCGATGACAT
ATGGACAACAGTTTGGTCCAACTTATTTGGATGGAGCTGATGTTACTAAAATAAA
ACCTCATAATTCGCATGAAGGTAAAACATTTTATGTTTTACCTAATGATGACACT
CTACGTGTTGAGGCTTTTGAGTACTACCACACAACTGATCCTTCGTTTCTGGGTA
GGTACATGTCGGCATTAAATCACACTAAAAAGTGGAAATACCCACAAGTTAATG
GTTTAACTTCGATTAAATGGGCAGATAACAACTGTTATCTTGCCACTGCATTGTT
AACACTCCAACAAATAGAGTTGAAGTTTAATCCACCTGCTCTACAAGATGCTTAT
TACAGAGCAAGGGCTGGTGAAGCTGCTAACTTTTGTGCACTTATCTTAGCCTACT
GTAATAAGACAGTAGGTGAGTTAGGTGATGTTAGAGAAACAATGTCGTACTTGTT
TCAACATGCCAATTTAGATTCGTGCAAAAGAGTCTTGAACGTGGTGTGTAAAACT
TGTGGACAACAGCAGACAACCCTTAAGGGTGTAGAAGCTGTTATGTACATGGGC
ACACTTTCGTATGAACAATTTAAGAAAGGTGTTCAGATACCTTGTACGTGTGGTA
AACAAGCTACAAAATATCTAGTACAACAGGAGTCGCCTTTTGTTATGATGTCGGC
ACCACCTGCTCAGTATGAACTTAAGCATGGTACATTTACTTGTGCTTCGGAGTAC
ACTGGTAATTACCAGTGTGGTCACTATAAACATATAACTTCGAAAGAAACTTTGT
ATTGCATAGACGGTGCTTTACTTACAAAGTCGTCGGAATACAAAGGTCCTATTAC
GGATGTTTTCTACAAAGAAAACTCGTACACAACAACCATAAAACCAGTTACTTAT
AAATTGGATGGTGTTGTTTGTACAGAAATTGACCCTAAGTTGGACAATTATTATA
AGAAAGACAATTCGTATTTCACAGAGCAACCAATTGATCTTGTACCAAACCAAC
CATATCCAAACGCATCGTTCGATAATTTTAAGTTTGTATGTGATAATATCAAATTT
GCTGATGATTTAAACCAGTTAACTGGTTATAAGAAACCTGCTTCGAGAGAGCTTA
AAGTTACATTTTTCCCTGACTTAAATGGTGATGTGGTGGCTATTGATTATAAACA
CTACACACCCTCGTTTAAGAAAGGAGCTAAATTGTTACATAAACCTATTGTTTGG
CATGTTAACAATGCAACTAATAAAGCCACGTATAAACCAAATACCTGGTGTATA
CGTTGTCTTTGGTCGACAAAACCAGTTGAAACATCGAATTCGTTTGATGTACTGA
AGTCGGAGGACGCGCAGGGAATGGATAATCTTGCCTGCGAAGATCTAAAACCAG
TCTCGGAAGAAGTAGTGGAAAATCCTACCATACAGAAAGACGTTCTTGAGTGTA
ATGTGAAAACTACCGAAGTTGTAGGAGACATTATACTTAAACCAGCAAATAATT
CGTTAAAAATTACAGAAGAGGTTGGCCACACAGATCTAATGGCTGCTTATGTAG
ACAATTCGTCGCTTACTATTAAGAAACCTAATGAATTATCGAGAGTATTAGGTTT
GAAAACCCTTGCTACTCATGGTTTAGCTGCTGTTAATTCGGTCCCTTGGGATACT
ATAGCTAATTATGCTAAGCCTTTTCTTAACAAAGTTGTTTCGACAACTACTAACAT
AGTTACACGGTGTTTAAACCGTGTTTGTACTAATTATATGCCTTATTTCTTTACTT
TATTGCTACAATTGTGTACTTTTACTAGATCGACAAATTCGAGAATTAAAGCATC
GATGCCGACTACTATAGCAAAGAATACTGTTAAGTCGGTCGGTAAATTTTGTCTA
GAGGCTTCGTTTAATTATTTGAAGTCGCCTAATTTTTCGAAACTGATAAATATTAT
AATTTGGTTTTTACTATTATCGGTTTGCCTAGGTTCGTTAATCTACTCGACCGCTG
CTTTAGGTGTTTTAATGTCGAATTTAGGCATGCCTTCGTACTGTACTGGTTACAGA
GAAGGCTATTTGAACTCGACTAATGTCACTATTGCAACCTACTGTACTGGTTCGA
TACCTTGTTCGGTTTGTCTTTCGGGTTTAGATTCGTTAGACACCTATCCTTCGTTA
GAAACTATACAAATTACCATTTCGTCGTTTAAATGGGATTTAACTGCTTTTGGCTT
AGTTGCAGAGTGGTTTTTGGCATATATTCTTTTCACTAGGTTTTTCTATGTACTTG
GATTGGCTGCAATCATGCAATTGTTTTTCTCGTATTTTGCAGTACATTTTATTTCG
AATTCGTGGCTTATGTGGTTAATAATTAATCTTGTACAAATGGCCCCGATTTCGG
CTATGGTTAGAATGTACATCTTCTTTGCATCGTTTTATTATGTATGGAAATCGTAT
GTGCATGTTGTAGACGGTTGTAATTCGTCGACTTGTATGATGTGTTACAAACGTA
ATAGAGCAACAAGAGTCGAATGTACAACTATTGTTAATGGTGTTAGAAGGTCGTT
TTATGTCTATGCTAATGGAGGTAAAGGCTTTTGCAAACTACACAATTGGAATTGT
GTTAATTGTGATACATTCTGTGCTGGTTCGACATTTATTTCGGATGAAGTTGCGAG
AGACTTGTCGCTACAGTTTAAAAGACCAATAAATCCTACTGACCAGTCGTCGTAC
ATCGTTGATTCGGTTACAGTGAAGAATGGTTCGATCCATCTTTACTTTGATAAAG
CTGGTCAAAAGACTTATGAAAGACATTCGCTCTCGCATTTTGTTAACTTAGACAA
CCTGAGAGCTAATAACACTAAAGGTTCGTTGCCTATTAATGTTATAGTTTTTGAT
GGTAAATCGAAATGTGAAGAATCGTCGGCAAAATCGGCGTCGGTTTACTACTCG
CAGCTTATGTGTCAACCTATACTGTTACTAGATCAGGCATTAGTGTCGGATGTTG
GTGATTCGGCGGAAGTTGCAGTTAAAATGTTTGATGCTTACGTTAATACGTTTTC
GTCGACTTTTAACGTACCAATGGAAAAACTCAAAACACTAGTTGCAACTGCAGA
AGCTGAACTTGCAAAGAATGTGTCGTTAGACAATGTCTTATCGACTTTTATTTCG
GCAGCTCGGCAAGGGTTTGTTGATTCGGATGTAGAAACTAAAGATGTTGTTGAAT
GTCTTAAATTGTCGCATCAATCGGACATAGAAGTTACTGGCGATTCGTGTAATAA
CTATATGCTCACCTATAACAAAGTTGAAAACATGACACCCCGTGACCTTGGTGCT
TGTATTGACTGTTCGGCGCGTCATATTAATGCGCAGGTAGCAAAATCGCACAACA
TTGCTTTGATATGGAACGTTAAAGATTTCATGTCGTTGTCGGAACAACTACGAAA
ACAAATACGTTCGGCTGCTAAAAAGAATAACTTACCTTTTAAGTTGACATGTGCA
ACTACTAGACAAGTTGTTAATGTTGTAACAACAAAGATAGCACTTAAGGGTGGT
AAAATTGTTAATAATTGGTTGAAGCAGTTAATTAAA
SEQ ID NO: 52 - Clone pCC1-4K-SARS-COV-2-160-7. All 3 fragments were
deoptimized. Deoptimized sequence between SanDI to PacI in ORF1a:
GGGTCCCACGTGCTTCGGCTAACATAGGTTGTAACCATACAGGTGTTG
TTGGAGAAGGTTCGGAAGGTCTTAATGACAACCTTCTTGAAATACTCCAAAAAG
AGAAAGTCAACATCAATATTGTTGGTGACTTTAAACTTAATGAAGAGATCGCCAT
TATTTTGGCATCGTTTTCGGCTTCGACATCGGCTTTTGTGGAAACTGTGAAAGGTT
TGGATTATAAAGCATTCAAACAAATTGTTGAATCGTGTGGTAATTTTAAAGTTAC
AAAAGGAAAAGCTAAAAAAGGTGCCTGGAATATTGGTGAACAGAAATCGATACT
GTCGCCTCTTTATGCATTTGCATCGGAGGCTGCTCGTGTTGTACGATCGATTTTCT
CGCGCACTCTTGAAACTGCTCAAAATTCGGTGCGTGTTTTACAGAAGGCCGCTAT
AACAATACTAGATGGAATTTCGCAGTATTCGCTGAGACTCATTGATGCTATGATG
TTCACATCGGATTTGGCTACTAACAATCTAGTTGTAATGGCCTACATTACAGGTG
GTGTTGTTCAGTTGACTTCGCAGTGGCTAACTAACATCTTTGGCACTGTTTATGAA
AAACTCAAACCCGTCCTTGATTGGCTTGAAGAGAAGTTTAAGGAAGGTGTAGAG
TTTCTTAGAGACGGTTGGGAAATTGTTAAATTTATCTCGACCTGTGCTTGTGAAAT
TGTCGGTGGACAAATTGTCACCTGTGCAAAGGAAATTAAGGAGTCGGTTCAGAC
ATTCTTTAAGCTTGTAAATAAATTTTTGGCTTTGTGTGCTGACTCGATCATTATTG
GTGGAGCTAAACTTAAAGCCTTGAATTTAGGTGAAACATTTGTCACGCACTCGAA
GGGATTGTACAGAAAGTGTGTTAAATCGAGAGAAGAAACTGGCCTACTCATGCC
TCTAAAAGCCCCAAAAGAAATTATCTTCTTAGAGGGAGAAACACTTCCCACAGA
AGTGTTAACAGAGGAAGTTGTCTTGAAAACTGGTGATTTACAACCATTAGAACA
ACCTACTTCGGAAGCTGTTGAAGCTCCATTGGTTGGTACACCAGTTTGTATTAAC
GGGCTTATGTTGCTCGAAATCAAAGACACAGAAAAGTACTGTGCCCTTGCACCTA
ATATGATGGTAACAAACAATACCTTCACACTCAAAGGCGGTGCACCAACAAAGG
TTACTTTTGGTGATGACACTGTGATAGAAGTGCAAGGTTACAAGTCGGTGAATAT
CACTTTTGAACTTGATGAAAGGATTGATAAAGTACTTAATGAGAAGTGCTCGGCC
TATACAGTTGAACTCGGTACAGAAGTAAATGAGTTCGCCTGTGTTGTGGCAGATG
CTGTCATAAAAACTTTGCAACCAGTATCGGAATTACTTACACCACTGGGCATTGA
TTTAGATGAGTGGTCGATGGCTACATACTACTTATTTGATGAGTCGGGTGAGTTT
AAATTGGCTTCGCATATGTATTGTTCGTTCTACCCTCCAGATGAGGATGAAGAAG
AAGGTGATTGTGAAGAAGAAGAGTTTGAGCCATCGACTCAATATGAGTATGGTA
CTGAAGATGATTACCAAGGTAAACCTTTGGAATTTGGTGCCACTTCGGCTGCTCT
TCAACCTGAAGAAGAGCAAGAAGAAGATTGGTTAGATGATGATTCGCAACAAAC
TGTTGGTCAACAAGACGGCTCGGAGGACAATCAGACAACTACTATTCAAACAAT
TGTTGAGGTTCAACCTCAATTAGAGATGGAACTTACACCAGTTGTTCAGACTATT
GAAGTGAATTCGTTTTCGGGTTATTTAAAACTTACTGACAATGTATACATTAAAA
ATGCAGACATTGTGGAAGAAGCTAAAAAGGTAAAACCAACAGTGGTTGTTAATG
CAGCCAATGTTTACCTTAAACATGGAGGAGGTGTTGCAGGAGCCTTAAATAAGG
CTACTAACAATGCCATGCAAGTTGAATCGGATGATTACATAGCTACTAATGGACC
ACTTAAAGTGGGTGGTTCGTGTGTTTTATCGGGACACAATCTTGCTAAACACTGT
CTTCATGTTGTCGGCCCAAATGTTAACAAAGGTGAAGACATTCAACTTCTTAAGT
CGGCTTATGAAAATTTTAATCAGCACGAAGTTCTACTTGCACCATTATTATCGGC
TGGTATTTTTGGTGCTGACCCTATACATTCGTTAAGAGTTTGTGTAGATACTGTTC
GCACAAATGTCTACTTAGCTGTCTTTGATAAAAATCTCTATGACAAACTTGTTTCG
TCGTTTTTGGAAATGAAGTCGGAAAAGCAAGTTGAACAAAAGATCGCTGAGATT
CCTAAAGAGGAAGTTAAGCCATTTATAACTGAATCGAAACCTTCGGTTGAACAG
AGAAAACAAGATGATAAGAAAATCAAAGCTTGTGTTGAAGAAGTTACAACAACT
CTGGAAGAAACTAAGTTCCTCACAGAAAACTTGTTACTTTATATTGACATTAATG
GCAATCTTCATCCAGATTCGGCCACTCTTGTTTCGGACATTGACATCACTTTCTTA
AAGAAAGATGCTCCATATATAGTGGGTGATGTTGTTCAAGAGGGTGTTTTAACTG
CTGTGGTTATACCTACTAAAAAGGCTGGTGGCACTACTGAAATGCTAGCGAAAG
CTTTGAGAAAAGTGCCAACAGACAATTATATAACCACTTACCCGGGTCAGGGTTT
AAATGGTTACACTGTAGAGGAGGCAAAGACAGTGCTTAAAAAGTGTAAATCGGC
CTTTTACATTCTACCATCGATTATCTCGAATGAGAAGCAAGAAATTCTTGGAACT
GTTTCGTGGAATTTGCGAGAAATGCTTGCACATGCAGAAGAAACACGCAAATTA
ATGCCTGTCTGTGTGGAAACTAAAGCCATAGTTTCGACTATACAGCGTAAATATA
AGGGTATTAAAATACAAGAGGGTGTGGTTGATTATGGTGCTAGATTTTACTTTTA
CACCTCGAAAACAACTGTAGCGTCGCTTATCAACACACTTAACGATCTAAATGAA
ACTCTTGTTACAATGCCACTTGGCTATGTAACACATGGCTTAAATTTGGAAGAAG
CTGCTCGGTATATGAGATCGCTCAAAGTGCCAGCTACAGTTTCGGTTTCGTCGCC
TGATGCTGTTACAGCGTATAATGGTTATCTTACTTCGTCGTCGAAAACACCTGAA
GAACATTTTATTGAAACCATCTCGCTTGCTGGTTCGTATAAAGATTGGTCGTATTC
GGGACAATCGACACAACTAGGTATAGAATTTCTTAAGAGAGGTGATAAATCGGT
ATATTACACTTCGAATCCTACCACATTCCACCTAGATGGTGAAGTTATCACCTTT
GACAATCTTAAGACACTTCTTTCGTTGAGAGAAGTGAGGACTATTAAGGTGTTTA
CAACAGTAGACAACATTAACCTCCACACGCAAGTTGTGGACATGTCGATGACAT
ATGGACAACAGTTTGGTCCAACTTATTTGGATGGAGCTGATGTTACTAAAATAAA
ACCTCATAATTCGCATGAAGGTAAAACATTTTATGTTTTACCTAATGATGACACT
CTACGTGTTGAGGCTTTTGAGTACTACCACACAACTGATCCTTCGTTTCTGGGTA
GGTACATGTCGGCATTAAATCACACTAAAAAGTGGAAATACCCACAAGTTAATG
GTTTAACTTCGATTAAATGGGCAGATAACAACTGTTATCTTGCCACTGCATTGTT
AACACTCCAACAAATAGAGTTGAAGTTTAATCCACCTGCTCTACAAGATGCTTAT
TACAGAGCAAGGGCTGGTGAAGCTGCTAACTTTTGTGCACTTATCTTAGCCTACT
GTAATAAGACAGTAGGTGAGTTAGGTGATGTTAGAGAAACAATGTCGTACTTGTT
TCAACATGCCAATTTAGATTCGTGCAAAAGAGTCTTGAACGTGGTGTGTAAAACT
TGTGGACAACAGCAGACAACCCTTAAGGGTGTAGAAGCTGTTATGTACATGGGC
ACACTTTCGTATGAACAATTTAAGAAAGGTGTTCAGATACCTTGTACGTGTGGTA
AACAAGCTACAAAATATCTAGTACAACAGGAGTCGCCTTTTGTTATGATGTCGGC
ACCACCTGCTCAGTATGAACTTAAGCATGGTACATTTACTTGTGCTTCGGAGTAC
ACTGGTAATTACCAGTGTGGTCACTATAAACATATAACTTCGAAAGAAACTTTGT
ATTGCATAGACGGTGCTTTACTTACAAAGTCGTCGGAATACAAAGGTCCTATTAC
GGATGTTTTCTACAAAGAAAACTCGTACACAACAACCATAAAACCAGTTACTTAT
AAATTGGATGGTGTTGTTTGTACAGAAATTGACCCTAAGTTGGACAATTATTATA
AGAAAGACAATTCGTATTTCACAGAGCAACCAATTGATCTTGTACCAAACCAAC
CATATCCAAACGCATCGTTCGATAATTTTAAGTTTGTATGTGATAATATCAAATTT
GCTGATGATTTAAACCAGTTAACTGGTTATAAGAAACCTGCTTCGAGAGAGCTTA
AAGTTACATTTTTCCCTGACTTAAATGGTGATGTGGTGGCTATTGATTATAAACA
CTACACACCCTCGTTTAAGAAAGGAGCTAAATTGTTACATAAACCTATTGTTTGG
CATGTTAACAATGCAACTAATAAAGCCACGTATAAACCAAATACCTGGTGTATA
CGTTGTCTTTGGTCGACAAAACCAGTTGAAACATCGAATTCGTTTGATGTACTGA
AGTCGGAGGACGCGCAGGGAATGGATAATCTTGCCTGCGAAGATCTAAAACCAG
TCTCGGAAGAAGTAGTGGAAAATCCTACCATACAGAAAGACGTTCTTGAGTGTA
ATGTGAAAACTACCGAAGTTGTAGGAGACATTATACTTAAACCAGCAAATAATT
CGTTAAAAATTACAGAAGAGGTTGGCCACACAGATCTAATGGCTGCTTATGTAG
ACAATTCGTCGCTTACTATTAAGAAACCTAATGAATTATCGAGAGTATTAGGTTT
GAAAACCCTTGCTACTCATGGTTTAGCTGCTGTTAATTCGGTCCCTTGGGATACT
ATAGCTAATTATGCTAAGCCTTTTCTTAACAAAGTTGTTTCGACAACTACTAACAT
AGTTACACGGTGTTTAAACCGTGTTTGTACTAATTATATGCCTTATTTCTTTACTT
TATTGCTACAATTGTGTACTTTTACTAGATCGACAAATTCGAGAATTAAAGCATC
GATGCCGACTACTATAGCAAAGAATACTGTTAAGTCGGTCGGTAAATTTTGTCTA
GAGGCTTCGTTTAATTATTTGAAGTCGCCTAATTTTTCGAAACTGATAAATATTAT
AATTTGGTTTTTACTATTATCGGTTTGCCTAGGTTCGTTAATCTACTCGACCGCTG
CTTTAGGTGTTTTAATGTCGAATTTAGGCATGCCTTCGTACTGTACTGGTTACAGA
GAAGGCTATTTGAACTCGACTAATGTCACTATTGCAACCTACTGTACTGGTTCGA
TACCTTGTTCGGTTTGTCTTTCGGGTTTAGATTCGTTAGACACCTATCCTTCGTTA
GAAACTATACAAATTACCATTTCGTCGTTTAAATGGGATTTAACTGCTTTTGGCTT
AGTTGCAGAGTGGTTTTTGGCATATATTCTTTTCACTAGGTTTTTCTATGTACTTG
GATTGGCTGCAATCATGCAATTGTTTTTCTCGTATTTTGCAGTACATTTTATTTCG
AATTCGTGGCTTATGTGGTTAATAATTAATCTTGTACAAATGGCCCCGATTTCGG
CTATGGTTAGAATGTACATCTTCTTTGCATCGTTTTATTATGTATGGAAATCGTAT
GTGCATGTTGTAGACGGTTGTAATTCGTCGACTTGTATGATGTGTTACAAACGTA
ATAGAGCAACAAGAGTCGAATGTACAACTATTGTTAATGGTGTTAGAAGGTCGTT
TTATGTCTATGCTAATGGAGGTAAAGGCTTTTGCAAACTACACAATTGGAATTGT
GTTAATTGTGATACATTCTGTGCTGGTTCGACATTTATTTCGGATGAAGTTGCGAG
AGACTTGTCGCTACAGTTTAAAAGACCAATAAATCCTACTGACCAGTCGTCGTAC
ATCGTTGATTCGGTTACAGTGAAGAATGGTTCGATCCATCTTTACTTTGATAAAG
CTGGTCAAAAGACTTATGAAAGACATTCGCTCTCGCATTTTGTTAACTTAGACAA
CCTGAGAGCTAATAACACTAAAGGTTCGTTGCCTATTAATGTTATAGTTTTTGAT
GGTAAATCGAAATGTGAAGAATCGTCGGCAAAATCGGCGTCGGTTTACTACTCG
CAGCTTATGTGTCAACCTATACTGTTACTAGATCAGGCATTAGTGTCGGATGTTG
GTGATTCGGCGGAAGTTGCAGTTAAAATGTTTGATGCTTACGTTAATACGTTTTC
GTCGACTTTTAACGTACCAATGGAAAAACTCAAAACACTAGTTGCAACTGCAGA
AGCTGAACTTGCAAAGAATGTGTCGTTAGACAATGTCTTATCGACTTTTATTTCG
GCAGCTCGGCAAGGGTTTGTTGATTCGGATGTAGAAACTAAAGATGTTGTTGAAT
GTCTTAAATTGTCGCATCAATCGGACATAGAAGTTACTGGCGATTCGTGTAATAA
CTATATGCTCACCTATAACAAAGTTGAAAACATGACACCCCGTGACCTTGGTGCT
TGTATTGACTGTTCGGCGCGTCATATTAATGCGCAGGTAGCAAAATCGCACAACA
TTGCTTTGATATGGAACGTTAAAGATTTCATGTCGTTGTCGGAACAACTACGAAA
ACAAATACGTTCGGCTGCTAAAAAGAATAACTTACCTTTTAAGTTGACATGTGCA
ACTACTAGACAAGTTGTTAATGTTGTAACAACAAAGATAGCACTTAAGGGTGGT
AAAATTGTTAATAATTGGTTGAAGCAGTTAATTAAA
SEQ ID NO: 53 - Clone pCC1-4K-SARS-COV-2-4N-1. Only fragment 3 has been
deoptimized:
GGGTCCCACGTGCTAGCGCTAACATAGGTTGTAACCATACAGGTGTTG
TTGGAGAAGGTTCCGAAGGTCTTAATGACAACCTTCTTGAAATACTCCAAAAAG
AGAAAGTCAACATCAATATTGTTGGTGACTTTAAACTTAATGAAGAGATCGCCAT
TATTTTGGCATCTTTTTCTGCTTCCACAAGTGCTTTTGTGGAAACTGTGAAAGGTT
TGGATTATAAAGCATTCAAACAAATTGTTGAATCCTGTGGTAATTTTAAAGTTAC
AAAAGGAAAAGCTAAAAAAGGTGCCTGGAATATTGGTGAACAGAAATCAATACT
GAGTCCTCTTTATGCATTTGCATCAGAGGCTGCTCGTGTTGTACGATCAATTTTCT
CCCGCACTCTTGAAACTGCTCAAAATTCTGTGCGTGTTTTACAGAAGGCCGCTAT
AACAATACTAGATGGAATTTCACAGTATTCACTGAGACTCATTGATGCTATGATG
TTCACATCTGATTTGGCTACTAACAATCTAGTTGTAATGGCCTACATTACAGGTG
GTGTTGTTCAGTTGACTTCGCAGTGGCTAACTAACATCTTTGGCACTGTTTATGAA
AAACTCAAACCCGTCCTTGATTGGCTTGAAGAGAAGTTTAAGGAAGGTGTAGAG
TTTCTTAGAGACGGTTGGGAAATTGTTAAATTTATCTCAACCTGTGCTTGTGAAAT
TGTCGGTGGACAAATTGTCACCTGTGCAAAGGAAATTAAGGAGAGTGTTCAGAC
ATTCTTTAAGCTTGTAAATAAATTTTTGGCTTTGTGTGCTGACTCTATCATTATTG
GTGGAGCTAAACTTAAAGCCTTGAATTTAGGTGAAACATTTGTCACGCACTCAAA
GGGATTGTACAGAAAGTGTGTTAAATCCAGAGAAGAAACTGGCCTACTCATGCC
TCTAAAAGCCCCAAAAGAAATTATCTTCTTAGAGGGAGAAACACTTCCCACAGA
AGTGTTAACAGAGGAAGTTGTCTTGAAAACTGGTGATTTACAACCATTAGAACA
ACCTACTAGTGAAGCTGTTGAAGCTCCATTGGTTGGTACACCAGTTTGTATTAAC
GGGCTTATGTTGCTCGAAATCAAAGACACAGAAAAGTACTGTGCCCTTGCACCTA
ATATGATGGTAACAAACAATACCTTCACACTCAAAGGCGGTGCACCAACAAAGG
TTACTTTTGGTGATGACACTGTGATAGAAGTGCAAGGTTACAAGAGTGTGAATAT
CACTTTTGAACTTGATGAAAGGATTGATAAAGTACTTAATGAGAAGTGCTCTGCC
TATACAGTTGAACTCGGTACAGAAGTAAATGAGTTCGCCTGTGTTGTGGCAGATG
CTGTCATAAAAACTTTGCAACCAGTATCTGAATTACTTACACCACTGGGCATTGA
TTTAGATGAGTGGAGTATGGCTACATACTACTTATTTGATGAGTCTGGTGAGTTT
AAATTGGCTTCACATATGTATTGTTCTTTCTACCCTCCAGATGAGGATGAAGAAG
AAGGTGATTGTGAAGAAGAAGAGTTTGAGCCATCAACTCAATATGAGTATGGTA
CTGAAGATGATTACCAAGGTAAACCTTTGGAATTTGGTGCCACTTCTGCTGCTCT
TCAACCTGAAGAAGAGCAAGAAGAAGATTGGTTAGATGATGATAGTCAACAAAC
TGTTGGTCAACAAGACGGCAGTGAGGACAATCAGACAACTACTATTCAAACAAT
TGTTGAGGTTCAACCTCAATTAGAGATGGAACTTACACCAGTTGTTCAGACTATT
GAAGTGAATAGTTTTAGTGGTTATTTAAAACTTACTGACAATGTATACATTAAAA
ATGCAGACATTGTGGAAGAAGCTAAAAAGGTAAAACCAACAGTGGTTGTTAATG
CAGCCAATGTTTACCTTAAACATGGAGGAGGTGTTGCAGGAGCCTTAAATAAGG
CTACTAACAATGCCATGCAAGTTGAATCTGATGATTACATAGCTACTAATGGACC
ACTTAAAGTGGGTGGTAGTTGTGTTTTAAGCGGACACAATCTTGCTAAACACTGT
CTTCATGTTGTCGGCCCAAATGTTAACAAAGGTGAAGACATTCAACTTCTTAAGA
GTGCTTATGAAAATTTTAATCAGCACGAAGTTCTACTTGCACCATTATTATCAGCT
GGTATTTTTGGTGCTGACCCTATACATTCTTTAAGAGTTTGTGTAGATACTGTTCG
CACAAATGTCTACTTAGCTGTCTTTGATAAAAATCTCTATGACAAACTTGTTTCAA
GCTTTTTGGAAATGAAGAGTGAAAAGCAAGTTGAACAAAAGATCGCTGAGATTC
CTAAAGAGGAAGTTAAGCCATTTATAACTGAAAGTAAACCTTCAGTTGAACAGA
GAAAACAAGATGATAAGAAAATCAAAGCTTGTGTTGAAGAAGTTACAACAACTC
TGGAAGAAACTAAGTTCCTCACAGAAAACTTGTTACTTTATATTGACATTAATGG
CAATCTTCATCCAGATTCTGCCACTCTTGTTAGTGACATTGACATCACTTTCTTAA
AGAAAGATGCTCCATATATAGTGGGTGATGTTGTTCAAGAGGGTGTTTTAACTGC
TGTGGTTATACCTACTAAAAAGGCTGGTGGCACTACTGAAATGCTAGCGAAAGC
TTTGAGAAAAGTGCCAACAGACAATTATATAACCACTTACCCGGGTCAGGGTTTA
AATGGTTACACTGTAGAGGAGGCAAAGACAGTGCTTAAAAAGTGTAAAAGTGCC
TTTTACATTCTACCATCTATTATCTCTAATGAGAAGCAAGAAATTCTTGGAACTGT
TTCTTGGAATTTGCGAGAAATGCTTGCACATGCAGAAGAAACACGCAAATTAAT
GCCTGTCTGTGTGGAAACTAAAGCCATAGTTTCAACTATACAGCGTAAATATAAG
GGTATTAAAATACAAGAGGGTGTGGTTGATTATGGTGCTAGATTTTACTTTTACA
CCAGTAAAACAACTGTAGCGTCACTTATCAACACACTTAACGATCTAAATGAAA
CTCTTGTTACAATGCCACTTGGCTATGTAACACATGGCTTAAATTTGGAAGAAGC
TGCTCGGTATATGAGATCTCTCAAAGTGCCAGCTACAGTTTCTGTTTCTTCACCTG
ATGCTGTTACAGCGTATAATGGTTATCTTACTTCTTCTTCTAAAACACCTGAAGAA
CATTTTATTGAAACCATCTCACTTGCTGGTTCCTATAAAGATTGGTCCTATTCTGG
ACAATCTACACAACTAGGTATAGAATTTCTTAAGAGAGGTGATAAAAGTGTATAT
TACACTAGTAATCCTACCACATTCCACCTAGATGGTGAAGTTATCACCTTTGACA
ATCTTAAGACACTTCTTTCTTTGAGAGAAGTGAGGACTATTAAGGTGTTTACAAC
AGTAGACAACATTAACCTCCACACGCAAGTTGTGGACATGTCAATGACATATGG
ACAACAGTTTGGTCCAACTTATTTGGATGGAGCTGATGTTACTAAAATAAAACCT
CATAATTCACATGAAGGTAAAACATTTTATGTTTTACCTAATGATGACACTCTAC
GTGTTGAGGCTTTTGAGTACTACCACACAACTGATCCTAGTTTTCTGGGTAGGTA
CATGTCAGCATTAAATCACACTAAAAAGTGGAAATACCCACAAGTTAATGGTTT
AACTTCTATTAAATGGGCAGATAACAACTGTTATCTTGCCACTGCATTGTTAACA
CTCCAACAAATAGAGTTGAAGTTTAATCCACCTGCTCTACAAGATGCTTATTACA
GAGCAAGGGCTGGTGAAGCTGCTAACTTTTGTGCACTTATCTTAGCCTACTGTAA
TAAGACAGTAGGTGAGTTAGGTGATGTTAGAGAAACAATGAGTTACTTGTTTCAA
CATGCCAATTTAGATTCTTGCAAAAGAGTCTTGAACGTGGTGTGTAAAACTTGTG
GACAACAGCAGACAACCCTTAAGGGTGTAGAAGCTGTTATGTACATGGGCACAC
TTTCTTATGAACAATTTAAGAAAGGTGTTCAGATACCTTGTACGTGTGGTAAACA
AGCTACAAAATATCTAGTACAACAGGAGTCACCTTTTGTTATGATGTCAGCACCA
CCTGCTCAGTATGAACTTAAGCATGGTACATTTACTTGTGCTAGTGAGTACACTG
GTAATTACCAGTGTGGTCACTATAAACATATAACTTCTAAAGAAACTTTGTATTG
CATAGACGGTGCTTTACTTACAAAGTCCTCAGAATACAAAGGTCCTATTACGGAT
GTTTTCTACAAAGAAAACAGTTACACAACAACCATAAAACCAGTTACTTATAAAT
TGGATGGTGTTGTTTGTACAGAAATTGACCCTAAGTTGGACAATTATTATAAGAA
AGACAATTCTTATTTCACAGAGCAACCAATTGATCTTGTACCAAACCAACCATAT
CCAAACGCAAGCTTCGATAATTTTAAGTTTGTATGTGATAATATCAAATTTGCTG
ATGATTTAAACCAGTTAACTGGTTATAAGAAACCTGCTTCAAGAGAGCTTAAAGT
TACATTTTTCCCTGACTTAAATGGTGATGTGGTGGCTATTGATTATAAACACTACA
CACCCTCTTTTAAGAAAGGAGCTAAATTGTTACATAAACCTATTGTTTGGCATGT
TAACAATGCAACTAATAAAGCCACGTATAAACCAAATACCTGGTGTATACGTTGT
CTTTGGAGCACAAAACCAGTTGAAACATCAAATTCGTTTGATGTACTGAAGTCAG
AGGACGCGCAGGGAATGGATAATCTTGCCTGCGAAGATCTAAAACCAGTCTCTG
AAGAAGTAGTGGAAAATCCTACCATACAGAAAGACGTTCTTGAGTGTAATGTGA
AAACTACCGAAGTTGTAGGAGACATTATACTTAAACCAGCAAATAATAGTTTAA
AAATTACAGAAGAGGTTGGCCACACAGATCTAATGGCTGCTTATGTAGACAATT
CTAGTCTTACTATTAAGAAACCTAATGAATTATCTAGAGTATTAGGTTTGAAAAC
CCTTGCTACTCATGGTTTAGCTGCTGTTAATAGTGTCCCTTGGGATACTATAGCTA
ATTATGCTAAGCCTTTTCTTAACAAAGTTGTTAGTACAACTACTAACATAGTTAC
ACGGTGTTTAAACCGTGTTTGTACTAATTATATGCCTTATTTCTTTACTTTATTGCT
ACAATTGTGTACTTTTACTAGAAGTACAAATTCTAGAATTAAAGCATCTATGCCG
ACTACTATAGCAAAGAATACTGTTAAGAGTGTCGGTAAATTTTGTCTAGAGGCTT
CATTTAATTATTTGAAGTCACCTAATTTTTCTAAACTGATAAATATTATAATTTGG
TTTTTACTATTAAGTGTTTGCCTAGGTTCTTTAATATACTCAACGGCGGCGTTAGG
TGTTTTAATGTCTAATTTAGGCATGCCGTCTTACTGTACGGGTTACAGAGAAGGC
TATTTGAACTCTACGAATGTCACGATAGCGACGTACTGTACGGGTTCTATACCGT
GTAGTGTTTGTCTTAGTGGTTTAGATTCTTTAGACACGTATCCGTCTTTAGAAACG
ATACAAATAACGATATCATCTTTTAAATGGGATTTAACGGCGTTTGGCTTAGTTG
CGGAGTGGTTTTTGGCGTATATTCTTTTCACGAGGTTTTTCTATGTACTTGGATTG
GCGGCGATAATGCAATTGTTTTTCAGCTATTTTGCGGTACATTTTATAAGTAATTC
TTGGCTTATGTGGTTAATAATAAATCTTGTACAAATGGCGCCGATATCAGCGATG
GTTAGAATGTACATATTCTTTGCGTCATTTTATTATGTATGGAAAAGTTATGTGCA
TGTTGTAGACGGTTGTAATTCATCAACGTGTATGATGTGTTACAAACGTAATAGA
GCGACGAGAGTCGAATGTACGACGATAGTTAATGGTGTTAGAAGGTCCTTTTATG
TCTATGCGAATGGAGGTAAAGGCTTTTGCAAACTACACAATTGGAATTGTGTTAA
TTGTGATACGTTCTGTGCGGGTAGTACGTTTATAAGTGATGAAGTTGCGAGAGAC
TTGTCACTACAGTTTAAAAGACCGATAAATCCGACGGACCAGTCTTCTTACATAG
TTGATAGTGTTACGGTGAAGAATGGTTCCATACATCTTTACTTTGATAAAGCGGG
TCAAAAGACGTATGAAAGACATTCTCTCTCTCATTTTGTTAACTTAGACAACCTG
AGAGCGAATAACACGAAAGGTTCATTGCCGATAAATGTTATAGTTTTTGATGGTA
AATCAAAATGTGAAGAATCATCTGCGAAATCAGCGTCTGTTTACTACAGTCAGCT
TATGTGTCAACCGATACTGTTACTAGATCAGGCGTTAGTGTCTGATGTTGGTGAT
AGTGCGGAAGTTGCGGTTAAAATGTTTGATGCGTACGTTAATACGTTTTCATCAA
CGTTTAACGTACCGATGGAAAAACTCAAAACGCTAGTTGCGACGGCGGAAGCGG
AACTTGCGAAGAATGTGTCCTTAGACAATGTCTTATCTACGTTTATATCAGCGGC
GCGGCAAGGGTTTGTTGATTCAGATGTAGAAACGAAAGATGTTGTTGAATGTCTT
AAATTGTCACATCAATCTGACATAGAAGTTACGGGCGATAGTTGTAATAACTATA
TGCTCACGTATAACAAAGTTGAAAACATGACGCCGCGTGACCTTGGTGCGTGTAT
AGACTGTAGTGCGCGTCATATAAATGCGCAGGTAGCGAAAAGTCACAACATAGC
GTTGATATGGAACGTTAAAGATTTCATGTCATTGTCTGAACAACTACGAAAACAA
ATACGTAGTGCGGCGAAAAAGAATAACTTACCGTTTAAGTTGACGTGTGCGACG
ACGAGACAAGTTGTTAATGTTGTAACGACGAAGATAGCGCTTAAGGGTGGTAAA
ATAGTTAATAATTGGTTGAAGCAGTTAATTAAA
SEQ ID NO: 54 - Clone pCC1-4K-SARS-COV-2-4N-2. Only fragment 2 has been
deoptimized.
SEQ ID NO: 55 - Clone pCC1-4K-SARS-COV-2-4N-3. Only fragment 1 has been
deoptimized.
SEQ ID NO: 56 - Clone pCC1-4K-SARS-COV-2-4N-4. Only fragments 1 and 2
have been deoptimized.
SEQ ID NO: 57 - Clone pCC1-4K-SARS-COV-2-4N-5. Only fragments 1 and 3
have been deoptimized.
SEQ ID NO: 58 - Clone pCC1-4K-SARS-COV-2-4N-6. Only fragments 2 and 3
have been deoptimized.
SEQ ID NO: 59 - Clone pCC1-4K-SARS-COV-2-4N-7. All 3 fragments were
deoptimized. Deoptimized sequence between SanDI to PacI in ORF1a:
GGGTCCCACGTGCGAGCGCGAACATAGGTTGTAACCATACGGGTGTT
GTTGGAGAAGGTTCCGAAGGTCTTAATGACAACCTTCTTGAAATACTCCAAAAA
GAGAAAGTCAACATAAATATAGTTGGTGACTTTAAACTTAATGAAGAGATAGCG
ATAATATTGGCGTCTTTTTCTGCGTCCACGAGTGCGTTTGTGGAAACGGTGAAAG
GTTTGGATTATAAAGCGTTCAAACAAATTGTTGAATCCTGTGGTAATTTTAAAGT
TACGAAAGGAAAAGCGAAAAAAGGTGCGTGGAATATAGGTGAACAGAAATCAA
TACTGAGTCCGCTTTATGCGTTTGCGTCAGAGGCGGCGCGTGTTGTACGATCAAT
ATTCTCCCGCACGCTTGAAACGGCGCAAAATTCTGTGCGTGTTTTACAGAAGGCG
GCGATAACGATACTAGATGGAATATCACAGTATTCACTGAGACTCATAGATGCG
ATGATGTTCACGTCTGATTTGGCGACGAACAATCTAGTTGTAATGGCGTACATAA
CGGGTGGTGTTGTTCAGTTGACGTCGCAGTGGCTAACGAACATATTTGGCACGGT
TTATGAAAAACTCAAACCGGTCCTTGATTGGCTTGAAGAGAAGTTTAAGGAAGG
TGTAGAGTTTCTTAGAGACGGTTGGGAAATAGTTAAATTTATATCAACGTGTGCG
TGTGAAATAGTCGGTGGACAAATAGTCACGTGTGCGAAGGAAATAAAGGAGAGT
GTTCAGACGTTCTTTAAGCTTGTAAATAAATTTTTGGCGTTGTGTGCGGACTCTAT
AATAATAGGTGGAGCGAAACTTAAAGCGTTGAATTTAGGTGAAACGTTTGTCAC
GCACTCAAAGGGATTGTACAGAAAGTGTGTTAAATCCAGAGAAGAAACGGGCCT
ACTCATGCCGCTAAAAGCGCCGAAAGAAATAATATTCTTAGAGGGAGAAACGCT
TCCGACGGAAGTGTTAACGGAGGAAGTTGTCTTGAAAACGGGTGATTTACAACC
GTTAGAACAACCGACGAGTGAAGCGGTTGAAGCGCCGTTGGTTGGTACGCCGGT
TTGTATAAACGGGCTTATGTTGCTCGAAATAAAAGACACGGAAAAGTACTGTGC
GCTTGCGCCGAATATGATGGTAACGAACAATACGTTCACGCTCAAAGGCGGTGC
GCCGACAAAGGTTACGTTTGGTGATGACACGGTGATAGAAGTGCAAGGTTACAA
GAGTGTGAATATAACGTTTGAACTTGATGAAAGGATAGATAAAGTACTTAATGA
GAAGTGCTCTGCGTATACGGTTGAACTCGGTACGGAAGTAAATGAGTTCGCGTGT
GTTGTGGCGGATGCGGTCATAAAAACGTTGCAACCGGTATCTGAATTACTTACGC
CGCTGGGCATAGATTTAGATGAGTGGAGTATGGCGACGTACTACTTATTTGATGA
GTCTGGTGAGTTTAAATTGGCGTCACATATGTATTGTTCTTTCTACCCGCCGGATG
AGGATGAAGAAGAAGGTGATTGTGAAGAAGAAGAGTTTGAGCCGTCAACGCAAT
ATGAGTATGGTACGGAAGATGATTACCAAGGTAAACCGTTGGAATTTGGTGCGA
CGTCTGCGGCGCTTCAACCGGAAGAAGAGCAAGAAGAAGATTGGTTAGATGATG
ATAGTCAACAAACGGTTGGTCAACAAGACGGCAGTGAGGACAATCAGACGACG
ACGATTCAAACGATAGTTGAGGTTCAACCGCAATTAGAGATGGAACTTACGCCG
GTTGTTCAGACGATAGAAGTGAATAGTTTTAGTGGTTATTTAAAACTTACGGACA
ATGTATACATAAAAAATGCGGACATAGTGGAAGAAGCGAAAAAGGTAAAACCG
ACGGTGGTTGTTAATGCGGCGAATGTTTACCTTAAACATGGAGGAGGTGTTGCGG
GAGCGTTAAATAAGGCGACGAACAATGCGATGCAAGTTGAATCTGATGATTACA
TAGCGACGAATGGACCGCTTAAAGTGGGTGGTAGTTGTGTTTTAAGCGGACACA
ATCTTGCGAAACACTGTCTTCATGTTGTCGGCCCGAATGTTAACAAAGGTGAAGA
CATACAACTTCTTAAGAGTGCGTATGAAAATTTTAATCAGCACGAAGTTCTACTT
GCGCCGTTATTATCAGCGGGTATATTTGGTGCGGACCCGATACATTCTTTAAGAG
TTTGTGTAGATACGGTTCGCACGAATGTCTACTTAGCGGTCTTTGATAAAAATCT
CTATGACAAACTTGTTTCAAGCTTTTTGGAAATGAAGAGTGAAAAGCAAGTTGAA
CAAAAGATAGCGGAGATACCGAAAGAGGAAGTTAAGCCGTTTATAACGGAAAG
TAAACCGTCAGTTGAACAGAGAAAACAAGATGATAAGAAAATAAAAGCGTGTGT
TGAAGAAGTTACGACGACGCTGGAAGAAACGAAGTTCCTCACGGAAAACTTGTT
ACTTTATATAGACATAAATGGCAATCTTCATCCGGATTCTGCGACGCTTGTTAGT
GACATAGACATAACGTTCTTAAAGAAAGATGCGCCGTATATAGTGGGTGATGTT
GTTCAAGAGGGTGTTTTAACGGCGGTGGTTATACCGACGAAAAAGGCGGGTGGC
ACGACGGAAATGCTAGCGAAAGCGTTGAGAAAAGTGCCGACGGACAATTATATA
ACGACGTACCCGGGTCAGGGTTTAAATGGTTACACGGTAGAGGAGGCGAAGACG
GTGCTTAAAAAGTGTAAAAGTGCGTTTTACATACTACCGTCTATAATATCTAATG
AGAAGCAAGAAATACTTGGAACGGTTTCTTGGAATTTGCGAGAAATGCTTGCGC
ATGCGGAAGAAACGCGCAAATTAATGCCGGTCTGTGTGGAAACGAAAGCGATAG
TTTCAACGATACAGCGTAAATATAAGGGTATAAAAATACAAGAGGGTGTGGTTG
ATTATGGTGCGAGATTTTACTTTTACACGAGTAAAACGACGGTAGCGTCACTTAT
AAACACGCTTAACGATCTAAATGAAACGCTTGTTACGATGCCGCTTGGCTATGTA
ACGCATGGCTTAAATTTGGAAGAAGCGGCGCGGTATATGAGATCTCTCAAAGTG
CCGGCGACGGTTTCTGTTTCTTCACCGGATGCGGTTACGGCGTATAATGGTTATC
TTACGTCTTCTTCTAAAACGCCGGAAGAACATTTTATTGAAACGATATCACTTGC
GGGTTCCTATAAAGATTGGTCCTATTCTGGACAATCTACGCAACTAGGTATAGAA
TTTCTTAAGAGAGGTGATAAAAGTGTATATTACACGAGTAATCCGACGACGTTCC
ACCTAGATGGTGAAGTTATAACGTTTGACAATCTTAAGACGCTTCTTTCTTTGAG
AGAAGTGAGGACGATAAAGGTGTTTACGACGGTAGACAACATAAACCTCCACAC
GCAAGTTGTGGACATGTCAATGACGTATGGACAACAGTTTGGTCCGACGTATTTG
GATGGAGCGGATGTTACGAAAATAAAACCGCATAATTCACATGAAGGTAAAACG
TTTTATGTTTTACCGAATGATGACACGCTACGTGTTGAGGCGTTTGAGTACTACC
ACACGACGGATCCGAGTTTTCTGGGTAGGTACATGTCAGCGTTAAATCACACGA
AAAAGTGGAAATACCCGCAAGTTAATGGTTTAACGTCTATAAAATGGGCGGATA
ACAACTGTTATCTTGCGACGGCGTTGTTAACGCTCCAACAAATAGAGTTGAAGTT
TAATCCGCCGGCGCTACAAGATGCGTATTACAGAGCGAGGGCGGGTGAAGCGGC
GAACTTTTGTGCGCTTATATTAGCGTACTGTAATAAGACGGTAGGTGAGTTAGGT
GATGTTAGAGAAACGATGAGTTACTTGTTTCAACATGCGAATTTAGATTCTTGCA
AAAGAGTCTTGAACGTGGTGTGTAAAACGTGTGGACAACAGCAGACGACGCTTA
AGGGTGTAGAAGCGGTTATGTACATGGGCACGCTTTCTTATGAACAATTTAAGAA
AGGTGTTCAGATACCGTGTACGTGTGGTAAACAAGCGACGAAATATCTAGTACA
ACAGGAGTCACCGTTTGTTATGATGTCAGCGCCGCCGGCGCAGTATGAACTTAAG
CATGGTACGTTTACGTGTGCGAGTGAGTACACGGGTAATTACCAGTGTGGTCACT
ATAAACATATAACGTCTAAAGAAACGTTGTATTGCATAGACGGTGCGTTACTTAC
GAAGTCCTCAGAATACAAAGGTCCGATAACGGATGTTTTCTACAAAGAAAACAG
TTACACGACGACGATAAAACCGGTTACGTATAAATTGGATGGTGTTGTTTGTACG
GAAATAGACCCGAAGTTGGACAATTATTATAAGAAAGACAATTCTTATTTCACGG
AGCAACCGATAGATCTTGTACCGAACCAACCGTATCCGAACGCGAGCTTCGATA
ATTTTAAGTTTGTATGTGATAATATAAAATTTGCGGATGATTTAAACCAGTTAAC
GGGTTATAAGAAACCGGCGTCAAGAGAGCTTAAAGTTACGTTTTTCCCGGACTTA
AATGGTGATGTGGTGGCGATAGATTATAAACACTACACGCCGTCTTTTAAGAAAG
GAGCGAAATTGTTACATAAACCGATAGTTTGGCATGTTAACAATGCGACGAATA
AAGCGACGTATAAACCGAATACGTGGTGTATACGTTGTCTTTGGAGCACGAAAC
CGGTTGAAACGTCAAATTCGTTTGATGTACTGAAGTCAGAGGACGCGCAGGGAA
TGGATAATCTTGCGTGCGAAGATCTAAAACCGGTCTCTGAAGAAGTAGTGGAAA
ATCCGACGATACAGAAAGACGTTCTTGAGTGTAATGTGAAAACGACGGAAGTTG
TAGGAGACATAATACTTAAACCGGCGAATAATAGTTTAAAAATAACGGAAGAGG
TTGGCCACACGGATCTAATGGCGGCGTATGTAGACAATTCTAGTCTTACGATAAA
GAAACCGAATGAATTATCTAGAGTATTAGGTTTGAAAACGCTTGCGACGCATGGT
TTAGCGGCGGTTAATAGTGTCCCGTGGGATACGATAGCGAATTATGCGAAGCCG
TTTCTTAACAAAGTTGTTAGTACGACGACGAACATAGTTACGCGGTGTTTAAACC
GTGTTTGTACGAATTATATGCCGTATTTCTTTACGTTATTGCTACAATTGTGTACG
TTTACGAGAAGTACGAATTCTAGAATAAAAGCGTCTATGCCGACGACGATAGCG
AAGAATACGGTTAAGAGTGTCGGTAAATTTTGTCTAGAGGCGTCATTTAATTATT
TGAAGTCACCGAATTTTTCTAAACTGATAAATATAATAATATGGTTTTTACTATTA
AGTGTTTGCCTAGGTTCTTTAATATACTCAACGGCGGCGTTAGGTGTTTTAATGTC
TAATTTAGGCATGCCGTCTTACTGTACGGGTTACAGAGAAGGCTATTTGAACTCT
ACGAATGTCACGATAGCGACGTACTGTACGGGTTCTATACCGTGTAGTGTTTGTC
TTAGTGGTTTAGATTCTTTAGACACGTATCCGTCTTTAGAAACGATACAAATAAC
GATATCATCTTTTAAATGGGATTTAACGGCGTTTGGCTTAGTTGCGGAGTGGTTTT
TGGCGTATATTCTTTTCACGAGGTTTTTCTATGTACTTGGATTGGCGGCGATAATG
CAATTGTTTTTCAGCTATTTTGCGGTACATTTTATAAGTAATTCTTGGCTTATGTG
GTTAATAATAAATCTTGTACAAATGGCGCCGATATCAGCGATGGTTAGAATGTAC
ATATTCTTTGCGTCATTTTATTATGTATGGAAAAGTTATGTGCATGTTGTAGACGG
TTGTAATTCATCAACGTGTATGATGTGTTACAAACGTAATAGAGCGACGAGAGTC
GAATGTACGACGATAGTTAATGGTGTTAGAAGGTCCTTTTATGTCTATGCGAATG
GAGGTAAAGGCTTTTGCAAACTACACAATTGGAATTGTGTTAATTGTGATACGTT
CTGTGCGGGTAGTACGTTTATAAGTGATGAAGTTGCGAGAGACTTGTCACTACAG
TTTAAAAGACCGATAAATCCGACGGACCAGTCTTCTTACATAGTTGATAGTGTTA
CGGTGAAGAATGGTTCCATACATCTTTACTTTGATAAAGCGGGTCAAAAGACGTA
TGAAAGACATTCTCTCTCTCATTTTGTTAACTTAGACAACCTGAGAGCGAATAAC
ACGAAAGGTTCATTGCCGATAAATGTTATAGTTTTTGATGGTAAATCAAAATGTG
AAGAATCATCTGCGAAATCAGCGTCTGTTTACTACAGTCAGCTTATGTGTCAACC
GATACTGTTACTAGATCAGGCGTTAGTGTCTGATGTTGGTGATAGTGCGGAAGTT
GCGGTTAAAATGTTTGATGCGTACGTTAATACGTTTTCATCAACGTTTAACGTAC
CGATGGAAAAACTCAAAACGCTAGTTGCGACGGCGGAAGCGGAACTTGCGAAG
AATGTGTCCTTAGACAATGTCTTATCTACGTTTATATCAGCGGCGCGGCAAGGGT
TTGTTGATTCAGATGTAGAAACGAAAGATGTTGTTGAATGTCTTAAATTGTCACA
TCAATCTGACATAGAAGTTACGGGCGATAGTTGTAATAACTATATGCTCACGTAT
AACAAAGTTGAAAACATGACGCCGCGTGACCTTGGTGCGTGTATAGACTGTAGT
GCGCGTCATATAAATGCGCAGGTAGCGAAAAGTCACAACATAGCGTTGATATGG
AACGTTAAAGATTTCATGTCATTGTCTGAACAACTACGAAAACAAATACGTAGTG
CGGCGAAAAAGAATAACTTACCGTTTAAGTTGACGTGTGCGACGACGAGACAAG
TTGTTAATGTTGTAACGACGAAGATAGCGCTTAAGGGTGGTAAAATAGTTAATAA
TTGGTTGAAGCAGTTAATTAAA
SEQ ID NO: 60 - Clone pCC1-4K-SARS-COV-2-7N-1. Only fragment 3 has been
deoptimized:
GGGTCCCACGTGCTAGCGCTAACATAGGTTGTAACCATACAGGTGTTG
TTGGAGAAGGTTCCGAAGGTCTTAATGACAACCTTCTTGAAATACTCCAAAAAG
AGAAAGTCAACATCAATATTGTTGGTGACTTTAAACTTAATGAAGAGATCGCCAT
TATTTTGGCATCTTTTTCTGCTTCCACAAGTGCTTTTGTGGAAACTGTGAAAGGTT
TGGATTATAAAGCATTCAAACAAATTGTTGAATCCTGTGGTAATTTTAAAGTTAC
AAAAGGAAAAGCTAAAAAAGGTGCCTGGAATATTGGTGAACAGAAATCAATACT
GAGTCCTCTTTATGCATTTGCATCAGAGGCTGCTCGTGTTGTACGATCAATTTTCT
CCCGCACTCTTGAAACTGCTCAAAATTCTGTGCGTGTTTTACAGAAGGCCGCTAT
AACAATACTAGATGGAATTTCACAGTATTCACTGAGACTCATTGATGCTATGATG
TTCACATCTGATTTGGCTACTAACAATCTAGTTGTAATGGCCTACATTACAGGTG
GTGTTGTTCAGTTGACTTCGCAGTGGCTAACTAACATCTTTGGCACTGTTTATGAA
AAACTCAAACCCGTCCTTGATTGGCTTGAAGAGAAGTTTAAGGAAGGTGTAGAG
TTTCTTAGAGACGGTTGGGAAATTGTTAAATTTATCTCAACCTGTGCTTGTGAAAT
TGTCGGTGGACAAATTGTCACCTGTGCAAAGGAAATTAAGGAGAGTGTTCAGAC
ATTCTTTAAGCTTGTAAATAAATTTTTGGCTTTGTGTGCTGACTCTATCATTATTG
GTGGAGCTAAACTTAAAGCCTTGAATTTAGGTGAAACATTTGTCACGCACTCAAA
GGGATTGTACAGAAAGTGTGTTAAATCCAGAGAAGAAACTGGCCTACTCATGCC
TCTAAAAGCCCCAAAAGAAATTATCTTCTTAGAGGGAGAAACACTTCCCACAGA
AGTGTTAACAGAGGAAGTTGTCTTGAAAACTGGTGATTTACAACCATTAGAACA
ACCTACTAGTGAAGCTGTTGAAGCTCCATTGGTTGGTACACCAGTTTGTATTAAC
GGGCTTATGTTGCTCGAAATCAAAGACACAGAAAAGTACTGTGCCCTTGCACCTA
ATATGATGGTAACAAACAATACCTTCACACTCAAAGGCGGTGCACCAACAAAGG
TTACTTTTGGTGATGACACTGTGATAGAAGTGCAAGGTTACAAGAGTGTGAATAT
CACTTTTGAACTTGATGAAAGGATTGATAAAGTACTTAATGAGAAGTGCTCTGCC
TATACAGTTGAACTCGGTACAGAAGTAAATGAGTTCGCCTGTGTTGTGGCAGATG
CTGTCATAAAAACTTTGCAACCAGTATCTGAATTACTTACACCACTGGGCATTGA
TTTAGATGAGTGGAGTATGGCTACATACTACTTATTTGATGAGTCTGGTGAGTTT
AAATTGGCTTCACATATGTATTGTTCTTTCTACCCTCCAGATGAGGATGAAGAAG
AAGGTGATTGTGAAGAAGAAGAGTTTGAGCCATCAACTCAATATGAGTATGGTA
CTGAAGATGATTACCAAGGTAAACCTTTGGAATTTGGTGCCACTTCTGCTGCTCT
TCAACCTGAAGAAGAGCAAGAAGAAGATTGGTTAGATGATGATAGTCAACAAAC
TGTTGGTCAACAAGACGGCAGTGAGGACAATCAGACAACTACTATTCAAACAAT
TGTTGAGGTTCAACCTCAATTAGAGATGGAACTTACACCAGTTGTTCAGACTATT
GAAGTGAATAGTTTTAGTGGTTATTTAAAACTTACTGACAATGTATACATTAAAA
ATGCAGACATTGTGGAAGAAGCTAAAAAGGTAAAACCAACAGTGGTTGTTAATG
CAGCCAATGTTTACCTTAAACATGGAGGAGGTGTTGCAGGAGCCTTAAATAAGG
CTACTAACAATGCCATGCAAGTTGAATCTGATGATTACATAGCTACTAATGGACC
ACTTAAAGTGGGTGGTAGTTGTGTTTTAAGCGGACACAATCTTGCTAAACACTGT
CTTCATGTTGTCGGCCCAAATGTTAACAAAGGTGAAGACATTCAACTTCTTAAGA
GTGCTTATGAAAATTTTAATCAGCACGAAGTTCTACTTGCACCATTATTATCAGCT
GGTATTTTTGGTGCTGACCCTATACATTCTTTAAGAGTTTGTGTAGATACTGTTCG
CACAAATGTCTACTTAGCTGTCTTTGATAAAAATCTCTATGACAAACTTGTTTCAA
GCTTTTTGGAAATGAAGAGTGAAAAGCAAGTTGAACAAAAGATCGCTGAGATTC
CTAAAGAGGAAGTTAAGCCATTTATAACTGAAAGTAAACCTTCAGTTGAACAGA
GAAAACAAGATGATAAGAAAATCAAAGCTTGTGTTGAAGAAGTTACAACAACTC
TGGAAGAAACTAAGTTCCTCACAGAAAACTTGTTACTTTATATTGACATTAATGG
CAATCTTCATCCAGATTCTGCCACTCTTGTTAGTGACATTGACATCACTTTCTTAA
AGAAAGATGCTCCATATATAGTGGGTGATGTTGTTCAAGAGGGTGTTTTAACTGC
TGTGGTTATACCTACTAAAAAGGCTGGTGGCACTACTGAAATGCTAGCGAAAGC
TTTGAGAAAAGTGCCAACAGACAATTATATAACCACTTACCCGGGTCAGGGTTTA
AATGGTTACACTGTAGAGGAGGCAAAGACAGTGCTTAAAAAGTGTAAAAGTGCC
TTTTACATTCTACCATCTATTATCTCTAATGAGAAGCAAGAAATTCTTGGAACTGT
TTCTTGGAATTTGCGAGAAATGCTTGCACATGCAGAAGAAACACGCAAATTAAT
GCCTGTCTGTGTGGAAACTAAAGCCATAGTTTCAACTATACAGCGTAAATATAAG
GGTATTAAAATACAAGAGGGTGTGGTTGATTATGGTGCTAGATTTTACTTTTACA
CCAGTAAAACAACTGTAGCGTCACTTATCAACACACTTAACGATCTAAATGAAA
CTCTTGTTACAATGCCACTTGGCTATGTAACACATGGCTTAAATTTGGAAGAAGC
TGCTCGGTATATGAGATCTCTCAAAGTGCCAGCTACAGTTTCTGTTTCTTCACCTG
ATGCTGTTACAGCGTATAATGGTTATCTTACTTCTTCTTCTAAAACACCTGAAGAA
CATTTTATTGAAACCATCTCACTTGCTGGTTCCTATAAAGATTGGTCCTATTCTGG
ACAATCTACACAACTAGGTATAGAATTTCTTAAGAGAGGTGATAAAAGTGTATAT
TACACTAGTAATCCTACCACATTCCACCTAGATGGTGAAGTTATCACCTTTGACA
ATCTTAAGACACTTCTTTCTTTGAGAGAAGTGAGGACTATTAAGGTGTTTACAAC
AGTAGACAACATTAACCTCCACACGCAAGTTGTGGACATGTCAATGACATATGG
ACAACAGTTTGGTCCAACTTATTTGGATGGAGCTGATGTTACTAAAATAAAACCT
CATAATTCACATGAAGGTAAAACATTTTATGTTTTACCTAATGATGACACTCTAC
GTGTTGAGGCTTTTGAGTACTACCACACAACTGATCCTAGTTTTCTGGGTAGGTA
CATGTCAGCATTAAATCACACTAAAAAGTGGAAATACCCACAAGTTAATGGTTT
AACTTCTATTAAATGGGCAGATAACAACTGTTATCTTGCCACTGCATTGTTAACA
CTCCAACAAATAGAGTTGAAGTTTAATCCACCTGCTCTACAAGATGCTTATTACA
GAGCAAGGGCTGGTGAAGCTGCTAACTTTTGTGCACTTATCTTAGCCTACTGTAA
TAAGACAGTAGGTGAGTTAGGTGATGTTAGAGAAACAATGAGTTACTTGTTTCAA
CATGCCAATTTAGATTCTTGCAAAAGAGTCTTGAACGTGGTGTGTAAAACTTGTG
GACAACAGCAGACAACCCTTAAGGGTGTAGAAGCTGTTATGTACATGGGCACAC
TTTCTTATGAACAATTTAAGAAAGGTGTTCAGATACCTTGTACGTGTGGTAAACA
AGCTACAAAATATCTAGTACAACAGGAGTCACCTTTTGTTATGATGTCAGCACCA
CCTGCTCAGTATGAACTTAAGCATGGTACATTTACTTGTGCTAGTGAGTACACTG
GTAATTACCAGTGTGGTCACTATAAACATATAACTTCTAAAGAAACTTTGTATTG
CATAGACGGTGCTTTACTTACAAAGTCCTCAGAATACAAAGGTCCTATTACGGAT
GTTTTCTACAAAGAAAACAGTTACACAACAACCATAAAACCAGTTACTTATAAAT
TGGATGGTGTTGTTTGTACAGAAATTGACCCTAAGTTGGACAATTATTATAAGAA
AGACAATTCTTATTTCACAGAGCAACCAATTGATCTTGTACCAAACCAACCATAT
CCAAACGCAAGCTTCGATAATTTTAAGTTTGTATGTGATAATATCAAATTTGCTG
ATGATTTAAACCAGTTAACTGGTTATAAGAAACCTGCTTCAAGAGAGCTTAAAGT
TACATTTTTCCCTGACTTAAATGGTGATGTGGTGGCTATTGATTATAAACACTACA
CACCCTCTTTTAAGAAAGGAGCTAAATTGTTACATAAACCTATTGTTTGGCATGT
TAACAATGCAACTAATAAAGCCACGTATAAACCAAATACCTGGTGTATACGTTGT
CTTTGGAGCACAAAACCAGTTGAAACATCAAATTCGTTTGATGTACTGAAGTCAG
AGGACGCGCAGGGAATGGATAATCTTGCCTGCGAAGATCTAAAACCAGTCTCTG
AAGAAGTAGTGGAAAATCCTACCATACAGAAAGACGTTCTTGAGTGTAATGTGA
AAACTACCGAAGTTGTAGGAGACATTATACTTAAACCAGCAAATAATAGTTTAA
AAATTACAGAAGAGGTTGGCCACACAGATCTAATGGCTGCTTATGTAGACAATT
CTAGTCTTACTATTAAGAAACCTAATGAATTATCTAGAGTATTAGGTTTGAAAAC
CCTTGCTACTCATGGTTTAGCTGCTGTTAATAGTGTCCCTTGGGATACTATAGCTA
ATTATGCTAAGCCTTTTCTTAACAAAGTTGTTAGTACAACTACTAACATAGTTAC
ACGGTGTTTAAACCGTGTTTGTACTAATTATATGCCTTATTTCTTTACTTTATTGCT
ACAATTGTGTACTTTTACTAGAAGTACAAATTCTAGAATTAAAGCATCTATGCCG
ACTACTATAGCAAAGAATACTGTTAAGAGTGTCGGTAAATTTTGTCTAGAGGCTT
CATTTAATTATTTGAAGTCACCTAATTTTTCTAAACTGATAAATATTATAATTTGG
TTTTTACTATTAAGTGTTTGCCTAGGTTCTTTAATCTACTCAACCGCGGCGTTAGG
TGTTTTAATGTCTAATTTAGGTATGCCGTCTTACTGTACGGGTTACCGTGAAGGTT
ATTTGAACTCTACGAATGTCACGATTGCGACGTACTGTACGGGTTCTATACCGTG
TAGTGTTTGTCTTAGTGGTTTAGATTCTTTAGACACGTATCCGTCTTTAGAAACGA
TACAAATTACGATTTCATCTTTTAAATGGGATTTAACGGCGTTTGGTTTAGTTGCG
GAGTGGTTTTTGGCGTATATTCTTTTCACGCGTTTTTTCTATGTACTTGGTTTGGCG
GCGATCATGCAATTGTTTTTCAGCTATTTTGCGGTACATTTTATTAGTAATTCTTG
GCTTATGTGGTTAATAATTAATCTTGTACAAATGGCGCCGATTTCAGCGATGGTT
AGAATGTACATCTTCTTTGCGTCATTTTATTATGTATGGAAAAGTTATGTGCATGT
TGTAGACGGTTGTAATTCATCAACGTGTATGATGTGTTACAAACGTAATAGAGCG
ACGCGTGTCGAATGTACGACGATTGTTAATGGTGTTAGACGTTCCTTTTATGTCTA
TGCGAATGGTGGTAAAGGTTTTTGCAAACTACACAATTGGAATTGTGTTAATTGT
GATACGTTCTGTGCGGGTAGTACGTTTATTAGTGATGAAGTTGCGCGTGACTTGT
CACTACAGTTTAAACGTCCGATAAATCCGACGGACCAGTCTTCTTACATCGTTGA
TAGTGTTACGGTGAAGAATGGTTCCATCCATCTTTACTTTGATAAAGCGGGTCAA
AAGACGTATGAACGTCATTCTCTCTCTCATTTTGTTAACTTAGACAACCTGCGTGC
GAATAACACGAAAGGTTCATTGCCGATTAATGTTATAGTTTTTGATGGTAAATCA
AAATGTGAAGAATCATCTGCGAAATCAGCGTCTGTTTACTACAGTCAGCTTATGT
GTCAACCGATACTGTTACTAGATCAGGCGTTAGTGTCTGATGTTGGTGATAGTGC
GGAAGTTGCGGTTAAAATGTTTGATGCGTACGTTAATACGTTTTCATCAACGTTT
AACGTACCGATGGAAAAACTCAAAACGCTAGTTGCGACGGCGGAAGCGGAACTT
GCGAAGAATGTGTCCTTAGACAATGTCTTATCTACGTTTATTTCAGCGGCGCGTC
AAGGTTTTGTTGATTCAGATGTAGAAACGAAAGATGTTGTTGAATGTCTTAAATT
GTCACATCAATCTGACATAGAAGTTACGGGTGATAGTTGTAATAACTATATGCTC
ACGTATAACAAAGTTGAAAACATGACGCCGCGTGACCTTGGTGCGTGTATTGACT
GTAGTGCGCGTCATATTAATGCGCAGGTAGCGAAAAGTCACAACATTGCGTTGA
TATGGAACGTTAAAGATTTCATGTCATTGTCTGAACAACTACGTAAACAAATACG
TAGTGCGGCGAAAAAGAATAACTTACCGTTTAAGTTGACGTGTGCGACGACGCG
TCAAGTTGTTAATGTTGTAACGACGAAGATAGCGCTTAAGGGTGGTAAAATTGTT
AATAATTGGTTGAAGCAATTAATTAAA
SEQ ID NO: 61 - Clone pCC1-4K-SARS-COV-2-7N-2. Only fragment 2 has been
deoptimized.
SEQ ID NO: 62 - Clone pCC1-4K-SARS-COV-2-7N-3. Only fragment 1 has been
deoptimized.
SEQ ID NO: 63 - Clone pCC1-4K-SARS-COV-2-7N-4. Only fragments 1 and 2
have been deoptimized.
SEQ ID NO: 64 - Clone pCC1-4K-SARS-COV-2-7N-5. Only fragments 1 and 3
have been deoptimized.
SEQ ID NO: 65 - Clone pCC1-4K-SARS-COV-2-7N-6. Only fragments 2 and 3
have been deoptimized.
SEQ ID NO: 66 - Clone pCC1-4K-SARS-COV-2-7N-7. All 3 fragments were
deoptimized. Deoptimized sequence between SanDI to PacI in ORF1a:
GGGTCCCGCGTGCGAGCGCGAACATAGGTTGTAACCATACGGGTGTT
GTTGGTGAAGGTTCCGAAGGTCTTAATGACAACCTTCTTGAAATACTCCAAAAAG
AGAAAGTCAACATAAATATAGTTGGTGACTTTAAACTTAATGAAGAGATAGCGA
TAATATTGGCGTCTTTTTCTGCGTCCACGAGTGCGTTTGTGGAAACAGTGAAAGG
TTTGGATTATAAAGCGTTCAAACAAATAGTTGAATCCTGTGGTAATTTTAAAGTT
ACGAAAGGTAAAGCGAAAAAAGGTGCGTGGAATATAGGTGAACAAAAATCAAT
ACTGAGTCCGCTTTATGCGTTTGCGTCAGAGGCGGCGCGTGTTGTACGTTCAATA
TTCTCCCGTACGCTTGAAACGGCGCAAAATTCTGTGCGTGTTTTACAAAAGGCGG
CGATAACGATACTAGATGGTATATCACAATATTCACTGCGTCTCATAGATGCGAT
GATGTTCACGTCTGATTTGGCGACGAACAATCTAGTTGTAATGGCGTACATAACG
GGTGGTGTTGTTCAATTGACGTCGCAATGGCTAACGAACATATTTGGTACGGTTT
ATGAAAAACTCAAACCGGTCCTTGATTGGCTTGAAGAGAAGTTTAAGGAAGGTG
TAGAGTTTCTTCGTGACGGTTGGGAAATAGTTAAATTTATATCAACGTGTGCGTG
TGAAATAGTCGGTGGTCAAATAGTCACGTGTGCGAAGGAAATAAAGGAGAGTGT
TCAAACGTTCTTTAAGCTTGTAAATAAATTTTTGGCGTTGTGTGCGGACTCTATAA
TAATAGGTGGTGCGAAACTTAAAGCGTTGAATTTAGGTGAAACGTTTGTCACGCA
CTCAAAGGGTTTGTACCGTAAGTGTGTTAAATCCCGTGAAGAAACGGGTCTACTC
ATGCCGCTAAAAGCGCCGAAAGAAATAATATTCTTAGAGGGTGAAACGCTTCCG
ACGGAAGTGTTAACGGAGGAAGTTGTCTTGAAAACGGGTGATTTACAACCGTTA
GAACAACCGACGAGTGAAGCGGTTGAAGCGCCGTTGGTTGGTACGCCGGTTTGT
ATAAACGGTCTTATGTTGCTCGAAATAAAAGACACGGAAAAGTACTGTGCGCTT
GCGCCGAATATGATGGTAACGAACAATACGTTCACGCTCAAAGGTGGTGCGCCG
ACGAAGGTTACGTTTGGTGATGACACGGTGATAGAAGTGCAAGGTTACAAGAGT
GTGAATATAACGTTTGAACTTGATGAACGTATAGATAAAGTACTTAATGAGAAGT
GCTCTGCGTATACGGTTGAACTCGGTACGGAAGTAAATGAGTTCGCGTGTGTTGT
GGCGGATGCGGTCATAAAAACGTTGCAACCGGTATCTGAATTACTTACGCCGCTG
GGTATAGATTTAGATGAGTGGAGTATGGCGACGTACTACTTATTTGATGAGTCTG
GTGAGTTTAAATTGGCGTCACATATGTATTGTTCTTTCTACCCGCCGGATGAGGA
TGAAGAAGAAGGTGATTGTGAAGAAGAAGAGTTTGAGCCGTCAACGCAATATGA
GTATGGTACGGAAGATGATTACCAAGGTAAACCGTTGGAATTTGGTGCGACGTCT
GCGGCGCTTCAACCGGAAGAAGAGCAAGAAGAAGATTGGTTAGATGATGATAGT
CAACAAACGGTTGGTCAACAAGACGGTAGTGAGGACAATCAAACGACGACGAT
ACAAACGATAGTTGAGGTTCAACCGCAATTAGAGATGGAACTTACGCCGGTTGT
TCAAACGATAGAAGTGAATAGTTTTAGTGGTTATTTAAAACTTACGGACAATGTA
TACATAAAAAATGCGGACATAGTGGAAGAAGCGAAAAAGGTAAAACCGACGGT
GGTTGTTAATGCGGCGAATGTTTACCTTAAACATGGTGGTGGTGTTGCGGGTGCG
TTAAATAAGGCGACGAACAATGCGATGCAAGTTGAATCTGATGATTACATAGCG
ACGAATGGTCCGCTTAAAGTGGGTGGTAGTTGTGTTTTAAGCGGTCACAATCTTG
CGAAACACTGTCTTCATGTTGTCGGTCCGAATGTTAACAAAGGTGAAGACATTCA
ACTTCTTAAGAGTGCGTATGAAAATTTTAATCAACACGAAGTTCTACTTGCGCCG
TTATTATCAGCGGGTATATTTGGTGCGGACCCGATACATTCTTTACGTGTTTGTGT
AGATACGGTTCGTACGAATGTCTACTTAGCGGTCTTTGATAAAAATCTCTATGAC
AAACTTGTTTCAAGCTTTTTGGAAATGAAGAGTGAAAAGCAAGTTGAACAAAAG
ATAGCAGAGATACCGAAAGAGGAAGTTAAGCCGTTTATAACGGAAAGTAAACCG
TCAGTTGAACAGCGTAAACAAGATGATAAGAAAATAAAAGCGTGTGTTGAAGAA
GTTACGACGACGCTGGAAGAAACGAAGTTCCTCACGGAAAACTTGTTACTTTATA
TAGACATAAATGGTAATCTTCATCCGGATTCTGCGACGCTTGTTAGTGACATAGA
CATAACGTTCTTAAAGAAAGATGCGCCGTATATAGTGGGTGATGTTGTTCAAGAG
GGTGTTTTAACGGCGGTGGTTATACCGACGAAAAAGGCGGGTGGTACGACGGAA
ATGCTAGCGAAAGCGTTGCGTAAAGTGCCGACGGACAATTATATAACGACGTAC
CCGGGTCAAGGTTTAAATGGTTACACGGTAGAGGAGGCGAAGACGGTGCTTAAA
AAGTGTAAAAGTGCGTTTTACATACTACCGTCTATAATATCTAATGAGAAGCAAG
AAATACTTGGTACGGTTTCTTGGAATTTGCGGGAAATGCTTGCGCATGCGGAAGA
AACGCGTAAATTAATGCCGGTCTGTGTGGAAACGAAAGCGATAGTTTCAACGAT
ACAGCGTAAATATAAGGGTATTAAAATACAAGAGGGTGTGGTTGATTATGGTGC
GCGTTTTTACTTTTACACGAGTAAAACGACGGTAGCGTCACTTATCAACACGCTT
AACGATCTAAATGAAACGCTTGTTACGATGCCGCTTGGTTATGTAACGCATGGTT
TAAATTTGGAAGAAGCGGCGCGTTATATGCGTTCTCTCAAAGTGCCGGCGACGGT
TTCTGTTTCTTCACCGGATGCGGTTACGGCGTATAATGGTTATCTTACGTCTTCTT
CTAAAACGCCGGAAGAACATTTTATTGAAACGATCTCACTTGCGGGTTCCTATAA
AGATTGGTCCTATTCTGGTCAATCTACGCAACTAGGTATAGAATTTCTTAAGCGT
GGTGATAAAAGTGTATATTACACGAGTAATCCGACGACGTTCCACCTAGATGGT
GAAGTTATCACGTTTGACAATCTTAAGACGCTTCTTTCTTTGCGTGAAGTGCGTAC
GATTAAGGTGTTTACGACGGTAGACAACATTAACCTCCACACGCAAGTTGTGGA
CATGTCAATGACGTATGGTCAACAGTTTGGTCCGACGTATTTGGATGGTGCGGAT
GTTACGAAAATAAAACCGCATAATTCACATGAAGGTAAAACGTTTTATGTTTTAC
CGAATGATGACACGCTACGTGTTGAGGCGTTTGAGTACTACCACACAACGGATC
CGAGTTTTCTGGGTCGTTACATGTCAGCGTTAAATCACACGAAAAAGTGGAAATA
CCCGCAAGTTAATGGTTTAACGTCTATTAAATGGGCGGATAACAACTGTTATCTT
GCGACGGCGTTGTTAACGCTCCAACAAATAGAGTTGAAGTTTAATCCGCCGGCG
CTACAAGATGCGTATTACCGTGCGCGTGCGGGTGAAGCGGCGAACTTTTGTGCGC
TTATCTTAGCGTACTGTAATAAGACGGTAGGTGAGTTAGGTGATGTTCGTGAAAC
GATGAGTTACTTGTTTCAACATGCGAATTTAGATTCTTGCAAACGTGTCTTGAAC
GTGGTGTGTAAAACGTGTGGTCAACAGCAAACGACGCTTAAGGGTGTAGAAGCG
GTTATGTACATGGGTACACTTTCTTATGAACAATTTAAGAAAGGTGTTCAAATAC
CGTGTACGTGTGGTAAACAAGCGACAAAATATCTAGTACAACAGGAGTCACCGT
TTGTTATGATGTCAGCGCCGCCGGCGCAGTATGAACTTAAGCATGGTACGTTTAC
GTGTGCGAGTGAGTACACGGGTAATTACCAGTGTGGTCACTATAAACATATAAC
GTCTAAAGAAACGTTGTATTGCATAGACGGTGCGTTACTTACGAAGTCCTCAGAA
TACAAAGGTCCGATTACGGATGTTTTCTACAAAGAAAACAGTTACACAACAACG
ATAAAACCGGTTACGTATAAATTGGATGGTGTTGTTTGTACGGAAATTGACCCGA
AGTTGGACAATTATTATAAGAAAGACAATTCTTATTTCACGGAGCAACCGATTGA
TCTTGTACCGAACCAACCGTATCCGAACGCGAGCTTCGATAATTTTAAGTTTGTA
TGTGATAATATCAAATTTGCGGATGATTTAAACCAATTAACGGGTTATAAGAAAC
CGGCGTCACGTGAGCTTAAAGTTACATTTTTCCCGGACTTAAATGGTGATGTGGT
GGCGATTGATTATAAACACTACACGCCGTCTTTTAAGAAAGGTGCGAAATTGTTA
CATAAACCGATTGTTTGGCATGTTAACAATGCGACGAATAAAGCGACGTATAAA
CCGAATACGTGGTGTATACGTTGTCTTTGGAGCACGAAACCGGTTGAAACATCAA
ATTCGTTTGATGTACTGAAGTCAGAGGACGCGCAGGGTATGGATAATCTTGCGTG
CGAAGATCTAAAACCGGTCTCTGAAGAAGTAGTGGAAAATCCGACGATACAGAA
AGACGTTCTTGAGTGTAATGTGAAAACGACGGAAGTTGTAGGTGACATTATACTT
AAACCGGCGAATAATAGTTTAAAAATTACGGAAGAGGTTGGTCACACAGATCTA
ATGGCGGCGTATGTAGACAATTCTAGTCTTACGATTAAGAAACCGAATGAATTAT
CTCGTGTATTAGGTTTGAAAACGCTTGCGACGCATGGTTTAGCGGCGGTTAATAG
TGTCCCGTGGGATACGATAGCGAATTATGCGAAGCCGTTTCTTAACAAAGTTGTT
AGTACGACGACGAACATAGTTACGCGTTGTTTAAACCGTGTTTGTACGAATTATA
TGCCGTATTTCTTTACGTTATTGCTACAATTGTGTACGTTTACGCGTAGTACGAAT
TCTCGTATTAAAGCGTCTATGCCGACGACGATAGCGAAGAATACGGTTAAGAGT
GTCGGTAAATTTTGTCTAGAGGCGTCATTTAATTATTTGAAGTCACCGAATTTTTC
TAAACTGATAAATATTATAATTTGGTTTTTACTATTAAGTGTTTGCCTAGGTTCTT
TAATCTACTCAACCGCGGCGTTAGGTGTTTTAATGTCTAATTTAGGTATGCCGTCT
TACTGTACGGGTTACCGTGAAGGTTATTTGAACTCTACGAATGTCACGATTGCGA
CGTACTGTACGGGTTCTATACCGTGTAGTGTTTGTCTTAGTGGTTTAGATTCTTTA
GACACGTATCCGTCTTTAGAAACGATACAAATTACGATTTCATCTTTTAAATGGG
ATTTAACGGCGTTTGGTTTAGTTGCGGAGTGGTTTTTGGCGTATATTCTTTTCACG
CGTTTTTTCTATGTACTTGGTTTGGCGGCGATCATGCAATTGTTTTTCAGCTATTTT
GCGGTACATTTTATTAGTAATTCTTGGCTTATGTGGTTAATAATTAATCTTGTACA
AATGGCGCCGATTTCAGCGATGGTTAGAATGTACATCTTCTTTGCGTCATTTTATT
ATGTATGGAAAAGTTATGTGCATGTTGTAGACGGTTGTAATTCATCAACGTGTAT
GATGTGTTACAAACGTAATAGAGCGACGCGTGTCGAATGTACGACGATTGTTAAT
GGTGTTAGACGTTCCTTTTATGTCTATGCGAATGGTGGTAAAGGTTTTTGCAAACT
ACACAATTGGAATTGTGTTAATTGTGATACGTTCTGTGCGGGTAGTACGTTTATT
AGTGATGAAGTTGCGCGTGACTTGTCACTACAGTTTAAACGTCCGATAAATCCGA
CGGACCAGTCTTCTTACATCGTTGATAGTGTTACGGTGAAGAATGGTTCCATCCA
TCTTTACTTTGATAAAGCGGGTCAAAAGACGTATGAACGTCATTCTCTCTCTCATT
TTGTTAACTTAGACAACCTGCGTGCGAATAACACGAAAGGTTCATTGCCGATTAA
TGTTATAGTTTTTGATGGTAAATCAAAATGTGAAGAATCATCTGCGAAATCAGCG
TCTGTTTACTACAGTCAGCTTATGTGTCAACCGATACTGTTACTAGATCAGGCGTT
AGTGTCTGATGTTGGTGATAGTGCGGAAGTTGCGGTTAAAATGTTTGATGCGTAC
GTTAATACGTTTTCATCAACGTTTAACGTACCGATGGAAAAACTCAAAACGCTAG
TTGCGACGGCGGAAGCGGAACTTGCGAAGAATGTGTCCTTAGACAATGTCTTATC
TACGTTTATTTCAGCGGCGCGTCAAGGTTTTGTTGATTCAGATGTAGAAACGAAA
GATGTTGTTGAATGTCTTAAATTGTCACATCAATCTGACATAGAAGTTACGGGTG
ATAGTTGTAATAACTATATGCTCACGTATAACAAAGTTGAAAACATGACGCCGC
GTGACCTTGGTGCGTGTATTGACTGTAGTGCGCGTCATATTAATGCGCAGGTAGC
GAAAAGTCACAACATTGCGTTGATATGGAACGTTAAAGATTTCATGTCATTGTCT
GAACAACTACGTAAACAAATACGTAGTGCGGCGAAAAAGAATAACTTACCGTTT
AAGTTGACGTGTGCGACGACGCGTCAAGTTGTTAATGTTGTAACGACGAAGATA
GCGCTTAAGGGTGGTAAAATTGTTAATAATTGGTTGAAGCAATTAATTAAA
SEQ ID NO: 67 - Clone pCCI-4K-SARS-COV-2-4N-1-delta, with notable
sequences underlined or otherwise identified (eg. restriction enzyme sites, nucelotide
differences):
GATGTGCTGCAAGGCGATTAAGTTGGGTAACGCCAGGGTTTTCCCAGT
CACGACGTTGTAAAACGACGGCCAGTGAATTGTAATACGACTCACTATAGGGCG
AATTCTAGTTATTAATAGTAATCAATTACGGGGTCATTAGTTCATAGCCCATATA
TGGAGTTCCGCGTTACATAACTTACGGTAAATGGCCCGCCTGGCTGACCGCCCAA
CGACCCCCGCCCATTGACGTCAATAATGACGTATGTTCCCATAGTAACGCCAATA
GGGACTTTCCATTGACGTCAATGGGTGGAGTATTTACGGTAAACTGCCCACTTGG
CAGTACATCAAGTGTATCATATGCCAAGTACGCCCCCTATTGACGTCAATGACGG
TAAATGGCCCGCCTGGCATTATGCCCAGTACATGACCTTATGGGACTTTCCTACT
TGGCAGTACATCTACGTATTAGTCATCGCTATTACCATGGTGATGCGGTTTTGGC
AGTACATCAATGGGCGTGGATAGCGGTTTGACTCACGGGGATTTCCAAGTCTCCA
CCCCATTGACGTCAATGGGAGTTTGTTTTGGCACCAAAATCAACGGGACTTTCCA
AAATGTCGTAACAACTCCGCCCCATTGACGCAAATGGGCGGTAGGCGTGTACGG
TGGGAGGTCTATATAAGCAGAGCTGGTTTAGTGAACCGTATTAAAGGTTTATACC
TTCCCAGGTAACAAACCAACCAACTTTCGATCTCTTGTAGATCTGTTCTCTAAAC
GAACTTTAAAATCTGTGTGGCTGTCACTCGGCTGCATGCTTAGTGCACTCACGCA
GTATAATTAATAACTAATTACTGTCGTTGACAGGACACGAGTAACTCGTCTATCT
TCTGCAGGCTGCTTACGGTTTCGTCCGTGTTGCAGCCGATCATCAGCACATCTAG
GTTTCGTCCGGGTGTGACCGAAAGGTAAGATGGAGAGCCTTGTCCCTGGTTTCAA
CGAGAAAACACACGTCCAACTCAGTTTGCCTGTTTTACAGGTTCGCGACGTGCTC
GTACGTGGCTTTGGAGACTCCGTGGAGGAGGTCTTATCAGAGGCACGTCAACAT
CTTAAAGATGGCACTTGTGGCTTAGTAGAAGTTGAAAAAGGCGTTTTGCCTCAAC
TTGAACAGCCCTATGTGTTCATCAAACGTTCGGATGCTCGAACTGCACCTCATGG
TCATGTTATGGTTGAGCTGGTAGCAGAACTCGAAGGCATTCAGTACGGTCGTAGT
GGTGAGACACTTGGTGTCCTTGTCCCTCATGTGGGCGAAATACCAGTGGCTTACC
GCAAGGTTCTTCTTCGTAAGAACGGTAATAAAGGAGCTGGTGGCCATAGTTACG
GCGCCGATCTAAAGTCATTTGACTTAGGCGACGAGCTTGGCACTGATCCTTATGA
AGATTTTCAAGAAAACTGGAACACTAAACATAGCAGTGGTGTTACCCGTGAACT
CATGCGTGAGCTTAACGGAGGGGCATACACTCGCTATGTCGATAACAACTTCTGT
GGCCCTGATGGCTACCCTCTTGAGTGCATTAAAGACCTTCTAGCACGTGCTGGTA
AAGCTTCATGCACTTTGTCCGAACAACTGGACTTTATTGACACTAAGAGGGGTGT
ATACTGCTGCCGTGAACATGAGCATGAAATTGCTTGGTACACGGAACGTTCTGAA
AAGAGCTATGAATTGCAGACACCTTTTGAAATTAAATTGGCAAAGAAATTTGAC
ACCTTCAATGGGGAATGTCCAAATTTTGTATTTCCCTTAAATTCCATAATCAAGA
CTATTCAACCAAGGGTTGAAAAGAAAAAGCTTGATGGCTTTATGGGTAGAATTC
GATCTGTCTATCCAGTTGCGTCACCAAATGAATGCAACCAAATGTGCCTTTCAAC
TCTCATGAAGTGTGATCATTGTGGTGAAACTTCATGGCAGACGGGCGATTTTGTT
AAAGCCACTTGCGAATTTTGTGGCACTGAGAATTTGACTAAAGAAGGTGCCACT
ACTTGTGGTTACTTACCCCAAAATGCTGTTGTTAAAATTTATTGTCCAGCATGTCA
CAATTCAGAAGTAGGACCTGAGCATAGTCTTGCCGAATACCATAATGAATCTGG
CTTGAAAACCATTCTTCGTAAGGGTGGTCGCACTATTGCCTTTGGAGGCTGTGTG
TTCTCTTATGTTGGTTGCCATAACAAGTGTGCCTATTGGGTCCCACGTGCTAGCG
CTAACATAGGTTGTAACCATACAGGTGTTGTTGGAGAAGGTTCCGAAGGTCTTAA
TGACAACCTTCTTGAAATACTCCAAAAAGAGAAAGTCAACATCAATATTGTTGGT
GACTTTAAACTTAATGAAGAGATCGCCATTATTTTGGCATCTTTTTCTGCTTCCAC
AAGTGCTTTTGTGGAAACTGTGAAAGGTTTGGATTATAAAGCATTCAAACAAATT
GTTGAATCCTGTGGTAATTTTAAAGTTACAAAAGGAAAAGCTAAAAAAGGTGCC
TGGAATATTGGTGAACAGAAATCAATACTGAGTCCTCTTTATGCATTTGCATCAG
AGGCTGCTCGTGTTGTACGATCAATTTTCTCCCGCACTCTTGAAACTGCTCAAAA
TTCTGTGCGTGTTTTACAGAAGGCCGCTATAACAATACTAGATGGAATTTCACAG
TATTCACTGAGACTCATTGATGCTATGATGTTCACATCTGATTTGGCTACTAACAA
TCTAGTTGTAATGGCCTACATTACAGGTGGTGTTGTTCAGTTGACTTCGCAGTGG
CTAACTAACATCTTTGGCACTGTTTATGAAAAACTCAAACCCGTCCTTGATTGGC
TTGAAGAGAAGTTTAAGGAAGGTGTAGAGTTTCTTAGAGACGGTTGGGAAATTG
TTAAATTTATCTCAACCTGTGCTTGTGAAATTGTCGGTGGACAAATTGTCACCTGT
GCAAAGGAAATTAAGGAGAGTGTTCAGACATTCTTTAAGCTTGTAAATAAATTTT
TGGCTTTGTGTGCTGACTCTATCATTATTGGTGGAGCTAAACTTAAAGCCTTGAAT
TTAGGTGAAACATTTGTCACGCACTCAAAGGGATTGTACAGAAAGTGTGTTAAAT
CCAGAGAAGAAACTGGCCTACTCATGCCTCTAAAAGCCCCAAAAGAAATTATCT
TCTTAGAGGGAGAAACACTTCCCACAGAAGTGTTAACAGAGGAAGTTGTCTTGA
AAACTGGTGATTTACAACCATTAGAACAACCTACTAGTGAAGCTGTTGAAGCTCC
ATTGGTTGGTACACCAGTTTGTATTAACGGGCTTATGTTGCTCGAAATCAAAGAC
ACAGAAAAGTACTGTGCCCTTGCACCTAATATGATGGTAACAAACAATACCTTCA
CACTCAAAGGCGGTGCACCAACAAAGGTTACTTTTGGTGATGACACTGTGATAG
AAGTGCAAGGTTACAAGAGTGTGAATATCACTTTTGAACTTGATGAAAGGATTG
ATAAAGTACTTAATGAGAAGTGCTCTGCCTATACAGTTGAACTCGGTACAGAAGT
AAATGAGTTCGCCTGTGTTGTGGCAGATGCTGTCATAAAAACTTTGCAACCAGTA
TCTGAATTACTTACACCACTGGGCATTGATTTAGATGAGTGGAGTATGGCTACAT
ACTACTTATTTGATGAGTCTGGTGAGTTTAAATTGGCTTCACATATGTATTGTTCT
TTCTACCCTCCAGATGAGGATGAAGAAGAAGGTGATTGTGAAGAAGAAGAGTTT
GAGCCATCAACTCAATATGAGTATGGTACTGAAGATGATTACCAAGGTAAACCT
TTGGAATTTGGTGCCACTTCTGCTGCTCTTCAACCTGAAGAAGAGCAAGAAGAAG
ATTGGTTAGATGATGATAGTCAACAAACTGTTGGTCAACAAGACGGCAGTGAGG
ACAATCAGACAACTACTATTCAAACAATTGTTGAGGTTCAACCTCAATTAGAGAT
GGAACTTACACCAGTTGTTCAGACTATTGAAGTGAATAGTTTTAGTGGTTATTTA
AAACTTACTGACAATGTATACATTAAAAATGCAGACATTGTGGAAGAAGCTAAA
AAGGTAAAACCAACAGTGGTTGTTAATGCAGCCAATGTTTACCTTAAACATGGA
GGAGGTGTTGCAGGAGCCTTAAATAAGGCTACTAACAATGCCATGCAAGTTGAA
TCTGATGATTACATAGCTACTAATGGACCACTTAAAGTGGGTGGTAGTTGTGTTT
TAAGCGGACACAATCTTGCTAAACACTGTCTTCATGTTGTCGGCCCAAATGTTAA
CAAAGGTGAAGACATTCAACTTCTTAAGAGTGCTTATGAAAATTTTAATCAGCAC
GAAGTTCTACTTGCACCATTATTATCAGCTGGTATTTTTGGTGCTGACCCTATACA
TTCTTTAAGAGTTTGTGTAGATACTGTTCGCACAAATGTCTACTTAGCTGTCTTTG
ATAAAAATCTCTATGACAAACTTGTTTCAAGCTTTTTGGAAATGAAGAGTGAAAA
GCAAGTTGAACAAAAGATCGCTGAGATTCCTAAAGAGGAAGTTAAGCCATTTAT
AACTGAAAGTAAACCTTCAGTTGAACAGAGAAAACAAGATGATAAGAAAATCA
AAGCTTGTGTTGAAGAAGTTACAACAACTCTGGAAGAAACTAAGTTCCTCACAG
AAAACTTGTTACTTTATATTGACATTAATGGCAATCTTCATCCAGATTCTGCCACT
CTTGTTAGTGACATTGACATCACTTTCTTAAAGAAAGATGCTCCATATATAGTGG
GTGATGTTGTTCAAGAGGGTGTTTTAACTGCTGTGGTTATACCTACTAAAAAGGC
TGGTGGCACTACTGAAATGCTAGCGAAAGCTTTGAGAAAAGTGCCAACAGACAA
TTATATAACCACTTACCCGGGTCAGGGTTTAAATGGTTACACTGTAGAGGAGGC
AAAGACAGTGCTTAAAAAGTGTAAAAGTGCCTTTTACATTCTACCATCTATTATC
TCTAATGAGAAGCAAGAAATTCTTGGAACTGTTTCTTGGAATTTGCGAGAAATGC
TTGCACATGCAGAAGAAACACGCAAATTAATGCCTGTCTGTGTGGAAACTAAAG
CCATAGTTTCAACTATACAGCGTAAATATAAGGGTATTAAAATACAAGAGGGTG
TGGTTGATTATGGTGCTAGATTTTACTTTTACACCAGTAAAACAACTGTAGCGTC
ACTTATCAACACACTTAACGATCTAAATGAAACTCTTGTTACAATGCCACTTGGC
TATGTAACACATGGCTTAAATTTGGAAGAAGCTGCTCGGTATATGAGATCTCTCA
AAGTGCCAGCTACAGTTTCTGTTTCTTCACCTGATGCTGTTACAGCGTATAATGGT
TATCTTACTTCTTCTTCTAAAACACCTGAAGAACATTTTATTGAAACCATCTCACT
TGCTGGTTCCTATAAAGATTGGTCCTATTCTGGACAATCTACACAACTAGGTATA
GAATTTCTTAAGAGAGGTGATAAAAGTGTATATTACACTAGTAATCCTACCACAT
TCCACCTAGATGGTGAAGTTATCACCTTTGACAATCTTAAGACACTTCTTTCTTTG
AGAGAAGTGAGGACTATTAAGGTGTTTACAACAGTAGACAACATTAACCTCCAC
ACGCAAGTTGTGGACATGTCAATGACATATGGACAACAGTTTGGTCCAACTTATT
TGGATGGAGCTGATGTTACTAAAATAAAACCTCATAATTCACATGAAGGTAAAA
CATTTTATGTTTTACCTAATGATGACACTCTACGTGTTGAGGCTTTTGAGTACTAC
CACACAACTGATCCTAGTTTTCTGGGTAGGTACATGTCAGCATTAAATCACACTA
AAAAGTGGAAATACCCACAAGTTAATGGTTTAACTTCTATTAAATGGGCAGATA
ACAACTGTTATCTTGCCACTGCATTGTTAACACTCCAACAAATAGAGTTGAAGTT
TAATCCACCTGCTCTACAAGATGCTTATTACAGAGCAAGGGCTGGTGAAGCTGCT
AACTTTTGTGCACTTATCTTAGCCTACTGTAATAAGACAGTAGGTGAGTTAGGTG
ATGTTAGAGAAACAATGAGTTACTTGTTTCAACATGCCAATTTAGATTCTTGCAA
AAGAGTCTTGAACGTGGTGTGTAAAACTTGTGGACAACAGCAGACAACCCTTAA
GGGTGTAGAAGCTGTTATGTACATGGGCACACTTTCTTATGAACAATTTAAGAAA
GGTGTTCAGATACCTTGTACGTGTGGTAAACAAGCTACAAAATATCTAGTACAAC
AGGAGTCACCTTTTGTTATGATGTCAGCACCACCTGCTCAGTATGAACTTAAGCA
TGGTACATTTACTTGTGCTAGTGAGTACACTGGTAATTACCAGTGTGGTCACTAT
AAACATATAACTTCTAAAGAAACTTTGTATTGCATAGACGGTGCTTTACTTACAA
AGTCCTCAGAATACAAAGGTCCTATTACGGATGTTTTCTACAAAGAAAACAGTTA
CACAACAACCATAAAACCAGTTACTTATAAATTGGATGGTGTTGTTTGTACAGAA
ATTGACCCTAAGTTGGACAATTATTATAAGAAAGACAATTCTTATTTCACAGAGC
AACCAATTGATCTTGTACCAAACCAACCATATCCAAACGCAAGCTTCGATAATTT
TAAGTTTGTATGTGATAATATCAAATTTGCTGATGATTTAAACCAGTTAACTGGTT
ATAAGAAACCTGCTTCAAGAGAGCTTAAAGTTACATTTTTCCCTGACTTAAATGG
TGATGTGGTGGCTATTGATTATAAACACTACACACCCTCTTTTAAGAAAGGAGCT
AAATTGTTACATAAACCTATTGTTTGGCATGTTAACAATGCAACTAATAAAGCCA
CGTATAAACCAAATACCTGGTGTATACGTTGTCTTTGGAGCACAAAACCAGTTGA
AACATCAAATTCGTTTGATGTACTGAAGTCAGAGGACGCGCAGGGAATGGATAA
TCTTGCCTGCGAAGATCTAAAACCAGTCTCTGAAGAAGTAGTGGAAAATCCTACC
ATACAGAAAGACGTTCTTGAGTGTAATGTGAAAACTACCGAAGTTGTAGGAGAC
ATTATACTTAAACCAGCAAATAATAGTTTAAAAATTACAGAAGAGGTTGGCCAC
ACAGATCTAATGGCTGCTTATGTAGACAATTCTAGTCTTACTATTAAGAAACCTA
ATGAATTATCTAGAGTATTAGGTTTGAAAACCCTTGCTACTCATGGTTTAGCTGCT
GTTAATAGTGTCCCTTGGGATACTATAGCTAATTATGCTAAGCCTTTTCTTAACAA
AGTTGTTAGTACAACTACTAACATAGTTACACGGTGTTTAAACCGTGTTTGTACT
AATTATATGCCTTATTTCTTTACTTTATTGCTACAATTGTGTACTTTTACTAGAAGT
ACAAATTCTAGAATTAAAGCATCTATGCCGACTACTATAGCAAAGAATACTGTTA
AGAGTGTCGGTAAATTTTGTCTAGAGGCTTCATTTAATTATTTGAAGTCACCTAAT
TTTTCTAAACTGATAAATATTATAATTTGGTTTTTACTATTAAGTGTTTGCCTAGG
TTCTTTAATATACTCAACGGCGGCGTTAGGTGTTTTAATGTCTAATTTAGGCATGC
CGTCTTACTGTACGGGTTACAGAGAAGGCTATTTGAACTCTACGAATGTCACGAT
AGCGACGTACTGTACGGGTTCTATACCGTGTAGTGTTTGTCTTAGTGGTTTAGATT
CTTTAGACACGTATCCGTCTTTAGAAACGATACAAATAACGATATCATCTTTTAA
ATGGGATTTAACGGCGTTTGGCTTAGTTGCGGAGTGGTTTTTGGCGTATATTCTTT
TCACGAGGTTTTTCTATGTACTTGGATTGGCGGCGATAATGCAATTGTTTTTCAGC
TATTTTGCGGTACATTTTATAAGTAATTCTTGGCTTATGTGGTTAATAATAAATCT
TGTACAAATGGCGCCGATATCAGCGATGGTTAGAATGTACATATTCTTTGCGTCA
TTTTATTATGTATGGAAAAGTTATGTGCATGTTGTAGACGGTTGTAATTCATCAAC
GTGTATGATGTGTTACAAACGTAATAGAGCGACGAGAGTCGAATGTACGACGAT
AGTTAATGGTGTTAGAAGGTCCTTTTATGTCTATGCGAATGGAGGTAAAGGCTTT
TGCAAACTACACAATTGGAATTGTGTTAATTGTGATACGTTCTGTGCGGGTAGTA
CGTTTATAAGTGATGAAGTTGCGAGAGACTTGTCACTACAGTTTAAAAGACCGAT
AAATCCGACGGACCAGTCTTCTTACATAGTTGATAGTGTTACGGTGAAGAATGGT
TCCATACATCTTTACTTTGATAAAGCGGGTCAAAAGACGTATGAAAGACATTCTC
TCTCTCATTTTGTTAACTTAGACAACCTGAGAGCGAATAACACGAAAGGTTCATT
GCCGATAAATGTTATAGTTTTTGATGGTAAATCAAAATGTGAAGAATCATCTGCG
AAATCAGCGTCTGTTTACTACAGTCAGCTTATGTGTCAACCGATACTGTTACTAG
ATCAGGCGTTAGTGTCTGATGTTGGTGATAGTGCGGAAGTTGCGGTTAAAATGTT
TGATGCGTACGTTAATACGTTTTCATCAACGTTTAACGTACCGATGGAAAAACTC
AAAACGCTAGTTGCGACGGCGGAAGCGGAACTTGCGAAGAATGTGTCCTTAGAC
AATGTCTTATCTACGTTTATATCAGCGGCGCGGCAAGGGTTTGTTGATTCAGATG
TAGAAACGAAAGATGTTGTTGAATGTCTTAAATTGTCACATCAATCTGACATAGA
AGTTACGGGCGATAGTTGTAATAACTATATGCTCACGTATAACAAAGTTGAAAAC
ATGACGCCGCGTGACCTTGGTGCGTGTATAGACTGTAGTGCGCGTCATATAAATG
CGCAGGTAGCGAAAAGTCACAACATAGCGTTGATATGGAACGTTAAAGATTTCA
TGTCATTGTCTGAACAACTACGAAAACAAATACGTAGTGCGGCGAAAAAGAATA
ACTTACCGTTTAAGTTGACGTGTGCGACGACGAGACAAGTTGTTAATGTTGTAAC
GACGAAGATAGCGCTTAAGGGTGGTAAAATAGTTAATAATTGGTTGAAGCAGTT
AATTAAAGTTACACTTGTGTTCCTTTTTGTTGCTGCTATTTTCTATTTAATAACAC
CTGTTCATGTCATGTCTAAACATACTGACTTTTCAAGTGAAATCATAGGATACAA
GGCTATTGATGGTGGTGTCACTCGTGACATAGCATCTACAGATACTTGTTTTGCT
AACAAACATGCTGATTTTGACACATGGTTTAGCCAGCGTGGTGGTAGTTATACTA
ATGACAAAGCTTGCCCATTGATTGCTGCAGTCATAACAAGAGAAGTGGGTTTTGT
CGTGCCTGGTTTGCCTGGCACGATATTACGCACAACTAATGGTGACTTTTTGCAT
TTCTTACCTAGAGTTTTTAGTGCAGTTGGTAACATCTGTTACACACCATCAAAACT
TATAGAGTACACTGACTTTGCAACATCAGCTTGTGTTTTGGCTGCTGAATGTACA
ATTTTTAAAGATGCTTCTGGTAAGCCAGTACCATATTGTTATGATACCAATGTACT
AGAAGGTTCTGTTGCTTATGAAAGTTTACGCCCTGACACACGTTATGTGCTCATG
GATGGCTCTATTATTCAATTTCCTAACACCTACCTTGAAGGTTCTGTTAGAGTGGT
AACAACTTTTGATTCTGAGTACTGTAGGCACGGCACTTGTGAAAGATCAGAAGCT
GGTGTTTGTGTATCTACTAGTGGTAGATGGGTACTTAACAATGATTATTACAGAT
CTTTACCAGGAGTTTTCTGTGGTGTAGATGCTGTAAATTTACTTACTAATATGTTT
ACACCACTAATTCAACCTATTGGTGCTTTGGACATATCAGCATCTATAGTAGCTG
GTGGTATTGTAGCTATCGTAGTAACATGCCTTGCCTACTATTTTATGAGGTTTAGA
AGAGCTTTTGGTGAATACAGTCATGTAGTTGCCTTTAATACTTTACTATTCCTTAT
GTCATTCACTGTACTCTGTTTAACACCAGTTTACTCATTCTTACCTGGTGTTTATTC
TGTTATTTACTTGTACTTGACATTTTATCTTACTAATGATGTTTCTTTTTTAGCACA
TATTCAGTGGATGGTTATGTTCACACCTTTAGTACCTTTCTGGATAACAATTGCTT
ATATCATTTGTATTTCCACAAAGCATTTCTATTGGTTCTTTAGTAATTACCTAAAG
AGACGTGTAGTCTTTAATGGTGTTTCCTTTAGTACTTTTGAAGAAGCTGCGCTGTG
CACCTTTTTGTTAAATAAAGAAATGTATCTAAAGTTGCGTAGTGATGTGCTATTA
CCTCTTACGCAATATAATAGATACTTAGCTCTTTATAATAAGTACAAGTATTTTAG
TGGAGCAATGGATACAACTAGCTACAGAGAAGCTGCTTGTTGTCATCTCGCAAA
GGCTCTCAATGACTTCAGTAACTCAGGTTCTGATGTTCTTTACCAACCACCACAA
ACCTCTATCACCTCAGCTGTTTTGCAGAGTGGTTTTAGAAAAATGGCATTCCCAT
CTGGTAAAGTTGAGGGTTGTATGGTACAAGTAACTTGTGGTACAACTACACTTAA
CGGTCTTTGGCTTGATGACGTAGTTTACTGTCCAAGACATGTGATCTGCACCTCT
GAAGACATGCTTAACCCTAATTATGAAGATTTACTCATTCGTAAGTCTAATCATA
ATTTCTTGGTACAGGCTGGTAATGTTCAACTCAGGGTTATTGGACATTCTATGCA
AAATTGTGTACTTAAGCTTAAGGTTGATACAGCCAATCCTAAGACACCTAAGTAT
AAGTTTGTTCGCATTCAACCAGGACAGACTTTTTCAGTGTTAGCTTGTTACAATG
GTTCACCATCTGGTGTTTACCAATGTGCTATGAGGCCCAATTTCACTATTAAGGG
TTCATTCCTTAATGGTTCATGTGGTAGTGTTGGTTTTAACATAGATTATGACTGTG
TCTCTTTTTGTTACATGCACCATATGGAATTACCAACTGGAGTTCATGCTGGCAC
AGACTTAGAAGGTAACTTTTATGGACCTTTTGTTGACAGGCAAACAGCACAAGC
AGCTGGTACGGACACAACTATTACAGTTAATGTTTTAGCTTGGTTGTACGCTGCT
GTTATAAATGGAGACAGGTGGTTTCTCAATCGATTTACCACAACTCTTAATGACT
TTAACCTTGTGGCTATGAAGTACAATTATGAACCTCTAACACAAGACCATGTTGA
CATACTAGGACCTCTTTCTGCTCAAACTGGAATTGCCGTTTTAGATATGTGTGCTT
CATTAAAAGAATTACTGCAAAATGGTATGAATGGACGTACCATATTGGGTAGTG
CTTTATTAGAAGATGAATTTACACCTTTTGATGTTGTTAGACAATGCTCAGGTGTT
ACTTTCCAAAGTGCAGTGAAAAGAACAATCAAGGGTACACACCACTGGTTGTTA
CTCACAATTTTGACTTCACTTTTAGTTTTAGTCCAGAGTACTCAATGGTCTTTGTT
CTTTTTTTTGTATGAAAATGCCTTTTTACCTTTTGCTATGGGTATTATTGCTATGTC
TGCTTTTGCAATGATGTTTGTCAAACATAAGCATGCATTTCTCTGTTTGTTTTTGTT
ACCTTCTCTTGCCACTGTAGCTTATTTTAATATGGTCTATATGCCTGCTAGTTGGG
TGATGCGTATTATGACATGGTTGGATATGGTTGATACTAGTTTGTCTGGTTTTAAG
CTAAAAGACTGTGTTATGTATGCATCAGCTGTAGTGTTACTAATCCTTATGACAG
CAAGAACTGTGTATGATGATGGTGCTAGGAGAGTGTGGACACTTATGAATGTCTT
GACACTCGTTTATAAAGTTTATTATGGTAATGCTTTAGATCAAGCCATTTCCATGT
GGGCTCTTATAATCTCTGTTACTTCTAACTACTCAGGTGTAGTTACAACTGTCATG
TTTTTGGCCAGAGGTATTGTTTTTATGTGTGTTGAGTATTGCCCTATTTTCTTCATA
ACTGGTAATACACTTCAGTGTATAATGCTAGTTTATTGTTTCTTAGGCTATTTTTG
TACTTGTTACTTTGGCCTCTTTTGTTTACTCAACCGCTACTTTAGACTGACTCTTGG
TGTTTATGATTACTTAGTTTCTACACAGGAGTTTAGATATATGAATTCACAGGGA
CTACTCCCACCCAAGAATAGCATAGATGCCTTCAAACTCAACATTAAATTGTTGG
GTGTTGGTGGCAAACCTTGTATCAAAGTAGCCACTGTACAGTCTAAAATGTCAGA
TGTAAAGTGCACATCAGTAGTCTTACTCTCAGTTTTGCAACAACTCAGAGTAGAA
TCATCATCTAAATTGTGGGCTCAATGTGTCCAGTTACACAATGACATTCTCTTAG
CTAAAGATACTACTGAAGCCTTTGAAAAAATGGTTTCACTACTTTCTGTTTTGCTT
TCCATGCAGGGTGCTGTAGACATAAACAAGCTTTGTGAAGAAATGCTGGACAAC
AGGGCAACCTTACAAGCTATAGCCTCAGAGTTTAGTTCCCTTCCATCATATGCAG
CTTTTGCTACTGCTCAAGAAGCTTATGAGCAGGCTGTTGCTAATGGTGATTCTGA
AGTTGTTCTTAAAAAGTTGAAGAAGTCTTTGAATGTGGCTAAATCTGAATTTGAC
CGTGATGCAGCCATGCAACGTAAGTTGGAAAAGATGGCTGATCAAGCTATGACC
CAAATGTATAAACAGGCTAGATCTGAGGACAAGAGGGCAAAAGTTACTAGTGCT
ATGCAGACAATGCTTTTCACTATGCTTAGAAAGTTGGATAATGATGCACTCAACA
ACATTATCAACAATGCAAGAGATGGTTGTGTTCCCTTGAACATAATACCTCTTAC
AACAGCAGCCAAACTAATGGTTGTCATACCAGACTATAACACATATAAAAATAC
GTGTGATGGTACAACATTTACTTATGCATCAGCATTGTGGGAAATCCAACAGGTT
GTAGATGCAGATAGTAAAATTGTTCAACTTAGTGAAATTAGTATGGACAATTCAC
CTAATTTAGCATGGCCTCTTATTGTAACAGCTTTAAGGGCCAATTCTGCTGTCAA
ATTACAGAATAATGAGCTTAGTCCTGTTGCACTACGACAGATGTCTTGTGCTGCC
GGTACTACACAAACTGCTTGCACTGATGACAATGCGTTAGCTTACTACAACACAA
CAAAGGGAGGTAGGTTTGTACTTGCACTGTTATCCGATTTACAGGATTTGAAATG
GGCTAGATTCCCTAAGAGTGATGGAACTGGTACTATCTATACAGAACTGGAACC
ACCTTGTAGGTTTGTTACAGACACACCTAAAGGTCCTAAAGTGAAGTATTTATAC
TTTATTAAAGGATTAAACAACCTAAATAGAGGTATGGTACTTGGTAGTTTAGCTG
CCACAGTACGTCTACAAGCTGGTAATGCAACAGAAGTGCCTGCCAATTCAACTGT
ATTATCTTTCTGTGCTTTTGCTGTAGATGCTGCTAAAGCTTACAAAGATTATCTAG
CTAGTGGGGGACAACCAATCACTAATTGTGTTAAGATGTTGTGTACACACACTGG
TACTGGTCAGGCAATAACAGTTACACCGGAAGCCAATATGGATCAAGAATCCTT
TGGTGGTGCATCGTGTTGTCTGTACTGCCGTTGCCACATAGATCATCCAAATCCT
AAAGGATTTTGTGACTTAAAAGGTAAGTATGTACAAATACCTACAACTTGTGCTA
ATGACCCTGTGGGTTTTACACTTAAAAACACAGTCTGTACCGTCTGCGGTATGTG
GAAAGGTTATGGCTGTAGTTGTGATCAACTCCGCGAACCCATGCTTCAGTCAGCT
GATGCACAATCGTTTTTAAACGGGTTTGCGGTGTAAGTGCAGCCCGTCTTACACC
GTGCGGCACAGGCACTAGTACTGATGTCGTATACAGGGCTTTTGACATCTACAAT
GATAAAGTAGCTGGTTTTGCTAAATTCCTAAAAACTAATTGTTGTCGCTTCCAAG
AAAAGGACGAAGATGACAATTTAATTGATTCTTACTTTGTAGTTAAGAGACACAC
TTTCTCTAACTACCAACATGAAGAAACAATTTATAATTTACTTAAGGATTGTCCA
GCTGTTGCTAAACATGACTTCTTTAAGTTTAGAATAGACGGTGACATGGTACCAC
ATATATCACGTCAACGTCTTACTAAATACACAATGGCAGACCTCGTCTATGCTTT
AAGGCATTTTGATGAAGGTAATTGTGACACATTAAAAGAAATACTTGTCACATAC
AATTGTTGTGATGATGATTATTTCAATAAAAAGGACTGGTATGATTTTGTAGAAA
ACCCAGATATATTACGCGTATACGCCAACTTAGGTGAACGTGTACGCCAAGCTT
TGTTAAAAACAGTACAATTCTGTGATGCCATGCGAAATGCTGGTATTGTTGGTGT
ACTGACATTAGATAATCAAGATCTCAATGGTAACTGGTATGATTTCGGTGATTTC
ATACAAACCACGCCAGGTAGTGGAGTTCCTGTTGTAGATTCTTATTATTCATTGTT
AATGCCTATATTAACCTTGACCAGGGCTTTAACTGCAGAGTCACATGTTGACACT
GACTTAACAAAGCCTTACATTAAGTGGGATTTGTTAAAATATGACTTCACGGAAG
AGAGGTTAAAACTCTTTGACCGTTATTTTAAATATTGGGATCAGACATACCACCC
AAATTGTGTTAACTGTTTGGATGACAGATGCATTCTGCATTGTGCAAACTTTAAT
GTTTTATTCTCTACAGTGTTCCCACCTACAAGTTTTGGACCACTAGTGAGAAAAA
TATTTGTTGATGGTGTTCCATTTGTAGTTTCAACTGGATACCACTTCAGAGAGCTA
GGTGTTGTACATAATCAGGATGTAAACTTACATAGCTCTAGACTTAGTTTTAAGG
AATTACTTGTGTATGCTGCTGACCCTGCTATGCACGCTGCTTCTGGTAATCTATTA
CTAGATAAACGCACTACGTGCTTTTCAGTAGCTGCACTTACTAACAATGTTGCTT
TTCAAACTGTCAAACCCGGTAATTTTAACAAAGACTTCTATGACTTTGCTGTGTCT
AAGGGTTTCTTTAAGGAAGGAAGTTCTGTTGAATTAAAACACTTCTTCTTTGCTC
AGGATGGTAATGCTGCTATCAGCGATTATGACTACTATCGTTATAATCTACCAAC
AATGTGTGATATCAGACAACTACTATTTGTAGTTGAAGTTGTTGATAAGTACTTT
GATTGTTACGATGGTGGCTGTATTAATGCTAACCAAGTCATCGTCAACAACCTAG
ACAAATCAGCTGGTTTTCCATTTAATAAATGGGGTAAGGCTAGACTTTATTATGA
TTCAATGAGTTATGAGGATCAAGATGCACTTTTCGCATATACAAAACGTAATGTC
ATCCCTACTATAACTCAAATGAATCTTAAGTATGCCATTAGTGCAAAGAATAGAG
CTCGCACCGTAGCTGGTGTCTCTATCTGTAGTACTATGACCAATAGACAGTTTCA
TCAAAAATTATTGAAATCAATAGCCGCCACTAGAGGAGCTACTGTAGTAATTGG
AACAAGCAAATTCTATGGTGGTTGGCACAACATGTTAAAAACTGTTTATAGTGAT
GTAGAAAACCCTCACCTTATGGGTTGGGATTATCCTAAATGTGATAGAGCCATGC
CTAACATGCTTAGAATTATGGCCTCACTTGTTCTTGCTCGCAAACATACAACGTG
TTGTAGCTTGTCACACCGTTTCTATAGATTAGCTAATGAGTGTGCTCAAGTATTGA
GTGAAATGGTCATGTGTGGCGGTTCACTATATGTTAAACCAGGTGGAACCTCATC
AGGAGATGCCACAACTGCTTATGCTAATAGTGTTTTTAACATTTGTCAAGCTGTC
ACGGCCAATGTTAATGCACTTTTATCTACTGATGGTAACAAAATTGCCGATAAGT
ATGTCCGCAATTTACAACACAGACTTTATGAGTGTCTCTATAGAAATAGAGATGT
TGACACAGACTTTGTGAATGAGTTTTACGCATATTTGCGTAAACATTTCTCAATG
ATGATACTCTCTGACGATGCTGTTGTGTGTTTCAATAGCACTTATGCATCTCAAGG
TCTAGTGGCTAGCATAAAGAACTTTAAGTCAGTTCTTTATTATCAAAACAATGTT
TTTATGTCTGAAGCAAAATGTTGGACTGAGACTGACCTTACTAAAGGACCTCATG
AATTTTGCTCTCAACATACAATGCTAGTTAAACAGGGTGATGATTATGTGTACCT
TCCTTACCCAGATCCATCAAGAATCCTAGGGGCCGGCTGTTTTGTAGATGATATC
GTAAAAACAGATGGTACACTTATGATTGAACGGTTCGTGTCTTTAGCTATAGATG
CTTACCCACTTACTAAACATCCTAATCAGGAGTATGCTGATGTCTTTCATTTGTAC
TTACAATACATAAGAAAGCTACATGATGAGTTAACAGGACACATGTTAGACATG
TATTCTGTTATGCTTACTAATGATAACACTTCAAGGTATTGGGAACCTGAGTTTTA
TGAGGCTATGTACACACCGCATACAGTCTTACAGGCTGTTGGGGCTTGTGTTCTT
TGCAATTCACAGACTTCATTAAGATGTGGTGCTTGCATACGTAGACCATTCTTAT
GTTGTAAATGCTGTTACGACCATGTCATATCAACATCACATAAATTAGTCTTGTCT
GTTAATCCGTATGTTTGCAATGCTCCAGGTTGTGATGTCACAGATGTGACTCAAC
TTTACTTAGGAGGTATGAGCTATTATTGTAAATCACATAAACCACCCATTAGTTTT
CCATTGTGTGCTAATGGACAAGTTTTTGGTTTATATAAAAATACATGTGTTGGTA
GCGATAATGTTACTGACTTTAATGCAATTGCAACATGTGACTGGACAAATGCTGG
TGATTACATTTTAGCTAACACCTGTACTGAAAGACTCAAGCTTTTTGCAGCAGAA
ACGCTCAAAGCTACTGAGGAGACATTTAAACTGTCTTATGGTATTGCTACTGTAC
GTGAAGTGCTGTCTGACAGAGAATTACATCTTTCATGGGAAGTTGGTAAACCTAG
ACCACCACTTAACCGAAATTATGTCTTTACTGGTTATCGTGTAACTAAAAACAGT
AAAGTACAAATAGGAGAGTACACCTTTGAAAAAGGTGACTATGGTGATGCTGTT
GTTTACCGAGGTACAACAACTTACAAATTAAATGTTGGTGATTATTTTGTGCTGA
CATCACATACAGTAATGCCATTAAGTGCACCTACACTAGTGCCACAAGAGCACT
ATGTTAGAATTACTGGCTTATACCCAACACTCAATATCTCAGATGAGTTTTCTAG
CAATGTTGCAAATTATCAAAAGGTTGGTATGCAAAAGTATTCTACACTCCAGGGA
CCACCTGGTACTGGTAAGAGTCATTTTGCTATTGGCCTAGCTCTCTACTACCCTTC
TGCTCGCATAGTGTATACAGCTTGCTCTCATGCCGCTGTTGATGCACTATGTGAG
AAGGCATTAAAATATTTGCCTATAGATAAATGTAGTAGAATTATACCTGCACGTG
CTCGTGTAGAGTGTTTTGATAAATTCAAAGTGAATTCAACATTAGAACAGTATGT
CTTTTGTACTGTAAATGCATTGCCTGAGACGACAGCAGATATAGTTGTCTTTGAT
GAAATTTCAATGGCCACAAATTATGATTTGAGTGTTGTCAATGCCAGATTACGTG
CTAAGCACTATGTGTACATTGGCGACCCTGCTCAATTACCTGCACCACGCACATT
GCTAACTAAGGGCACACTAGAACCAGAATATTTCAATTCAGTGTGTAGACTTATG
AAAACTATAGGTCCAGACATGTTCCTCGGAACTTGTCGGCGTTGTCCTGCTGAAA
TTGTTGACACTGTGAGTGCTTTGGTTTATGATAATAAGCTTAAAGCACATAAAGA
CAAATCAGCTCAATGCTTTAAAATGTTTTATAAGGGTGTTATCACGCATGATGTT
TCATCTGCAATTAACAGGCCACAAATAGGCGTGGTAAGAGAATTCCTTACACGT
AACCCTGCTTGGAGAAAAGCTGTCTTTATTTCACCTTATAATTCACAGAATGCTG
TAGCCTCAAAGATTTTGGGACTACCAACTCAAACTGTTGATTCATCACAGGGCTC
AGAATATGACTATGTCATATTCACTCAAACCACTGAAACAGCTCACTCTTGTAAT
GTAAACAGATTTAATGTTGCTATTACCAGAGCAAAAGTAGGCATACTTTGCATAA
TGTCTGATAGAGACCTTTATGACAAGTTGCAATTTACAAGTCTTGAAATTCCACG
TAGGAATGTGGCAACTTTACAAGCTGAAAATGTAACAGGACTCTTTAAAGATTGT
AGTAAGGTAATCACTGGGTTACATCCTACACAGGCACCTACACACCTCAGTGTTG
ACACTAAATTCAAAACTGAAGGTTTATGTGTTGACATACCTGGCATACCTAAGG
ACATGACCTATAGAAGACTCATCTCTATGATGGGTTTTAAAATGAATTATCAAGT
TAATGGTTACCCTAACATGTTTATCACCCGCGAAGAAGCTATAAGACATGTACGT
GCATGGATTGGCTTCGATGTCGAGGGGTGTCATGCTACTAGAGAAGCTGTTGGTA
CCAATTTACCTTTACAGCTAGGTTTTTCTACAGGTGTTAACCTAGTTGCTGTACCT
ACAGGTTATGTTGATACACCTAATAATACAGATTTTTCCAGAGTTAGTGCTAAAC
CACCGCCTGGAGATCAATTTAAACACCTCATACCACTTATGTACAAAGGACTTCC
TTGGAATGTAGTGCGTATAAAGATTGTACAAATGTTAAGTGACACACTTAAAAAT
CTCTCTGACAGAGTCGTATTTGTCTTATGGGCACATGGCTTTGAGTTGACATCTAT
GAAGTATTTTGTGAAAATAGGACCTGAGCGCACCTGTTGTCTATGTGATAGACGT
GCCACATGCTTTTCCACTGCTTCAGACACTTATGCCTGTTGGCATCATTCTATTGG
ATTTGATTACGTCTATAATCCGTTTATGATTGATGTTCAACAATGGGGTTTTACAG
GTAACCTACAAAGCAACCATGATCTGTATTGTCAAGTCCATGGTAATGCACATGT
AGCTAGTTGTGATGCAATCATGACTAGGTGTCTAGCTGTCCACGAGTGCTTTGTT
AAGCGTGTTGACTGGACTATTGAATATCCTATAATTGGTGATGAACTGAAGATTA
ATGCGGCTTGTAGAAAGGTTCAACACATGGTTGTTAAAGCTGCATTATTAGCAGA
CAAATTCCCAGTTCTTCACGACATTGGTAACCCTAAAGCTATTAAGTGTGTACCT
CAAGCTGATGTAGAATGGAAGTTCTATGATGCACAGCCTTGTAGTGACAAAGCTT
ATAAAATAGAAGAATTATTCTATTCTTATGCCACACATTCTGACAAATTCACAGA
TGGTGTATGCCTATTTTGGAATTGCAATGTCGATAGATATCCTGCTAATTCCATTG
TTTGTAGATTTGACACTAGAGTGCTATCTAACCTTAACTTGCCTGGTTGTGATGGT
GGCAGTTTGTATGTAAATAAACATGCATTCCACACACCAGCTTTTGATAAAAGTG
CTTTTGTTAATTTAAAACAATTACCATTTTTCTATTACTCTGACAGTCCATGTGAG
TCTCATGGAAAACAAGTAGTGTCAGATATAGATTATGTACCACTAAAGTCTGCTA
CGTGTATAACACGTTGCAATTTAGGTGGTGCTGTCTGTAGACATCATGCTAATGA
GTACAGATTGTATCTCGATGCTTATAACATGATGATCTCAGCTGGCTTTAGCTTGT
GGGTTTACAAACAATTTGATACTTATAACCTCTGGAACACTTTTACAAGACTTCA
GAGTTTAGAAAATGTGGCTTTTAATGTTGTAAATAAGGGACACTTTGATGGACAA
CAGGGTGAAGTACCAGTTTCTATCATTAATAACACTGTTTACACAAAAGTTGATG
GTGTTGATGTAGAATTGTTTGAAAATAAAACAACATTACCTGTTAATGTAGCATT
TGAGCTTTGGGCTAAGCGCAACATTAAACCAGTACCAGAGGTGAAAATACTCAA
TAATTTGGGTGTGGACATTGCTGCTAATACTGTGATCTGGGACTACAAAAGAGAT
GCTCCAGCACATATATCTACTATTGGTGTTTGTTCTATGACTGACATAGCCAAGA
AACCAACTGAAACGATTTGTGCACCACTCACTGTCTTTTTTGATGGTAGAGTTGA
TGGTCAAGTAGACTTATTTAGAAATGCCCGTAATGGTGTTCTTATTACAGAAGGT
AGTGTTAAAGGTTTACAACCATCTGTAGGTCCCAAACAAGCTAGTCTTAATGGAG
TCACATTAATTGGAGAAGCCGTAAAAACACAGTTCAATTATTATAAGAAAGTTG
ATGGTGTTGTCCAACAATTACCTGAAACTTACTTTACTCAGAGTAGAAATTTACA
AGAATTTAAACCCAGGAGTCAAATGGAAATTGATTTCTTAGAATTAGCTATGGAT
GAATTCATTGAACGGTATAAATTAGAAGGCTATGCCTTCGAACATATCGTTTATG
GAGATTTTAGTCATAGTCAGTTAGGTGGTTTACATCTACTGATTGGACTAGCTAA
ACGTTTTAAGGAATCACCTTTTGAATTAGAAGATTTTATTCCTATGGACAGTACA
GTTAAAAACTATTTCATAACAGATGCGCAAACAGGTTCATCTAAGTGTGTGTGTT
CTGTTATTGATTTATTACTTGATGATTTTGTTGAAATAATAAAATCCCAAGATTTA
TCTGTAGTTTCTAAGGTTGTCAAAGTGACTATTGACTATACAGAAATTTCATTTAT
GCTTTGGTGTAAAGATGGCCATGTAGAAACATTTTACCCAAAATTACAATCTAGT
CAAGCGTGGCAACCGGGTGTTGCTATGCCTAATCTTTACAAAATGCAAAGAATG
CTATTAGAAAAGTGTGACCTTCAAAATTATGGTGATAGTGCAACATTACCTAAAG
GCATAATGATGAATGTCGCAAAATATACTCAACTGTGTCAATATTTAAACACATT
AACATTAGCTGTACCCTATAATATGAGAGTTATACATTTTGGTGCTGGTTCTGAT
AAAGGAGTTGCACCAGGTACAGCTGTTTTAAGACAGTGGTTGCCTACGGGTACG
CTGCTTGTCGATTCAGATCTTAATGACTTTGTCTCTGATGCAGATTCAACTTTGAT
TGGTGATTGTGCAACTGTACATACAGCTAATAAATGGGATCTCATTATTAGTGAT
ATGTACGACCCTAAGACTAAAAATGTTACAAAAGAAAATGACTCTAAAGAGGGT
TTTTTCACTTACATTTGTGGGTTTATACAACAAAAGCTAGCTCTTGGAGGTTCCGT
GGCTATAAAGATAACAGAACATTCTTGGAATGCTGATCTTTATAAGCTCATGGGA
CACTTCGCATGGTGGACAGCCTTTGTTACTAATGTGAATGCGTCATCATCTGAAG
CATTTTTAATTGGATGTAATTATCTTGGCAAACCACGCGAACAAATAGATGGTTA
TGTCATGCATGCAAATTACATATTTTGGAGGAATACAAATCCAATTCAGTTGTCT
TCCTATTCTTTATTTGACATGAGTAAATTTCCCCTTAAATTAAGGGGTACTGCTGT
TATGTCTTTAAAAGAAGGTCAAATCAATGATATGATTTTATCTCTTCTTAGTAAA
GGTAGACTTATAATTAGAGAAAACAACAGAGTTGTTATTTCTAGTGATGTTCTTG
TTAACAACTAAACGAACAATGTTTGTTTTTCTTGTTTTATTGCCACTAGTCTCTAG
TCAGTGTGTTAATCTTACAACCAGAACTCAATTACCCCCTGCATACACTAATTCTT
TCACACGTGGTGTTTATTACCCTGACAAAGTTTTCAGATCCTCAGTTTTACATTCA
ACTCAGGACTTGTTCTTACCTTTCTTTTCCAATGTTACTTGGTTCCATGCTATACAT
GTCTCTGGGACCAATGGTACTAAGAGGTTTGATAACCCTGTCCTACCATTTAATG
ATGGTGTTTATTTTGCTTCCAtTGAGAAGTCTAACATAATAAGAGGCTGGATTTTT
GGTACTACTTTAGAcTCGAAGACCCAGTCCCTACTTATTGTTAATAACGCTACTAA
TGTTGTTATTAAAGTCTGTGAATTTCAATTTTGTAATGATCCATTTTTGGaTGTTTA
TTACCACAAAAACAACAAAAGTTGGATGaAAAGTGAGTTCAGAGTTTATTCTAGT
GCGAATAATTGCACTTTTGAATATGTCTCTCAGCCTTTTCTTATGGACCTTGAAGG
AAAACAGGGTAATTTCAAAAATCTTAGGGAATTTGTGTTTAAGAATATTGATGGT
TATTTTAAAATATATTCTAAGCACACGCCTATTAATTTAGTGCGTGATCTCCCtCA
GGGTTTTTCGGCTTTAGAACCATTGGTAGATTTGCCAATAGGTATTAACATCACT
AGGTTTCAAACTTTACTTGCTTTACATAGAAGTTATTTGACTCCTGGTGATTCTTC
TTCAGGTTGGACAGCTGGTGCTGCAGCTTATTATGTGGGTTATCTTCAACCTAGG
ACTTTTCTATTAAAATATAATGAAAATGGAACCATTACAGATGCTGTAGACTGTG
CACTTGACCCTCTCTCAGAAACAAAGTGTACGTTGAAATCCTTCACTGTAGAAAA
AGGAATCTATCAAACTTCTAACTTTAGAGTCCAACCAACAGAATCTATTGTTAGA
TTTCCTAATATTACAAACTTGTGCCCTTTTGGTGAAGTTTTTAACGCCACCAGATT
TGCATCTGTTTATGCTTGGAACAGGAAGAGAATCAGCAACTGTGTTGCTGATTAT
TCTGTCCTATATAATTCCGCATCATTTTCCACTTTTAAGTGTTATGGAGTGTCTCC
TACTAAATTAAATGATCTCTGCTTTACTAATGTCTATGCAGATTCATTTGTAATTA
GAGGTGATGAAGTCAGACAAATCGCTCCAGGGCAAACTGGAAAGATTGCTGATT
ATAATTATAAATTACCAGATGATTTTACAGGCTGCGTTATAGCTTGGAATTCTAA
CAATCTTGATTCTAAGGTTGGTGGTAATTATAATTACCgGTATAGATTGTTTAGGA
AGTCTAATCTCAAACCTTTTGAGAGAGATATTTCAACTGAAATCTATCAGGCCGG
TAGCACACCTTGTAATGGTGTTcAAGGTTTTAATTGTTACTTTCCTTTACAATCAT
ATGGTTTCCAACCCACTAATGGTGTTGGTTACCAACCATACAGAGTAGTAGTACT
TTCTTTTGAACTTCTACATGCACCAGCAACTGTTTGTGGACCTAAAAAGTCTACT
AATTTGGTTAAAAACAAATGTGTCAATTTCAACTTCAATGGTTTAACAGGCACAG
GTGTTCTTACTGAGTCTAACAAAAAGTTTCTGCCTTTCCAACAATTTGGCAGAGA
CATTGCTGACACTACTGATGCTGTCCGTGATCCACAGACACTTGAGATTCTTGAC
ATTACACCATGTTCTTTTGGTGGTGTCAGTGTTATAACACCAGGAACAAATACTT
CTAACCAGGTTGCTGTTCTTTATCAGGgTGTTAACTGCACAGAAGTCCCTGTTGCT
ATTCATGCAGATCAACTTACTCCTACTTGGCGTGTTTATTCTACAGGTTCTAATGT
TTTTCAAACACGTGCAGGCTGTTTAATAGGGGCTGAACATGTCAACAACTCATAT
GAGTGTGACATACCCATTGGTGCAGGTATATGCGCTAGTTATCAGACTCAGACTA
ATTCTCgTCGGCGGGCACGTAGTGTAGCTAGTCAATCCATCATTGCCTACACTAT
GTCACTTGGTGCAGAAAATTCAGTTGCTTACTCTAATAACTCTATTGCCATACCC
ACAAATTTTACTATTAGTGTTACCACAGAAATTCTACCAGTGTCTATGACCAAGA
CATCAGTAGATTGTACAATGTACATTTGTGGTGATTCAACTGAATGCAGCAATCT
TTTGTTGCAATATGGCAGTTTTTGTACACAATTAAACCGTGCTTTAACTGGAATA
GCTGTTGAACAAGACAAAAACACCCAAGAAGTTTTTGCACAAGTCAAACAAATT
TACAAAACACCACCAATTAAAGATTTTGGTGGTTTTAATTTTTCACAAATATTAC
CAGATCCATCAAAACCAAGCAAGAGGTCATTTATTGAAGATCTACTTTTCAACAA
AGTGACACTTGCAGATGCTGGCTTCATCAAACAATATGGTGATTGCCTTGGTGAT
ATTGCTGCTAGAGACCTCATTTGTGCACAAAAGTTTAACGGCCTTACTGTTTTGC
CACCTTTGCTCACAGATGAAATGATTGCTCAATACACTTCTGCACTGTTAGCGGG
TACAATCACTTCTGGTTGGACCTTTGGTGCAGGTGCTGCATTACAAATACCATTT
GCTATGCAAATGGCTTATAGGTTTAATGGTATTGGAGTTACACAGAATGTTCTCT
ATGAGAACCAAAAATTGATTGCCAACCAATTTAATAGTGCTATTGGCAAAATTCA
AGACTCACTTTCTTCCACAGCAAGTGCACTTGGAAAACTTCAAGATGTGGTCAAC
CAAAATGCACAAGCTTTAAACACGCTTGTTAAACAACTTAGCTCCAATTTTGGTG
CAATTTCAAGTGTTTTAAATGATATCCTTTCACGTCTTGACAAAGTTGAGGCTGA
AGTGCAAATTGATAGGTTGATCACAGGCAGACTTCAAAGTTTGCAGACATATGTG
ACTCAACAATTAATTAGAGCTGCAGAAATCAGAGCTTCTGCTAATCTTGCTGCTA
CTAAAATGTCAGAGTGTGTACTTGGACAATCAAAAAGAGTTGATTTTTGTGGAAA
GGGCTATCATCTTATGTCCTTCCCTCAGTCAGCACCTCATGGTGTAGTCTTCTTGC
ATGTGACTTATGTCCCTGCACAAGAAAAGAACTTCACAACTGCTCCTGCCATTT
GTCATGATGGAAAAGCACACTTTCCTCGTGAAGGTGTCTTTGTTTCAAATGGCAC
ACACTGGTTTGTAACACAAAGGAATTTTTATGAACCACAAATCATTACTACAGAC
AACACATTTGTGTCTGGTAACTGTGATGTTGTAATAGGAATTGTCAACAACACAG
TTTATGATCCTTTGCAACCTGAATTAGACTCATTCAAGGAGGAGTTAGATAAATA
TTTTAAGAATCATACATCACCAGATGTTGATTTAGGTGACATCTCTGGCATTAAT
GCTTCAGTTGTAAACATTCAAAAAGAAATTGACCGCCTCAATGAGGTTGCCAAG
AATTTAAATGAATCTCTCATCGATCTCCAAGAACTTGGAAAGTATGAGCAGTATA
TAAAATGGCCATGGTACATTTGGCTAGGTTTTATAGCTGGCTTGATTGCCATAGT
AATGGTGACAATTATGCTTTGCTGTATGACCAGTTGCTGTAGTTGTCTCAAGGGC
TGTTGTTCTTGTGGATCCTGCTGCAAATTTGATGAAGACGACTCTGAGCCAGTGC
TCAAAGGAGTCAAATTACATTACACATAAACGAACTTATGGATTTGTTTATGAGA
ATCTTCACAATTGGAACTGTAACTTTGAAGCAAGGTGAAATCAAGGATGCTACTC
CTTCAGATTTTGTTCGCGCTACTGCAACGATACCGATACAAGCCTCACTCCCTTTC
GGATGGCTTATTGTTGGCGTTGCACTTCTTGCTGTTTTTCAGAGCGCTTCCAAAAT
CATAACCCTCAAAAAGAGATGGCAACTAGCACTCTCCAAGGGTGTTCACTTTGTT
TGCAACTTGCTGTTGTTGTTTGTAACAGTTTACTCACACCTTTTGCTCGTTGCTGC
TGGCCTTGAAGCCCCTTTTCTCTATCTTTATGCTTTAGTCTACTTCTTGCAGAGTAT
AAACTTTGTAAGAATAATAATGAGGCTTTGGCTTTGCTGGAAATGCCGTTCCAAA
AACCCATTACTTTATGATGCCAACTATTTTCTTTGCTGGCATACTAATTGTTACGA
CTATTGTATACCTTACAATAGTGTAACTTCTTCAATTGTCATTACTTCAGGTGATG
GCACAACAAGTCCTATTTCTGAACATGACTACCAGATTGGTGGTTATACTGAAAA
ATGGGAATCTGGAGTAAAAGACTGTGTTGTATTACACAGTTACTTCACTTCAGAC
TATTACCAGCTGTACTCAACTCAATTGAGTACAGACACTGGTGTTGAACATGTTA
CCTTCTTCATCTACAATAAAATTGTTGATGAGCCTGAAGAACATGTCCAAATTCA
CACAATCGACGGTTCATCCGGAGTTGTTAATCCAGTAATGGAACCAATTTATGAT
GAACCGACGACGACTACTAGCGTGCCTTTGTAAGCACAAGCTGATGAGTACGAA
CTTATGTACTCATTCGTTTCGGAAGAGACAGGTACGTTAATAGTTAATAGCGTAC
TTCTTTTTCTTGCTTTCGTGGTATTCTTGCTAGTTACACTAGCCATCCTTACTGCGC
TTCGATTGTGTGCGTACTGCTGCAATATTGTTAACGTGAGTCTTGTAAAACCTTCT
TTTTACGTTTACTCTCGTGTTAAAAATCTGAATTCTTCTAGAGTTCCTGATCTTCT
GGTCTAAACGAACTAAATATTATATTAGTTTTTCTGTTTGGAACTTTAATTTTAGC
CATGGCAGATTCCAACGGTACTATTACCGTTGAAGAGCTTAAAAAGCTCCTTGAA
CAATGGAACCTAGTAATAGGTTTCCTATTCCTTACATGGATTTGTCTTCTACAATT
TGCCTATGCCAACAGGAATAGGTTTTTGTATATAATTAAGTTAATTTTCCTCTGGC
TGTTATGGCCAGTAACTTTAGCTTGTTTTGTGCTTGCTGCTGTTTACAGAATAAAT
TGGATCACCGGTGGAATTGCTATCGCAATGGCTTGTCTTGTAGGCTTGATGTGGC
TCAGCTACTTCATTGCTTCTTTCAGACTGTTTGCGCGTACGCGATCCATGTGGTCA
TTCAATCCAGAAACTAACATTCTTCTCAACGTGCCACTCCATGGCACTATTCTGA
CCAGACCGCTTCTAGAAAGTGAACTCGTAATCGGAGCTGTGATCCTTCGTGGACA
TCTTCGTATTGCTGGACACCATCTAGGACGCTGTGACATCAAGGACCTGCCTAAA
GAAATCACTGTTGCTACATCACGAACGCTTTCTTATTACAAATTGGGAGCTTCGC
AGCGTGTAGCAGGTGACTCAGGTTTTGCTGCATACAGTCGCTACAGGATTGGCAA
CTATAAATTAAACACAGACCATTCCAGTAGCAGTGACAATATTGCTTTGCTTGTA
CAGTAAGTGACAACAGATGTTTCATCTCGTTGACTTTCAGGTTACTATAGCAGAG
ATATTACTAATTATTATGAGGACTTTTAAAGTTTCCATTTGGAATCTTGATTACAT
CATAAACCTCATAATTAAAAATTTATCTAAGTCACTAACTGAGAATAAATATTCT
CAATTAGATGAAGAGCAACCAATGGAGATTGATTAAACGAACATGAAAATTATT
CTTTTCTTGGCACTGATAACACTCGCTACTTGTGAGCTTTATCACTACCAAGAGTG
TGTTAGAGGTACAACAGTACTTTTAAAAGAACCTTGCTCTTCTGGAACATACGAG
GGCAATTCACCATTTCATCCTCTAGCTGATAACAAATTTGCACTGACTTGCTTTAG
CACTCAATTTGCTTTTGCTTGTCCTGACGGCGTAAAACACGTCTATCAGTTACGTG
CCAGATCAGTTTCACCTAAACTGTTCATCAGACAAGAGGAAGTTCAAGAACTTTA
CTCTCCAATTTTTCTTATTGTTGCGGCAATAGTGTTTATAACACTTTGCTTCACAC
TCAAAAGAAAGACAGAATGATTGAACTTTCATTAATTGACTTCTATTTGTGCTTTT
TAGCCTTTCTGCTATTCCTTGTTTTAATTATGCTTATTATCTTTTGGTTCTCACTTG
AACTGCAAGATCATAATGAAACTTGTCACGCCTAAACGAACATGAAATTTCTTGT
TTTCTTAGGAATCATCACAACTGTAGCTGCATTTCACCAAGAATGTAGTTTACAG
TCATGTACTCAACATCAACCATATGTAGTTGATGACCCGTGTCCTATTCACTTCTA
TTCTAAATGGTATATTAGAGTAGGAGCTAGAAAATCAGCACCTTTAATTGAATTG
TGCGTGGATGAGGCTGGTTCTAAATCACCCATTCAGTACATCGATATCGGTAATT
ATACAGTTTCCTGTTTACCTTTTACAATTAATTGCCAGGAACCTAAATTGGGTAGT
CTTGTAGTGCGTTGTTCGTTCTATGAAGACTTTTTAGAGTATCATGACGTTCGTGT
TGTTTTAGATTTCATCTAAACGAACAAACTAAAATGTCTGATAATGGACCCCAAA
ATCAGCGAAATGCACCCCGCATTACGTTTGGTGGACCCTCAGATTCAACTGGCAG
TAACCAGAATGGAGAACGCAGTGGGGCGCGATCAAAACAACGTCGGCCCCAAG
GTTTACCCAATAATACTGCGTCTTGGTTCACCGCTCTCACTCAACATGGCAAGGA
AGACCTTAAATTCCCTCGAGGACAAGGCGTTCCAATTAACACCAATAGCAGTCC
AGATGACCAAATTGGCTACTACCGAAGAGCTACCAGACGAATTCGTGGTGGTGA
CGGTAAAATGAAAGATCTCAGTCCAAGATGGTATTTCTACTACCTAGGAACTGG
GCCAGAAGCTGGACTTCCCTATGGTGCTAACAAAGACGGCATCATATGGGTTGC
AACTGAGGGAGCCTTGAATACACCAAAAGATCACATTGGCACCCGCAATCCTGC
TAACAATGCTGCAATCGTGCTACAACTTCCTCAAGGAACAACATTGCCAAAAGG
CTTCTACGCAGAAGGGAGCAGAGGCGGCAGTCAAGCCTCTTCTCGTTCCTCATCA
CGTAGTCGCAACAGTTCAAGAAATTCAACTCCAGGCAGCAGTAGGGGAACTTCT
CCTGCTAGAATGGCTGGCAATGGCGGTGATGCTGCTCTTGCTTTGCTGCTGCTTG
ACAGATTGAACCAGCTTGAGAGCAAAATGTCTGGTAAAGGCCAACAACAACAAG
GCCAAACTGTCACTAAGAAATCTGCTGCTGAGGCTTCTAAGAAGCCTCGGCAAA
AACGTACTGCCACTAAAGCATACAATGTAACACAAGCTTTCGGCAGACGTGGTC
CAGAACAAACCCAAGGAAATTTTGGGGACCAGGAACTAATCAGACAAGGAACT
GATTACAAACATTGGCCGCAAATTGCACAATTTGCCCCCAGCGCTTCAGCGTTCT
TCGGAATGTCGCGCATTGGCATGGAAGTCACACCTTCGGGAACGTGGTTGACCTA
CACAGGTGCCATCAAATTGGATGACAAAGATCCAAATTTCAAAGATCAAGTCAT
TTTGCTGAATAAGCATATTGACGCATACAAAACATTCCCACCAACAGAGCCTAA
AAAGGACAAAAAGAAGAAGGCTGATGAAACTCAAGCCTTACCGCAGAGACAGA
AGAAACAGCAAACTGTGACTCTTCTTCCTGCTGCAGATTTGGATGATTTCTCCAA
ACAATTGCAACAATCCATGAGCAGTGCTGACTCAACTCAGGCCTAAACTCATGC
AGACCACACAAGGCAGATGGGCTATATAAACGTTTTCGCTTTTCCGTTTACGATA
TATAGTCTACTCTTGTGCAGAATGAATTCTCGTAACTACATAGCACAAGTAGATG
TAGTTAACTTTAATCTCACATAGCAATCTTTAATCAGTGTGTAACATTAGGGAGG
ACTTGAAAGAGCCACCACATTTTCACCGAGGCCACGCGGAGTACGATCGAGTGT
ACAGTGAACAATGCTAGGGAGAGCTGCCTATATGGAAGAGCCCTAATGTGTAAA
ATTAATTTTAGTAGTGCTATCCCCATGTGATTTTAATAGCTTCTTAGGAGAATGAC
AAAAAAAAAAAAAAAAAAAAAAAAAAAAAAAAAAAAAAAAAAAAAAAAAAAA
AAAAAAAGGGTCGGCATGGCATCTCCACCTCCTCGCGGTCCGACCTGGGCATCC
GAAGGAGGACGCACGTCCACTCGGATGGCTAAGGGAGCAGCACACTGGCGGCC
GTTACTAGGGCCGCGCCCTTCCCAACAGTTGCGCAGCCTGAATGGCGAATGGAG
ATCCAATTTTTAAGTGTATAATGTGTTAAACTACTGATTCTAATTGTTTGTGTATT
TTAGATTCACAGTCCCAAGGCTCATTTCAGGCCCCTCAGTCCTCACAGTCTGTTC
ATGATCATAATCAGCCATACCACATTTGTAGAGGTTTTACTTGCTTTAAAAAACC
TCCCACACCTCCCCCTGAACCTGAAACATAAAATGAATGCAATTGTTGTTGTTAA
CTTGTTTATTGCAGCTTATAATGGTTACAAATAAAGCAATAGCATCACAAATTTC
ACAAATAAAGCATTTTTTTCACTGCATTCTAGTTGTGGTTTGTCCAAACTCATCAA
TGTATCTTAAAGCTTGAGTATTCTATAGTCTCACCTAAATAGCTTGGCGTAATCAT
GGTCATAGCTGTTTCCTGTGTGAAATTGTTATCCGCTCACAATTCCACACAACAT
ACGAGCCGGAAGCATAAAGTGTAAAGCCTGGGGTGCCTAATGAGTGAGCTAACT
CACATTAATTGCGTTGCGCTCACTGCCCGCTTTCCAGTCGGGAAACCTGTCGTGC
CAGCTGCATTAATGAATCGGCCAACGCGAACCCCTTGCGGCCGCCCGGGCCGTC
GACCAATTCTCATGTTTGACAGCTTATCATCGAATTTCTGCCATTCATCCGCTTAT
TATCACTTATTCAGGCGTAGCAACCAGGCGTTTAAGGGCACCAATAACTGCCTTA
AAAAAATTACGCCCCGCCCTGCCACTCATCGCAGTACTGTTGTAATTCATTAAGC
ATTCTGCCGACATGGAAGCCATCACAAACGGCATGATGAACCTGAATCGCCAGC
GGCATCAGCACCTTGTCGCCTTGCGTATAATATTTGCCCATGGTGAAAACGGGGG
CGAAGAAGTTGTCCATATTGGCCACGTTTAAATCAAAACTGGTGAAACTCACCCA
GGGATTGGCTGAGACGAAAAACATATTCTCAATAAACCCTTTAGGGAAATAGGC
CAGGTTTTCACCGTAACACGCCACATCTTGCGAATATATGTGTAGAAACTGCCGG
AAATCGTCGTGGTATTCACTCCAGAGCGATGAAAACGTTTCAGTTTGCTCATGGA
AAACGGTGTAACAAGGGTGAACACTATCCCATATCACCAGCTCACCGTCTTTCAT
TGCCATACGAAATTCCGGATGAGCATTCATCAGGCGGGCAAGAATGTGAATAAA
GGCCGGATAAAACTTGTGCTTATTTTTCTTTACGGTCTTTAAAAAGGCCGTAATAT
CCAGCTGAACGGTCTGGTTATAGGTACATTGAGCAACTGACTGAAATGCCTCAA
AATGTTCTTTACGATGCCATTGGGATATATCAACGGTGGTATATCCAGTGATTTTT
TTCTCCATTTTAGCTTCCTTAGCTCCTGAAAATCTCGATAACTCAAAAAATACGCC
CGGTAGTGATCTTATTTCATTATGGTGAAAGTTGGAACCTCTTACGTGCCGATCA
ACGTCTCATTTTCGCCAAAAGTTGGCCCAGGGCTTCCCGGTATCAACAGGGACAC
CAGGATTTATTTATTCTGCGAAGTGATCTTCCGTCACAGGTATTTATTCGCGATAA
GCTCATGGAGCGGCGTAACCGTCGCACAGGAAGGACAGAGAAAGCGCGGATCT
GGGAAGTGACGGACAGAACGGTCAGGACCTGGATTGGGGAGGCGGTTGCCGCC
GCTGCTGCTGACGGTGTGACGTTCTCTGTTCCGGTCACACCACATACGTTCCGCC
ATTCCTATGCGATGCACATGCTGTATGCCGGTATACCGCTGAAAGTTCTGCAAAG
CCTGATGGGACATAAGTCCATCAGTTCAACGGAAGTCTACACGAAGGTTTTTGCG
CTGGATGTGGCTGCCCGGCACCGGGTGCAGTTTGCGATGCCGGAGTCTGATGCG
GTTGCGATGCTGAAACAATTATCCTGAGAATAAATGCCTTGGCCTTTATATGGAA
ATGTGGAACTGAGTGGATATGCTGTTTTTGTCTGTTAAACAGAGAAGCTGGCTGT
TATCCACTGAGAAGCGAACGAAACAGTCGGGAAAATCTCCCATTATCGTAGAGA
TCCGCATTATTAATCTCAGGAGCCTGTGTAGCGTTTATAGGAAGTAGTGTTCTGT
CATGATGCCTGCAAGCGGTAACGAAAACGATTTGAATATGCCTTCAGGAACAAT
AGAAATCTTCGTGCGGTGTTACGTTGAAGTGGAGCGGATTATGTCAGCAATGGAC
AGAACAACCTAATGAACACAGAACCATGATGTGGTCTGTCCTTTTACAGCCAGTA
GTGCTCGCCGCAGTCGAGCGACAGGGCGAAGCCCTCGGCTGGTTGCCCTCGCCG
CTGGGCTGGCGGCCGTCTATGGCCCTGCAAACGCGCCAGAAACGCCGTCGAAGC
CGTGTGCGAGACACCGCGGCCGGCCGCCGGCGTTGTGGATACCTCGCGGAAAAC
TTGGCCCTCACTGACAGATGAGGGGCGGACGTTGACACTTGAGGGGCCGACTCA
CCCGGCGCGGCGTTGACAGATGAGGGGCAGGCTCGATTTCGGCCGGCGACGTGG
AGCTGGCCAGCCTCGCAAATCGGCGAAAACGCCTGATTTTACGCGAGTTTCCCAC
AGATGATGTGGACAAGCCTGGGGATAAGTGCCCTGCGGTATTGACACTTGAGGG
GCGCGACTACTGACAGATGAGGGGCGCGATCCTTGACACTTGAGGGGCAGAGTG
CTGACAGATGAGGGGCGCACCTATTGACATTTGAGGGGCTGTCCACAGGCAGAA
AATCCAGCATTTGCAAGGGTTTCCGCCCGTTTTTCGGCCACCGCTAACCTGTCTTT
TAACCTGCTTTTAAACCAATATTTATAAACCTTGTTTTTAACCAGGGCTGCGCCCT
GTGCGCGTGACCGCGCACGCCGAAGGGGGGTGCCCCCCCTTCTCGAACCCTCCC
GGTCGAGTGAGCGAGGAAGCACCAGGGAACAGCACTTATATATTCTGCTTACAC
ACGATGCCTGAAAAAACTTCCCTTGGGGTTATCCACTTATCCACGGGGATATTTT
TATAATTATTTTTTTTATAGTTTTTAGATCTTCTTTTTTAGAGCGCCTTGTAGGCCT
TTATCCATGCTGGTTCTAGAGAAGGTGTTGTGACAAATTGCCCTTTCAGTGTGAC
AAATCACCCTCAAATGACAGTCCTGTCTGTGACAAATTGCCCTTAACCCTGTGAC
AAATTGCCCTCAGAAGAAGCTGTTTTTTCACAAAGTTATCCCTGCTTATTGACTCT
TTTTTATTTAGTGTGACAATCTAAAAACTTGTCACACTTCACATGGATCTGTCATG
GCGGAAACAGCGGTTATCAATCACAAGAAACGTAAAAATAGCCCGCGAATCGTC
CAGTCAAACGACCTCACTGAGGCGGCATATAGTCTCTCCCGGGATCAAAAACGT
ATGCTGTATCTGTTCGTTGACCAGATCAGAAAATCTGATGGCACCCTACAGGAAC
ATGACGGTATCTGCGAGATCCATGTTGCTAAATATGCTGAAATATTCGGATTGAC
CTCTGCGGAAGCCAGTAAGGATATACGGCAGGCATTGAAGAGTTTCGCGGGGAA
GGAAGTGGTTTTTTATCGCCCTGAAGAGGATGCCGGCGATGAAAAAGGCTATGA
ATCTTTTCCTTGGTTTATCAAACGTGCGCACAGTCCATCCAGAGGGCTTTACAGT
GTACATATCAACCCATATCTCATTCCCTTCTTTATCGGGTTACAGAACCGGTTTAC
GCAGTTTCGGCTTAGTGAAACAAAAGAAATCACCAATCCGTATGCCATGCGTTTA
TACGAATCCCTGTGTCAGTATCGTAAGCCGGATGGCTCAGGCATCGTCTCTCTGA
AAATCGACTGGATCATAGAGCGTTACCAGCTGCCTCAAAGTTACCAGCGTATGCC
TGACTTCCGCCGCCGCTTCCTGCAGGTCTGTGTTAATGAGATCAACAGCAGAACT
CCAATGCGCCTCTCATACATTGAGAAAAAGAAAGGCCGCCAGACGACTCATATC
GTATTTTCCTTCCGCGATATCACTTCCATGACGACAGGATAGTCTGAGGGTTATC
TGTCACAGATTTGAGGGTGGTTCGTCACATTTGTTCTGACCTACTGAGGGTAATTT
GTCACAGTTTTGCTGTTTCCTTCAGCCTGCATGGATTTTCTCATACTTTTTGAACT
GTAATTTTTAAGGAAGCCAAATTTGAGGGCAGTTTGTCACAGTTGATTTCCTTCTC
TTTCCCTTCGTCATGTGACCTGATATCGGGGGTTAGTTCGTCATCATTGATGAGGG
TTGATTATCACAGTTTATTACTCTGAATTGGCTATCCGCGTGTGTACCTCTACCTG
GAGTTTTTCCCACGGTGGATATTTCTTCTTGCGCTGAGCGTAAGAGCTATCTGAC
AGAACAGTTCTTCTTTGCTTCCTCGCCAGTTCGCTCGCTATGCTCGGTTACACGGC
TGCGGCG
SEQ ID NO: 68 - Clone pCCI-4K-SARS-COV-2-7N-1-delta, with notable
sequences underlined or otherwise identified (eg. restriction enzyme sites, nucelotide
differences):
GATGTGCTGCAAGGCGATTAAGTTGGGTAACGCCAGGGTTTTCCCAGT
CACGACGTTGTAAAACGACGGCCAGTGAATTGTAATACGACTCACTATAGGGCG
AATTCTAGTTATTAATAGTAATCAATTACGGGGTCATTAGTTCATAGCCCATATA
TGGAGTTCCGCGTTACATAACTTACGGTAAATGGCCCGCCTGGCTGACCGCCCAA
CGACCCCCGCCCATTGACGTCAATAATGACGTATGTTCCCATAGTAACGCCAATA
GGGACTTTCCATTGACGTCAATGGGTGGAGTATTTACGGTAAACTGCCCACTTGG
CAGTACATCAAGTGTATCATATGCCAAGTACGCCCCCTATTGACGTCAATGACGG
TAAATGGCCCGCCTGGCATTATGCCCAGTACATGACCTTATGGGACTTTCCTACT
TGGCAGTACATCTACGTATTAGTCATCGCTATTACCATGGTGATGCGGTTTTGGC
AGTACATCAATGGGCGTGGATAGCGGTTTGACTCACGGGGATTTCCAAGTCTCCA
CCCCATTGACGTCAATGGGAGTTTGTTTTGGCACCAAAATCAACGGGACTTTCCA
AAATGTCGTAACAACTCCGCCCCATTGACGCAAATGGGCGGTAGGCGTGTACGG
TGGGAGGTCTATATAAGCAGAGCTGGTTTAGTGAACCGTATTAAAGGTTTATACC
TTCCCAGGTAACAAACCAACCAACTTTCGATCTCTTGTAGATCTGTTCTCTAAAC
GAACTTTAAAATCTGTGTGGCTGTCACTCGGCTGCATGCTTAGTGCACTCACGCA
GTATAATTAATAACTAATTACTGTCGTTGACAGGACACGAGTAACTCGTCTATCT
TCTGCAGGCTGCTTACGGTTTCGTCCGTGTTGCAGCCGATCATCAGCACATCTAG
GTTTCGTCCGGGTGTGACCGAAAGGTAAGATGGAGAGCCTTGTCCCTGGTTTCAA
CGAGAAAACACACGTCCAACTCAGTTTGCCTGTTTTACAGGTTCGCGACGTGCTC
GTACGTGGCTTTGGAGACTCCGTGGAGGAGGTCTTATCAGAGGCACGTCAACAT
CTTAAAGATGGCACTTGTGGCTTAGTAGAAGTTGAAAAAGGCGTTTTGCCTCAAC
TTGAACAGCCCTATGTGTTCATCAAACGTTCGGATGCTCGAACTGCACCTCATGG
TCATGTTATGGTTGAGCTGGTAGCAGAACTCGAAGGCATTCAGTACGGTCGTAGT
GGTGAGACACTTGGTGTCCTTGTCCCTCATGTGGGCGAAATACCAGTGGCTTACC
GCAAGGTTCTTCTTCGTAAGAACGGTAATAAAGGAGCTGGTGGCCATAGTTACG
GCGCCGATCTAAAGTCATTTGACTTAGGCGACGAGCTTGGCACTGATCCTTATGA
AGATTTTCAAGAAAACTGGAACACTAAACATAGCAGTGGTGTTACCCGTGAACT
CATGCGTGAGCTTAACGGAGGGGCATACACTCGCTATGTCGATAACAACTTCTGT
GGCCCTGATGGCTACCCTCTTGAGTGCATTAAAGACCTTCTAGCACGTGCTGGTA
AAGCTTCATGCACTTTGTCCGAACAACTGGACTTTATTGACACTAAGAGGGGTGT
ATACTGCTGCCGTGAACATGAGCATGAAATTGCTTGGTACACGGAACGTTCTGAA
AAGAGCTATGAATTGCAGACACCTTTTGAAATTAAATTGGCAAAGAAATTTGAC
ACCTTCAATGGGGAATGTCCAAATTTTGTATTTCCCTTAAATTCCATAATCAAGA
CTATTCAACCAAGGGTTGAAAAGAAAAAGCTTGATGGCTTTATGGGTAGAATTC
GATCTGTCTATCCAGTTGCGTCACCAAATGAATGCAACCAAATGTGCCTTTCAAC
TCTCATGAAGTGTGATCATTGTGGTGAAACTTCATGGCAGACGGGCGATTTTGTT
AAAGCCACTTGCGAATTTTGTGGCACTGAGAATTTGACTAAAGAAGGTGCCACT
ACTTGTGGTTACTTACCCCAAAATGCTGTTGTTAAAATTTATTGTCCAGCATGTCA
CAATTCAGAAGTAGGACCTGAGCATAGTCTTGCCGAATACCATAATGAATCTGG
CTTGAAAACCATTCTTCGTAAGGGTGGTCGCACTATTGCCTTTGGAGGCTGTGTG
TTCTCTTATGTTGGTTGCCATAACAAGTGTGCCTATTGGGTCCCACGTGCTAGCG
CTAACATAGGTTGTAACCATACAGGTGTTGTTGGAGAAGGTTCCGAAGGTCTTAA
TGACAACCTTCTTGAAATACTCCAAAAAGAGAAAGTCAACATCAATATTGTTGGT
GACTTTAAACTTAATGAAGAGATCGCCATTATTTTGGCATCTTTTTCTGCTTCCAC
AAGTGCTTTTGTGGAAACTGTGAAAGGTTTGGATTATAAAGCATTCAAACAAATT
GTTGAATCCTGTGGTAATTTTAAAGTTACAAAAGGAAAAGCTAAAAAAGGTGCC
TGGAATATTGGTGAACAGAAATCAATACTGAGTCCTCTTTATGCATTTGCATCAG
AGGCTGCTCGTGTTGTACGATCAATTTTCTCCCGCACTCTTGAAACTGCTCAAAA
TTCTGTGCGTGTTTTACAGAAGGCCGCTATAACAATACTAGATGGAATTTCACAG
TATTCACTGAGACTCATTGATGCTATGATGTTCACATCTGATTTGGCTACTAACAA
TCTAGTTGTAATGGCCTACATTACAGGTGGTGTTGTTCAGTTGACTTCGCAGTGG
CTAACTAACATCTTTGGCACTGTTTATGAAAAACTCAAACCCGTCCTTGATTGGC
TTGAAGAGAAGTTTAAGGAAGGTGTAGAGTTTCTTAGAGACGGTTGGGAAATTG
TTAAATTTATCTCAACCTGTGCTTGTGAAATTGTCGGTGGACAAATTGTCACCTGT
GCAAAGGAAATTAAGGAGAGTGTTCAGACATTCTTTAAGCTTGTAAATAAATTTT
TGGCTTTGTGTGCTGACTCTATCATTATTGGTGGAGCTAAACTTAAAGCCTTGAAT
TTAGGTGAAACATTTGTCACGCACTCAAAGGGATTGTACAGAAAGTGTGTTAAAT
CCAGAGAAGAAACTGGCCTACTCATGCCTCTAAAAGCCCCAAAAGAAATTATCT
TCTTAGAGGGAGAAACACTTCCCACAGAAGTGTTAACAGAGGAAGTTGTCTTGA
AAACTGGTGATTTACAACCATTAGAACAACCTACTAGTGAAGCTGTTGAAGCTCC
ATTGGTTGGTACACCAGTTTGTATTAACGGGCTTATGTTGCTCGAAATCAAAGAC
ACAGAAAAGTACTGTGCCCTTGCACCTAATATGATGGTAACAAACAATACCTTCA
CACTCAAAGGCGGTGCACCAACAAAGGTTACTTTTGGTGATGACACTGTGATAG
AAGTGCAAGGTTACAAGAGTGTGAATATCACTTTTGAACTTGATGAAAGGATTG
ATAAAGTACTTAATGAGAAGTGCTCTGCCTATACAGTTGAACTCGGTACAGAAGT
AAATGAGTTCGCCTGTGTTGTGGCAGATGCTGTCATAAAAACTTTGCAACCAGTA
TCTGAATTACTTACACCACTGGGCATTGATTTAGATGAGTGGAGTATGGCTACAT
ACTACTTATTTGATGAGTCTGGTGAGTTTAAATTGGCTTCACATATGTATTGTTCT
TTCTACCCTCCAGATGAGGATGAAGAAGAAGGTGATTGTGAAGAAGAAGAGTTT
GAGCCATCAACTCAATATGAGTATGGTACTGAAGATGATTACCAAGGTAAACCT
TTGGAATTTGGTGCCACTTCTGCTGCTCTTCAACCTGAAGAAGAGCAAGAAGAAG
ATTGGTTAGATGATGATAGTCAACAAACTGTTGGTCAACAAGACGGCAGTGAGG
ACAATCAGACAACTACTATTCAAACAATTGTTGAGGTTCAACCTCAATTAGAGAT
GGAACTTACACCAGTTGTTCAGACTATTGAAGTGAATAGTTTTAGTGGTTATTTA
AAACTTACTGACAATGTATACATTAAAAATGCAGACATTGTGGAAGAAGCTAAA
AAGGTAAAACCAACAGTGGTTGTTAATGCAGCCAATGTTTACCTTAAACATGGA
GGAGGTGTTGCAGGAGCCTTAAATAAGGCTACTAACAATGCCATGCAAGTTGAA
TCTGATGATTACATAGCTACTAATGGACCACTTAAAGTGGGTGGTAGTTGTGTTT
TAAGCGGACACAATCTTGCTAAACACTGTCTTCATGTTGTCGGCCCAAATGTTAA
CAAAGGTGAAGACATTCAACTTCTTAAGAGTGCTTATGAAAATTTTAATCAGCAC
GAAGTTCTACTTGCACCATTATTATCAGCTGGTATTTTTGGTGCTGACCCTATACA
TTCTTTAAGAGTTTGTGTAGATACTGTTCGCACAAATGTCTACTTAGCTGTCTTTG
ATAAAAATCTCTATGACAAACTTGTTTCAAGCTTTTTGGAAATGAAGAGTGAAAA
GCAAGTTGAACAAAAGATCGCTGAGATTCCTAAAGAGGAAGTTAAGCCATTTAT
AACTGAAAGTAAACCTTCAGTTGAACAGAGAAAACAAGATGATAAGAAAATCA
AAGCTTGTGTTGAAGAAGTTACAACAACTCTGGAAGAAACTAAGTTCCTCACAG
AAAACTTGTTACTTTATATTGACATTAATGGCAATCTTCATCCAGATTCTGCCACT
CTTGTTAGTGACATTGACATCACTTTCTTAAAGAAAGATGCTCCATATATAGTGG
GTGATGTTGTTCAAGAGGGTGTTTTAACTGCTGTGGTTATACCTACTAAAAAGGC
TGGTGGCACTACTGAAATGCTAGCGAAAGCTTTGAGAAAAGTGCCAACAGACAA
TTATATAACCACTTACCCGGGTCAGGGTTTAAATGGTTACACTGTAGAGGAGGC
AAAGACAGTGCTTAAAAAGTGTAAAAGTGCCTTTTACATTCTACCATCTATTATC
TCTAATGAGAAGCAAGAAATTCTTGGAACTGTTTCTTGGAATTTGCGAGAAATGC
TTGCACATGCAGAAGAAACACGCAAATTAATGCCTGTCTGTGTGGAAACTAAAG
CCATAGTTTCAACTATACAGCGTAAATATAAGGGTATTAAAATACAAGAGGGTG
TGGTTGATTATGGTGCTAGATTTTACTTTTACACCAGTAAAACAACTGTAGCGTC
ACTTATCAACACACTTAACGATCTAAATGAAACTCTTGTTACAATGCCACTTGGC
TATGTAACACATGGCTTAAATTTGGAAGAAGCTGCTCGGTATATGAGATCTCTCA
AAGTGCCAGCTACAGTTTCTGTTTCTTCACCTGATGCTGTTACAGCGTATAATGGT
TATCTTACTTCTTCTTCTAAAACACCTGAAGAACATTTTATTGAAACCATCTCACT
TGCTGGTTCCTATAAAGATTGGTCCTATTCTGGACAATCTACACAACTAGGTATA
GAATTTCTTAAGAGAGGTGATAAAAGTGTATATTACACTAGTAATCCTACCACAT
TCCACCTAGATGGTGAAGTTATCACCTTTGACAATCTTAAGACACTTCTTTCTTTG
AGAGAAGTGAGGACTATTAAGGTGTTTACAACAGTAGACAACATTAACCTCCAC
ACGCAAGTTGTGGACATGTCAATGACATATGGACAACAGTTTGGTCCAACTTATT
TGGATGGAGCTGATGTTACTAAAATAAAACCTCATAATTCACATGAAGGTAAAA
CATTTTATGTTTTACCTAATGATGACACTCTACGTGTTGAGGCTTTTGAGTACTAC
CACACAACTGATCCTAGTTTTCTGGGTAGGTACATGTCAGCATTAAATCACACTA
AAAAGTGGAAATACCCACAAGTTAATGGTTTAACTTCTATTAAATGGGCAGATA
ACAACTGTTATCTTGCCACTGCATTGTTAACACTCCAACAAATAGAGTTGAAGTT
TAATCCACCTGCTCTACAAGATGCTTATTACAGAGCAAGGGCTGGTGAAGCTGCT
AACTTTTGTGCACTTATCTTAGCCTACTGTAATAAGACAGTAGGTGAGTTAGGTG
ATGTTAGAGAAACAATGAGTTACTTGTTTCAACATGCCAATTTAGATTCTTGCAA
AAGAGTCTTGAACGTGGTGTGTAAAACTTGTGGACAACAGCAGACAACCCTTAA
GGGTGTAGAAGCTGTTATGTACATGGGCACACTTTCTTATGAACAATTTAAGAAA
GGTGTTCAGATACCTTGTACGTGTGGTAAACAAGCTACAAAATATCTAGTACAAC
AGGAGTCACCTTTTGTTATGATGTCAGCACCACCTGCTCAGTATGAACTTAAGCA
TGGTACATTTACTTGTGCTAGTGAGTACACTGGTAATTACCAGTGTGGTCACTAT
AAACATATAACTTCTAAAGAAACTTTGTATTGCATAGACGGTGCTTTACTTACAA
AGTCCTCAGAATACAAAGGTCCTATTACGGATGTTTTCTACAAAGAAAACAGTTA
CACAACAACCATAAAACCAGTTACTTATAAATTGGATGGTGTTGTTTGTACAGAA
ATTGACCCTAAGTTGGACAATTATTATAAGAAAGACAATTCTTATTTCACAGAGC
AACCAATTGATCTTGTACCAAACCAACCATATCCAAACGCAAGCTTCGATAATTT
TAAGTTTGTATGTGATAATATCAAATTTGCTGATGATTTAAACCAGTTAACTGGTT
ATAAGAAACCTGCTTCAAGAGAGCTTAAAGTTACATTTTTCCCTGACTTAAATGG
TGATGTGGTGGCTATTGATTATAAACACTACACACCCTCTTTTAAGAAAGGAGCT
AAATTGTTACATAAACCTATTGTTTGGCATGTTAACAATGCAACTAATAAAGCCA
CGTATAAACCAAATACCTGGTGTATACGTTGTCTTTGGAGCACAAAACCAGTTGA
AACATCAAATTCGTTTGATGTACTGAAGTCAGAGGACGCGCAGGGAATGGATAA
TCTTGCCTGCGAAGATCTAAAACCAGTCTCTGAAGAAGTAGTGGAAAATCCTACC
ATACAGAAAGACGTTCTTGAGTGTAATGTGAAAACTACCGAAGTTGTAGGAGAC
ATTATACTTAAACCAGCAAATAATAGTTTAAAAATTACAGAAGAGGTTGGCCAC
ACAGATCTAATGGCTGCTTATGTAGACAATTCTAGTCTTACTATTAAGAAACCTA
ATGAATTATCTAGAGTATTAGGTTTGAAAACCCTTGCTACTCATGGTTTAGCTGCT
GTTAATAGTGTCCCTTGGGATACTATAGCTAATTATGCTAAGCCTTTTCTTAACAA
AGTTGTTAGTACAACTACTAACATAGTTACACGGTGTTTAAACCGTGTTTGTACT
AATTATATGCCTTATTTCTTTACTTTATTGCTACAATTGTGTACTTTTACTAGAAGT
ACAAATTCTAGAATTAAAGCATCTATGCCGACTACTATAGCAAAGAATACTGTTA
AGAGTGTCGGTAAATTTTGTCTAGAGGCTTCATTTAATTATTTGAAGTCACCTAAT
TTTTCTAAACTGATAAATATTATAATTTGGTTTTTACTATTAAGTGTTTGCCTAGG
TTCTTTAATCTACTCAACCGCGGCGTTAGGTGTTTTAATGTCTAATTTAGGTATGC
CGTCTTACTGTACGGGTTACCGTGAAGGTTATTTGAACTCTACGAATGTCACGAT
TGCGACGTACTGTACGGGTTCTATACCGTGTAGTGTTTGTCTTAGTGGTTTAGATT
CTTTAGACACGTATCCGTCTTTAGAAACGATACAAATTACGATTTCATCTTTTAAA
TGGGATTTAACGGCGTTTGGTTTAGTTGCGGAGTGGTTTTTGGCGTATATTCTTTT
CACGCGTTTTTTCTATGTACTTGGTTTGGCGGCGATCATGCAATTGTTTTTCAGCT
ATTTTGCGGTACATTTTATTAGTAATTCTTGGCTTATGTGGTTAATAATTAATCTT
GTACAAATGGCGCCGATTTCAGCGATGGTTAGAATGTACATCTTCTTTGCGTCAT
TTTATTATGTATGGAAAAGTTATGTGCATGTTGTAGACGGTTGTAATTCATCAAC
GTGTATGATGTGTTACAAACGTAATAGAGCGACGCGTGTCGAATGTACGACGATT
GTTAATGGTGTTAGACGTTCCTTTTATGTCTATGCGAATGGTGGTAAAGGTTTTTG
CAAACTACACAATTGGAATTGTGTTAATTGTGATACGTTCTGTGCGGGTAGTACG
TTTATTAGTGATGAAGTTGCGCGTGACTTGTCACTACAGTTTAAACGTCCGATAA
ATCCGACGGACCAGTCTTCTTACATCGTTGATAGTGTTACGGTGAAGAATGGTTC
CATCCATCTTTACTTTGATAAAGCGGGTCAAAAGACGTATGAACGTCATTCTCTC
TCTCATTTTGTTAACTTAGACAACCTGCGTGCGAATAACACGAAAGGTTCATTGC
CGATTAATGTTATAGTTTTTGATGGTAAATCAAAATGTGAAGAATCATCTGCGAA
ATCAGCGTCTGTTTACTACAGTCAGCTTATGTGTCAACCGATACTGTTACTAGAT
CAGGCGTTAGTGTCTGATGTTGGTGATAGTGCGGAAGTTGCGGTTAAAATGTTTG
ATGCGTACGTTAATACGTTTTCATCAACGTTTAACGTACCGATGGAAAAACTCAA
AACGCTAGTTGCGACGGCGGAAGCGGAACTTGCGAAGAATGTGTCCTTAGACAA
TGTCTTATCTACGTTTATTTCAGCGGCGCGTCAAGGTTTTGTTGATTCAGATGTAG
AAACGAAAGATGTTGTTGAATGTCTTAAATTGTCACATCAATCTGACATAGAAGT
TACGGGTGATAGTTGTAATAACTATATGCTCACGTATAACAAAGTTGAAAACATG
ACGCCGCGTGACCTTGGTGCGTGTATTGACTGTAGTGCGCGTCATATTAATGCGC
AGGTAGCGAAAAGTCACAACATTGCGTTGATATGGAACGTTAAAGATTTCATGTC
ATTGTCTGAACAACTACGTAAACAAATACGTAGTGCGGCGAAAAAGAATAACTT
ACCGTTTAAGTTGACGTGTGCGACGACGCGTCAAGTTGTTAATGTTGTAACGACG
AAGATAGCGCTTAAGGGTGGTAAAATTGTTAATAATTGGTTGAAGCAATTAATT
AAAGTTACACTTGTGTTCCTTTTTGTTGCTGCTATTTTCTATTTAATAACACCTGTT
CATGTCATGTCTAAACATACTGACTTTTCAAGTGAAATCATAGGATACAAGGCTA
TTGATGGTGGTGTCACTCGTGACATAGCATCTACAGATACTTGTTTTGCTAACAA
ACATGCTGATTTTGACACATGGTTTAGCCAGCGTGGTGGTAGTTATACTAATGAC
AAAGCTTGCCCATTGATTGCTGCAGTCATAACAAGAGAAGTGGGTTTTGTCGTGC
CTGGTTTGCCTGGCACGATATTACGCACAACTAATGGTGACTTTTTGCATTTCTTA
CCTAGAGTTTTTAGTGCAGTTGGTAACATCTGTTACACACCATCAAAACTTATAG
AGTACACTGACTTTGCAACATCAGCTTGTGTTTTGGCTGCTGAATGTACAATTTTT
AAAGATGCTTCTGGTAAGCCAGTACCATATTGTTATGATACCAATGTACTAGAAG
GTTCTGTTGCTTATGAAAGTTTACGCCCTGACACACGTTATGTGCTCATGGATGG
CTCTATTATTCAATTTCCTAACACCTACCTTGAAGGTTCTGTTAGAGTGGTAACAA
CTTTTGATTCTGAGTACTGTAGGCACGGCACTTGTGAAAGATCAGAAGCTGGTGT
TTGTGTATCTACTAGTGGTAGATGGGTACTTAACAATGATTATTACAGATCTTTAC
CAGGAGTTTTCTGTGGTGTAGATGCTGTAAATTTACTTACTAATATGTTTACACCA
CTAATTCAACCTATTGGTGCTTTGGACATATCAGCATCTATAGTAGCTGGTGGTA
TTGTAGCTATCGTAGTAACATGCCTTGCCTACTATTTTATGAGGTTTAGAAGAGCT
TTTGGTGAATACAGTCATGTAGTTGCCTTTAATACTTTACTATTCCTTATGTCATT
CACTGTACTCTGTTTAACACCAGTTTACTCATTCTTACCTGGTGTTTATTCTGTTAT
TTACTTGTACTTGACATTTTATCTTACTAATGATGTTTCTTTTTTAGCACATATTCA
GTGGATGGTTATGTTCACACCTTTAGTACCTTTCTGGATAACAATTGCTTATATCA
TTTGTATTTCCACAAAGCATTTCTATTGGTTCTTTAGTAATTACCTAAAGAGACGT
GTAGTCTTTAATGGTGTTTCCTTTAGTACTTTTGAAGAAGCTGCGCTGTGCACCTT
TTTGTTAAATAAAGAAATGTATCTAAAGTTGCGTAGTGATGTGCTATTACCTCTT
ACGCAATATAATAGATACTTAGCTCTTTATAATAAGTACAAGTATTTTAGTGGAG
CAATGGATACAACTAGCTACAGAGAAGCTGCTTGTTGTCATCTCGCAAAGGCTCT
CAATGACTTCAGTAACTCAGGTTCTGATGTTCTTTACCAACCACCACAAACCTCT
ATCACCTCAGCTGTTTTGCAGAGTGGTTTTAGAAAAATGGCATTCCCATCTGGTA
AAGTTGAGGGTTGTATGGTACAAGTAACTTGTGGTACAACTACACTTAACGGTCT
TTGGCTTGATGACGTAGTTTACTGTCCAAGACATGTGATCTGCACCTCTGAAGAC
ATGCTTAACCCTAATTATGAAGATTTACTCATTCGTAAGTCTAATCATAATTTCTT
GGTACAGGCTGGTAATGTTCAACTCAGGGTTATTGGACATTCTATGCAAAATTGT
GTACTTAAGCTTAAGGTTGATACAGCCAATCCTAAGACACCTAAGTATAAGTTTG
TTCGCATTCAACCAGGACAGACTTTTTCAGTGTTAGCTTGTTACAATGGTTCACC
ATCTGGTGTTTACCAATGTGCTATGAGGCCCAATTTCACTATTAAGGGTTCATTCC
TTAATGGTTCATGTGGTAGTGTTGGTTTTAACATAGATTATGACTGTGTCTCTTTT
TGTTACATGCACCATATGGAATTACCAACTGGAGTTCATGCTGGCACAGACTTAG
AAGGTAACTTTTATGGACCTTTTGTTGACAGGCAAACAGCACAAGCAGCTGGTAC
GGACACAACTATTACAGTTAATGTTTTAGCTTGGTTGTACGCTGCTGTTATAAAT
GGAGACAGGTGGTTTCTCAATCGATTTACCACAACTCTTAATGACTTTAACCTTG
TGGCTATGAAGTACAATTATGAACCTCTAACACAAGACCATGTTGACATACTAGG
ACCTCTTTCTGCTCAAACTGGAATTGCCGTTTTAGATATGTGTGCTTCATTAAAAG
AATTACTGCAAAATGGTATGAATGGACGTACCATATTGGGTAGTGCTTTATTAGA
AGATGAATTTACACCTTTTGATGTTGTTAGACAATGCTCAGGTGTTACTTTCCAAA
GTGCAGTGAAAAGAACAATCAAGGGTACACACCACTGGTTGTTACTCACAATTTT
GACTTCACTTTTAGTTTTAGTCCAGAGTACTCAATGGTCTTTGTTCTTTTTTTTGTA
TGAAAATGCCTTTTTACCTTTTGCTATGGGTATTATTGCTATGTCTGCTTTTGCAA
TGATGTTTGTCAAACATAAGCATGCATTTCTCTGTTTGTTTTTGTTACCTTCTCTTG
CCACTGTAGCTTATTTTAATATGGTCTATATGCCTGCTAGTTGGGTGATGCGTATT
ATGACATGGTTGGATATGGTTGATACTAGTTTGTCTGGTTTTAAGCTAAAAGACT
GTGTTATGTATGCATCAGCTGTAGTGTTACTAATCCTTATGACAGCAAGAACTGT
GTATGATGATGGTGCTAGGAGAGTGTGGACACTTATGAATGTCTTGACACTCGTT
TATAAAGTTTATTATGGTAATGCTTTAGATCAAGCCATTTCCATGTGGGCTCTTAT
AATCTCTGTTACTTCTAACTACTCAGGTGTAGTTACAACTGTCATGTTTTTGGCCA
GAGGTATTGTTTTTATGTGTGTTGAGTATTGCCCTATTTTCTTCATAACTGGTAAT
ACACTTCAGTGTATAATGCTAGTTTATTGTTTCTTAGGCTATTTTTGTACTTGTTAC
TTTGGCCTCTTTTGTTTACTCAACCGCTACTTTAGACTGACTCTTGGTGTTTATGAT
TACTTAGTTTCTACACAGGAGTTTAGATATATGAATTCACAGGGACTACTCCCAC
CCAAGAATAGCATAGATGCCTTCAAACTCAACATTAAATTGTTGGGTGTTGGTGG
CAAACCTTGTATCAAAGTAGCCACTGTACAGTCTAAAATGTCAGATGTAAAGTGC
ACATCAGTAGTCTTACTCTCAGTTTTGCAACAACTCAGAGTAGAATCATCATCTA
AATTGTGGGCTCAATGTGTCCAGTTACACAATGACATTCTCTTAGCTAAAGATAC
TACTGAAGCCTTTGAAAAAATGGTTTCACTACTTTCTGTTTTGCTTTCCATGCAGG
GTGCTGTAGACATAAACAAGCTTTGTGAAGAAATGCTGGACAACAGGGCAACCT
TACAAGCTATAGCCTCAGAGTTTAGTTCCCTTCCATCATATGCAGCTTTTGCTACT
GCTCAAGAAGCTTATGAGCAGGCTGTTGCTAATGGTGATTCTGAAGTTGTTCTTA
AAAAGTTGAAGAAGTCTTTGAATGTGGCTAAATCTGAATTTGACCGTGATGCAGC
CATGCAACGTAAGTTGGAAAAGATGGCTGATCAAGCTATGACCCAAATGTATAA
ACAGGCTAGATCTGAGGACAAGAGGGCAAAAGTTACTAGTGCTATGCAGACAAT
GCTTTTCACTATGCTTAGAAAGTTGGATAATGATGCACTCAACAACATTATCAAC
AATGCAAGAGATGGTTGTGTTCCCTTGAACATAATACCTCTTACAACAGCAGCCA
AACTAATGGTTGTCATACCAGACTATAACACATATAAAAATACGTGTGATGGTAC
AACATTTACTTATGCATCAGCATTGTGGGAAATCCAACAGGTTGTAGATGCAGAT
AGTAAAATTGTTCAACTTAGTGAAATTAGTATGGACAATTCACCTAATTTAGCAT
GGCCTCTTATTGTAACAGCTTTAAGGGCCAATTCTGCTGTCAAATTACAGAATAA
TGAGCTTAGTCCTGTTGCACTACGACAGATGTCTTGTGCTGCCGGTACTACACAA
ACTGCTTGCACTGATGACAATGCGTTAGCTTACTACAACACAACAAAGGGAGGT
AGGTTTGTACTTGCACTGTTATCCGATTTACAGGATTTGAAATGGGCTAGATTCC
CTAAGAGTGATGGAACTGGTACTATCTATACAGAACTGGAACCACCTTGTAGGTT
TGTTACAGACACACCTAAAGGTCCTAAAGTGAAGTATTTATACTTTATTAAAGGA
TTAAACAACCTAAATAGAGGTATGGTACTTGGTAGTTTAGCTGCCACAGTACGTC
TACAAGCTGGTAATGCAACAGAAGTGCCTGCCAATTCAACTGTATTATCTTTCTG
TGCTTTTGCTGTAGATGCTGCTAAAGCTTACAAAGATTATCTAGCTAGTGGGGGA
CAACCAATCACTAATTGTGTTAAGATGTTGTGTACACACACTGGTACTGGTCAGG
CAATAACAGTTACACCGGAAGCCAATATGGATCAAGAATCCTTTGGTGGTGCAT
CGTGTTGTCTGTACTGCCGTTGCCACATAGATCATCCAAATCCTAAAGGATTTTG
TGACTTAAAAGGTAAGTATGTACAAATACCTACAACTTGTGCTAATGACCCTGTG
GGTTTTACACTTAAAAACACAGTCTGTACCGTCTGCGGTATGTGGAAAGGTTATG
GCTGTAGTTGTGATCAACTCCGCGAACCCATGCTTCAGTCAGCTGATGCACAATC
GTTTTTAAACGGGTTTGCGGTGTAAGTGCAGCCCGTCTTACACCGTGCGGCACAG
GCACTAGTACTGATGTCGTATACAGGGCTTTTGACATCTACAATGATAAAGTAGC
TGGTTTTGCTAAATTCCTAAAAACTAATTGTTGTCGCTTCCAAGAAAAGGACGAA
GATGACAATTTAATTGATTCTTACTTTGTAGTTAAGAGACACACTTTCTCTAACTA
CCAACATGAAGAAACAATTTATAATTTACTTAAGGATTGTCCAGCTGTTGCTAAA
CATGACTTCTTTAAGTTTAGAATAGACGGTGACATGGTACCACATATATCACGTC
AACGTCTTACTAAATACACAATGGCAGACCTCGTCTATGCTTTAAGGCATTTTGA
TGAAGGTAATTGTGACACATTAAAAGAAATACTTGTCACATACAATTGTTGTGAT
GATGATTATTTCAATAAAAAGGACTGGTATGATTTTGTAGAAAACCCAGATATAT
TACGCGTATACGCCAACTTAGGTGAACGTGTACGCCAAGCTTTGTTAAAAACAG
TACAATTCTGTGATGCCATGCGAAATGCTGGTATTGTTGGTGTACTGACATTAGA
TAATCAAGATCTCAATGGTAACTGGTATGATTTCGGTGATTTCATACAAACCACG
CCAGGTAGTGGAGTTCCTGTTGTAGATTCTTATTATTCATTGTTAATGCCTATATT
AACCTTGACCAGGGCTTTAACTGCAGAGTCACATGTTGACACTGACTTAACAAAG
CCTTACATTAAGTGGGATTTGTTAAAATATGACTTCACGGAAGAGAGGTTAAAAC
TCTTTGACCGTTATTTTAAATATTGGGATCAGACATACCACCCAAATTGTGTTAAC
TGTTTGGATGACAGATGCATTCTGCATTGTGCAAACTTTAATGTTTTATTCTCTAC
AGTGTTCCCACCTACAAGTTTTGGACCACTAGTGAGAAAAATATTTGTTGATGGT
GTTCCATTTGTAGTTTCAACTGGATACCACTTCAGAGAGCTAGGTGTTGTACATA
ATCAGGATGTAAACTTACATAGCTCTAGACTTAGTTTTAAGGAATTACTTGTGTA
TGCTGCTGACCCTGCTATGCACGCTGCTTCTGGTAATCTATTACTAGATAAACGC
ACTACGTGCTTTTCAGTAGCTGCACTTACTAACAATGTTGCTTTTCAAACTGTCAA
ACCCGGTAATTTTAACAAAGACTTCTATGACTTTGCTGTGTCTAAGGGTTTCTTTA
AGGAAGGAAGTTCTGTTGAATTAAAACACTTCTTCTTTGCTCAGGATGGTAATGC
TGCTATCAGCGATTATGACTACTATCGTTATAATCTACCAACAATGTGTGATATC
AGACAACTACTATTTGTAGTTGAAGTTGTTGATAAGTACTTTGATTGTTACGATG
GTGGCTGTATTAATGCTAACCAAGTCATCGTCAACAACCTAGACAAATCAGCTGG
TTTTCCATTTAATAAATGGGGTAAGGCTAGACTTTATTATGATTCAATGAGTTATG
AGGATCAAGATGCACTTTTCGCATATACAAAACGTAATGTCATCCCTACTATAAC
TCAAATGAATCTTAAGTATGCCATTAGTGCAAAGAATAGAGCTCGCACCGTAGCT
GGTGTCTCTATCTGTAGTACTATGACCAATAGACAGTTTCATCAAAAATTATTGA
AATCAATAGCCGCCACTAGAGGAGCTACTGTAGTAATTGGAACAAGCAAATTCT
ATGGTGGTTGGCACAACATGTTAAAAACTGTTTATAGTGATGTAGAAAACCCTCA
CCTTATGGGTTGGGATTATCCTAAATGTGATAGAGCCATGCCTAACATGCTTAGA
ATTATGGCCTCACTTGTTCTTGCTCGCAAACATACAACGTGTTGTAGCTTGTCACA
CCGTTTCTATAGATTAGCTAATGAGTGTGCTCAAGTATTGAGTGAAATGGTCATG
TGTGGCGGTTCACTATATGTTAAACCAGGTGGAACCTCATCAGGAGATGCCACA
ACTGCTTATGCTAATAGTGTTTTTAACATTTGTCAAGCTGTCACGGCCAATGTTAA
TGCACTTTTATCTACTGATGGTAACAAAATTGCCGATAAGTATGTCCGCAATTTA
CAACACAGACTTTATGAGTGTCTCTATAGAAATAGAGATGTTGACACAGACTTTG
TGAATGAGTTTTACGCATATTTGCGTAAACATTTCTCAATGATGATACTCTCTGAC
GATGCTGTTGTGTGTTTCAATAGCACTTATGCATCTCAAGGTCTAGTGGCTAGCA
TAAAGAACTTTAAGTCAGTTCTTTATTATCAAAACAATGTTTTTATGTCTGAAGCA
AAATGTTGGACTGAGACTGACCTTACTAAAGGACCTCATGAATTTTGCTCTCAAC
ATACAATGCTAGTTAAACAGGGTGATGATTATGTGTACCTTCCTTACCCAGATCC
ATCAAGAATCCTAGGGGCCGGCTGTTTTGTAGATGATATCGTAAAAACAGATGGT
ACACTTATGATTGAACGGTTCGTGTCTTTAGCTATAGATGCTTACCCACTTACTAA
ACATCCTAATCAGGAGTATGCTGATGTCTTTCATTTGTACTTACAATACATAAGA
AAGCTACATGATGAGTTAACAGGACACATGTTAGACATGTATTCTGTTATGCTTA
CTAATGATAACACTTCAAGGTATTGGGAACCTGAGTTTTATGAGGCTATGTACAC
ACCGCATACAGTCTTACAGGCTGTTGGGGCTTGTGTTCTTTGCAATTCACAGACT
TCATTAAGATGTGGTGCTTGCATACGTAGACCATTCTTATGTTGTAAATGCTGTTA
CGACCATGTCATATCAACATCACATAAATTAGTCTTGTCTGTTAATCCGTATGTTT
GCAATGCTCCAGGTTGTGATGTCACAGATGTGACTCAACTTTACTTAGGAGGTAT
GAGCTATTATTGTAAATCACATAAACCACCCATTAGTTTTCCATTGTGTGCTAATG
GACAAGTTTTTGGTTTATATAAAAATACATGTGTTGGTAGCGATAATGTTACTGA
CTTTAATGCAATTGCAACATGTGACTGGACAAATGCTGGTGATTACATTTTAGCT
AACACCTGTACTGAAAGACTCAAGCTTTTTGCAGCAGAAACGCTCAAAGCTACT
GAGGAGACATTTAAACTGTCTTATGGTATTGCTACTGTACGTGAAGTGCTGTCTG
ACAGAGAATTACATCTTTCATGGGAAGTTGGTAAACCTAGACCACCACTTAACCG
AAATTATGTCTTTACTGGTTATCGTGTAACTAAAAACAGTAAAGTACAAATAGGA
GAGTACACCTTTGAAAAAGGTGACTATGGTGATGCTGTTGTTTACCGAGGTACAA
CAACTTACAAATTAAATGTTGGTGATTATTTTGTGCTGACATCACATACAGTAAT
GCCATTAAGTGCACCTACACTAGTGCCACAAGAGCACTATGTTAGAATTACTGGC
TTATACCCAACACTCAATATCTCAGATGAGTTTTCTAGCAATGTTGCAAATTATC
AAAAGGTTGGTATGCAAAAGTATTCTACACTCCAGGGACCACCTGGTACTGGTA
AGAGTCATTTTGCTATTGGCCTAGCTCTCTACTACCCTTCTGCTCGCATAGTGTAT
ACAGCTTGCTCTCATGCCGCTGTTGATGCACTATGTGAGAAGGCATTAAAATATT
TGCCTATAGATAAATGTAGTAGAATTATACCTGCACGTGCTCGTGTAGAGTGTTT
TGATAAATTCAAAGTGAATTCAACATTAGAACAGTATGTCTTTTGTACTGTAAAT
GCATTGCCTGAGACGACAGCAGATATAGTTGTCTTTGATGAAATTTCAATGGCCA
CAAATTATGATTTGAGTGTTGTCAATGCCAGATTACGTGCTAAGCACTATGTGTA
CATTGGCGACCCTGCTCAATTACCTGCACCACGCACATTGCTAACTAAGGGCACA
CTAGAACCAGAATATTTCAATTCAGTGTGTAGACTTATGAAAACTATAGGTCCAG
ACATGTTCCTCGGAACTTGTCGGCGTTGTCCTGCTGAAATTGTTGACACTGTGAG
TGCTTTGGTTTATGATAATAAGCTTAAAGCACATAAAGACAAATCAGCTCAATGC
TTTAAAATGTTTTATAAGGGTGTTATCACGCATGATGTTTCATCTGCAATTAACAG
GCCACAAATAGGCGTGGTAAGAGAATTCCTTACACGTAACCCTGCTTGGAGAAA
AGCTGTCTTTATTTCACCTTATAATTCACAGAATGCTGTAGCCTCAAAGATTTTGG
GACTACCAACTCAAACTGTTGATTCATCACAGGGCTCAGAATATGACTATGTCAT
ATTCACTCAAACCACTGAAACAGCTCACTCTTGTAATGTAAACAGATTTAATGTT
GCTATTACCAGAGCAAAAGTAGGCATACTTTGCATAATGTCTGATAGAGACCTTT
ATGACAAGTTGCAATTTACAAGTCTTGAAATTCCACGTAGGAATGTGGCAACTTT
ACAAGCTGAAAATGTAACAGGACTCTTTAAAGATTGTAGTAAGGTAATCACTGG
GTTACATCCTACACAGGCACCTACACACCTCAGTGTTGACACTAAATTCAAAACT
GAAGGTTTATGTGTTGACATACCTGGCATACCTAAGGACATGACCTATAGAAGA
CTCATCTCTATGATGGGTTTTAAAATGAATTATCAAGTTAATGGTTACCCTAACAT
GTTTATCACCCGCGAAGAAGCTATAAGACATGTACGTGCATGGATTGGCTTCGAT
GTCGAGGGGTGTCATGCTACTAGAGAAGCTGTTGGTACCAATTTACCTTTACAGC
TAGGTTTTTCTACAGGTGTTAACCTAGTTGCTGTACCTACAGGTTATGTTGATACA
CCTAATAATACAGATTTTTCCAGAGTTAGTGCTAAACCACCGCCTGGAGATCAAT
TTAAACACCTCATACCACTTATGTACAAAGGACTTCCTTGGAATGTAGTGCGTAT
AAAGATTGTACAAATGTTAAGTGACACACTTAAAAATCTCTCTGACAGAGTCGTA
TTTGTCTTATGGGCACATGGCTTTGAGTTGACATCTATGAAGTATTTTGTGAAAAT
AGGACCTGAGCGCACCTGTTGTCTATGTGATAGACGTGCCACATGCTTTTCCACT
GCTTCAGACACTTATGCCTGTTGGCATCATTCTATTGGATTTGATTACGTCTATAA
TCCGTTTATGATTGATGTTCAACAATGGGGTTTTACAGGTAACCTACAAAGCAAC
CATGATCTGTATTGTCAAGTCCATGGTAATGCACATGTAGCTAGTTGTGATGCAA
TCATGACTAGGTGTCTAGCTGTCCACGAGTGCTTTGTTAAGCGTGTTGACTGGAC
TATTGAATATCCTATAATTGGTGATGAACTGAAGATTAATGCGGCTTGTAGAAAG
GTTCAACACATGGTTGTTAAAGCTGCATTATTAGCAGACAAATTCCCAGTTCTTC
ACGACATTGGTAACCCTAAAGCTATTAAGTGTGTACCTCAAGCTGATGTAGAATG
GAAGTTCTATGATGCACAGCCTTGTAGTGACAAAGCTTATAAAATAGAAGAATT
ATTCTATTCTTATGCCACACATTCTGACAAATTCACAGATGGTGTATGCCTATTTT
GGAATTGCAATGTCGATAGATATCCTGCTAATTCCATTGTTTGTAGATTTGACACT
AGAGTGCTATCTAACCTTAACTTGCCTGGTTGTGATGGTGGCAGTTTGTATGTAA
ATAAACATGCATTCCACACACCAGCTTTTGATAAAAGTGCTTTTGTTAATTTAAA
ACAATTACCATTTTTCTATTACTCTGACAGTCCATGTGAGTCTCATGGAAAACAA
GTAGTGTCAGATATAGATTATGTACCACTAAAGTCTGCTACGTGTATAACACGTT
GCAATTTAGGTGGTGCTGTCTGTAGACATCATGCTAATGAGTACAGATTGTATCT
CGATGCTTATAACATGATGATCTCAGCTGGCTTTAGCTTGTGGGTTTACAAACAA
TTTGATACTTATAACCTCTGGAACACTTTTACAAGACTTCAGAGTTTAGAAAATG
TGGCTTTTAATGTTGTAAATAAGGGACACTTTGATGGACAACAGGGTGAAGTACC
AGTTTCTATCATTAATAACACTGTTTACACAAAAGTTGATGGTGTTGATGTAGAA
TTGTTTGAAAATAAAACAACATTACCTGTTAATGTAGCATTTGAGCTTTGGGCTA
AGCGCAACATTAAACCAGTACCAGAGGTGAAAATACTCAATAATTTGGGTGTGG
ACATTGCTGCTAATACTGTGATCTGGGACTACAAAAGAGATGCTCCAGCACATAT
ATCTACTATTGGTGTTTGTTCTATGACTGACATAGCCAAGAAACCAACTGAAACG
ATTTGTGCACCACTCACTGTCTTTTTTGATGGTAGAGTTGATGGTCAAGTAGACTT
ATTTAGAAATGCCCGTAATGGTGTTCTTATTACAGAAGGTAGTGTTAAAGGTTTA
CAACCATCTGTAGGTCCCAAACAAGCTAGTCTTAATGGAGTCACATTAATTGGAG
AAGCCGTAAAAACACAGTTCAATTATTATAAGAAAGTTGATGGTGTTGTCCAACA
ATTACCTGAAACTTACTTTACTCAGAGTAGAAATTTACAAGAATTTAAACCCAGG
AGTCAAATGGAAATTGATTTCTTAGAATTAGCTATGGATGAATTCATTGAACGGT
ATAAATTAGAAGGCTATGCCTTCGAACATATCGTTTATGGAGATTTTAGTCATAG
TCAGTTAGGTGGTTTACATCTACTGATTGGACTAGCTAAACGTTTTAAGGAATCA
CCTTTTGAATTAGAAGATTTTATTCCTATGGACAGTACAGTTAAAAACTATTTCAT
AACAGATGCGCAAACAGGTTCATCTAAGTGTGTGTGTTCTGTTATTGATTTATTA
CTTGATGATTTTGTTGAAATAATAAAATCCCAAGATTTATCTGTAGTTTCTAAGGT
TGTCAAAGTGACTATTGACTATACAGAAATTTCATTTATGCTTTGGTGTAAAGAT
GGCCATGTAGAAACATTTTACCCAAAATTACAATCTAGTCAAGCGTGGCAACCG
GGTGTTGCTATGCCTAATCTTTACAAAATGCAAAGAATGCTATTAGAAAAGTGTG
ACCTTCAAAATTATGGTGATAGTGCAACATTACCTAAAGGCATAATGATGAATGT
CGCAAAATATACTCAACTGTGTCAATATTTAAACACATTAACATTAGCTGTACCC
TATAATATGAGAGTTATACATTTTGGTGCTGGTTCTGATAAAGGAGTTGCACCAG
GTACAGCTGTTTTAAGACAGTGGTTGCCTACGGGTACGCTGCTTGTCGATTCAGA
TCTTAATGACTTTGTCTCTGATGCAGATTCAACTTTGATTGGTGATTGTGCAACTG
TACATACAGCTAATAAATGGGATCTCATTATTAGTGATATGTACGACCCTAAGAC
TAAAAATGTTACAAAAGAAAATGACTCTAAAGAGGGTTTTTTCACTTACATTTGT
GGGTTTATACAACAAAAGCTAGCTCTTGGAGGTTCCGTGGCTATAAAGATAACA
GAACATTCTTGGAATGCTGATCTTTATAAGCTCATGGGACACTTCGCATGGTGGA
CAGCCTTTGTTACTAATGTGAATGCGTCATCATCTGAAGCATTTTTAATTGGATGT
AATTATCTTGGCAAACCACGCGAACAAATAGATGGTTATGTCATGCATGCAAATT
ACATATTTTGGAGGAATACAAATCCAATTCAGTTGTCTTCCTATTCTTTATTTGAC
ATGAGTAAATTTCCCCTTAAATTAAGGGGTACTGCTGTTATGTCTTTAAAAGAAG
GTCAAATCAATGATATGATTTTATCTCTTCTTAGTAAAGGTAGACTTATAATTAG
AGAAAACAACAGAGTTGTTATTTCTAGTGATGTTCTTGTTAACAACTAAACGAAC
AATGTTTGTTTTTCTTGTTTTATTGCCACTAGTCTCTAGTCAGTGTGTTAATCTTA
CAACCAGAACTCAATTACCCCCTGCATACACTAATTCTTTCACACGTGGTGTTTA
TTACCCTGACAAAGTTTTCAGATCCTCAGTTTTACATTCAACTCAGGACTTGTTCT
TACCTTTCTTTTCCAATGTTACTTGGTTCCATGCTATACATGTCTCTGGGACCAAT
GGTACTAAGAGGTTTGATAACCCTGTCCTACCATTTAATGATGGTGTTTATTTTGC
TTCCAtTGAGAAGTCTAACATAATAAGAGGCTGGATTTTTGGTACTACTTTAGACT
CGAAGACCCAGTCCCTACTTATTGTTAATAACGCTACTAATGTTGTTATTAAAGT
CTGTGAATTTCAATTTTGTAATGATCCATTTTTGGaTGTTTATTACCACAAAAACA
ACAAAAGTTGGATGaAAAGTGAGTTCAGAGTTTATTCTAGTGCGAATAATTGCAC
TTTTGAATATGTCTCTCAGCCTTTTCTTATGGACCTTGAAGGAAAACAGGGTAATT
TCAAAAATCTTAGGGAATTTGTGTTTAAGAATATTGATGGTTATTTTAAAATATA
TTCTAAGCACACGCCTATTAATTTAGTGCGTGATCTCCCtCAGGGTTTTTCGGCTT
TAGAACCATTGGTAGATTTGCCAATAGGTATTAACATCACTAGGTTTCAAACTTT
ACTTGCTTTACATAGAAGTTATTTGACTCCTGGTGATTCTTCTTCAGGTTGGACAG
CTGGTGCTGCAGCTTATTATGTGGGTTATCTTCAACCTAGGACTTTTCTATTAAAA
TATAATGAAAATGGAACCATTACAGATGCTGTAGACTGTGCACTTGACCCTCTCT
CAGAAACAAAGTGTACGTTGAAATCCTTCACTGTAGAAAAAGGAATCTATCAAA
CTTCTAACTTTAGAGTCCAACCAACAGAATCTATTGTTAGATTTCCTAATATTACA
AACTTGTGCCCTTTTGGTGAAGTTTTTAACGCCACCAGATTTGCATCTGTTTATGC
TTGGAACAGGAAGAGAATCAGCAACTGTGTTGCTGATTATTCTGTCCTATATAAT
TCCGCATCATTTTCCACTTTTAAGTGTTATGGAGTGTCTCCTACTAAATTAAATGA
TCTCTGCTTTACTAATGTCTATGCAGATTCATTTGTAATTAGAGGTGATGAAGTCA
GACAAATCGCTCCAGGGCAAACTGGAAAGATTGCTGATTATAATTATAAATTAC
CAGATGATTTTACAGGCTGCGTTATAGCTTGGAATTCTAACAATCTTGATTCTAA
GGTTGGTGGTAATTATAATTACCgGTATAGATTGTTTAGGAAGTCTAATCTCAAA
CCTTTTGAGAGAGATATTTCAACTGAAATCTATCAGGCCGGTAGCACACCTTGTA
ATGGTGTTcAAGGTTTTAATTGTTACTTTCCTTTACAATCATATGGTTTCCAACCC
ACTAATGGTGTTGGTTACCAACCATACAGAGTAGTAGTACTTTCTTTTGAACTTCT
ACATGCACCAGCAACTGTTTGTGGACCTAAAAAGTCTACTAATTTGGTTAAAAAC
AAATGTGTCAATTTCAACTTCAATGGTTTAACAGGCACAGGTGTTCTTACTGAGT
CTAACAAAAAGTTTCTGCCTTTCCAACAATTTGGCAGAGACATTGCTGACACTAC
TGATGCTGTCCGTGATCCACAGACACTTGAGATTCTTGACATTACACCATGTTCTT
TTGGTGGTGTCAGTGTTATAACACCAGGAACAAATACTTCTAACCAGGTTGCTGT
TCTTTATCAGGgTGTTAACTGCACAGAAGTCCCTGTTGCTATTCATGCAGATCAA
CTTACTCCTACTTGGCGTGTTTATTCTACAGGTTCTAATGTTTTTCAAACACGTGC
AGGCTGTTTAATAGGGGCTGAACATGTCAACAACTCATATGAGTGTGACATACCC
ATTGGTGCAGGTATATGCGCTAGTTATCAGACTCAGACTAATTCTCgTCGGCGGG
CACGTAGTGTAGCTAGTCAATCCATCATTGCCTACACTATGTCACTTGGTGCAGA
AAATTCAGTTGCTTACTCTAATAACTCTATTGCCATACCCACAAATTTTACTATTA
GTGTTACCACAGAAATTCTACCAGTGTCTATGACCAAGACATCAGTAGATTGTAC
AATGTACATTTGTGGTGATTCAACTGAATGCAGCAATCTTTTGTTGCAATATGGC
AGTTTTTGTACACAATTAAACCGTGCTTTAACTGGAATAGCTGTTGAACAAGACA
AAAACACCCAAGAAGTTTTTGCACAAGTCAAACAAATTTACAAAACACCACCAA
TTAAAGATTTTGGTGGTTTTAATTTTTCACAAATATTACCAGATCCATCAAAACCA
AGCAAGAGGTCATTTATTGAAGATCTACTTTTCAACAAAGTGACACTTGCAGATG
CTGGCTTCATCAAACAATATGGTGATTGCCTTGGTGATATTGCTGCTAGAGACCT
CATTTGTGCACAAAAGTTTAACGGCCTTACTGTTTTGCCACCTTTGCTCACAGATG
AAATGATTGCTCAATACACTTCTGCACTGTTAGCGGGTACAATCACTTCTGGTTG
GACCTTTGGTGCAGGTGCTGCATTACAAATACCATTTGCTATGCAAATGGCTTAT
AGGTTTAATGGTATTGGAGTTACACAGAATGTTCTCTATGAGAACCAAAAATTGA
TTGCCAACCAATTTAATAGTGCTATTGGCAAAATTCAAGACTCACTTTCTTCCAC
AGCAAGTGCACTTGGAAAACTTCAAGATGTGGTCAACCAAAATGCACAAGCTTT
AAACACGCTTGTTAAACAACTTAGCTCCAATTTTGGTGCAATTTCAAGTGTTTTA
AATGATATCCTTTCACGTCTTGACAAAGTTGAGGCTGAAGTGCAAATTGATAGGT
TGATCACAGGCAGACTTCAAAGTTTGCAGACATATGTGACTCAACAATTAATTAG
AGCTGCAGAAATCAGAGCTTCTGCTAATCTTGCTGCTACTAAAATGTCAGAGTGT
GTACTTGGACAATCAAAAAGAGTTGATTTTTGTGGAAAGGGCTATCATCTTATGT
CCTTCCCTCAGTCAGCACCTCATGGTGTAGTCTTCTTGCATGTGACTTATGTCCC
TGCACAAGAAAAGAACTTCACAACTGCTCCTGCCATTTGTCATGATGGAAAAGC
ACACTTTCCTCGTGAAGGTGTCTTTGTTTCAAATGGCACACACTGGTTTGTAACA
CAAAGGAATTTTTATGAACCACAAATCATTACTACAGACAACACATTTGTGTCTG
GTAACTGTGATGTTGTAATAGGAATTGTCAACAACACAGTTTATGATCCTTTGCA
ACCTGAATTAGACTCATTCAAGGAGGAGTTAGATAAATATTTTAAGAATCATACA
TCACCAGATGTTGATTTAGGTGACATCTCTGGCATTAATGCTTCAGTTGTAAACA
TTCAAAAAGAAATTGACCGCCTCAATGAGGTTGCCAAGAATTTAAATGAATCTCT
CATCGATCTCCAAGAACTTGGAAAGTATGAGCAGTATATAAAATGGCCATGGTA
CATTTGGCTAGGTTTTATAGCTGGCTTGATTGCCATAGTAATGGTGACAATTATG
CTTTGCTGTATGACCAGTTGCTGTAGTTGTCTCAAGGGCTGTTGTTCTTGTGGATC
CTGCTGCAAATTTGATGAAGACGACTCTGAGCCAGTGCTCAAAGGAGTCAAATT
ACATTACACATAAACGAACTTATGGATTTGTTTATGAGAATCTTCACAATTGGAA
CTGTAACTTTGAAGCAAGGTGAAATCAAGGATGCTACTCCTTCAGATTTTGTTCG
CGCTACTGCAACGATACCGATACAAGCCTCACTCCCTTTCGGATGGCTTATTGTT
GGCGTTGCACTTCTTGCTGTTTTTCAGAGCGCTTCCAAAATCATAACCCTCAAAA
AGAGATGGCAACTAGCACTCTCCAAGGGTGTTCACTTTGTTTGCAACTTGCTGTT
GTTGTTTGTAACAGTTTACTCACACCTTTTGCTCGTTGCTGCTGGCCTTGAAGCCC
CTTTTCTCTATCTTTATGCTTTAGTCTACTTCTTGCAGAGTATAAACTTTGTAAGA
ATAATAATGAGGCTTTGGCTTTGCTGGAAATGCCGTTCCAAAAACCCATTACTTT
ATGATGCCAACTATTTTCTTTGCTGGCATACTAATTGTTACGACTATTGTATACCT
TACAATAGTGTAACTTCTTCAATTGTCATTACTTCAGGTGATGGCACAACAAGTC
CTATTTCTGAACATGACTACCAGATTGGTGGTTATACTGAAAAATGGGAATCTGG
AGTAAAAGACTGTGTTGTATTACACAGTTACTTCACTTCAGACTATTACCAGCTG
TACTCAACTCAATTGAGTACAGACACTGGTGTTGAACATGTTACCTTCTTCATCT
ACAATAAAATTGTTGATGAGCCTGAAGAACATGTCCAAATTCACACAATCGACG
GTTCATCCGGAGTTGTTAATCCAGTAATGGAACCAATTTATGATGAACCGACGAC
GACTACTAGCGTGCCTTTGTAAGCACAAGCTGATGAGTACGAACTTATGTACTCA
TTCGTTTCGGAAGAGACAGGTACGTTAATAGTTAATAGCGTACTTCTTTTTCTTGC
TTTCGTGGTATTCTTGCTAGTTACACTAGCCATCCTTACTGCGCTTCGATTGTGTG
CGTACTGCTGCAATATTGTTAACGTGAGTCTTGTAAAACCTTCTTTTTACGTTTAC
TCTCGTGTTAAAAATCTGAATTCTTCTAGAGTTCCTGATCTTCTGGTCTAAACGAA
CTAAATATTATATTAGTTTTTCTGTTTGGAACTTTAATTTTAGCCATGGCAGATTC
CAACGGTACTATTACCGTTGAAGAGCTTAAAAAGCTCCTTGAACAATGGAACCT
AGTAATAGGTTTCCTATTCCTTACATGGATTTGTCTTCTACAATTTGCCTATGCCA
ACAGGAATAGGTTTTTGTATATAATTAAGTTAATTTTCCTCTGGCTGTTATGGCCA
GTAACTTTAGCTTGTTTTGTGCTTGCTGCTGTTTACAGAATAAATTGGATCACCGG
TGGAATTGCTATCGCAATGGCTTGTCTTGTAGGCTTGATGTGGCTCAGCTACTTCA
TTGCTTCTTTCAGACTGTTTGCGCGTACGCGATCCATGTGGTCATTCAATCCAGAA
ACTAACATTCTTCTCAACGTGCCACTCCATGGCACTATTCTGACCAGACCGCTTCT
AGAAAGTGAACTCGTAATCGGAGCTGTGATCCTTCGTGGACATCTTCGTATTGCT
GGACACCATCTAGGACGCTGTGACATCAAGGACCTGCCTAAAGAAATCACTGTT
GCTACATCACGAACGCTTTCTTATTACAAATTGGGAGCTTCGCAGCGTGTAGCAG
GTGACTCAGGTTTTGCTGCATACAGTCGCTACAGGATTGGCAACTATAAATTAAA
CACAGACCATTCCAGTAGCAGTGACAATATTGCTTTGCTTGTACAGTAAGTGACA
ACAGATGTTTCATCTCGTTGACTTTCAGGTTACTATAGCAGAGATATTACTAATTA
TTATGAGGACTTTTAAAGTTTCCATTTGGAATCTTGATTACATCATAAACCTCATA
ATTAAAAATTTATCTAAGTCACTAACTGAGAATAAATATTCTCAATTAGATGAAG
AGCAACCAATGGAGATTGATTAAACGAACATGAAAATTATTCTTTTCTTGGCACT
GATAACACTCGCTACTTGTGAGCTTTATCACTACCAAGAGTGTGTTAGAGGTACA
ACAGTACTTTTAAAAGAACCTTGCTCTTCTGGAACATACGAGGGCAATTCACCAT
TTCATCCTCTAGCTGATAACAAATTTGCACTGACTTGCTTTAGCACTCAATTTGCT
TTTGCTTGTCCTGACGGCGTAAAACACGTCTATCAGTTACGTGCCAGATCAGTTT
CACCTAAACTGTTCATCAGACAAGAGGAAGTTCAAGAACTTTACTCTCCAATTTT
TCTTATTGTTGCGGCAATAGTGTTTATAACACTTTGCTTCACACTCAAAAGAAAG
ACAGAATGATTGAACTTTCATTAATTGACTTCTATTTGTGCTTTTTAGCCTTTCTG
CTATTCCTTGTTTTAATTATGCTTATTATCTTTTGGTTCTCACTTGAACTGCAAGAT
CATAATGAAACTTGTCACGCCTAAACGAACATGAAATTTCTTGTTTTCTTAGGAA
TCATCACAACTGTAGCTGCATTTCACCAAGAATGTAGTTTACAGTCATGTACTCA
ACATCAACCATATGTAGTTGATGACCCGTGTCCTATTCACTTCTATTCTAAATGGT
ATATTAGAGTAGGAGCTAGAAAATCAGCACCTTTAATTGAATTGTGCGTGGATGA
GGCTGGTTCTAAATCACCCATTCAGTACATCGATATCGGTAATTATACAGTTTCC
TGTTTACCTTTTACAATTAATTGCCAGGAACCTAAATTGGGTAGTCTTGTAGTGCG
TTGTTCGTTCTATGAAGACTTTTTAGAGTATCATGACGTTCGTGTTGTTTTAGATT
TCATCTAAACGAACAAACTAAAATGTCTGATAATGGACCCCAAAATCAGCGAAA
TGCACCCCGCATTACGTTTGGTGGACCCTCAGATTCAACTGGCAGTAACCAGAAT
GGAGAACGCAGTGGGGCGCGATCAAAACAACGTCGGCCCCAAGGTTTACCCAAT
AATACTGCGTCTTGGTTCACCGCTCTCACTCAACATGGCAAGGAAGACCTTAAAT
TCCCTCGAGGACAAGGCGTTCCAATTAACACCAATAGCAGTCCAGATGACCAAA
TTGGCTACTACCGAAGAGCTACCAGACGAATTCGTGGTGGTGACGGTAAAATGA
AAGATCTCAGTCCAAGATGGTATTTCTACTACCTAGGAACTGGGCCAGAAGCTG
GACTTCCCTATGGTGCTAACAAAGACGGCATCATATGGGTTGCAACTGAGGGAG
CCTTGAATACACCAAAAGATCACATTGGCACCCGCAATCCTGCTAACAATGCTGC
AATCGTGCTACAACTTCCTCAAGGAACAACATTGCCAAAAGGCTTCTACGCAGA
AGGGAGCAGAGGCGGCAGTCAAGCCTCTTCTCGTTCCTCATCACGTAGTCGCAAC
AGTTCAAGAAATTCAACTCCAGGCAGCAGTAGGGGAACTTCTCCTGCTAGAATG
GCTGGCAATGGCGGTGATGCTGCTCTTGCTTTGCTGCTGCTTGACAGATTGAACC
AGCTTGAGAGCAAAATGTCTGGTAAAGGCCAACAACAACAAGGCCAAACTGTCA
CTAAGAAATCTGCTGCTGAGGCTTCTAAGAAGCCTCGGCAAAAACGTACTGCCA
CTAAAGCATACAATGTAACACAAGCTTTCGGCAGACGTGGTCCAGAACAAACCC
AAGGAAATTTTGGGGACCAGGAACTAATCAGACAAGGAACTGATTACAAACATT
GGCCGCAAATTGCACAATTTGCCCCCAGCGCTTCAGCGTTCTTCGGAATGTCGCG
CATTGGCATGGAAGTCACACCTTCGGGAACGTGGTTGACCTACACAGGTGCCATC
AAATTGGATGACAAAGATCCAAATTTCAAAGATCAAGTCATTTTGCTGAATAAGC
ATATTGACGCATACAAAACATTCCCACCAACAGAGCCTAAAAAGGACAAAAAGA
AGAAGGCTGATGAAACTCAAGCCTTACCGCAGAGACAGAAGAAACAGCAAACT
GTGACTCTTCTTCCTGCTGCAGATTTGGATGATTTCTCCAAACAATTGCAACAATC
CATGAGCAGTGCTGACTCAACTCAGGCCTAAACTCATGCAGACCACACAAGGCA
GATGGGCTATATAAACGTTTTCGCTTTTCCGTTTACGATATATAGTCTACTCTTGT
GCAGAATGAATTCTCGTAACTACATAGCACAAGTAGATGTAGTTAACTTTAATCT
CACATAGCAATCTTTAATCAGTGTGTAACATTAGGGAGGACTTGAAAGAGCCAC
CACATTTTCACCGAGGCCACGCGGAGTACGATCGAGTGTACAGTGAACAATGCT
AGGGAGAGCTGCCTATATGGAAGAGCCCTAATGTGTAAAATTAATTTTAGTAGTG
CTATCCCCATGTGATTTTAATAGCTTCTTAGGAGAATGACAAAAAAAAAAAAAA
AAAAAAAAAAAAAAAAAAAAAAAAAAAAAAAAAAAAAAAAAAAAAGGGTCGG
CATGGCATCTCCACCTCCTCGCGGTCCGACCTGGGCATCCGAAGGAGGACGCAC
GTCCACTCGGATGGCTAAGGGAGCAGCACACTGGCGGCCGTTACTAGGGCCGCG
CCCTTCCCAACAGTTGCGCAGCCTGAATGGCGAATGGAGATCCAATTTTTAAGTG
TATAATGTGTTAAACTACTGATTCTAATTGTTTGTGTATTTTAGATTCACAGTCCC
AAGGCTCATTTCAGGCCCCTCAGTCCTCACAGTCTGTTCATGATCATAATCAGCC
ATACCACATTTGTAGAGGTTTTACTTGCTTTAAAAAACCTCCCACACCTCCCCCTG
AACCTGAAACATAAAATGAATGCAATTGTTGTTGTTAACTTGTTTATTGCAGCTT
ATAATGGTTACAAATAAAGCAATAGCATCACAAATTTCACAAATAAAGCATTTTT
TTCACTGCATTCTAGTTGTGGTTTGTCCAAACTCATCAATGTATCTTAAAGCTTGA
GTATTCTATAGTCTCACCTAAATAGCTTGGCGTAATCATGGTCATAGCTGTTTCCT
GTGTGAAATTGTTATCCGCTCACAATTCCACACAACATACGAGCCGGAAGCATA
AAGTGTAAAGCCTGGGGTGCCTAATGAGTGAGCTAACTCACATTAATTGCGTTGC
GCTCACTGCCCGCTTTCCAGTCGGGAAACCTGTCGTGCCAGCTGCATTAATGAAT
CGGCCAACGCGAACCCCTTGCGGCCGCCCGGGCCGTCGACCAATTCTCATGTTTG
ACAGCTTATCATCGAATTTCTGCCATTCATCCGCTTATTATCACTTATTCAGGCGT
AGCAACCAGGCGTTTAAGGGCACCAATAACTGCCTTAAAAAAATTACGCCCCGC
CCTGCCACTCATCGCAGTACTGTTGTAATTCATTAAGCATTCTGCCGACATGGAA
GCCATCACAAACGGCATGATGAACCTGAATCGCCAGCGGCATCAGCACCTTGTC
GCCTTGCGTATAATATTTGCCCATGGTGAAAACGGGGGCGAAGAAGTTGTCCAT
ATTGGCCACGTTTAAATCAAAACTGGTGAAACTCACCCAGGGATTGGCTGAGAC
GAAAAACATATTCTCAATAAACCCTTTAGGGAAATAGGCCAGGTTTTCACCGTAA
CACGCCACATCTTGCGAATATATGTGTAGAAACTGCCGGAAATCGTCGTGGTATT
CACTCCAGAGCGATGAAAACGTTTCAGTTTGCTCATGGAAAACGGTGTAACAAG
GGTGAACACTATCCCATATCACCAGCTCACCGTCTTTCATTGCCATACGAAATTC
CGGATGAGCATTCATCAGGCGGGCAAGAATGTGAATAAAGGCCGGATAAAACTT
GTGCTTATTTTTCTTTACGGTCTTTAAAAAGGCCGTAATATCCAGCTGAACGGTCT
GGTTATAGGTACATTGAGCAACTGACTGAAATGCCTCAAAATGTTCTTTACGATG
CCATTGGGATATATCAACGGTGGTATATCCAGTGATTTTTTTCTCCATTTTAGCTT
CCTTAGCTCCTGAAAATCTCGATAACTCAAAAAATACGCCCGGTAGTGATCTTAT
TTCATTATGGTGAAAGTTGGAACCTCTTACGTGCCGATCAACGTCTCATTTTCGCC
AAAAGTTGGCCCAGGGCTTCCCGGTATCAACAGGGACACCAGGATTTATTTATTC
TGCGAAGTGATCTTCCGTCACAGGTATTTATTCGCGATAAGCTCATGGAGCGGCG
TAACCGTCGCACAGGAAGGACAGAGAAAGCGCGGATCTGGGAAGTGACGGACA
GAACGGTCAGGACCTGGATTGGGGAGGCGGTTGCCGCCGCTGCTGCTGACGGTG
TGACGTTCTCTGTTCCGGTCACACCACATACGTTCCGCCATTCCTATGCGATGCAC
ATGCTGTATGCCGGTATACCGCTGAAAGTTCTGCAAAGCCTGATGGGACATAAGT
CCATCAGTTCAACGGAAGTCTACACGAAGGTTTTTGCGCTGGATGTGGCTGCCCG
GCACCGGGTGCAGTTTGCGATGCCGGAGTCTGATGCGGTTGCGATGCTGAAACA
ATTATCCTGAGAATAAATGCCTTGGCCTTTATATGGAAATGTGGAACTGAGTGGA
TATGCTGTTTTTGTCTGTTAAACAGAGAAGCTGGCTGTTATCCACTGAGAAGCGA
ACGAAACAGTCGGGAAAATCTCCCATTATCGTAGAGATCCGCATTATTAATCTCA
GGAGCCTGTGTAGCGTTTATAGGAAGTAGTGTTCTGTCATGATGCCTGCAAGCGG
TAACGAAAACGATTTGAATATGCCTTCAGGAACAATAGAAATCTTCGTGCGGTGT
TACGTTGAAGTGGAGCGGATTATGTCAGCAATGGACAGAACAACCTAATGAACA
CAGAACCATGATGTGGTCTGTCCTTTTACAGCCAGTAGTGCTCGCCGCAGTCGAG
CGACAGGGCGAAGCCCTCGGCTGGTTGCCCTCGCCGCTGGGCTGGCGGCCGTCT
ATGGCCCTGCAAACGCGCCAGAAACGCCGTCGAAGCCGTGTGCGAGACACCGCG
GCCGGCCGCCGGCGTTGTGGATACCTCGCGGAAAACTTGGCCCTCACTGACAGA
TGAGGGGCGGACGTTGACACTTGAGGGGCCGACTCACCCGGCGCGGCGTTGACA
GATGAGGGGCAGGCTCGATTTCGGCCGGCGACGTGGAGCTGGCCAGCCTCGCAA
ATCGGCGAAAACGCCTGATTTTACGCGAGTTTCCCACAGATGATGTGGACAAGC
CTGGGGATAAGTGCCCTGCGGTATTGACACTTGAGGGGCGCGACTACTGACAGA
TGAGGGGCGCGATCCTTGACACTTGAGGGGCAGAGTGCTGACAGATGAGGGGCG
CACCTATTGACATTTGAGGGGCTGTCCACAGGCAGAAAATCCAGCATTTGCAAG
GGTTTCCGCCCGTTTTTCGGCCACCGCTAACCTGTCTTTTAACCTGCTTTTAAACC
AATATTTATAAACCTTGTTTTTAACCAGGGCTGCGCCCTGTGCGCGTGACCGCGC
ACGCCGAAGGGGGGTGCCCCCCCTTCTCGAACCCTCCCGGTCGAGTGAGCGAGG
AAGCACCAGGGAACAGCACTTATATATTCTGCTTACACACGATGCCTGAAAAAA
CTTCCCTTGGGGTTATCCACTTATCCACGGGGATATTTTTATAATTATTTTTTTTAT
AGTTTTTAGATCTTCTTTTTTAGAGCGCCTTGTAGGCCTTTATCCATGCTGGTTCT
AGAGAAGGTGTTGTGACAAATTGCCCTTTCAGTGTGACAAATCACCCTCAAATGA
CAGTCCTGTCTGTGACAAATTGCCCTTAACCCTGTGACAAATTGCCCTCAGAAGA
AGCTGTTTTTTCACAAAGTTATCCCTGCTTATTGACTCTTTTTTATTTAGTGTGACA
ATCTAAAAACTTGTCACACTTCACATGGATCTGTCATGGCGGAAACAGCGGTTAT
CAATCACAAGAAACGTAAAAATAGCCCGCGAATCGTCCAGTCAAACGACCTCAC
TGAGGCGGCATATAGTCTCTCCCGGGATCAAAAACGTATGCTGTATCTGTTCGTT
GACCAGATCAGAAAATCTGATGGCACCCTACAGGAACATGACGGTATCTGCGAG
ATCCATGTTGCTAAATATGCTGAAATATTCGGATTGACCTCTGCGGAAGCCAGTA
AGGATATACGGCAGGCATTGAAGAGTTTCGCGGGGAAGGAAGTGGTTTTTTATC
GCCCTGAAGAGGATGCCGGCGATGAAAAAGGCTATGAATCTTTTCCTTGGTTTAT
CAAACGTGCGCACAGTCCATCCAGAGGGCTTTACAGTGTACATATCAACCCATAT
CTCATTCCCTTCTTTATCGGGTTACAGAACCGGTTTACGCAGTTTCGGCTTAGTGA
AACAAAAGAAATCACCAATCCGTATGCCATGCGTTTATACGAATCCCTGTGTCAG
TATCGTAAGCCGGATGGCTCAGGCATCGTCTCTCTGAAAATCGACTGGATCATAG
AGCGTTACCAGCTGCCTCAAAGTTACCAGCGTATGCCTGACTTCCGCCGCCGCTT
CCTGCAGGTCTGTGTTAATGAGATCAACAGCAGAACTCCAATGCGCCTCTCATAC
ATTGAGAAAAAGAAAGGCCGCCAGACGACTCATATCGTATTTTCCTTCCGCGATA
TCACTTCCATGACGACAGGATAGTCTGAGGGTTATCTGTCACAGATTTGAGGGTG
GTTCGTCACATTTGTTCTGACCTACTGAGGGTAATTTGTCACAGTTTTGCTGTTTC
CTTCAGCCTGCATGGATTTTCTCATACTTTTTGAACTGTAATTTTTAAGGAAGCCA
AATTTGAGGGCAGTTTGTCACAGTTGATTTCCTTCTCTTTCCCTTCGTCATGTGAC
CTGATATCGGGGGTTAGTTCGTCATCATTGATGAGGGTTGATTATCACAGTTTATT
ACTCTGAATTGGCTATCCGCGTGTGTACCTCTACCTGGAGTTTTTCCCACGGTGG
ATATTTCTTCTTGCGCTGAGCGTAAGAGCTATCTGACAGAACAGTTCTTCTTTGCT
TCCTCGCCAGTTCGCTCGCTATGCTCGGTTACACGGCTGCGGCG

### DETAILED DESCRIPTION

The present inventors have primarily developed a (partly) codon deoptimized (CD) SARS-COV-2 genome for use as a vaccine. The vaccine can prevent infection with SARS-CoV-2 virus and the complications that arise following infection (acute respiratory distress syndrome).

Using codon deoptimization (CD) technology, the inventors inserted a number of codon changes in the genome of the virus (wild-type SARS-COV-2, Wuhan strain, https://www.ncbi.nlm.nih.gov/nuccore/1798174254) with the objective of decreasing replication efficiency in mammalian cells and rendering the virus attenuated compared to wild-type SARS-COV-2. Using this strategy, the resulting viruses would be strongly attenuated but still produce viral proteins with properties similar to those produced by a wild-type virus. Thus, using CD technology, the inventors are able to generate live attenuated SARS-COV-2 vaccine candidates.

By inserting a substantial number of changes into each vaccine candidate, the chance of reversion to wild-type is negligible, which is a crucial safety feature of the vaccines. This represents a substantial competitive advantage over vaccines with only a small number of mutations.

CD in case of SARS-COV-2 presumably results in slower non-structural polyprotein translation leading to its reduced production, slower replication and, as a result, in attenuation of the virus, compared with wild-type SARS-COV-2. For some embodiments, such vaccine candidates have virtually no risk of de-attenuation (the chance of reversion to wild-type is negligible) because of too many substitutions, all of which have, taken alone, minimal effect on virus, have been made in the coding sequence.

CD, as used herein, involves substituting normal codons in the wild-type SARS-CoV-2 genome with synonymous codons used less frequently in the host (e.g. humans), so that the resulting virus proteins are identical to wild-type virus proteins. Moreover, the resulting virus is highly attenuated, but protein function is not compromised. CD entails genetically engineering the virus.

Non-limiting embodiments of the invention are defined below.

According to a first embodiment of the present invention, there is provided live attenuated severe acute respiratory syndrome coronavirus 2 (SARS-COV-2), SARS-COV-2. SARS-COV-2 particle or SARS-COV-2 nucleic acid comprising a partly codon deoptimized SARS-COV-2 genome.

According to a second embodiment of the present invention, there is provided a recombinant, isolated or substantially purified nucleic acid comprising a partly codon deoptimized SARS-COV-2 genome or partly codon deoptimized region thereof.

According to a third embodiment of the present invention, there is provided a vector, plasmid or genetic construct comprising the nucleic acid of the second embodiment.

According to a fourth embodiment of the present invention, there is provided a cell or isolate containing the live attenuated SARS-COV-2, SARS-COV-2, SARS-COV-2 particle or SARS-COV-2 nucleic acid of the first embodiment, the nucleic acid of the second embodiment, or the vector, plasmid or genetic construct of the third embodiment.

According to a fifth embodiment of the present invention, there is provided a vaccine comprising the live attenuated SARS-COV-2, SARS-COV-2, SARS-COV-2 particle or SARS-COV-2 nucleic acid of the first embodiment, the recombinant, isolated or substantially purified nucleic acid of the second embodiment, the vector, plasmid or genetic construct of the third embodiment, or the cell or isolate of the fourth embodiment.

According to a sixth embodiment of the present invention, there is provided a pharmaceutical preparation comprising the live attenuated SARS-COV-2, SARS-COV-2, SARS-COV-2 particle or SARS-COV-2 nucleic acid of the first embodiment, the recombinant, isolated or substantially purified nucleic acid of the second embodiment, the vector, plasmid or genetic construct of the third embodiment, or the cell or isolate of the fourth embodiment.

According to a seventh embodiment of the present invention, there is provided an immunogenic composition comprising the live attenuated SARS-COV-2, SARS-COV-2, SARS-COV-2 particle or SARS-COV-2 nucleic acid of the first embodiment, the recombinant, isolated or substantially purified nucleic acid of the second embodiment, the vector, plasmid or genetic construct of the third embodiment, or the cell or isolate of the fourth embodiment.

According to an eighth embodiment of the present invention, there is provided a method of: (1) vaccinating a subject; (2) prophylactically immunizing a subject against SARS-CoV-2 or SARS-COV-2-like virus; (3) preventing a subject from contracting a SARS-COV-2 infection naturally or a SARS-COV-2-like infection naturally; (4) reducing the severity of a natural SARS-COV-2 disease or natural SARS-COV-2-like disease in a subject; or (5) treating a subject having a natural SARS-COV-2 infection or natural SARS-COV-2-like infection, said method comprising the step of administering to the subject: the live attenuated SARS-COV-2, SARS-COV-2, SARS-COV-2 particle or SARS-COV-2 nucleic acid of the first embodiment; the recombinant, isolated or substantially purified nucleic acid of the second embodiment; the vector, plasmid or genetic construct of the third embodiment; the cell or isolate of the fourth embodiment; the vaccine of the fifth embodiment; the pharmaceutical preparation of the sixth embodiment; or the immunogenic composition of the seventh embodiment.

According to a ninth embodiment of the present invention, there is provided use of: the live attenuated SARS-COV-2, SARS-COV-2, SARS-COV-2 particle or SARS-COV-2 nucleic acid of the first embodiment; the recombinant, isolated or substantially purified nucleic acid of the second embodiment; the vector, plasmid or genetic construct of the third embodiment; the cell or isolate of the fourth embodiment; the vaccine of the fifth embodiment; the pharmaceutical preparation of the sixth embodiment; or the immunogenic composition of the seventh embodiment, in the preparation of a medicament for: (1) vaccinating a subject; (2) prophylactically immunizing a subject against SARS-COV-2 or SARS-COV-2-like virus; (3) preventing a subject from contracting a SARS-COV-2 infection naturally or a SARS-COV-2-like infection naturally; (4) reducing the severity of a natural SARS-COV-2 disease or natural SARS-COV-2-like disease in a subject; or (5) treating a subject having a natural SARS-COV-2 infection or natural SARS-COV-2-like infection.

According to a tenth embodiment of the present invention, there is provided: a live attenuated SARS-COV-2, SARS-COV-2, SARS-COV-2 particle or SARS-COV-2 nucleic acid of the first embodiment; a recombinant, isolated or substantially purified nucleic acid of the second embodiment; a vector, plasmid or genetic construct of the third embodiment; a cell or isolate of the fourth embodiment; a vaccine of the fifth embodiment; a pharmaceutical preparation of the sixth embodiment; or an immunogenic composition of the seventh embodiment, for use in: (1) vaccinating a subject; (2) prophylactically immunizing a subject against SARS-COV-2 or SARS-COV-2-like virus; (3) preventing a subject from contracting a SARS-COV-2 infection naturally or a SARS-COV-2-like infection naturally; (4) reducing the severity of a natural SARS-COV-2 disease or natural SARS-COV-2-like disease in a subject; or (5) treating a subject having a natural SARS-COV-2 infection or natural SARS-COV-2-like infection.

According to an eleventh embodiment of the present invention, there is provided a method of generating a live attenuated SARS-COV-2 vaccine, SARS-COV-2, SARS-COV-2 particle or SARS-COV-2 nucleic acid, or recombinant, isolated or substantially purified nucleic acid comprising a partly codon deoptimized SARS-COV-2 genome or partly codon deoptimized region thereof, comprising the step of partly codon deoptimizing a SARS-COV-2 genome.

According to a twelfth embodiment of the present invention, there is provided a method of preparing a vaccine comprising live attenuated SARS-COV-2, said method comprising the steps of: (1) codon deoptimizing a SARS-COV-2 genome to produce a partly codon deoptimized live attenuated SARS-COV-2; and (2) enabling the partly codon deoptimized live attenuated SARS-COV-2 to replicate.

According to a thirteenth embodiment of the present invention, there is provided a method of preparing a vaccine comprising codon deoptimized SARS-COV-2, said method comprising the steps of: optionally, (1) codon deoptimizing a SARS-COV-2 genome to produce a partly codon deoptimized live attenuated SARS-COV-2; (2) enabling the partly codon deoptimized live attenuated SARS-COV-2 to replicate; and (3) preparing a vaccine dose containing the replicated SARS-COV-2 of step (2).

According to a fourteenth embodiment of the present invention, there is provided a method of eliciting an immune response in a subject, said method comprising the step of administering a live attenuated SARS-COV-2, SARS-COV-2, SARS-COV-2 particle or SARS-CoV-2 nucleic acid of the first embodiment; the recombinant, isolated or substantially purified nucleic acid of the second embodiment; the vector, plasmid or genetic construct of the third embodiment; the cell or isolate of the fourth embodiment; the vaccine of the fifth embodiment; the pharmaceutical preparation of the sixth embodiment; or the immunogenic composition of the seventh embodiment to the subject to thereby elicit an immune response.

It is to be appreciated that, context permitting, features of the above fourteen invention embodiments can be found elsewhere in this specification, including below.

By ‘live attenuated’ it is meant that the virus demonstrates substantially reduced or preferably no clinical signs of disease when administered to a subject, compared with wild-type SARS-COV-2.

‘Wild-type SARS-COV-2’ refers to the Wuhan strain, found at https://www.ncbi.nlm.nih.gov/nuccore/1798174254.

It is to be appreciated that, context permitting, wild-type SARS-COV-2 can include natural variants (present and future) of the Wuhan strain, including: Alpha, Pango lineage B.1.1.7; Beta, Pango lincages B.1.351, B.1.351.2, B.1.351.3; Gamma, Pango lineages P.1, P.1.1, P.1.2; Delta, Pango lincages B.1.617.2, AY.1, AY.2; Eta, Pango lineage B.1.525; Iota, Pango lineage B.1.526; Kappa, Pango lincage B.1.617.1; Lambda, Pango lineage C.37; and, Pango lincages B.1.427, B.1.429, P.2, P.3, R.1, R.2, B.1.466.2, B.1.621, AV.1, B.1.1.318, B.1.1.519, AT.1, C.36.3, C.36.3.1, B.1.214.2.

Any suitable region or regions of the SARS-COV-2 genome can be codon deoptimized. The wild-type Wuhan SARS-COV-2 genome sequence, gene sequences and protein sequences can be found in GenBank as entry NCBI Reference Sequence: NC_045512.2 (Severe acute respiratory syndrome coronavirus 2 isolate Wuhan-Hu-1, complete genome). Those sequence are incorporated herein by reference.

In some embodiments the ORF1a region is codon deoptimized. The wild-type ORF1a sequence can be found in GenBank as entry NCBI Reference Sequence: NC_045512.2. The genome sequence, gene sequences and protein sequences are incorporated herein by reference.

In some embodiments the ORF1a region is codon deoptimized, but excluding/truncating the 5′ region by one or more nucleotides. In some embodiments the ORF1a region is codon deoptimized, but excluding/truncating the 3′ region of ORF1a by one or more nucleotides, thereby excluding the ribosomal frameshift region. In some embodiments, this corresponds between about nucleotide position 1534 to about nucleotide position 8586 of the Wuhan virus genome, but this need not be the case. These positions were chosen by the inventors in view of the cloning strategy. Other positions can be readily determined by the skilled person based on NCBI Reference Sequence: NC_045512.2. In some embodiments, only part of the ORF1a region of the viral genome is codon deoptimized or different parts or sub-regions of the ORF1a region of the viral genome are codon deoptimized.

Any suitable number of codon changes can be made. In some embodiments, CD results in no less than about 10 codon changes in ORF1a. In some embodiments, CD results in no more than about 1850 codon changes in ORF1a (with the upper limit for substitution being where the virus does not usually grow at all). In some embodiments, codon deoptimization results in between about 10 and about 1850 codon changes in ORF1a and all sub-ranges there between. This 10 to 1850 codon change range includes all integers between 10 and 1850 including 11, 12 . . . 1849 codon changes. In some embodiments, some or all of the codon changes can be situated immediately next to one another, in sequence. In some embodiments, some or all of the codon changes can be spaced apart from each other such that they are not situated immediately next to one another, in sequence—e.g. 3 to 4 codon (triplet) spacing. In some embodiments, some of the codon changes can be spaced apart from each other and some of the codon changes can be situated immediately next to one another.

In some embodiments, CD occurs in no more than about a 12 kbp nucleotide region of ORF1a. This can include an about 0.5, 1, 1.5, 2, 2.5, 3, 3.5, 4, 4.5, 5, 5.5, 6, 6.5, 7, 7.5, 8, 8.5, 9, 9.5, 10, 10.5, 11, 11.5 or 12 kbp nucleotide codon deoptimized region. The region can be continuous/contiguous or not. In some embodiments, CD occurs in a continuous ORF1a region with a length of about 12 kbp. In some embodiments, CD results in about an 11,186 nucleotide region of ORF1a, preferably with no less than about 10 codon changes within that nucleotide region. In some embodiments, about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50% of ORF1a is codon deoptimized. In some embodiments, about 35% of ORF1a is codon deoptimized. In some embodiments, every 3^{rd} or 4^{th} codon is deoptimized along ORF1a.

Most amino acids are encoded by more than one codon. For instance, leucine, serine and arginine are encoded by six different codons, while only tryptophan and methionine have unique codons. ‘Synonymous’ codons are codons that encode the same amino acid. For example, CTT, CTC, CTA, CTG, TTA and TTG are synonymous codons that code for leucine. Synonymous codons are not used with equal frequency. In generally, the most frequently used codons in a particular organism are those for which the cognate tRNA is abundant, and the use of these codons enhances the rate and/or accuracy of protein translation. Conversely, tRNAs for the rarely used codons are found at relatively low levels, and the use of rare codons is thought to reduce translation rate and/or accuracy.

As used herein, a ‘rare’ codon is one of at least two synonymous codons encoding a particular amino acid that is present in an mRNA at a significantly lower frequency that the most frequently used codon for that amino acid. Conversely, a ‘frequent’ codon is one of at least two synonymous codons encoding a particular amino acid that is present in an mRNA at a significantly higher frequency that the least frequently used codon for that amino acid. For example, human genes use the leucine codon CTG 3.9% of the time, but use the synonymous codon CTA only 0.7% of the time. See Table 1a. Thus, CTG is a frequent codon, whereas CTA is a rare codon.

TABLE 1a
Codon usage in Homo sapiens (source:
http://www.kazusa.or.jp/codon/)

| Amino Acid | Codon | Frequency % |
|---|---|---|
| Gly | GGG | 1.65 |
| Gly | GGA | 1.65 |
| Gly | GGT | 1.08 |
| Gly | GGC | 2.22 |
| Glu | GAG | 3.96 |
| Glu | GAA | 2.90 |
| Asp | GAT | 2.18 |
| Asp | GAC | 2.51 |
| Val | GTG | 2.81 |
| Val | GTA | 0.71 |
| Val | GTT | 1.10 |
| Val | GTC | 1.45 |
| Ala | GCG | 0.74 |
| Ala | GCA | 1.58 |
| Ala | GCT | 1.85 |
| Ala | GCC | 2.77 |
| End | AGG | 1.20 |
| End | AGA | 1.22 |
| Ser | AGT | 1.21 |
| Ser | AGC | 1.95 |
| Lys | AAG | 3.19 |
| Lys | AAA | 2.44 |
| Asn | AAT | 1.70 |
| Asn | AAC | 1.91 |
| Met | ATG | 2.20 |
| Met | ATA | 0.75 |
| Ile | ATT | 1.60 |
| Ile | ATC | 2.08 |
| Thr | ACG | 0.61 |
| Thr | ACA | 1.51 |
| Thr | ACT | 1.31 |
| Thr | ACC | 1.89 |
| Trp | TGG | 1.32 |
| Trp | TGA | 0.16 |
| Cys | TGT | 1.06 |
| Cys | TGC | 1.26 |
| End | TAG | 0.08 |
| End | TAA | 0.10 |
| Tyr | TAT | 1.22 |
| Tyr | TAC | 1.53 |
| Leu | TTG | 1.29 |
| Leu | TTA | 0.77 |
| Phe | TTT | 1.76 |
| Phe | TTC | 2.03 |
| Ser | TCG | 0.44 |
| Ser | TCA | 1.22 |
| Ser | TCT | 1.52 |
| Ser | TCC | 1.77 |
| Arg | CGG | 1.14 |
| Arg | CGA | 0.62 |
| Arg | CGT | 0.45 |
| Arg | CGC | 1.04 |
| Gln | CAG | 3.42 |
| Gln | CAA | 1.23 |
| His | CAT | 1.09 |
| His | CAC | 1.51 |
| Leu | CTG | 3.96 |
| Leu | CTA | 0.72 |
| Leu | CTT | 1.32 |
| Leu | CTC | 1.96 |
| Pro | CCG | 0.69 |
| Pro | CCA | 1.69 |
| Pro | CCT | 1.75 |
| Pro | CCC | 1.98 |

‘Rare codons’ have a frequency of less than 0.5%. For example, TAA, TGA, TCG and CGT are rare codons. ‘Less rare’ codons have a frequency of less than 0.8%. For example, AUA, ACG, CGA, CCG, CTA, CTA, GCG, ATA, TTA are less rare codons. ‘Moderate codons’ have a frequency of less than 2%. For example, CGC, TGT, GGT, CAT, GTT, CGG, AGG, AGT, AGA, TAT, TCA, CAA, TGC, TTG, ACT, TGG, CTT, GTC, CAC, ACA, TCT, TAC, GCA, ATT, GGG, GGA, CCA, AAT, CCT, TTT, TCC, GCT, ACC, AAC, AGC, CTC and CCC are moderate codons. ‘Frequent codons’ have a frequency of more than 2%. For example, TTC, ATC, GAT, ATG, GGC, AAA, GAC, GCC, GTG, GAA, AAG, CAG, GAG and CTG are frequent codons.

The propensity for highly expressed genes to use frequent codons is called ‘codon bias’. A gene for a ribosomal protein might use only the 20 to 25 most frequent of the 61 codons, and have a high codon bias (a codon bias close to 1), while a poorly expressed gene might use all 61 codons, and have little or no codon bias (a codon bias close to 0). It is thought that the frequently used codons are codons where larger amounts of the cognate tRNA are expressed, and that use of these codons allows translation to proceed more rapidly, or more accurately, or both.

In some embodiments, the CD results in slower non-structural polyprotein translation leading to slower replication and, as a result, in attenuation of the virus. In some embodiments, every codon in the wild-type ORF1a or region thereof was analysed in terms of its usage frequency in Homo sapiens, and if the codon was frequent then it was changed in the viral genome to a least frequently or less frequently used synonymous codon. In some embodiments, a codon for an amino acid with codon degeneracy was changed only if the synonymous codons for that amino acid occurred in significantly different frequencies of usage in the genome of Homo sapiens. In some embodiments, Asp, and Asn codons of the viral genome are left unchanged. In some embodiments, a codon for an amino acid with high codon degeneracy was changed to a synonymous codon that was rarely, less rarely or moderately used in the genome of Homo sapiens. In some embodiments, a viral region most rich in codons that can be substituted for rare codon variants is CD.

In some embodiments, CD results in replacement with one or more rare codons.

In some embodiments, CD results in replacement with one or more less rare codons.

In some embodiments, CD results in replacement with one or more moderate codons.

In some embodiments, CD results in replacement with one or more rare codons, one or more less rare codons, or one or more moderate codons, or any combination of these.

In some embodiments, CD results in replacement with one or more CpG dinucleotides (CpG elements).

In some embodiments, CD results in replacement with one or more UpA (TA) dinucleotides (UpA elements).

In some embodiments, CD results in replacement with one or more CpG and one or more UpA dinucleotides/elements, or any combination of these.

In some embodiments, CD results in replacement with one or more rare codons, one or more less rare codons, or one or more moderate codons, one or more CpG dinucleotides/elements, one or more UpA dinucleotides/elements, or any combination of these.

UpA and CpG dinucleotides can act as a vaccine adjuvant as they are important immunoregulators for the RNA virus immune response. In some embodiments, CpG and/or UpA improve the function of antigen-presenting cells, boost the generation of a vaccine-specific immune response and increase the immunogenicity of administered vaccines. Rare serine codon TCG contains a CpG dinucleotide. Less rare codons CTA, CCG, ACG, GTA, ATA and GCG contain UpA or CpG dinucleotides/elements.

In some embodiments, one or more serine codons are changed. In some embodiments, one or more serine codons are changed to the rare TCG codon. (This codon has the CpG element.)

In some embodiments, one or more proline codons are changed. In some embodiments, one or more proline codons are changed to the less rare CCG codon. (This codon has the CpG element.)

In some embodiments, one or more threonine codons are changed. In some embodiments, one or more threonine codons are changed to the less rare ACG codon. (This codon has the CpG element.)

In some embodiments, one or more isoleucine codons are changed. In some embodiments, one or more isoleucine codons are changed to the less rare ATA codon. (This codon has the UpA element.)

In some embodiments, one or more alanine codons are changed. In some embodiments, one or more alanine codons are changed to the less rare GCG codon. (This codon has the CpG element.)

In some embodiments, one or more arginine codons are changed. In some embodiments, one or more arginine codons are changed to the rare CGT codon or less rare CGA codon. (These codons have the CpG elements.)

In some embodiments, one or more serine codons are changed, one or more proline codons are changed, one or more threonine codons are changed, one or more isoleucine codons are changed, one or more alanine codons are changed, one or more arginine codons are changed, or any combination of these.

In some embodiments, a region between about nucleotide positions 1534 and 8586 of the SARS-COV-2 wild-type genome (the ORF1a region) can be codon deoptimized, or any subrange/subregion located between 1534 and 8586. Any suitable number of amino acid codon changes can be made. In some embodiments, a total of at least about 24 codons are changed. In some embodiments, a total of up to about 546 codons are changed. This includes all whole numbers between 24 and 546, including 24, 25 etc. This also includes all subranges between 24-546, such as 24-50, 50-75, 75-100, etc.

For example, one or more of the amino acid codons shown in Table 11 can be changed/mutated—either individually or in any suitable combination with each other. For example, one or more of the following amino acid codons shown in Table 1b below can be changed/mutated—either individually or in any suitable combination with each other (including different codons for the same amino acid and/or with codons for different amino acids).

TABLE 1b

| | | Number of codon changes in | Percentage of codon changes in |
|---|---|---|---|
| Amino | Change to | the 1534-8586 region for | the 1534-8586 region for that |
| acid | codon | that particular amino acid | particular amino acid |
| Ser | TCG, TCC | Approximately 0 to 160, | Approximately 0-100%, including |
| | TCT, TCA | including all integers between | all percentages between 0 and |
| | and/or AGT | 0 and 160, preferably at least | 100, preferably at least about |
| | | about 24, preferably no more | 15%, preferably no more than |
| | | than about 160. | about 100%. |
| Arg | CGT, AGG, | Approximately 0 to 43, | Approximately 0-100%, including |
| | CGG, CGC | including all integers between | all percentages between 0 and |
| | and/or CGA | 0 and 43, preferably at least | 100, preferably at least about |
| | | about 11, preferably no more | 26%, preferably no more than |
| | | than about 40. | about 93%. |
| Thr | ACG, ACA | Approximately 0 to 187, | Approximately 0-100%, including |
| | and/or ACT | including all integers between | all percentages between 0 and |
| | | 0 and 187, preferably at least | 100, preferably at least about |
| | | about 33, preferably no more | 18%, preferably no more than |
| | | than about 186. | about 99%. |
| Pro | CCG, CCT | Approximately 0 to 82, | Approximately 0-100%, including |
| | and/or CCA | including all integers between | all percentages between 0 and |
| | | 0 and 82, preferably at least | 100, preferably at least about |
| | | about 10, preferably no more | 12%, preferably no more than |
| | | than about 82. | about 100%. |
| Val | GTA, GTC | Approximately 0 to 165, | Approximately 0-100%, including |
| | and/or GTT | including all integers between | all percentages between 0 and |
| | | 0 and 165. | 100. |
| Leu | CTA, CTC, | Approximately 0 to 199, | Approximately 0-100%, including |
| | CTT, TTG | including all integers between | all percentages between 0 and |
| | and/or TTA | 0 and 199. | 100. |
| Ala | GCG, GCT | Approximately 0 to 147, | Approximately 0-100%, including |
| | and/or GCA | including all integers between | all percentages between 0 and |
| | | 0 and 147, preferably at least | 100, preferably at least about |
| | | about 34, preferably no more | 23%, preferably no more than |
| | | than about 147. | about 100%. |
| Ile | ATA and/or | Approximately 0 to 87, | Approximately 0-100%, including |
| | ATT | including all integers between | all percentages between 0 and |
| | | 0 and 87, preferably no more | 100, preferably no more than |
| | | than about 82. | about 94%. |
| Cys | TGT | Approximately 0 to 6, | Approximately 0-100%, including |
| | | including all integers between | all percentages between 0 and |
| | | 0 and 6. | 100. |
| Gly | GGT | Approximately 0 to 41, | Approximately 0-100%, including |
| | | including all integers between | all percentages between 0 and |
| | | 0 and 41, preferably at least | 100, preferably at least about |
| | | about 8, preferably no more | 20%, preferably no more than |
| | | than about 41. | about 100%. |
| His | CAT | Approximately 0 to 13, | Approximately 0-100%, including |
| | | including all integers between | all percentages between 0 and |
| | | 0 and 13. | 100. |
| Tyr | TAT | Approximately 0 to 43 | Approximately 0-100%, including |
| | | including all integers between | all percentages between 0 and |
| | | 0 and 43. | 100. |
| Gln | CAA | Approximately 0 to 28, | Approximately 0-100%, including |
| | | including all integers between | all percentages between 0 and |
| | | 0 and 28, preferably at least | 100, preferably at least about 4%, |
| | | about 1, preferably no more | preferably no more than about |
| | | than about 14. | 50%. |
| Trp | TGG | 0 | 0% |
| Asn | AAT | Approximately 0 to 38, | Approximately 0-100%, including |
| | | including all integers between | all percentages between 0 and |
| | | 0 and 38. | 100. |
| Phe | TTT | Approximately 0 to 22, | Approximately 0-100%, including |
| | | including all integers between | all percentages between 0 and |
| | | 0 and 22. | 100. |
| Asp | GAT | Approximately 0 to 39, | Approximately 0-100%, including |
| | | including all integers between | all percentages between 0 and |
| | | 0 and 39. | 100. |
| Met | ATG | 0 | 0% |
| Lys | AAA | Approximately 0 to 56, | Approximately 0-100%, including |
| | | including all integers between | all percentages between 0 and |
| | | 0 and 56. | 100. |
| Glu | GAA | Approximately 0 to 41, | Approximately 0-100%, including |
| | | including all integers between | all percentages between 0 and |
| | | 0 and 41. | 100. |

For example, in some embodiments, the 1534-8586 region can have about 24, 28, 25, 48, 53, 59, 77 or 160 Ser changes to Ser TCG.

For example, in some embodiments, the 1534-8586 region can have about 97 codon changes comprising: about 19 Ile changes to Ile ATA, about 10 Pro changes to Pro CCG, about 34 Thr changes to Thr ACG, and about 34 Ala changes to Ala GCG.

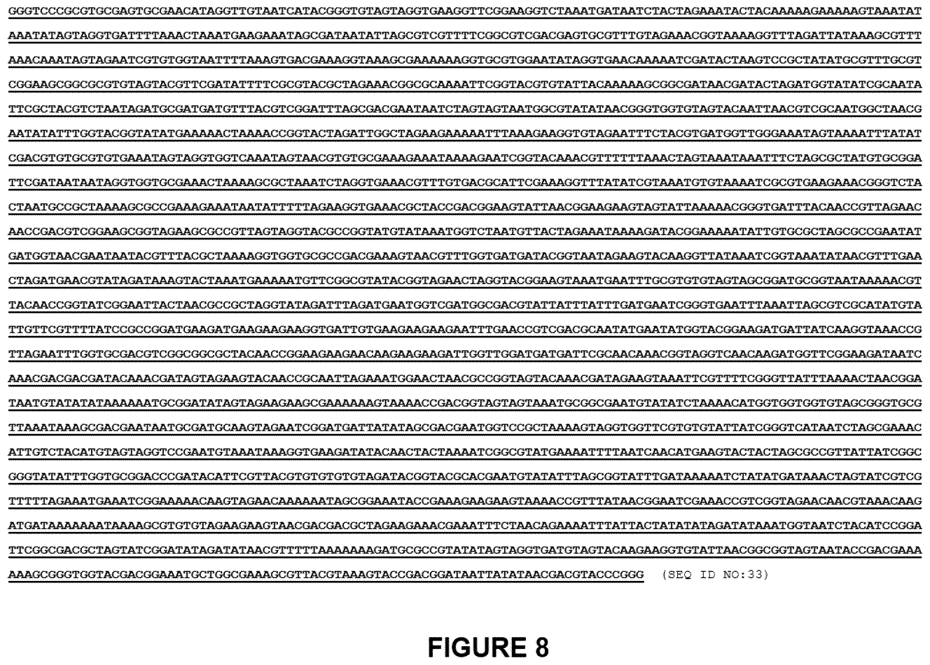

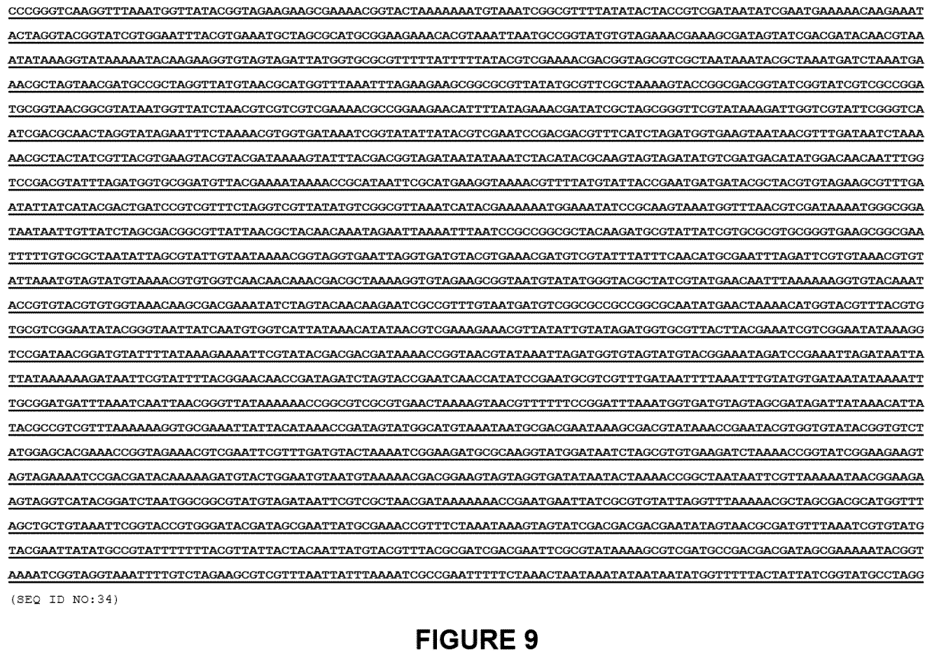

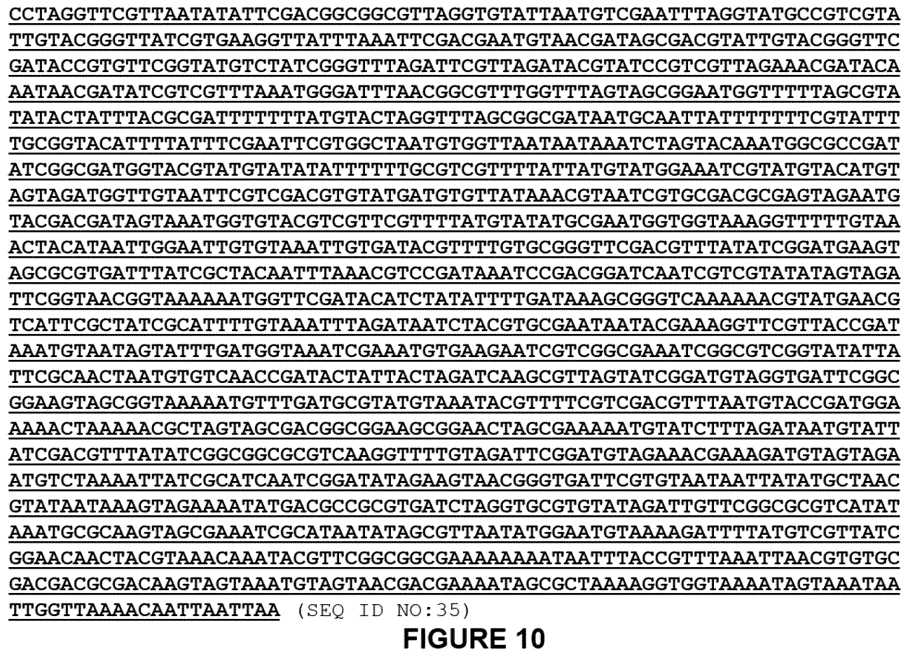

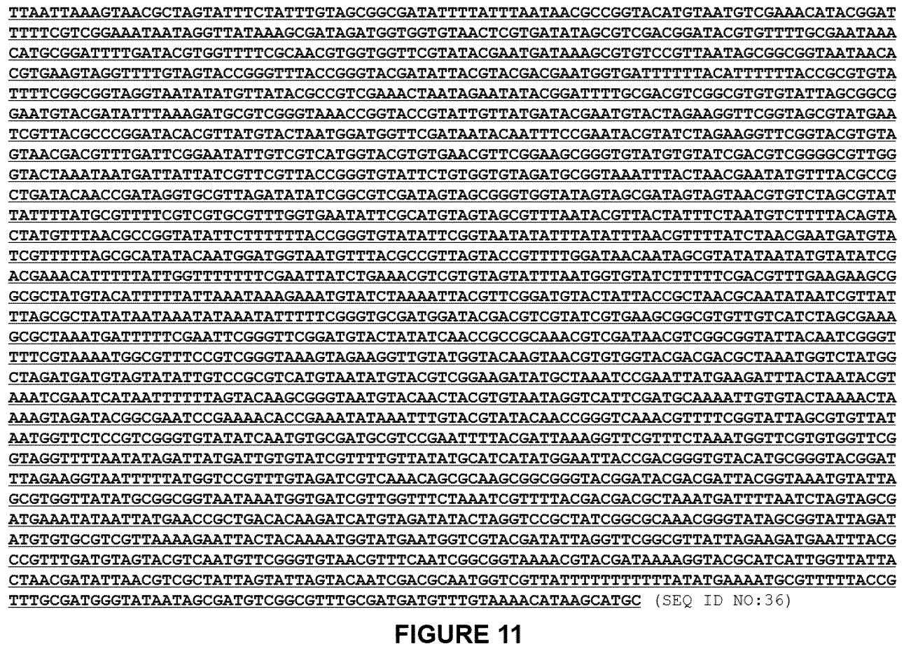

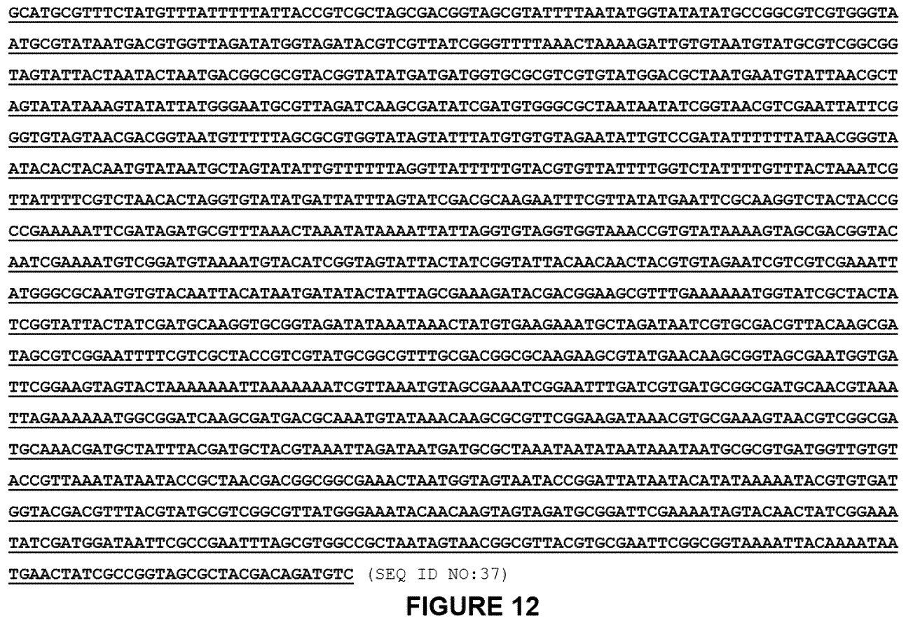

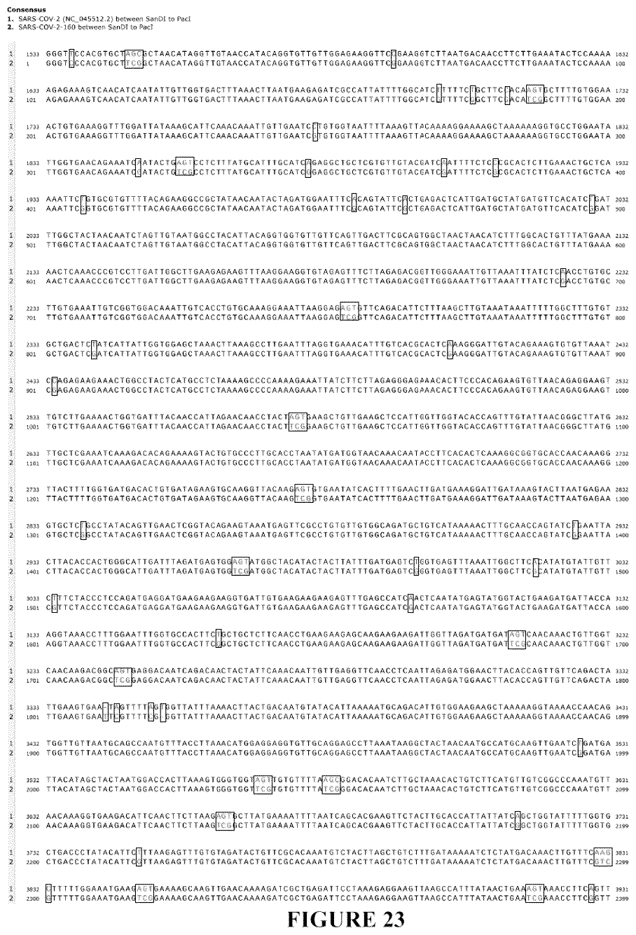

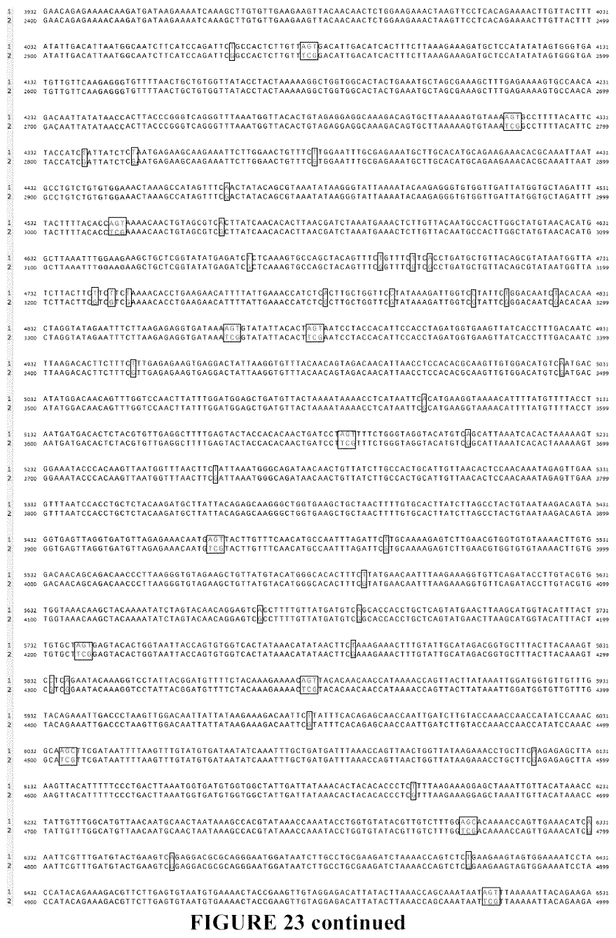

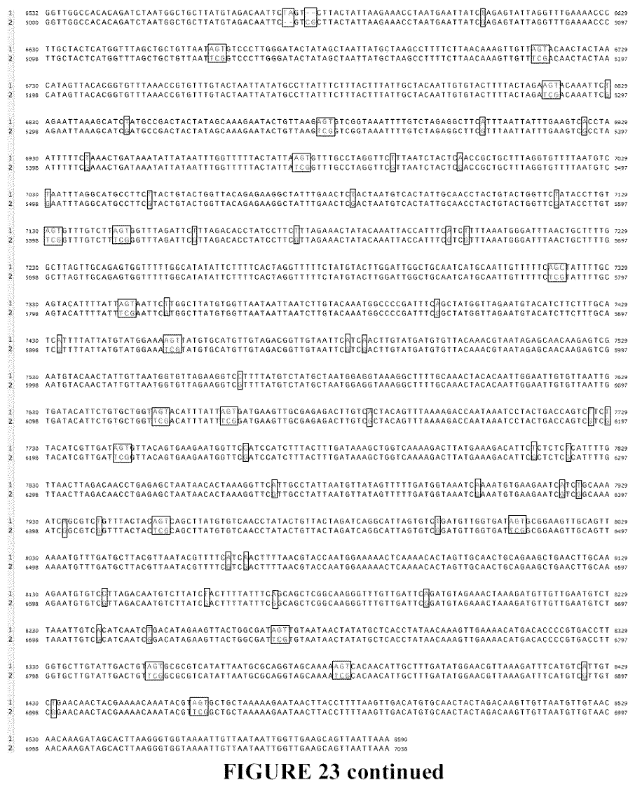

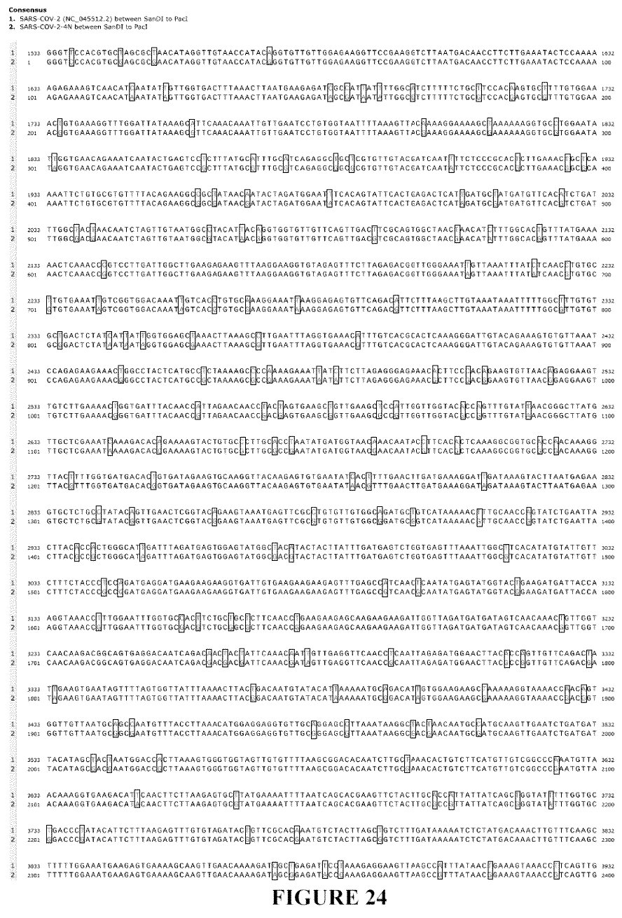

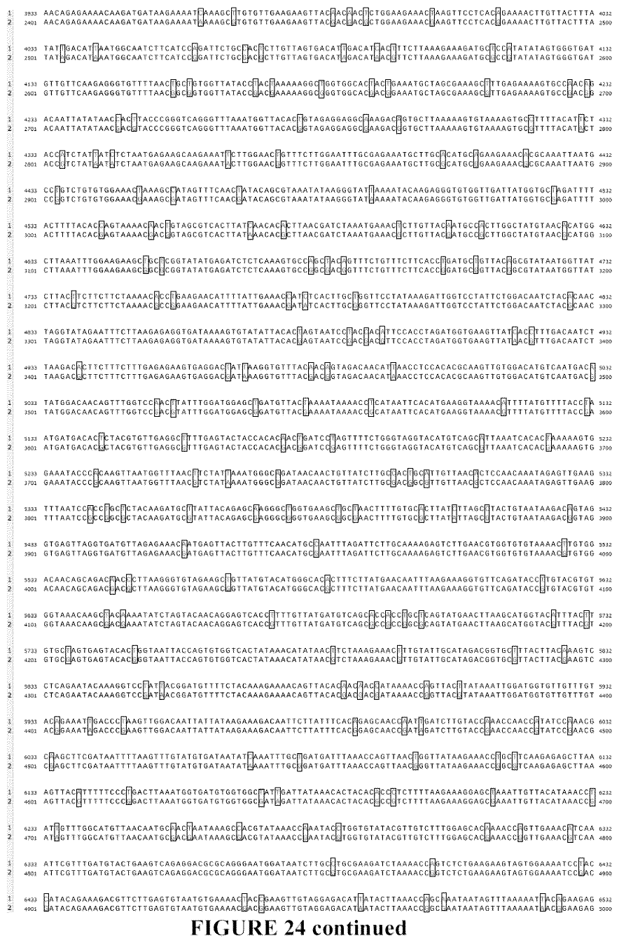

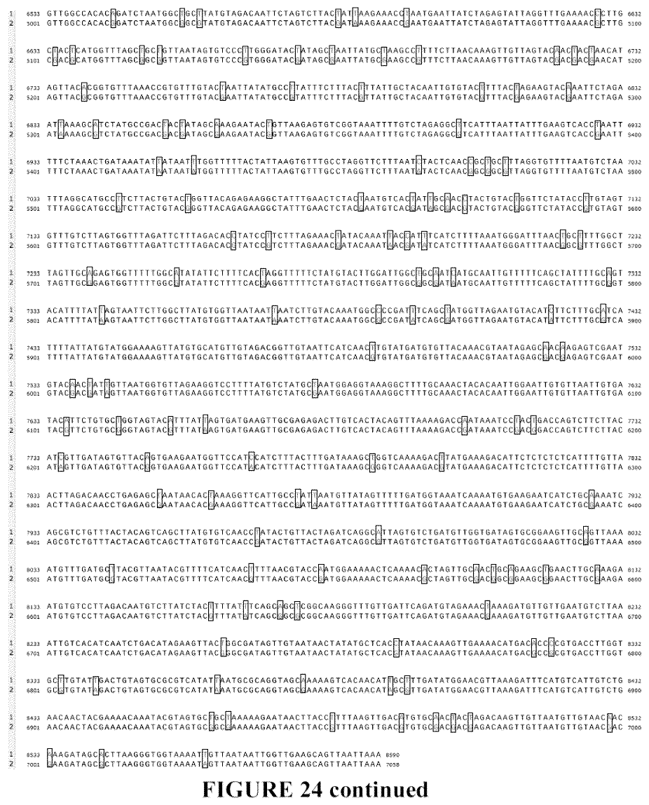

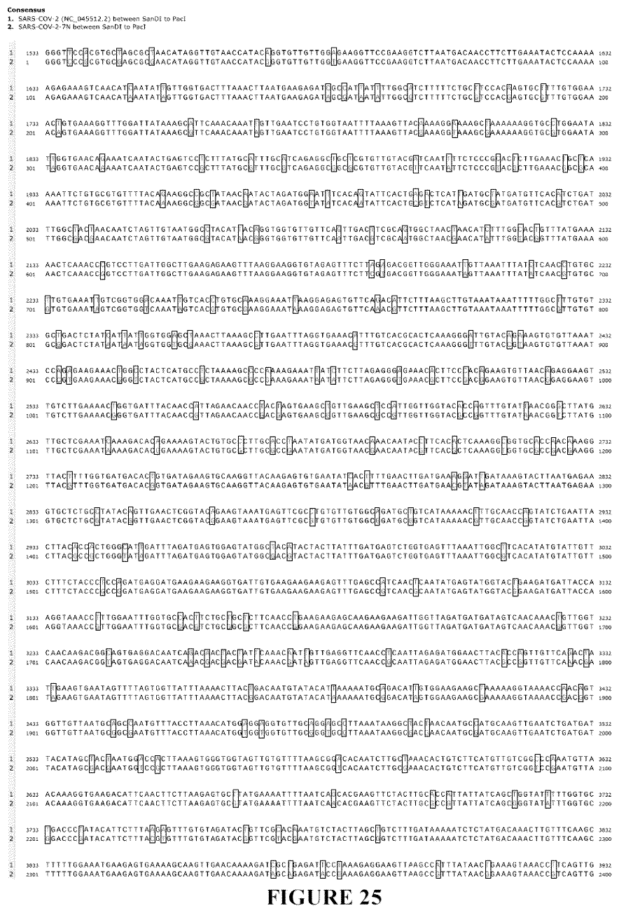

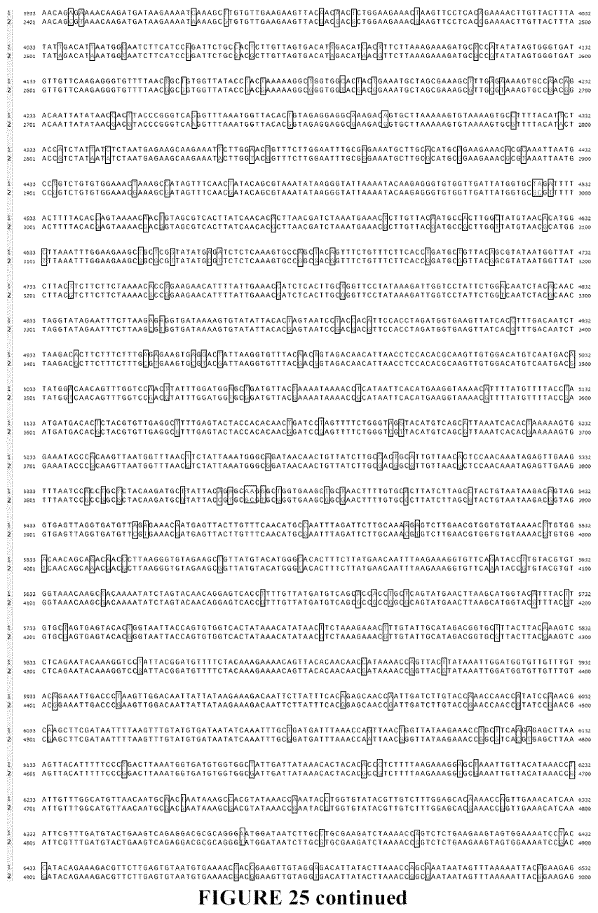

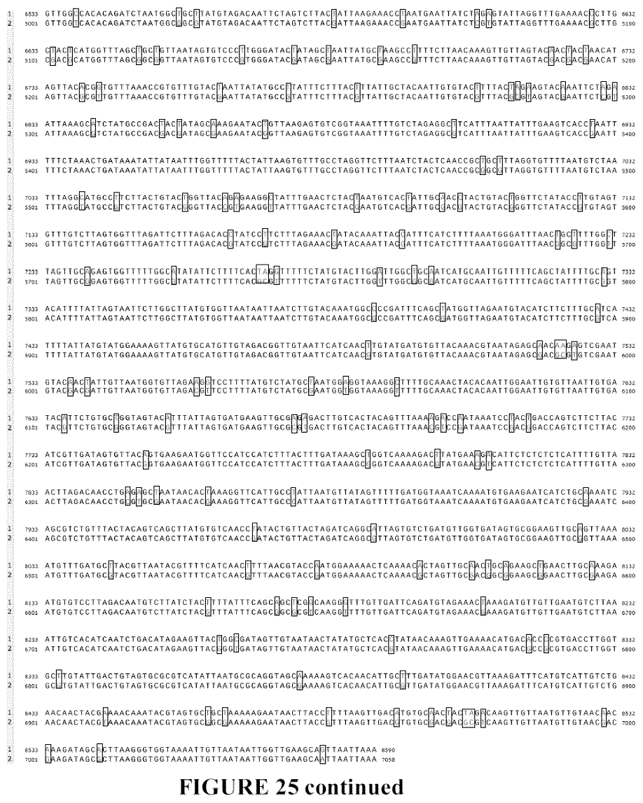

For example, in some embodiments, the codon deoptimized genome can have the deoptimized codons of the fragments/clones/vaccine candidates as shown or as substantially shown in any one of SEQ ID NO:1-31, 33-37 and 39-68, and as shown or as substantially shown in any one of FIGS. 8 to 12 and 22 to 25.

For example, in some embodiments, the codon deoptimized genome can have at least the deoptimized codons of the fragments/clones/vaccine candidates as shown in any one of SEQ ID NO:1-31, 33-37 and 39-68, and as shown or as substantially shown in any one of FIGS. 8 to 12 and 22 to 25.

For example, in some embodiments, the codon deoptimized genome can have fewer than the deoptimized codons of the fragments/clones/vaccine candidates as shown in any one of SEQ ID NO: 1-31, 33-37 and 39-68, and as shown or as substantially shown in any one of FIGS. 8 to 12 and 22 to 25.

For example, in some embodiments, the codon deoptimized genome can have anywhere between about 10% and about 100% of the deoptimized codons of the fragments/clones/vaccine candidates as shown in any one of SEQ ID NO: 1-31, 33-37 and 39-68, and as shown in any one of FIGS. 8 to 12 and 22 to 25, said 10% to 100% range including all integers between 10 and 100, including 11, 12 etc.

In some embodiments, the codon deoptimized genome has the deoptimized region or genomic sequence or substantially the same deoptimized region or genomic sequence of clone SARS-COV-2-77-1, SARS-COV-2-77-2, SARS-COV-2-77-3, SARS-COV-2-77-4, SARS-COV-2-77-5, SARS-COV-2-77-6, SARS-COV-2-77-7, SARS-COV-2-160-1, SARS-COV-2-160-2, SARS-COV-2-160-3, SARS-COV-2-160-4, SARS-COV-2-160-5, SARS-COV-2-160-6, SARS-COV-2-160-7, SARS-COV-2-4N-1 or SARS-COV-2-7N-1, or variant thereof.

In some embodiments, the codon deoptimized genome has the deoptimized region or genomic sequence of clone SARS-COV-2-4N-1, SARS-COV-2-7N-1, SARS-COV-2-77-7, SARS-COV-2-160-4 or SARS-COV-2-160-7, or substantially the same deoptimized region or genomic sequence as clone SARS-COV-2-4N-1, SARS-COV-2-7N-1, SARS-COV-2-77-7, SARS-COV-2-160-4 or SARS-COV-2-160-7, or variant thereof.

In some embodiments, the codon deoptimized genome has the deoptimized region or genomic sequence of clone SARS-COV-2-7N-1 or substantially the same deoptimized region or genomic sequence as clone SARS-COV-2-7N-1, or variant thereof.

In some embodiments, apart from the deoptimized region, a genomic remainder, or part thereof, of the live attenuated severe acute respiratory syndrome coronavirus 2 (SARS-CoV-2), SARS-COV-2, SARS-COV-2 particle or SARS-COV-2 nucleic acid etc can comprise a sequence identical to, substantially identical to or similar to wild-type SARS-COV-2—i.e. the Wuhan isolate or variant thereof. Suitable variants include present and future variants of the Wuhan strain, including: Alpha, Pango lineage B.1.1.7; Beta, Pango lineages B.1.351, B.1.351.2, B.1.351.3; Gamma, Pango lincages P.1, P.1.1, P.1.2; Delta, Pango lineages B.1.617.2, AY.1, AY.2; Eta, Pango lineage B.1.525; Iota, Pango lineage B.1.526; Kappa, Pango lineage B.1.617.1; Lambda, Pango lineage C.37; and, Pango lineages B.1.427, B.1.429, P.2, P.3, R.1, R.2, B.1.466.2, B.1.621, AV.1, B.1.1.318, B.1.1.519, AT.1, C.36.3, C.36.3.1, B.1.214.2. Typically, a variant will include a mutated Spike gene.

In some embodiments of the genomic remainder, for example, for the live attenuated severe acute respiratory syndrome coronavirus 2 (SARS-COV-2), SARS-COV-2, SARS-COV-2 particle or SARS-COV-2 nucleic acid etc, the Spike gene (or part thereof) of the Wuhan strain can be replaced with the Spike gene of Alpha, Beta, Gamma or Delta variants. All of these can have the Wuhan isolate backbone with the only changes being in the sequence of the Spike gene.

In some embodiments, apart from the deoptimized region, a genomic remainder, or part thereof, of the live attenuated severe acute respiratory syndrome coronavirus 2 (SARS-CoV-2), SARS-COV-2, SARS-COV-2 particle or SARS-COV-2 nucleic acid etc can comprise a sequence of a genetically modified, recombinant or manufactured SARS-COV-2 genome.

In some embodiments, the chance of deattenuation to wild-type SARS-COV-2 is negligible.

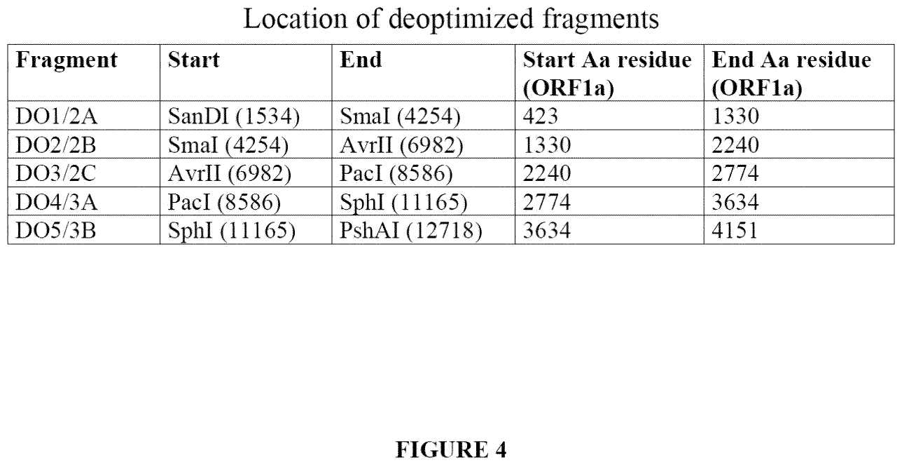

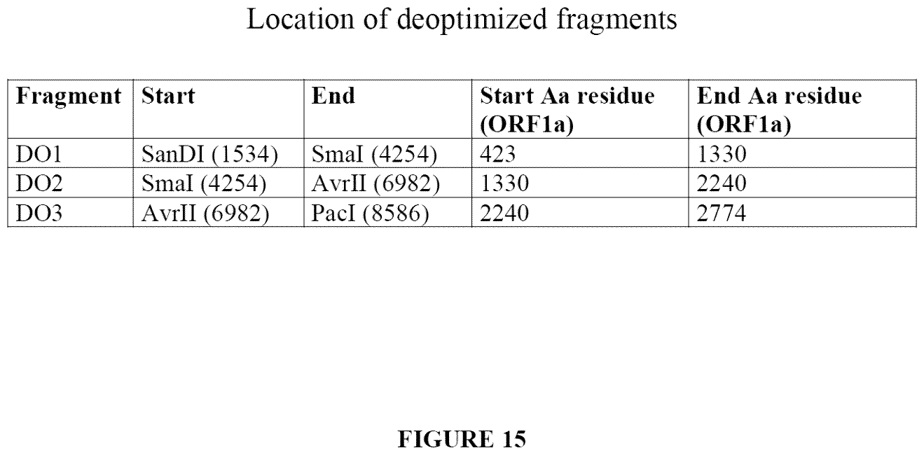

In some embodiments, a recombinant/recombined ORF1a region can be used. In some embodiments, the ORF1a region can be cleaved into at least two, three, four, five, six, seven, eight, nine, ten or more fragments. Preferably, the ORF1a region can be cleaved into at least about three fragments. These ORF1a fragments can be generated using, for example, restriction enzymes. Suitable restriction enzymes include, for example, SanDI, SmaI, AvrII, PacI, SphI and PshAI. Deoptimized fragments can be generated using gene synthesis and restriction enzyme sites as described in FIGS. 4 and 15. Other restriction enzyme sites within ORF1a can be used for generating fragments and can be identified using sequence analysis software.

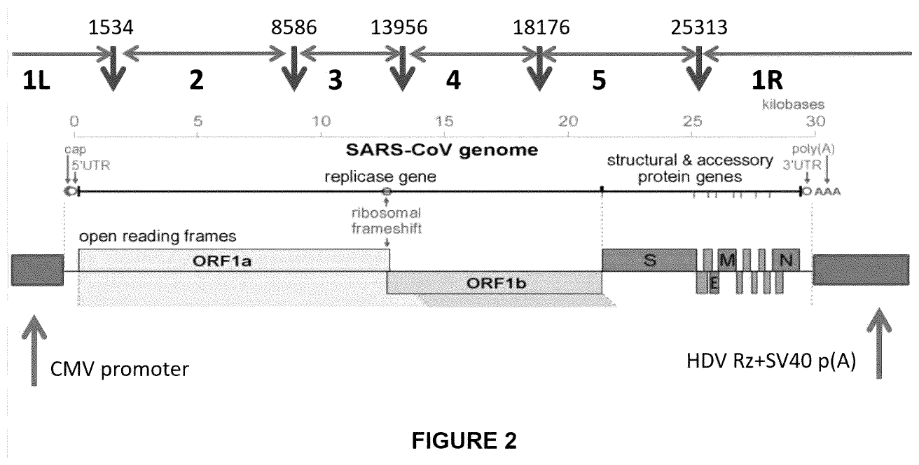

Any one or more of the ORF1a fragments can be codon deoptimized to any suitable degree. ORF1a can be codon deoptimized prior to being fragmented. Alternatively, ORF1a can be codon deoptimized after being fragmented (or sub-fragmented). A suitable fragment or sub-fragment of ORF1a or fragments or sub-fragments of ORF1a that can be codon deoptimized are shown in FIGS. 2, 3, 14 and 15.

The wild-type fragments and/codon deoptimized fragments can be assembled/ligated together in their natural five to three prime order to create a recombinant/genetically engineered ORF1a having 1, 2, 3 or more codon deoptimized fragment regions. For example, three wild-type and three codon deoptimized fragments can be assembled in different combinations to generate 7 different ORF1a fragment combinations (in addition to wildtype).

In some embodiments, the vaccine can comprise a single clone/vaccine candidate, for example, having the sequence shown in any one of SEQ ID NOs:39-68. In some embodiments, the vaccine can comprise a combination of 2, 3, 4, 5, 6, 7, 8, 9, 10, 11, 12, 13, 14. 15, 16, 17, 18, 19, 20, 21, 22, 23, 24, 25, 26, 27 or 28 clones/vaccine candidates having, for example, sequences shown in any one of SEQ ID NOs:39-68.

In some embodiments, the vaccine can comprise, for example, one or more of clones SARS-COV-2-77-1, SARS-COV-2-77-2, SARS-COV-2-77-3, SARS-COV-2-77-4, SARS-COV-2-77-5, SARS-COV-2-77-6, SARS-COV-2-77-7, SARS-COV-2-160-1, SARS-COV-2-160-2. SARS-COV-2-160-3, SARS-COV-2-160-4, SARS-COV-2-160-5, SARS-COV-2-160-6, SARS-COV-2-160-7. SARS-COV-2-4N-1 and SARS-COV-2-7N-1, or variant thereof.

In some embodiments, the vaccine can comprise one or more of clones SARS-CoV-2-4N-1 and SARS-COV-2-7N-1, or variant thereof.

As mentioned, any suitable region or regions of the SARS-COV-2 genome can be codon deoptimized. In some embodiments, the region of the SARS-COV-2 genome encoding the envelope structural protein (E protein) is codon deoptimized. (Wild type E protein is shown as SEQ ID NO:38.) In some embodiments, both E protein and ORF1a are codon deoptimized. In some embodiments, only part of E protein or all of E protein of the viral genome is codon deoptimized or different parts or sub-regions of E protein of the viral genome are codon deoptimized.

In some embodiments, the E protein putative transmembrane domain is codon deoptimized. In some embodiments, the E protein putative C-terminal protein-protein interaction motif is codon deoptimized. In some embodiments, both the E protein putative transmembrane domain and putative C-terminal protein-protein interaction motif are codon deoptimized.

In some embodiments, CD results in between about 1 and about 75 codon changes in E protein. The 1 to 75 codon change range includes all integers between 1 and 75 including 2, 3 . . . 74 codon changes. In some embodiments, some or all of the codon changes can be situated immediately next to one another, in sequence. In some embodiments, some or all of the codon changes can be spaced apart from each other such that they are not situated immediately next to one another, in sequence—e.g. 3 to 4 codon (triplet) spacing. In some embodiments, some of the codon changes can be spaced apart from each other and some of the codon changes can be situated immediately next to one another.

In some embodiments, CD results in between 1 and about 160 codon changes in E protein. The 1 to 160 codon change range includes all integers between 1 and 160 including 2. 3 . . . 159 codon changes. In some embodiments, some or all of the codon changes are of serine to TCG. Preferably there are 77 codon or 160 codon changes of serine to TCG.

In some embodiments, CD results in between 1 and about 496 codon changes in E protein. The 1 to 496 codon change range includes all integers between 1 and 496 including 2, 3 . . . 495 codon changes. In some embodiments, some of the codon changes are of proline to CCG, and preferably there are 81 proline codons deoptimized to CCG. In some embodiments, some of the codon changes are of threonine to ACG, and preferably there are 186 threonine codons deoptimized to ACG. In some embodiments, some of the codon changes are of isoleucine to ATA, and preferably there are 82 isoleucine codons deoptimized to ATA. In some embodiments, some of the codon changes are of alanine to GCG, and preferably there are 147 alanine codons deoptimized to GCG. In some embodiments, the codon changes are any combination thereof.

In some embodiments, CD results in between 1 and about 546 codon changes in E protein. The 1 to 546 codon change range includes all integers between 1 and 546 including 2. 3 . . . 545 codon changes. In some embodiments, some of the codon changes are of proline to CCG, and preferably there are 82 proline codons deoptimized to CCG. In some embodiments, some of the codon changes are of threonine to ACG, and preferably there are 178 threonine codons deoptimized to ACG. In some embodiments, some of the codon changes are of isoleucine to ATA, and preferably there are 44 isoleucine codons deoptimized to ATA. In some embodiments, some of the codon changes are of alanine to GCG, and preferably there are 147 alanine codons deoptimized to GCG. In some embodiments, some of the codon changes are of arginine to CGT, and preferably there are 40 arginine codons deoptimized to CGT. In some embodiments, some of the codon changes are of glycine to GGT, and preferably there are 41 glycine codons deoptimized to GGT. In some embodiments, some of the codon changes are of glutamine to CAA, and preferably there are 14 glutamine codons deoptimized to CAA. In some embodiments, the codon changes are any combination thereof.

In some embodiments, CD of E protein results in reduced neurovirulence.

The live attenuated SARS-COV-2, SARS-COV-2, SARS-COV-2 particle or SARS-CoV-2 nucleic acid comprising a partly codon deoptimized SARS-COV-2 genome can be of any suitable form and can be prepared in any suitable way. Likewise, the recombinant, isolated or substantially purified nucleic acid comprising a partly codon deoptimized SARS-COV-2 genome or partly codon deoptimized region thereof can be prepared in any suitable way. Such techniques are described elsewhere in this specification (e.g. see below), the entire contents of which are incorporated herein by way of reference.

Likewise, a vaccine, vaccination dose, pharmaceutical preparation or immunogenic composition comprising the above can be of any suitable form and can be prepared in any suitable way. Such techniques are described elsewhere in this specification, the entire contents of which are incorporated herein by way of reference.

In addition to a live attenuated SARS-COV-2 vaccine, pharmaceutical preparation or immunogenic composition, the present invention encompasses SARS-COV-2 particles, nucleic acid and genetic vaccines that comprise a partly codon deoptimized SARS-COV-2 genome in the form of a nucleic acid. The nucleic acid can be DNA or RNA that is self-replicating/self-amplifying once used for vaccination. The nucleic acid can relate to the SARS-CoV-2 genome or SARS-COV-2 anti-genome. The nucleic acid can relate to positive-sense genomic RNA, negative-strand genomic RNA, or cDNA encoding the SARS-COV-2 genome. Such techniques are described in the following references, the entire contents of which are incorporated herein by way of cross-reference: (Karl Ljungberg & Peter Liljeström (2015) Self-replicating alphavirus RNA vaccines, Expert Review of Vaccines, 14:2, 177-194, DOI: 10.1586/14760584.2015.965690; Rodríguez-Gascón A, del Pozo-Rodríguez A, Solinís MA (2014) Development of nucleic acid vaccines: use of self-amplifying RNA in lipid nanoparticles. Int J Nanomedicine. 9: 1833-1843; US 2014/0112979 A1.

The vaccine, pharmaceutical preparation or immunogenic composition can comprise live virus or temporarily inactivated virus, provided that it is self-replicating/self-amplifying after vaccination. If inactivated, it can be inactivated in any suitable way (e.g. using high or low temperatures, radiation or chemically).

The vaccine, pharmaceutical preparation or immunogenic composition can comprise a delivery system or carrier or aid, and these can be of any suitable form and can be prepared in any suitable way. Suitable examples include a plasmid, genetic construct or vector to assist with self-replication/self-amplification, an RNA nanocarrier for RNA delivery, and lipid-based formulations for delivery, including liposomes, nanoemulsions and solid lipid nanoparticles.

In some embodiments, the vaccine can be prepared by way of passing SARS-COV-2 through a filter, such as a 0.22 μm hydrophilic PVDF membrane or hydrophilic Polyethersulfone membrane.

Manufacturing a vaccine can comprise growing/propagating the virus in Vero cells or Vero E6 cells. These cells can be used in large-scale bioreactors. However, it may be possible to grow the virus using other cell types, tissue culture methods and mediums.

In some embodiments, the vaccine can be stored long term and remain viable at a temperature of between about 2° C. and about −80° C. (including all 1 degree increments between 2 and −80, including 1, 0, −1, −2 . . . −79). By “long-term” it is meant that the vaccine can remain viable for at least 30, 31, 32, 33, 34, 35, 36, 37, 38, 39, 40, 41, 42, 43, 44, 45, 46, 47, 48, 49, 50, 51, 52, 53, 54, 55, 56, 57, 58, 59 or 60 days. In some embodiments, it is possible that the vaccine can remain viable for more than 60 days. In some embodiments, it is possible that the vaccine can remain viable for 1 year, 2 years or more, especially if freeze dried and stored at 2-8 degrees Celsius.

The live attenuated virus can be in the form of an isolate. The isolate may comprise cells, such as mammalian or other types of cells—e.g. Vero cells.

The method of preventing the subject from contracting a viral infection, treating a subject having a viral infection, or reducing the severity of a viral disease, can be carried out in any suitable way.

A ‘SARS-COV-2-like virus’ as used herein refers to a virus closely related to SARS-COV-2. SARS-COV-2 natural variants, SARS COV 1, MERS-COV and other human coronaviruses, especially betacoronaviruses, may be closely related viruses.

A ‘SARS-COV-2-like infection’ as used herein refers to an infection caused by a virus closely related to SARS-COV-2.

A ‘SARS-COV-2-like disease’ as used herein refers to a disease caused by a virus closely related to SARS-COV-2, including betacoronaviruses. Severe acute respiratory syndrome 1 (SARS 1) and Middle-East respiratory syndrome (MERS) are examples of SARS-CoV-2-like diseases.

The vaccine, live attenuated virus, pharmaceutical preparation and immunogenic composition (described hereafter as “the compositions”) can be administered independently, either systemically or locally, by any method standard in the art, for example, subcutaneously, intravenously, parenterally, intraperitoneally, intradermally, intramuscularly, topically, orally or nasally. The compositions are preferably administered subcutaneously.

The compositions can comprise conventional non-toxic, physiologically or pharmaceutically acceptable ingredients or vehicles suitable for the method of administration and are well known to an individual having ordinary skill in this art. If required, the compositions can, for example, comprise an adjuvant. The adjuvant can be, for example, an aluminium salt (e.g. aluminium hydroxide), monophosphoryl lipid A, or emulsion of water and oil (e.g. MF59). In some embodiments, no adjuvant is required. The term “pharmaceutically acceptable carrier” as used herein is intended to include diluents such as saline and aqueous buffer solutions. The compositions can be in aqueous, lyophilized, freeze-dried or frozen form. If freeze-dried, the composition can be reconstituted with diluent.

A variety of devices are known in the art for delivery of the compositions including, but not limited to, syringe and needle injection, bifurcated needle administration, administration by intradermal patches or pumps, intradermal needle-free jet delivery (intradermal etc.), intradermal particle delivery, or aerosol powder delivery.

The compositions can be administered independently one or more times to achieve, maintain or improve upon a desired effect/result. It is well within the skill of an artisan to determine dosage or whether a suitable dosage of the composition comprises a single administered dose or multiple administered doses. An appropriate dosage depends on the subject's health, the induction of immune response and/or prevention of infection caused by the SARS-COV-2, the route of administration and the formulation used. For example, a therapeutically active amount of the compound may vary according to factors such as the disease state, age, sex, and weight of the subject, and the ability of the composition to elicit a desired response in the subject. Dosage regime may be adjusted to provide the optimum therapeutic response. For example, a subject may be administered a ‘booster’ vaccination one, two, three, four or more weeks following the initial administration. For example, a subject may be administered a titre of 10⁴ PFU attenuated virus per dose.

The vector, plasmid or genetic construct can also be prepared in any suitable way. Suitably, the genetic construct is in the form of, or comprises genetic components of, a plasmid, bacteriophage, a cosmid, a yeast or bacterial artificial chromosome as are well understood in the art. Genetic constructs may also be suitable for maintenance and propagation of the nucleic acid in bacteria or other host cells, for manipulation by recombinant DNA technology. For the purposes of protein expression, the genetic construct is an expression construct. Suitably, the expression construct comprises the one or more nucleic acids operably linked to one or more additional sequences, such as heterologous sequences, in an expression vector. An “expression vector” may be either a self-replicating extra-chromosomal vector such as a plasmid, or a vector that integrates into a host genome. By “operably linked” is meant that said additional nucleotide sequence(s) is/are positioned relative to the nucleic acid of the invention preferably to initiate, regulate or otherwise control transcription. Regulatory nucleotide sequences will generally be appropriate for the host cell or tissue where expression is required. Numerous types of appropriate expression vectors and suitable regulatory sequences are known in the art for a variety of host cells. Typically, said one or more regulatory nucleotide sequences may include, but are not limited to, promoter sequences, leader or signal sequences, ribosomal binding sites, transcriptional start and termination sequences, translational start and termination sequences, and enhancer or activator sequences. Constitutive or inducible promoters as known in the art are contemplated by the invention. The expression construct may also include an additional nucleotide sequence encoding a fusion partner (typically provided by the expression vector) so that the recombinant protein of the invention is expressed as a fusion protein. In some embodiments the genetic construct is suitable for virus production and in other embodiments for DNA vaccination of a mammal, such as a human.

The cell (mammalian or other) or isolate comprising the vector, plasmid, genetic construct or virus can be prepared in any suitable way.

Suitable protocols for carrying out one or more of the above-mentioned techniques can be found in “Current Protocols in Molecular Biology”. July 2008, JOHN WILEY AND SONS; D. M. WEIR ANDCC BLACKWELL, “Handbook Of Experimental Immunology”, vol. I-IV. 1986; JOHN E. COLIGAN, ADA M. KRUISBEEK, DAVID H. MARGULIES, ETHAN M. SHEVACH, WARREN STROBER, “Current Protocols in Immunology”, 2001, JOHN WILEY & SONS; “Immunochemical Methods In Cell And Molecular Biology”, 1987, ACADEMIC PRESS; SAMBROOK ET AL., “Molecular Cloning: A Laboratory Manual, 3d cd.,”, 2001, COLD SPRING HARBOR LABORATORY PRESS; “Vaccine Design, Methods and Protocols”, Volume 2, Vaccines for Veterinary Diseases, Sunil Thomas in Methods in Molecular Biology (2016); and, “Vaccine Design, Methods and Protocols”, Volume 1: Vaccines for Human Diseases, Sunil Thomas in Methods in Molecular Biology (2016), the entire contents of which are incorporated herein by way of reference.

Any suitable type of subject can be used. The subject can be any suitable mammal. Mammals include humans, primates, livestock and farm animals (e.g. horses, sheep and pigs), companion animals (e.g. dogs and cats), and laboratory test animals including rats, mice, rabbits, hamsters and ferrets, including transgenic animals (e.g. human ACE2 receptor transgenic mice). The subject can be a bat, pangolin or other wild animal that could be a host for the coronavirus. The subject is preferably human.

‘Nucleic acid’ as used herein includes ‘polynucleotide’, ‘oligonucleotide’, and ‘nucleic acid molecule’, and generally means a polymer of DNA or RNA, which can be single-stranded or double-stranded, synthesized or obtained (e.g. isolated and/or purified) from natural sources, which can contain natural, non-natural or altered nucleotides, and which can contain a natural, non-natural or altered internucleotide linkage, such as a phosphoroamidate linkage or a phosphorothioate linkage, instead of the phosphodiester found between the nucleotides of an unmodified oligonucleotide.

As used herein, context permitting, the term ‘recombinant’ refers to (i) molecules that are constructed outside living cells by joining natural or synthetic nucleic acid segments to nucleic acid molecules that can replicate in a living cell, or (ii) molecules that result from the replication of those described in (i) above. For purposes herein, the replication can be in vitro replication or in vivo replication. As used herein, context permitting, the term ‘recombinant’ refers to the condition of having been genetically modified. That is, a ‘recombinant virus genome’ means that the virus genome has been genetically engineered. In this sense: live attenuated SARS-COV-2 (comprising a partly codon deoptimized SARS-COV-2 genome) can be called recombinant live attenuated SARS-COV-2; SARS-COV-2 can be called recombinant SARS-COV-2; SARS-COV-2 particle can be called recombinant SARS-CoV-2 particle; and SARS-COV-2 nucleic acid can be called recombinant SARS-COV-2 nucleic acid.

The terms ‘isolated’ or ‘purified’ as used herein mean essentially free of association with other biological components/contaminants, e.g. as a naturally occurring protein that has been separated from cellular and other contaminants by the use of antibodies or other methods or as a purification product of a recombinant host cell culture. By ‘substantially the same’ or ‘substantially as shown’, it is meant that it is different yet essentially the same, differing in a minor way to make no significant practical or functional difference.

Amino acids are referred to herein interchangably by their name, IUPAC code or three letter code. See Table 1C.

TABLE 1C

| | | Three |
|---|---|---|
| | IUPAC | letter |
| Amino Acid | code | code |
| Alanine | A | Ala |
| Cysteine | C | Cys |
| Aspartic Acid | D | Asp |
| Glutamic Acid | E | Glu |
| Phenylalanine | F | Phe |
| Glycine | G | Gly |
| Histidine | H | His |
| Isoleucine | I | Ile |
| Lysine | K | Lys |
| Leucine | L | Leu |
| Methionine | M | Met |
| Asparagine | N | Asn |
| Proline | P | Pro |
| Glutamine | Q | Gln |
| Arginine | R | Arg |
| Serine | S | Ser |
| Threonine | T | Thr |
| Valine | V | Val |
| Tryptophan | W | Trp |
| Tyrosine | Y | Tyr |

Preferred embodiments of the invention are defined in the following numbered paragraphs:
1. Live attenuated severe acute respiratory syndrome coronavirus 2 (SARS-COV-2), SARS-COV-2, SARS-COV-2 particle or SARS-COV-2 nucleic acid comprising a partly codon deoptimized SARS-COV-2 genome.
2. A recombinant, isolated or substantially purified nucleic acid comprising a partly codon deoptimized SARS-COV-2 genome or partly codon deoptimized region thereof.
3. A vector (or plasmid or genetic construct) containing the nucleic acid of the second paragraph.
4. A cell or isolate containing the live attenuated SARS-COV-2, SARS-COV-2, SARS-COV-2 particle or SARS-COV-2 nucleic acid of the first paragraph, the nucleic acid of the second paragraph, or the vector (or plasmid or genetic construct) of the third paragraph.
5. A vaccine comprising the live attenuated SARS-COV-2, SARS-COV-2, SARS-CoV-2 particle or SARS-COV-2 nucleic acid of the first paragraph, the recombinant, isolated or substantially purified nucleic acid of the second paragraph, the vector (or plasmid or genetic construct) of the third paragraph, or the cell or isolate of the fourth paragraph.
6. A pharmaceutical preparation comprising the live attenuated SARS-COV-2, SARS-COV-2, SARS-COV-2 particle or SARS-COV-2 nucleic acid of the first paragraph, the recombinant, isolated or substantially purified nucleic acid of the second paragraph, the vector (or plasmid or genetic construct) of the third paragraph, or the cell or isolate of the fourth paragraph.
7. An immunogenic composition comprising the live attenuated SARS-COV-2, SARS-COV-2, SARS-COV-2 particle or SARS-COV-2 nucleic acid of the first paragraph, the recombinant, isolated or substantially purified nucleic acid of the second paragraph, the vector (or plasmid or genetic construct) of the third paragraph, or the cell or isolate of the fourth paragraph.
8. A method of: (1) vaccinating a subject; (2) prophylactically immunizing a subject against SARS-COV-2 or SARS-COV-2-like virus; (3) preventing a subject from contracting a SARS-COV-2 infection naturally or a SARS-COV-2-like infection naturally; (4) reducing the severity of a natural SARS-COV-2 disease or natural SARS-COV-2-like disease in a subject; or (5) treating a subject having a natural SARS-COV-2 infection or natural SARS-CoV-2-like infection, said method comprising the step of administering to the subject: the live attenuated SARS-COV-2, SARS-COV-2, SARS-COV-2 particle or SARS-COV-2 nucleic acid of the first paragraph; the recombinant, isolated or substantially purified nucleic acid of the second paragraph; the vector (or plasmid or genetic construct) of the third paragraph; the cell or isolate of the fourth paragraph; the vaccine of the fifth paragraph; the pharmaceutical preparation of the sixth paragraph; or the immunogenic composition of the seventh paragraph.
9. Use of: the live attenuated SARS-COV-2, SARS-COV-2, SARS-COV-2 particle or SARS-COV-2 nucleic acid of the first paragraph; the recombinant, isolated or substantially purified nucleic acid of the second paragraph; the vector (or plasmid or genetic construct) of the third paragraph; the cell or isolate of the fourth paragraph; the vaccine of the fifth paragraph; the pharmaceutical preparation of the sixth paragraph; or the immunogenic composition of the seventh paragraph, in the preparation of a medicament for: (1) vaccinating a subject; (2) prophylactically immunizing a subject against SARS-COV-2 or SARS-COV-2-like virus; (3) preventing a subject from contracting a SARS-COV-2 infection naturally or a SARS-COV-2-like infection naturally; (4) reducing the severity of a natural SARS-COV-2 disease or natural SARS-COV-2-like disease in a subject; or (5) treating a subject having a natural SARS-COV-2 infection or natural SARS-COV-2-like infection.
10. Live attenuated SARS-COV-2, SARS-COV-2, SARS-COV-2 particle or SARS-CoV-2 nucleic acid of the first paragraph; a recombinant, isolated or substantially purified nucleic acid of the second paragraph; a vector (or plasmid or genetic construct) of the third paragraph; a cell or isolate of the fourth paragraph; a vaccine of the fifth paragraph; a pharmaceutical preparation of the sixth paragraph; or an immunogenic composition of the seventh paragraph, for use in: (1) vaccinating a subject; (2) prophylactically immunizing a subject against SARS-COV-2 or SARS-COV-2-like virus; (3) preventing a subject from contracting a SARS-COV-2 infection naturally or a SARS-COV-2-like infection naturally; (4) reducing the severity of a natural SARS-COV-2 disease or natural SARS-COV-2-like disease in a subject; or (5) treating a subject having a natural SARS-COV-2 infection or natural SARS-CoV-2-like infection.
11. A method of generating a live attenuated SARS-COV-2 vaccine, SARS-COV-2, SARS-COV-2 particle or SARS-COV-2 nucleic acid, or recombinant, isolated or substantially purified nucleic acid comprising a partly codon deoptimized SARS-COV-2 genome or partly codon deoptimized region thereof, comprising the step of partly codon deoptimizing a SARS-COV-2 genome.
12. A method of preparing a vaccine comprising live attenuated SARS-COV-2, said method comprising the steps of: (1) codon deoptimizing a SARS-COV-2 genome to produce a partly codon deoptimized live attenuated SARS-COV-2; and (2) enabling the partly codon deoptimized live attenuated SARS-COV-2 to replicate.
13. A method of preparing a vaccine comprising codon deoptimized SARS-COV-2, said method comprising the steps of: optionally, (1) codon deoptimizing a SARS-COV-2 genome to produce a partly codon deoptimized live attenuated SARS-COV-2; (2) enabling the partly codon deoptimized live attenuated SARS-COV-2 to replicate; and (3) preparing a vaccine dose containing the replicated SARS-COV-2 of step (2).
14. A method of eliciting an immune response in a subject, said method including the step of administering to the subject the live attenuated SARS-COV-2, SARS-COV-2, SARS-CoV-2 particle or SARS-COV-2 nucleic acid of the first paragraph; the recombinant, isolated or substantially purified nucleic acid of the second paragraph; the vector (or plasmid or genetic construct) of the third paragraph; the cell or isolate of the fourth paragraph; the vaccine of the fifth paragraph; the pharmaceutical preparation of the sixth paragraph; or the immunogenic composition of the seventh paragraph, to thereby elicit an immune response.
15. The invention as defined in any one or more of the preceding paragraphs (context permitting), wherein the partly codon deoptimized SARS-COV-2 genome comprises or consists of changes within the ORF1a region of the virus.
16. The invention as defined in any one or more of the preceding paragraphs (context permitting), wherein the partly codon deoptimized SARS-COV-2 genome comprises or consists of changes within the ORF1a region of the virus, excluding the 5′ region and/or 3′ region of ORF1a.
17. The invention as defined in any one or more of the preceding paragraphs (context permitting), wherein the partly codon deoptimized SARS-COV-2 genome comprises or consists of changes within the ORF1a region of the virus, wherein preferably the changes comprise one or more fragments or sub-regions of ORF1a being codon deoptimized.
18. The invention as defined in any one or more of the preceding paragraphs (context permitting), wherein at least one codon for an amino acid with high codon degeneracy is changed to a synonymous codon that is used least frequently, moderately, less rarely, or rarely in the genome of Homo sapiens.
19. The invention as defined in any one or more of the preceding paragraphs (context permitting), wherein codon deoptimization results in between about 10 and about 1850 codon changes in ORF1a.
20. The invention as defined in any one or more of the preceding paragraphs (context permitting), wherein codon deoptimization results in replacement with one or more rare codons, one or more less rare codons, or one or more moderate codons, one or more CpG dinucleotides/elements, one or more UpA dinucleotides/elements, or any combination of these.
21. The invention as defined in any one or more of the preceding paragraphs (context permitting), wherein codon deoptimization results in one or more serine codons being changed, one or more proline codons being changed, one or more threonine codons being changed, one or more isoleucine codons being changed, one or more alanine codons being changed, one or more arginine codons being changed, or any combination of these.
22. The invention as defined in any one or more of the preceding paragraphs (context permitting), wherein codon deoptimization results in one or more serine codons being changed to the rare TCG codon.
23. The invention as defined in any one or more of the preceding paragraphs (context permitting), wherein codon deoptimization results in one or more proline codons being changed to the less rare CCG codon.
24. The invention as defined in any one or more of the preceding paragraphs (context permitting), wherein codon deoptimization results in one or more threonine codons being changed to the less rare ACG codon.
25. The invention as defined in any one or more of the preceding paragraphs (context permitting), wherein codon deoptimization results in one or more isoleucine codons being changed to the less rare ATA codon.
26. The invention as defined in any one or more of the preceding paragraphs (context permitting), wherein codon deoptimization results in one or more alanine codons being changed to the less rare GCG codon.
27. The invention as defined in any one or more of the preceding paragraphs (context permitting), wherein codon deoptimization results in one or more arginine codons being changed to the rare CGT codon.
28. The invention as defined in any one or more of the preceding paragraphs (context permitting), wherein the partly codon deoptimized SARS-COV-2 genome comprises the deoptimized codons of the clones/vaccine candidates as shown or as substantially shown in any one of SEQ ID NO:39-68, and as shown or as substantially shown in any one of FIGS. 8 to 12 and 22 to 25.
29. The invention as defined in any one or more of the preceding paragraphs (context permitting), wherein the partly codon deoptimized SARS-COV-2 genome comprises at least the deoptimized codons of the clones/vaccine candidates as shown or as substantially shown in any one of SEQ ID NO:39-68, and as shown or as substantially shown in any one of FIGS. 8 to 12 and 22 to 25.
30. The invention as defined in any one or more of the preceding paragraphs (context permitting), wherein the partly codon deoptimized SARS-COV-2 genome comprises fewer than the deoptimized codons of the clones/vaccine candidates as shown or as substantially shown in any one of SEQ ID NO:39-68, and as shown or as substantially shown in any one of FIGS. 8 to 12 and 22 to 25.
31. The invention as defined in any one or more of the preceding paragraphs (context permitting), wherein the partly codon deoptimized SARS-COV-2 genome comprises anywhere between about 10% and about 100% of the deoptimized codons of the clones/vaccine candidates as shown or as substantially shown in any one of SEQ ID NO:39-68, and as shown or as substantially shown in any one of FIGS. 8 to 12 and 22 to 25.
32. The invention as defined in any one or more of the preceding paragraphs (context permitting), wherein the partly codon deoptimized SARS-COV-2 genome comprises one or more codon changes as listed in Table 1b or in Table 11.
33. The invention as defined in any one or more of the preceding paragraphs (context permitting), wherein the partly codon deoptimized SARS-COV-2 genome comprises the sequence or substantially the same sequence of clone SARS-COV-2-77-1, SARS-COV-2-77-2, SARS-COV-2-77-3, SARS-COV-2-77-4, SARS-COV-2-77-5, SARS-COV-2-77-6, SARS-COV-2-77-7, SARS-COV-2-160-1, SARS-COV-2-160-2, SARS-COV-2-160-3, SARS-COV-2-160-4, SARS-COV-2-160-5, SARS-COV-2-160-6, SARS-COV-2-160-7, SARS-COV-2-4N-1 or SARS-COV-2-7N-1 or any variant thereof (eg. SARS-COV-2-4N-1-Alpha (B1.1.7), SARS-COV-2-4N-1-Beta (B1.351), SARS-COV-2-4N-1-Gamma (P1), SARS-CoV-2-4N-1-Delta, SARS-COV-2-7N-1-Alpha (B1.1.7), SARS-COV-2-7N-1-Beta (B1.351), SARS-COV-2-7N-1-Gamma (P1), or SARS-COV-2-7N-1-Delta).
34. The invention as defined in any one or more of the preceding paragraphs (context permitting), wherein the partly codon deoptimized SARS-COV-2 genome comprises the sequence of clone SARS-COV-2-4N-1 or variant thereof or clone SARS-COV-2-7N-1 or variant thereof, or substantially the same sequence as clone SARS-COV-2-4N-1 or variant thereof or clone SARS-COV-2-7N-1 or variant thereof (eg. SARS-COV-2-4N-1-Alpha (B1.1.7), SARS-COV-2-4N-1-Beta (B1.351), SARS-COV-2-4N-1-Gamma (P1), SARS-COV-2-4N-1-Delta, SARS-COV-2-7N-1-Alpha (B1.1.7), SARS-COV-2-7N-1-Beta (B1.351), SARS-COV-2-7N-1-Gamma (P1), or SARS-COV-2-7N-1-Delta).
35. The invention as defined in any one or more of the preceding paragraphs (context permitting), wherein the vaccine, the pharmaceutical preparation, or the immunogenic composition comprises freeze-dried/lyophilized infectious virus that can be reconstituted prior to administration.
36. The invention as defined in any one or more of the preceding paragraphs (context permitting), wherein the vaccine is administered by subcutaneous injection or inhalation, intranasally or orally.
37. The invention as defined in any one or more of the preceding paragraphs (context permitting), wherein the vaccine, the pharmaceutical preparation, or the immunogenic composition comprises a single clone/vaccine candidate having the sequence shown in any one of SEQ ID NOs:39-68.
38. The invention as defined in any one or more of the preceding paragraphs (context permitting), wherein the vaccine, the pharmaceutical preparation, or the immunogenic composition comprises a combination of 2, 3, 4, 5, 6, 7, 8, 9, 10, 11, 12, 13, 14, 15, 16, 17, 18, 19, 20, 21, 22, 23, 24, 25, 26, 27 or 28 clones/vaccine candidates with sequences shown in any one of SEQ ID NOs:39-68.
39. The invention as defined in any one or more of the preceding paragraphs (context permitting), wherein the vaccine, the pharmaceutical preparation, or the immunogenic composition comprises one or more of clones SARS-COV-2-77-1. SARS-COV-2-77-2. SARS-COV-2-77-3, SARS-COV-2-77-4, SARS-COV-2-77-5, SARS-COV-2-77-6, SARS-COV-2-77-7, SARS-COV-2-160-1, SARS-COV-2-160-2, SARS-COV-2-160-3, SARS-COV-2-160-4, SARS-COV-2-160-5, SARS-COV-2-160-6, SARS-COV-2-160-7, SARS-COV-2-4N-1 and SARS-COV-2-7N-1 or any variant thereof (eg. SARS-COV-2-4N-1-Alpha (B1.1.7), SARS-COV-2-4N-1-Beta (B1.351), SARS-COV-2-4N-1-Gamma (P1), SARS-COV-2-4N-1-Delta, SARS-COV-2-7N-1-Alpha (B1.1.7), SARS-COV-2-7N-1-Beta (B1.351), SARS-COV-2-7N-1-Gamma (P1), or SARS-COV-2-7N-1-Delta).
40. The invention as defined in any one or more of the preceding paragraphs (context permitting), wherein the vaccine, the pharmaceutical preparation, or the immunogenic composition comprises one or more of clones SARS-COV-2-4N-1 and SARS-COV-2-7N-1 or any variant thereof (eg. SARS-COV-2-4N-1-Alpha (B1.1.7), SARS-COV-2-4N-1-Beta (B1.351), SARS-COV-2-4N-1-Gamma (P1), SARS-COV-2-4N-1-Delta, SARS-COV-2-7N-1-Alpha (B1.1.7), SARS-COV-2-7N-1-Beta (B1.351), SARS-COV-2-7N-1-Gamma (P1), or SARS-COV-2-7N-1-Delta).
41. The invention as defined in any one or more of the preceding paragraphs (context permitting), wherein a genomic remainder has the sequence of the Wuhan strain or a natural variant thereof, or a genetically modified variant thereof.
42. The invention as defined in any one or more of the preceding paragraphs, wherein the genomic remainder has the sequence of any one of Alpha, Pango lineage B.1.1.7; Beta, Pango lineages B.1.351, B.1.351.2, B.1.351.3; Gamma, Pango lincages P.1, P.1.1, P.1.2; Delta, Pango lineages B.1.617.2, AY.1, AY.2; Eta, Pango lineage B.1.525; Iota, Pango lineage B.1.526; Kappa, Pango lineage B.1.617.1; Lambda, Pango lineage C.37; and, Pango lineages B.1.427, B.1.429, P.2, P.3, R.1, R.2, B.1.466.2, B.1.621, AV.1, B.1.1.318, B.1.1.519, AT.1, C.36.3, C.36.3.1, B.1.214.2.
43. The invention as defined in any one or more of the preceding paragraphs, wherein the vaccine, the pharmaceutical preparation, or the immunogenic composition comprises one or more of clones SARS-COV-2-4N-1-Alpha (B1.1.7), SARS-COV-2-4N-1-Beta (B1.351), SARS-COV-2-4N-1-Gamma (P1), SARS-COV-2-4N-1-Delta, SARS-COV-2-7N-1-Alpha (B1.1.7), SARS-COV-2-7N-1-Beta (B1.351), SARS-COV-2-7N-1-Gamma (P1), or SARS-COV-2-7N-1-Delta.
44. The invention as defined in any one or more of the preceding paragraphs (context permitting), wherein the envelope structural protein (E protein) is codon deoptimized or further codon deoptimized.

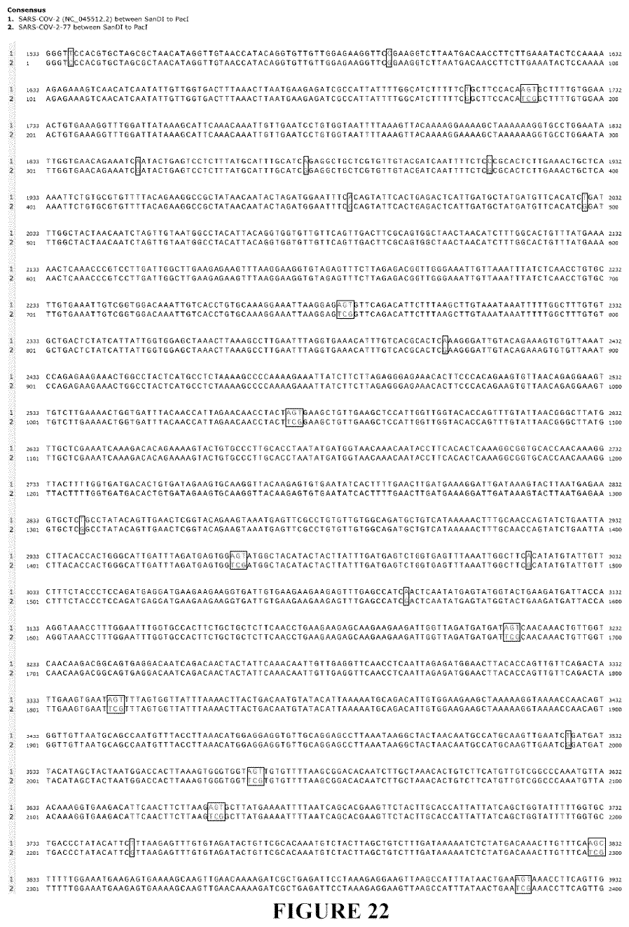

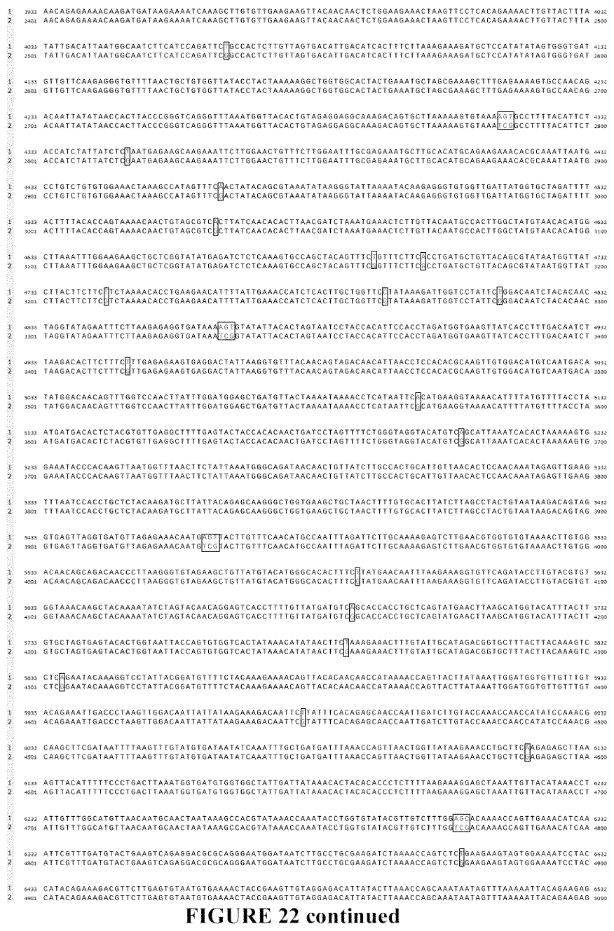

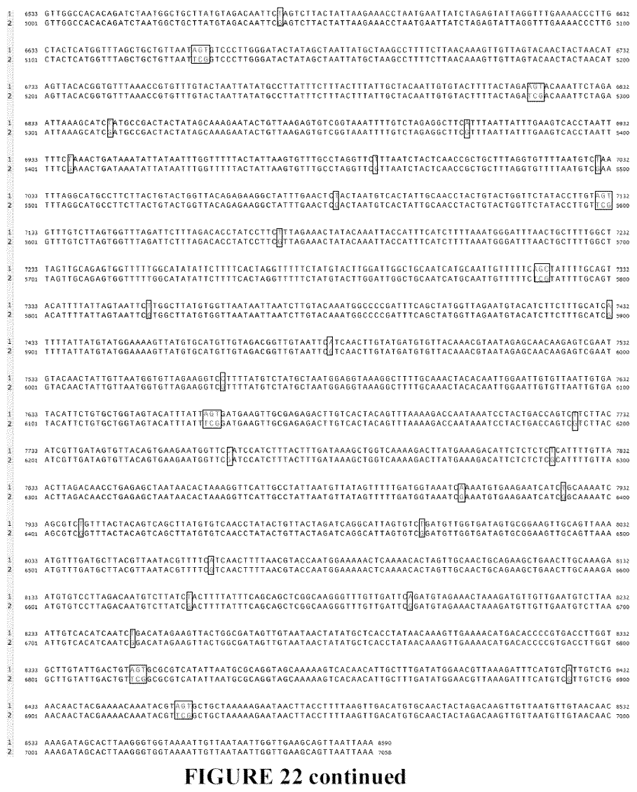

Yet further preferred embodiments of the invention are defined in the following numbered paragraphs:
1. A live attenuated severe acute respiratory syndrome coronavirus 2 (SARS-COV-2), SARS-COV-2, SARS-COV-2 particle or SARS-COV-2 nucleic acid comprising a partly codon deoptimized SARS-COV-2 genome.
2. A recombinant, isolated or substantially purified nucleic acid comprising a partly codon deoptimized SARS-COV-2 genome or partly codon deoptimized region thereof.
3. A vector, plasmid or genetic construct comprising the nucleic acid of paragraph 2.
4. A cell or isolate containing the live attenuated SARS-COV-2, SARS-COV-2, SARS-COV-2 particle or SARS-COV-2 nucleic acid of paragraph 1, the nucleic acid of paragraph 2, or the vector, plasmid or genetic construct of paragraph 3.
5. A vaccine comprising the live attenuated SARS-COV-2, SARS-COV-2, SARS-CoV-2 particle or SARS-COV-2 nucleic acid of paragraph 1, the recombinant, isolated or substantially purified nucleic acid of paragraph 2, the vector, plasmid or genetic construct of paragraph 3, or the cell or isolate of paragraph 4.
6. A pharmaceutical preparation comprising the live attenuated SARS-COV-2, SARS-COV-2, SARS-COV-2 particle or SARS-COV-2 nucleic acid of paragraph 1, the recombinant, isolated or substantially purified nucleic acid of paragraph 2, the vector, plasmid or genetic construct of paragraph 3, or the cell or isolate of paragraph 4.
7. An immunogenic composition comprising the live attenuated SARS-COV-2, SARS-COV-2, SARS-COV-2 particle or SARS-COV-2 nucleic acid of paragraph 1, the recombinant, isolated or substantially purified nucleic acid of paragraph 2, the vector, plasmid or genetic construct of paragraph 3, or the cell or isolate of the paragraph 4.
8. A method of: (1) vaccinating a subject; (2) prophylactically immunizing a subject against SARS-COV-2 or SARS-COV-2-like virus; (3) preventing a subject from contracting a SARS-COV-2 infection naturally or a SARS-COV-2-like infection naturally; (4) reducing the severity of a natural SARS-COV-2 disease or natural SARS-COV-2-like disease in a subject; (5) treating a subject having a natural SARS-COV-2 infection or natural SARS-CoV-2-like infection; or (6) eliciting an immune response in a subject, said method comprising the step of administering to the subject: the live attenuated SARS-COV-2, SARS-COV-2, SARS-COV-2 particle or SARS-COV-2 nucleic acid of paragraph 1; the recombinant, isolated or substantially purified nucleic acid of paragraph 2; the vector, plasmid or genetic construct of paragraph 3; the cell or isolate of the paragraph 4; the vaccine of paragraph 5; the pharmaceutical preparation of paragraph 6; or the immunogenic composition of paragraph 7.
9. Use of: the live attenuated SARS-COV-2, SARS-COV-2, SARS-COV-2 particle or SARS-COV-2 nucleic acid of paragraph 1; the recombinant, isolated or substantially purified nucleic acid of paragraph 2; the vector, plasmid or genetic construct of paragraph 3; the cell or isolate of paragraph 4; the vaccine of paragraph 5; the pharmaceutical preparation of paragraph 6; or the immunogenic composition of paragraph 7, in the preparation of a medicament for: (1) vaccinating a subject; (2) prophylactically immunizing a subject against SARS-COV-2 or SARS-COV-2-like virus; (3) preventing a subject from contracting a SARS-COV-2 infection naturally or a SARS-COV-2-like infection naturally; (4) reducing the severity of a natural SARS-COV-2 disease or natural SARS-COV-2-like disease in a subject; (5) treating a subject having a natural SARS-COV-2 infection or natural SARS-COV-2-like infection; or (6) eliciting an immune response in a subject.
10. A live attenuated SARS-COV-2, SARS-COV-2, SARS-COV-2 particle or SARS-COV-2 nucleic acid of paragraph 1; a recombinant, isolated or substantially purified nucleic acid of paragraph 2; a vector, plasmid or genetic construct of paragraph 3; a cell or isolate of paragraph 4; a vaccine of paragraph 5; a pharmaceutical preparation of paragraph 6; or an immunogenic composition of paragraph 7, for use in: (1) vaccinating a subject; (2) prophylactically immunizing a subject against SARS-COV-2 or SARS-COV-2-like virus; (3) preventing a subject from contracting a SARS-COV-2 infection naturally or a SARS-COV-2-like infection naturally; (4) reducing the severity of a natural SARS-COV-2 disease or natural SARS-COV-2-like disease in a subject; (5) treating a subject having a natural SARS-COV-2 infection or natural SARS-COV-2-like infection; or (6) eliciting an immune response in a subject.
11. A method of generating a live attenuated SARS-COV-2 vaccine, SARS-COV-2, SARS-COV-2 particle or SARS-COV-2 nucleic acid, or recombinant, isolated or substantially purified nucleic acid comprising a partly codon deoptimized SARS-COV-2 genome or partly codon deoptimized region thereof, comprising the step of partly codon deoptimizing a SARS-COV-2 genome.
12. A method of preparing a vaccine comprising live attenuated SARS-COV-2, said method comprising the steps of: (1) codon deoptimizing a SARS-COV-2 genome to produce a partly codon deoptimized live attenuated SARS-COV-2; and (2) enabling the partly codon deoptimized live attenuated SARS-COV-2 to replicate.
13. A method of preparing a vaccine comprising codon deoptimized SARS-COV-2, said method comprising the steps of: optionally, (1) codon deoptimizing a SARS-COV-2 genome to produce a partly codon deoptimized live attenuated SARS-COV-2; (2) enabling the partly codon deoptimized live attenuated SARS-COV-2 to replicate; and (3) preparing a vaccine dose containing the replicated SARS-COV-2 of step (2).
14. The live attenuated SARS-COV-2, SARS-COV-2, SARS-COV-2 particle or SARS-COV-2 nucleic acid of paragraph 1; the recombinant, isolated or substantially purified nucleic acid of paragraph 2; the vector, plasmid or genetic construct of paragraph 3; the cell or isolate of paragraph 4; the vaccine of paragraph 5; the pharmaceutical preparation of paragraph 6; the immunogenic composition of paragraph 7; the method of paragraph 8; the use of paragraph 9; the live attenuated SARS-COV-2, SARS-COV-2, SARS-COV-2 particle or SARS-CoV-2 nucleic acid, the recombinant, isolated or substantially purified nucleic acid, the vector, plasmid or genetic construct, the cell or isolate, the vaccine, the pharmaceutical preparation or the immunogenic composition for use in paragraph 10; the method of paragraph 11; the method of paragraph 12; or, the method of paragraph 13,
wherein the partly codon deoptimized SARS-COV-2 genome comprises or consists of codon changes within the ORF1a region of SARS-COV-2.
15. The live attenuated SARS-COV-2, SARS-COV-2, SARS-COV-2 particle or SARS-COV-2 nucleic acid of paragraph 14; the recombinant, isolated or substantially purified nucleic acid of paragraph 14; the vector, plasmid or genetic construct of paragraph 14; the cell or isolate of paragraph 14; the vaccine of paragraph 14; the pharmaceutical preparation of paragraph 14; the immunogenic composition of paragraph 14; the use of paragraph 14; the live attenuated SARS-COV-2, SARS-COV-2, SARS-COV-2 particle or SARS-COV-2 nucleic acid, the recombinant, isolated or substantially purified nucleic acid, the vector, plasmid or genetic construct, the cell or isolate, the vaccine, the pharmaceutical preparation or the immunogenic composition for use in paragraph 14; or, the method of paragraph 14,
wherein the partly codon deoptimized SARS-COV-2 genome comprises or consists of codon changes within the ORF1a region of SARS-COV-2, excluding the 5′ region of ORF1 and/or excluding the 3′ region of ORF1a corresponding to the ribosomal frameshift region; or
wherein the partly codon deoptimized SARS-COV-2 genome comprises or consists of codon changes within the ORF1a region of SARS-COV-2 corresponding to between about nucleotide position 1534 and about nucleotide position 8586 of the wild-type Wuhan SARS-CoV-2 genome.
16. The live attenuated SARS-COV-2, SARS-COV-2, SARS-COV-2 particle or SARS-COV-2 nucleic acid of paragraph 14 or 15; the recombinant, isolated or substantially purified nucleic acid of paragraph 14 or 15; the vector, plasmid or genetic construct of paragraph 14; the cell or isolate of paragraph 14 or 15; the vaccine of paragraph 14 or 15; the pharmaceutical preparation of paragraph 14 or 15; the immunogenic composition of paragraph 14 or 15; the use of paragraph 14 or 15; the live attenuated SARS-COV-2, SARS-COV-2, SARS-COV-2 particle or SARS-COV-2 nucleic acid, the recombinant, isolated or substantially purified nucleic acid, the vector, plasmid or genetic construct, the cell or isolate, the vaccine, the pharmaceutical preparation or the immunogenic composition for use in paragraph 14 or 15; or, the method of paragraph 14 or 15,
wherein the partly codon deoptimized SARS-COV-2 genome comprises or consists of codon changes within the ORF1a region of SARS-COV-2 corresponding to:
(1) between about nucleotide position 1534 and about nucleotide position 4254 of the wild-type Wuhan SARS-COV-2 genome;
(2) between about nucleotide position 4254 and about nucleotide position 6982 of the wild-type Wuhan SARS-COV-2 genome;
(3) between about nucleotide position 6982 and about nucleotide position 8586 of the wild-type Wuhan SARS-COV-2 genome;
(4) between about nucleotide position 8586 and about nucleotide position 11165 of the wild-type Wuhan SARS-COV-2 genome;
(5) between about nucleotide position 11165 and about nucleotide position 12718 of the wild-type Wuhan SARS-COV-2 genome; or
(6) any combination of (1) to (5).
17. The live attenuated SARS-COV-2, SARS-COV-2, SARS-COV-2 particle or SARS-COV-2 nucleic acid of any one of paragraphs 14-16; the recombinant, isolated or substantially purified nucleic acid of any one of paragraphs 14-16; the vector, plasmid or genetic construct of any one of paragraphs 14-16; the cell or isolate of any one of paragraphs 14-16; the vaccine of any one of paragraphs 14-16; the pharmaceutical preparation of any one of paragraphs 14-16; the immunogenic composition of any one of paragraphs 14-16; the use of any one of paragraphs 14-16; the live attenuated SARS-COV-2, SARS-COV-2, SARS-COV-2 particle or SARS-COV-2 nucleic acid, the recombinant, isolated or substantially purified nucleic acid, the vector, plasmid or genetic construct, the cell or isolate, the vaccine, the pharmaceutical preparation or the immunogenic composition for use in any one of paragraphs 14-16; or, the method of any one of paragraphs 14-16,
wherein the partly codon deoptimized SARS-COV-2 genome comprises or consists of between about 10 and about 1850 codon changes within the ORF1a region; or
wherein the partly codon deoptimized SARS-COV-2 genome comprises or consists of between about 24 and about 546 codon changes within the ORF1a region.
18. The live attenuated SARS-COV-2, SARS-COV-2, SARS-COV-2 particle or SARS-COV-2 nucleic acid of any one of paragraphs 14-17; the recombinant, isolated or substantially purified nucleic acid of any one of paragraphs 14-17; the vector, plasmid or genetic construct of any one of paragraphs 14-17; the cell or isolate of any one of paragraphs 14-17; the vaccine of any one of paragraphs 14-17; the pharmaceutical preparation of any one of paragraphs 14-17; the immunogenic composition of any one of paragraphs 14-17; the use of any one of paragraphs 14-17; the live attenuated SARS-COV-2, SARS-COV-2, SARS-COV-2 particle or SARS-COV-2 nucleic acid, the recombinant, isolated or substantially purified nucleic acid, the vector, plasmid or genetic construct, the cell or isolate, the vaccine, the pharmaceutical preparation or the immunogenic composition for use in any one of paragraphs 14-17; or, the method of any one of paragraphs 14-17,
wherein the partly codon deoptimized SARS-COV-2 genome comprises or consists of codon changes to synonymous codons that are used less frequently, moderately, less rarely, and/or rarely in the genome of Homo sapiens; or
wherein the partly codon deoptimized SARS-COV-2 genome comprises or consists of codon changes to one or more rare codons, one or more less rare codons, one or more moderate codons, one or more codons containing CG (CpG) dinucleotides, one or more codons containing UA (UpA) dinucleotides, or any combination thereof.
19. The live attenuated SARS-COV-2, SARS-COV-2, SARS-COV-2 particle or SARS-COV-2 nucleic acid of any one of paragraphs 14-18; the recombinant, isolated or substantially purified nucleic acid of any one of paragraphs 14-18; the vector, plasmid or genetic construct of any one of paragraphs 14-18; the cell or isolate of any one of paragraphs 14-18; the vaccine of any one of paragraphs 14-18; the pharmaceutical preparation of any one of paragraphs 14-18; the immunogenic composition of any one of paragraphs 14-18; the use of any one of paragraphs 14-18; the live attenuated SARS-COV-2, SARS-COV-2, SARS-COV-2 particle or SARS-COV-2 nucleic acid, the recombinant, isolated or substantially purified nucleic acid, the vector, plasmid or genetic construct, the cell or isolate, the vaccine, the pharmaceutical preparation or the immunogenic composition for use in any one of paragraphs 14-18; or, the method of any one of paragraphs 14-18,
wherein the partly codon deoptimized SARS-COV-2 genome comprises or consists of synonymous codon changes whereby one or more Ser codons are changed, one or more Arg codons are changed, one or more Thr codons are changed, one or more Pro codons are changed, one or more Val codons are changed, one or more Leu codons are changed, one or more Ala codons are changed, one or more Ile codons are changed, one or more Cys codons are changed, one or more Gly codons are changed, one or more His codons are changed, one or more Gln codons are changed, one or more Trp codons are changed, one or more Asn codons are changed, one or more Phe codons are changed, one or more Asp codons are changed, one or more Phe codons are changed, one or more Lys codons are changed, one or more Glu codons are changed, or any combination thereof.
20. The live attenuated SARS-COV-2, SARS-COV-2, SARS-COV-2 particle or SARS-COV-2 nucleic acid of any one of paragraphs 14-19; the recombinant, isolated or substantially purified nucleic acid of any one of paragraphs 14-19; the vector, plasmid or genetic construct of any one of paragraphs 14-19; the cell or isolate of any one of paragraphs 14-19; the vaccine of any one of paragraphs 14-19; the pharmaceutical preparation of any one of paragraphs 14-19; the immunogenic composition of any one of paragraphs 14-19; the use of any one of paragraphs 14-19; the live attenuated SARS-COV-2, SARS-COV-2, SARS-COV-2 particle or SARS-COV-2 nucleic acid, the recombinant, isolated or substantially purified nucleic acid, the vector, plasmid or genetic construct, the cell or isolate, the vaccine, the pharmaceutical preparation or the immunogenic composition for use in any one of paragraphs 14-19; or, the method of any one of paragraphs 14-19,
wherein the partly codon deoptimized SARS-COV-2 genome comprises or consists of synonymous codon changes whereby:
one or more Ser codons are changed to the rare TCG codon;
one or more proline codons are changed to the less rare CCG codon;
one or more threonine codons are changed to the less rare ACG codon;
one or more isoleucine codons are changed to the less rare ATA codon;
one or more alanine codons are changed to the less rare GCG codon; and/or
one or more arginine codons are changed to the rare CGT codon or less rare CGA codon.
21. The live attenuated SARS-COV-2, SARS-COV-2, SARS-COV-2 particle or SARS-COV-2 nucleic acid of any one of paragraphs 14-20; the recombinant, isolated or substantially purified nucleic acid of any one of paragraphs 14-20; the vector, plasmid or genetic construct of any one of paragraphs 14-20; the cell or isolate of any one of paragraphs 14-20; the vaccine of any one of paragraphs 14-20; the pharmaceutical preparation of any one of paragraphs 14-20; the immunogenic composition of any one of paragraphs 14-20; the use of any one of paragraphs 14-20; the live attenuated SARS-COV-2, SARS-COV-2, SARS-COV-2 particle or SARS-COV-2 nucleic acid, the recombinant, isolated or substantially purified nucleic acid, the vector, plasmid or genetic construct, the cell or isolate, the vaccine, the pharmaceutical preparation or the immunogenic composition for use in any one of paragraphs 14-20; or, the method of any one of paragraphs 14-20,
wherein the partly codon deoptimized SARS-COV-2 genome comprises or consists of codon changes within the ORF1a region of SARS-COV-2 comprising:
a deoptimized nucleotide sequence as shown or substantially as shown in any one of SEQ ID NO:33-37;
a deoptimized nucleotide sequence as shown or substantially as shown in any one of SEQ ID NO:39-68;
a deoptimized nucleotide sequence as shown or substantially as shown in any one of FIGS. 22 to 25;
a deoptimized nucleotide sequence as shown in any one of SEQ ID NO:33-37 but with up to 10% fewer or up to 10% (including 1, 2, 3, 4, 5, 6, 7, 8, 9 or 10%) more codon changes than shown;
a deoptimized nucleotide sequence as shown in any one of SEQ ID NO:39-68 but with up to 10% fewer or up to 10% (including 1, 2, 3, 4, 5, 6, 7, 8, 9 or 10%) more codon changes than shown; or
a deoptimized nucleotide sequence as shown in any one of FIGS. 22 to 25 but with up to 10% fewer or up to 10% (including 1, 2, 3, 4, 5, 6, 7, 8, 9 or 10%) more codon changes than shown.
22. The live attenuated SARS-COV-2, SARS-COV-2, SARS-COV-2 particle or SARS-COV-2 nucleic acid of any one of paragraphs 14-20; the recombinant, isolated or substantially purified nucleic acid of any one of paragraphs 14-20; the vector, plasmid or genetic construct of any one of paragraphs 14-20; the cell or isolate of any one of paragraphs 14-20; the vaccine of any one of paragraphs 14-20; the pharmaceutical preparation of any one of paragraphs 14-20; the immunogenic composition of any one of paragraphs 14-20; the use of any one of paragraphs 14-20; the live attenuated SARS-COV-2, SARS-COV-2, SARS-COV-2 particle or SARS-COV-2 nucleic acid, the recombinant, isolated or substantially purified nucleic acid, the vector, plasmid or genetic construct, the cell or isolate, the vaccine, the pharmaceutical preparation or the immunogenic composition for use in any one of paragraphs 14-20; or, the method of any one of paragraphs 14-20,
wherein the partly codon deoptimized SARS-COV-2 genome comprises or consists of codon changes as listed in Table 1b, either individually or in combination with each other, or in Table 11, either individually or in combination with each other.
23. The live attenuated SARS-COV-2, SARS-COV-2, SARS-COV-2 particle or SARS-COV-2 nucleic acid of any one of paragraphs 14-20; the recombinant, isolated or substantially purified nucleic acid of any one of paragraphs 14-20; the vector, plasmid or genetic construct of any one of paragraphs 14-20; the cell or isolate of any one of paragraphs 14-20; the vaccine of any one of paragraphs 14-20; the pharmaceutical preparation of any one of paragraphs 14-20; the immunogenic composition of any one of paragraphs 14-20; the use of any one of paragraphs 14-20; the live attenuated SARS-COV-2, SARS-COV-2, SARS-COV-2 particle or SARS-COV-2 nucleic acid, the recombinant, isolated or substantially purified nucleic acid, the vector, plasmid or genetic construct, the cell or isolate, the vaccine, the pharmaceutical preparation or the immunogenic composition for use in any one of paragraphs 14-20; or, the method of any one of paragraphs 14-20,
wherein the partly codon deoptimized SARS-COV-2 genome comprises the nucleotide sequence or substantially the same nucleotide sequence as clone SARS-COV-2-77-1 (or the sequence of SEQ ID NO:39), SARS-COV-2-77-2 (or the sequence of SEQ ID NO:40), SARS-COV-2-77-3 (or the sequence of SEQ ID NO:41), SARS-COV-2-77-4 (or the sequence of SEQ ID NO:42), SARS-COV-2-77-5 (or the sequence of SEQ ID NO:43), SARS-COV-2-77-6 (or the sequence of SEQ ID NO:44), SARS-COV-2-77-7 (or the sequence of SEQ ID NO:45), SARS-COV-2-160-1 (or the sequence of SEQ ID NO:46), SARS-COV-2-160-2 (or the sequence of SEQ ID NO:47), SARS-COV-2-160-3 (or the sequence of SEQ ID NO:48), SARS-COV-2-160-4 (or the sequence of SEQ ID NO:49), SARS-COV-2-160-5 (or the sequence of SEQ ID NO:50), SARS-COV-2-160-6 (or the sequence of SEQ ID NO:51), SARS-COV-2-160-7 (or the sequence of SEQ ID NO:52), SARS-COV-2-4N-1 (or the sequence of SEQ ID NO:53) or SARS-COV-2-7N-1 (or the sequence of SEQ ID NO:60), or any variant thereof (eg. SARS-COV-2-4N-1-Alpha (B1.1.7), SARS-COV-2-4N-1-Beta (B1.351), SARS-CoV-2-4N-1-Gamma (P1), SARS-COV-2-4N-1-Delta, SARS-COV-2-7N-1-Alpha (B1.1.7), SARS-COV-2-7N-1-Beta (B1.351), SARS-COV-2-7N-1-Gamma (P1), or SARS-COV-2-7N-1-Delta).
24. The live attenuated SARS-COV-2, SARS-COV-2, SARS-COV-2 particle or SARS-COV-2 nucleic acid of any one of paragraphs 14-20; the recombinant, isolated or substantially purified nucleic acid of any one of paragraphs 14-20; the vector, plasmid or genetic construct of any one of paragraphs 14-20; the cell or isolate of any one of paragraphs 14-20; the vaccine of any one of paragraphs 14-20; the pharmaceutical preparation of any one of paragraphs 14-20; the immunogenic composition of any one of paragraphs 14-20; the use of any one of paragraphs 14-20; the live attenuated SARS-COV-2, SARS-COV-2, SARS-COV-2 particle or SARS-COV-2 nucleic acid, the recombinant, isolated or substantially purified nucleic acid, the vector, plasmid or genetic construct, the cell or isolate, the vaccine, the pharmaceutical preparation or the immunogenic composition for use in any one of paragraphs 14-20; or, the method of any one of paragraphs 14-20,
wherein the partly codon deoptimized SARS-COV-2 genome comprises the nucleotide sequence of clone SARS-COV-2-7N-1, or substantially the same nucleotide sequence as clone SARS-COV-2-7N-1, or any variant thereof (eg. SARS-COV-2-7N-1-Alpha (B1.1.7), SARS-COV-2-7N-1-Beta (B1.351), SARS-COV-2-7N-1-Gamma (P1), or SARS-CoV-2-7N-1-Delta).
25. A vaccine comprising live attenuated SARS-COV-2, SARS-COV-2, SARS-CoV-2 particle or SARS-COV-2 nucleic acid comprising a partly codon deoptimized SARS-CoV-2 genome, wherein the partly codon deoptimized SARS-COV-2 genome comprises the nucleotide sequence of clone SARS-COV-2-7N-1 or the sequence of SEQ ID NO:60, or substantially the same nucleotide sequence as clone SARS-COV-2-7N-1 or the sequence of SEQ ID NO:60, or any variant thereof (eg. SARS-COV-2-7N-1-Alpha (B1.1.7), SARS-COV-2-7N-1-Beta (B1.351), SARS-COV-2-7N-1-Gamma (P1), or SARS-COV-2-7N-1-Delta).
26. The live attenuated SARS-COV-2, SARS-COV-2, SARS-COV-2 particle or SARS-COV-2 nucleic acid of any one of paragraphs 14-20; the recombinant, isolated or substantially purified nucleic acid of any one of paragraphs 14-20; the vector, plasmid or genetic construct of any one of paragraphs 14-20; the cell or isolate of any one of paragraphs 14-20; the vaccine of any one of paragraphs 14-20; the pharmaceutical preparation of any one of paragraphs 14-20; the immunogenic composition of any one of paragraphs 14-20; the use of any one of paragraphs 14-20; the live attenuated SARS-COV-2, SARS-COV-2, SARS-COV-2 particle or SARS-COV-2 nucleic acid, the recombinant, isolated or substantially purified nucleic acid, the vector, plasmid or genetic construct, the cell or isolate, the vaccine, the pharmaceutical preparation or the immunogenic composition for use in any one of paragraphs 14-20; or, the method of any one of paragraphs 14-20,
wherein a genomic remainder has the sequence of the Wuhan strain or a natural variant thereof, or a genetically modified variant thereof, such as the sequence of any one of Alpha, Pango lineage B.1.1.7; Beta, Pango lineages B.1.351, B.1.351.2, B.1.351.3; Gamma, Pango lineages P.1, P.1.1, P.1.2; Delta, Pango lineages B.1.617.2, AY.1, AY.2; Eta, Pango lineage B.1.525; Iota, Pango lineage B.1.526; Kappa, Pango lineage B.1.617.1; Lambda, Pango lineage C.37; and, Pango lincages B.1.427, B.1.429, P.2, P.3, R.1, R.2, B.1.466.2, B.1.621, AV.1, B.1.1.318, B.1.1.519, AT.1, C.36.3, C.36.3.1, B.1.214.2, or
wherein the vaccine, the pharmaceutical preparation, or the immunogenic composition comprises one or more of clones SARS-COV-2-4N-1-Alpha (B1.1.7), SARS-CoV-2-4N-1-Beta (B1.351), SARS-COV-2-4N-1-Gamma (P1), SARS-COV-2-4N-1-Delta, SARS-COV-2-7N-1-Alpha (B1.1.7), SARS-COV-2-7N-1-Beta (B1.351), SARS-COV-2-7N-1-Gamma (P1), or SARS-COV-2-7N-1-Delta.

Any of the features described herein can be combined in any combination with any one or more of the other features described herein within the scope of the invention.

### EXAMPLES

### Example 1—Construction of First-Generation Codon-Deoptimized SARS-COV-2 Vaccines

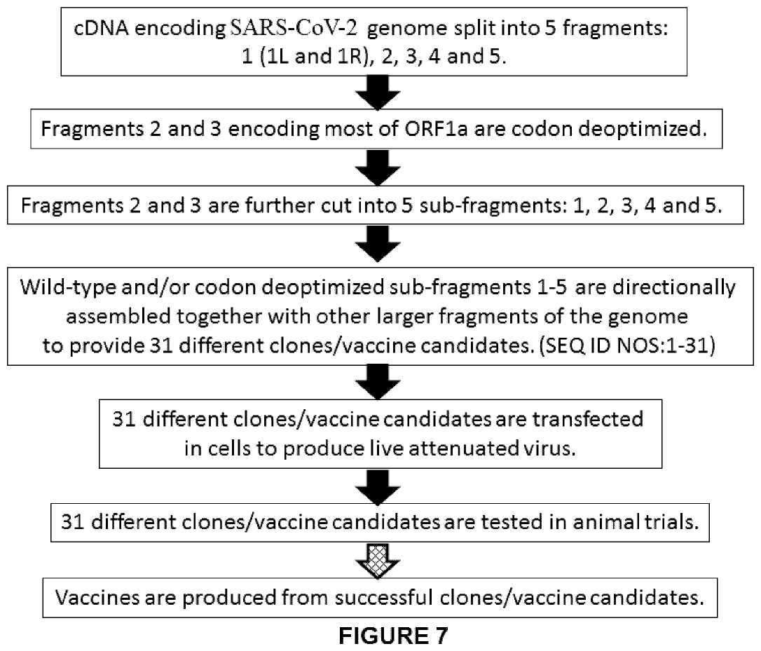

The experimental steps described in Examples 1-5 are summarised in the flowchart of FIG. 7.

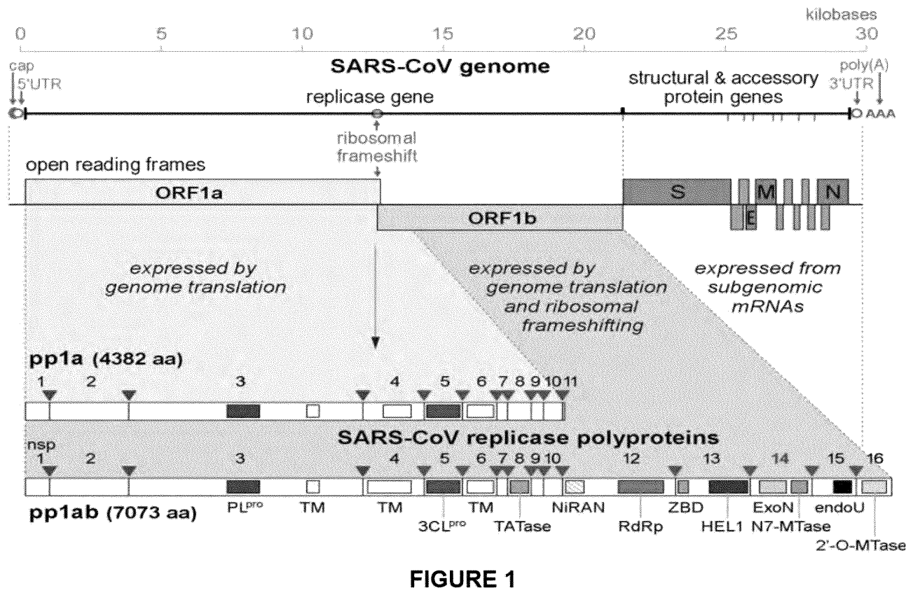

We are using codon deoptimization technology to make multiple mutations in the non-structural proteins (ORF1a region, N-terminal part of replicase) of SARS-COV-2, representing the virus isolate from Wuhan. See FIG. 1.

The ORF1a region was chosen because its deoptimization automatically results in the reduction of ORF1b expression as well while at the same time there is no change in the ratio of ORF1a/ORF1b products.

Regions required for known RNA based replication and expression (e.g. packaging, frame shift) are protected or excluded from deoptimization as this may hamper vital functions of the virus. Proteins expressed via subgenomic RNAs were considered as bad targets for deoptimization due to possible misfolding of protein (e.g. Spike-protein) and possibility of compensation of translation defect by increase of corresponding subgenomic RNA synthesis. The packaging signal of coronaviruses is outside ORF1a (in the 3′ of ORF1b), so it is not affected by this approach. The region encoding major antigens and structural protein has a complex expression pattern, which is a characteristic of the order Nidovirales. Codon deoptimization in this region is a possibility, but is not the first option as this region may not tolerate such modifications.

We are using a compact strategy (deoptimized codons in regions close to each other and not scattered over a large region) as this is technically the most straightforward approach.

We have designed clones/vaccine candidates to allow for substitution of the Spike-protein region, which is the main viral antigen. For design of deoptimized fragments, we know from our previous experience with Zika virus (Mutso M, Saul S, Rausalu K, et al. Reverse genetic system, genetically stable reporter viruses and packaged subgenomic replicon based on a Brazilian Zika virus isolate. J Gen Virol. 2017; 98(11):2712-2724. doi: 10.1099/jgv.0.000938) that deoptimization of 35% of the viral genome resulted in severely attenuated virus. Based on this experience, for SARS-COV-2 the deoptimized region should be approximately 12 kbp. We have prepared clones/vaccine candidates with deoptimized regions of about 2, 4, 6, 8 and 12 kb regions in length.

In contrast to an optimization process, which can be done using free software or online tools, there is no publicly available program for CD. Therefore, it was done manually. Every codon in ORF1a was analyzed in terms of its usage frequency in Homo sapiens. If the codon was frequent it was manually changed to a synonymous but the less used one. For instance, amino acid Leucine (Leu) can be encoded by six different codons with the following frequencies: UUA—15%, UUG—12%, CUU—12%, CUC—10%, CUA—5%, and CUG —46%. If the Leu codon in the original sequence was represented by highly abundant CUG (46%), it was changed to rare CUA (5%). Some codons were left unchanged: Methionine (Met) and Tryptophan (Trp) as both of them are encoded by only one codon; and, Asparagine (Asn) and Aspartic acid (Asn) as their codons are used at almost the same frequency.

The positions of nucleotides changed due to the deoptimization of ORF1a are seen in SEQ ID NOS: 1-31 as well as in the FIGS. 8-12.

### Example 2—Generation of First Generation SARS-COV-2 Constructs

See FIGS. 1-4, FIG. 3 in particular. The cDNA encoding the virus genome was split into 5 fragments: 1 (shown as 1L and 1R), 2, 3, 4 and 5. The fragments were obtained from GenScript, and are flanked by unique restriction sites: SanD1 (position 1524), PacI (position 8586), Mlu1 (position 13956), Bsu36I (position 18176) and BamHI (position 25313) which allowed ordered assembly of a full construct using the two methods described below in Example 3.

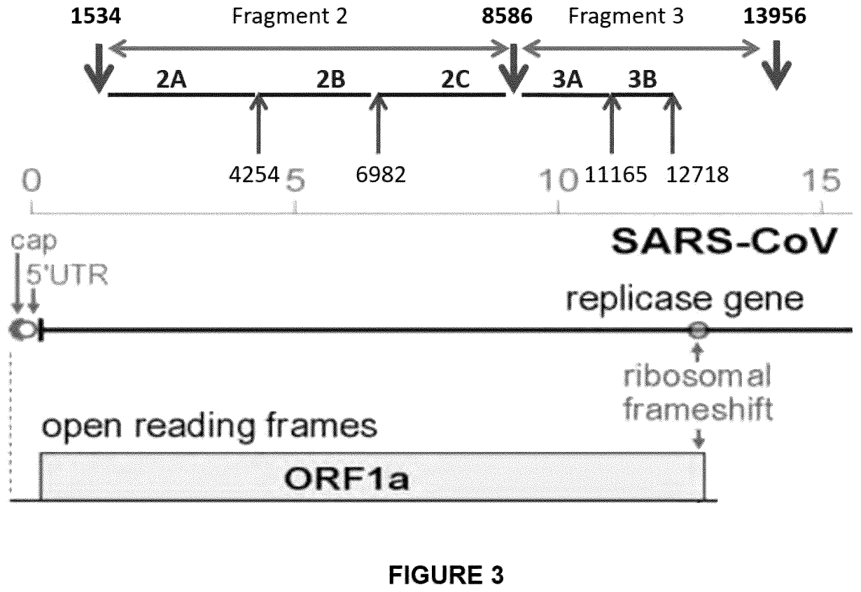

The ORF1a region was selected for codon deoptimization, but excluding the 5′ and 3′ regions (ribosomal frameshift region). Fragment 2 and the 5′ region of fragment 3 were codon deoptimized. See FIG. 3. Combined, the length of the deoptimized region is 11,186 bp/nucleotides long.

As it is not known how much deoptimization the virus can tolerate and how much is necessary for attenuation, the ORF1a region was split into 5 sub-fragments. Deoptimized and wild-type/non-deoptimized sub-fragments alike can be directionally joined/assembled in different combinations using enzymes SmaII (position 4254), AvrII (position 6982), PacI (position 8586), SphI (position 11165) and PshAI (position 12718) cleavage sites. See FIGS. 3 and 4. From five wildtype (‘W’) and five deoptimized (‘D’) sub-fragments, one can generate 31 different combinations (in addition to wild-type) from sub-fragments 2A, 2B, 2C, 3A and 3B. The 31 recombinant clones/vaccine candidates that were generated are described below. ‘D’ denotes deoptimized, and ‘W’ denotes wildtype and therefore not deoptimized.
Clone pCCI-4K-SARS-COV-2-DDDDD. All five sub-fragments (2A, 2B, 2C, 3A, 3B) were deoptimized.
Clone pCCI-4K-SARS-COV-2-DDDDW. The first four sub-fragments (2A, 2B, 2C, 3A) were deoptimized.
Clone pCCI-4K-SARS-COV-2-DDDWD. Sub-fragments one, two, three, and five (2A, 2B, 2C. 3B) were deoptimized.
Clone pCCI-4K-SARS-COV-2-DDDWW. The first three sub-fragments (2A, 2B, 2C) were deoptimized.
Clone pCCI-4K-SARS-COV-2-DDWDD. Sub-fragments one, two, four, and five (2A, 2B, 3A, 3B) were deoptimized.
Clone pCCI-4K-SARS-COV-2-DDWDW. Sub-fragments one, two, and four (2A, 2B, 3A) were deoptimized.
Clone pCCI-4K-SARS-COV-2-DDWWD, Sub-fragments one, two, and five (2A, 2B, 3B) were deoptimized.
Clone pCCI-4K-SARS-COV-2-DDWWW. The first two sub-fragments (2A, 2B) were deoptimized.
Clone pCCI-4K-SARS-COV-2-DWDDD. The first, third, fourth, and fifth sub-fragments (2A, 2C, 3A, 3B) were deoptimized.
Clone pCCI-4K-SARS-COV-2-DWDDW. The first, third, and fourth sub-fragments (2A, 2C, 3A) were deoptimized.
Clone pCCI-4K-SARS-COV-2-DWDWD. The first, third, and fifth sub-fragments (2A, 2C, 3B) were deoptimized.
Clone pCCI-4K-SARS-COV-2-DWDWW. The first and third sub-fragments (2A, 2C) were deoptimized.
Clone pCCI-4K-SARS-COV-2-DWWDD. The first, fourth, and fifth sub-fragments (2A, 3A, 3B) were deoptimized.
Clone pCCI-4K-SARS-COV-2-DWWDW. The first and fourth sub-fragments (2A, 3A) were deoptimized.
Clone pCCI-4K-SARS-COV-2-DWWWD. The first and fifth sub-fragments (2A, 3B) were deoptimized.
Clone pCCI-4K-SARS-COV-2-DWWWW. The first sub-fragment (2A) was deoptimized.
Clone pCCI-4K-SARS-COV-2-WDDDW. The second, third, and fourth sub-fragments (2B, 2C, 3A) were deoptimized.
Clone pCCI-4K-SARS-COV-2-WDDWD. The second, third, and fifth sub-fragments (2B, 2C, 3B) were deoptimized.
Clone pCCI-4K-SARS-COV-2-WDDWW. The second and third sub-fragments (2B, 2C) were deoptimized.
Clone pCCI-4K-SARS-COV-2-WDWDD. The second, fourth, and fifth sub-fragments (2B, 3A, 3B) were deoptimized.
Clone pCCI-4K-SARS-COV-2-WDWDW. The second and fourth sub-fragments (2B, 3A) were deoptimized.
Clone pCCI-4K-SARS-COV-2-WDWWD. The second and fifth sub-fragments (2B, 3B) were deoptimized.
Clone pCCI-4K-SARS-COV-2-WDWWW. The second sub-fragment (2B) was deoptimized.
Clone pCCI-4K-SARS-COV-2-WWDDD. The last three sub-fragments (2C, 3A, 3B) were deoptimized.
Clone pCCI-4K-SARS-COV-2-WWDDW. The third and fourth sub-fragments (2C, 3A) were deoptimized.
Clone pCCI-4K-SARS-COV-2-WWDWD. The third and fifth sub-fragments (2C, 3B) were deoptimized.
Clone pCCI-4K-SARS-COV-2-WWDWW. The third sub-fragment (2C) was deoptimized.
Clone pCCI-4K-SARS-COV-2-WWWDD. The last two sub-fragments (3A, 3B) were deoptimized.
Clone pCCI-4K-SARS-COV-2-WWWDW. The fourth sub-fragment (3A) was deoptimized.
Clone pCCI-4K-SARS-COV-2-WWWWD. The last sub-fragment (3B) was deoptimized.
Clone pCCI-4K-SARS-COV-2-WDDDD. The last four sub-fragments (2B, 2C, 3A, 3B) were deoptimized.
Clone pCCI-4K-SARS-COV-2. No sub-fragment was deoptimized.

### Example 3—Transfection Strategy for Obtaining Infectious First Generation Virus or Vaccine Candidates

See FIG. 2. Five fragments of the SARS-COV-2 genome (containing the 5 sub-fragments of ORF1a) were either (1) ligated within a bacterial artificial chromosome (BAC) vector pCC1-4K to form a genetic construct and expressed to obtain infectious virus or vaccine candidates, or (2) five fragments of the SARS-COV-2 genome (containing the 5 sub-fragments) were ligated together without cloning and expressed to obtain infectious virus or vaccine candidates. For (1), cytomegalovirus (CMV) promoter was placed at the 5′ end of SARS-COV-2 clone and hepatitis delta virus ribozyme (HDV Rz) and simian virus 40 terminator (SV40 p(A)) were placed at the 3′ end. These elements are needed for transcription in transfected cells and to rescue infectious virus and vaccine candidates from cDNA plasmid.

That is, we are using two ways to generate infectious virus or vaccine candidates: i) assemble all 5 sub-fragments into a single clone/genetic construct/vector (containing all fragments of the SARS-COV-2 genome) and use it for transfection (this is the preferred option); or, ii) ligate all 5 sub-fragments into a single clone (containing all fragments of the SARS-CoV-2 genome) and use the ligated product for transfection without cloning (this is the backup option). For option i) a full-length infectious clone of SARS-COV-2 was assembled in a bacterial artificial chromosome as previously described for SARS-COV-1 (Enjuanes L, Zuñiga S, Castaño-Rodriguez C, Gutierrez-Alvarez J, Canton J, Sola I. Molecular Basis of Coronavirus Virulence and Vaccine Development. Adv Virus Res. 2016; 96:245-286. doi: 10.1016/bs.aivir.2016.08.003) and for Zika virus (Mutso M. Saul S, Rausalu K. et al. Reverse genetic system, genetically stable reporter viruses and packaged subgenomic replicon based on a Brazilian Zika virus isolate. J Gen Virol. 2017; 98(11):2712-2724. doi: 10.1099/jgv.0.000938). For option ii) a split system as described by Scobey and colleagues (Scobey T, Yount B L, Sims A C, et al. Reverse genetics with a full-length infectious cDNA of the Middle East respiratory syndrome coronavirus. Proc Natl Acad Sci USA. 2013; 110(40): 16157-16162. doi: 10.1073/pnas. 1311542110) was used.

Minimising the passaging (viral production in cells) of mutant virus will reduce the chances of reversions. Genetic stability testing can be carried out, for example, by testing the phenotype of the mutant virus after 10 rounds of passage in vitro. Sequencing also will demonstrate the base changes in the virus genome over this time. We have inserted multiple mutations into the vaccine candidate, which means that the chance of complete reversion is negligible.

### Example 4—Test results

The deoptimized SARS-COV-2 infectious first generation vaccine candidate clones of the earlier Example were respectively transfected into BHK21 cells using lipofectamine LTX according to manufacturer's instructions. The BHK21 cells were cultured overnight at 37° C. with 5% CO₂ for 1 hour and then transferred to Vero E6 cells. However, no viruses could be rescued from any of the deoptimized infectious clones, suggesting the infectious clones were over deoptimized. For this reason, further vaccine CD candidates were constructed.

### Example 5—Further Construction of Second Generation SARS-COV-2 Vaccine Candidates Using Codon-Deoptimized Technology

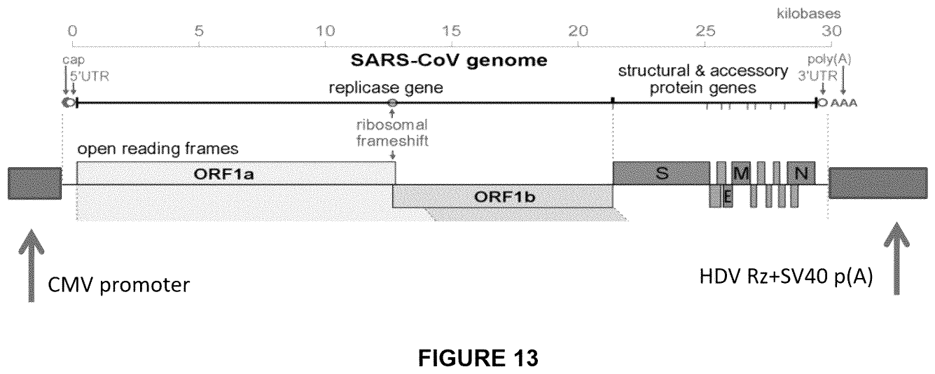

The experimental results of Example 4 showed that the virus cannot tolerate a certain degree or type of deoptimization. In view of the failure of these first generation candidates, further second generation clones/vaccine candidates were constructed and tested using four different CD strategies. See FIGS. 13-16. The positions of nucleotides changed due to the deoptimization of ORF1a are seen in SEQ ID NOS:39-68 as well as in FIGS. 22 to 25.

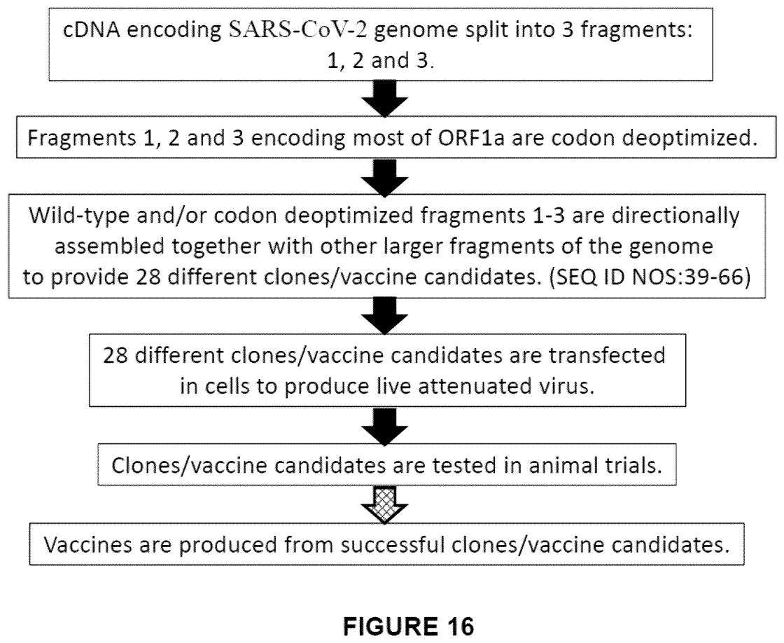

The experimental steps described in the following Examples are summarised in the flowchart of FIG. 16.

### Example 6—Generation of Second Generation SARS-COV-2 Constructs

See FIGS. 13 to 15. The cDNA encoding the virus genome was split into 3 fragments: 1, 2 and 3. The fragments were obtained from GenScript, and are flanked by unique restriction sites: SanDI (position 1534), SmaI (position 4254), AvrII (position 6982) and PacI (position 8586) which allowed ordered assembly of a full construct using the two methods described below.

The ORF1a region was selected for codon deoptimization, but excluding the 5′ and 3′ regions (ribosomal frameshift region).

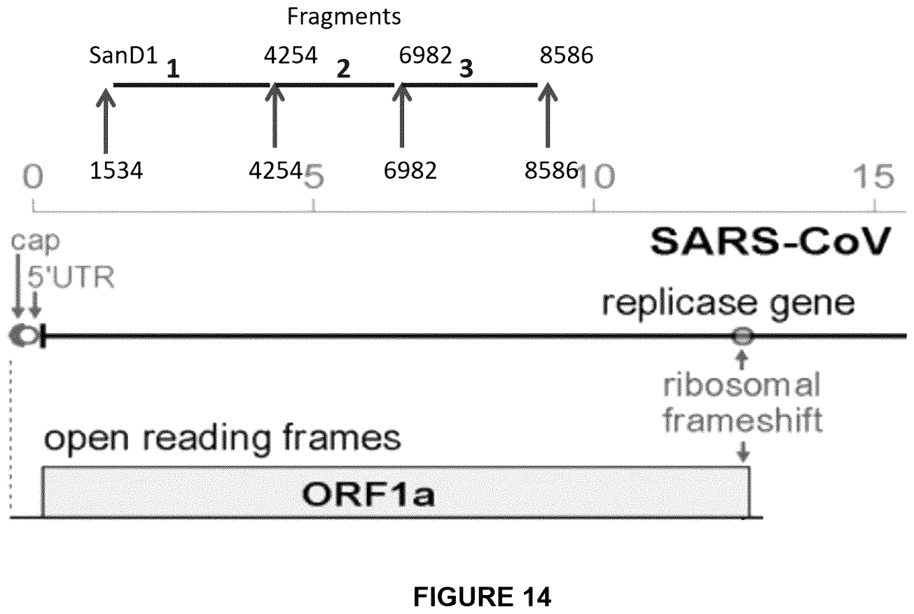

Deoptimized and wild-type/non-deoptimized fragments alike can be directionally joined/assembled in different combinations using enzymes SmaI (position 4254) and AvrII (position 6982) cleavage sites. See FIGS. 14 and 15. From three wildtype (‘W’) and three deoptimized (‘D’) fragments, one can generate 7 different combinations (in addition to wild-type).

### Strategy 1—Generation of Construct SARS-COV-2-77

This deoptimization strategy was used to bring in the rarest codon in Homo species, and increase the CpG element that was shown to be an important immunoregulator for RNA virus immune response.

About half of the serine amino acids present in the deoptimization region were targeted to replace with the rare serine codon TCG. 77 (of 160) codons of serine were selected to be deoptimized to TCG. TCG codons have a CpG element that is known to be an important immunoregulator for RNA virus immune response. Thus, the deoptimization of serine to TCG also increased the frequency of the CpG element in the fragment.

This resulted in the generation of 7 recombinant clones/vaccine candidates:
Clone SARS-COV-2-77-1 (‘77-1’): Variation 1: deoptimization between AvrII to PacI.
Clone SARS-COV-2-77-2 (‘77-2’): Variation 2: deoptimization between SmaI to AvrII.
Clone SARS-COV-2-77-3 (‘77-3’): Variation 3: deoptimization between SanDI to SmaI.
Clone SARS-COV-2-77-4 (‘77-4’): Variation 4: deoptimization between SanDI to AvrII.
Clone SARS-COV-2-77-5 (‘77-5’): Variation 5: deoptimization between SanDI to SmaI and AvrII to PacI.
Clone SARS-COV-2-77-6 (‘77-6’): Variation 6: deoptimization between SmaI to PacI.
Clone SARS-COV-2-77-7 (‘77-7’): Variation 7: deoptimization between SanDI to PacI.
Tentatively the best vaccine candidate using this strategy is SARS-COV-2-77-7 (‘77-7’) (SEQ ID NO:45). Fragments 1, 2 and 3 of SARS-COV-2-77-7 have been deoptimized.

### Strategy 2—Generation of Construct SARS-COV-2-160

This deoptimization strategy was used to bring in the rarest codon in Homo species, and increase the CpG element that was shown to be an important immunoregulator for RNA virus immune response.

All of the serine amino acids present in the deoptimization region were targeted to replace with the rare codon TCG. 160 codons of serine were selected to be deoptimized to TCG.

This resulted in the generation of 7 recombinant clones/vaccine candidates:
SARS-COV-2-160-1 (‘160-1’): Variation 1: deoptimization between AvrII to PacI.
SARS-COV-2-160-2 (‘160-2’): Variation 2: deoptimization between SmaI to AvrII.
SARS-COV-2-160-3 (‘160-3’): Variation 3: deoptimization between SanDI to SmaI.
SARS-COV-2-160-4 (‘160-4’): Variation 4: deoptimization between SanDI to AvrII.
SARS-COV-2-160-5 (‘160-5’): Variation 5: deoptimization between SanDI to SmaI and AvrII to PacI.
SARS-COV-2-160-6 (‘160-6’): Variation 6: deoptimization between SmaI to PacI.
SARS-COV-2-160-7 (‘160-7’): Variation 7: deoptimization between SanDI to PacI.
Tentatively the best vaccine candidate using this strategy is SARS-COV-2-160-7 (‘160-7’) (SEQ ID NO:52). SARS-COV-2-160-7 has CD in fragments 1, 2 and 3.

### Strategy 3—Generation of Construct SARS-COV-2-4N

This deoptimization strategy was used to bring in the less rare codons in Homo species, and increase the CpG element that was shown to be important immunoregulators for RNA virus immune response.

The amino acids isoleucine, proline, threonine and alanine present in the deoptimization region were targeted to replace with less rare codons. 81 codons for proline were selected to be deoptimized to CCG. 186 codons for threonine were selected to be deoptimized to ACG. 82 codons for isoleucine were selected to be deoptimized to ATA. 147 codons for alanine were selected to be deoptimized to GCG.

If all 3 fragments were deoptimized there would be a total of 496 codon changes. This resulted in the generation of 7 recombinant clones/vaccine candidates:
SARS-COV-2-4N-1 (‘4N-1’): Variation 1: deoptimization between AvrII to PacI.
SARS-COV-2-4N-2 (‘4N-2’): Variation 2: deoptimization between SmaI to AvrII.
SARS-COV-2-4N-3 (‘4N-3’): Variation 3: deoptimization between SanDI to SmaI.
SARS-COV-2-4N-4 (‘4N-4’): Variation 4: deoptimization between SanDI to AvrII.
SARS-COV-2-4N-5 (‘4N-5’): Variation 5: deoptimization between SanDI to SmaI and AvrII to PacI.
SARS-COV-2-4N-6 (‘4N-6’): Variation 6: deoptimization between SmaI to PacI.
SARS-COV-2-4N-7 (‘4N-7’): Variation 7: deoptimization between SanDI to PacI.

However, only one candidate could be rescued using this strategy, being SARS-CoV-2-4N-1 (‘4N-1’) (SEQ ID NO:53) The remaining 6 were dead clones. Only fragment 3 of SARS-COV-2-4N-1 has been deoptimized, there were 97 codon changes.

### Strategy 4—Generation of Construct SARS-COV-2-7N

This deoptimization strategy was used to selectively bring in less rare codons in Homo species, and increase the CpG element that was shown to be an important immunoregulator for RNA virus immune response.

The amino acids isoleucine, proline, threonine, alanine, arginine, glycine and glutamine present in the deoptimization region were targeted to replace with moderate codons. 82 codons for proline were selected to be deoptimized to CCG. 178 codons for threonine were selected to be deoptimized to ACG. 44 codons for isoleucine were selected to be deoptimized to ATA. 147 codons for alanine were selected to be deoptimized to GCG. 40 codons of arginine were selected to be deoptimized to CGT. 41 codons of glycine were selected to be deoptimized to GGT. 14 codons of glutamine were selected to be deoptimized to CAA.

If all 3 fragments were deoptimized there would be a total of 546 codon changes. This resulted in the generation of 7 recombinant clones/vaccine candidates:
SARS-COV-2-7N-1 (‘7N-1’): Variation 1: deoptimization between AvrII to PacI.
SARS-COV-2-7N-2 (‘7N-2’): Variation 2: deoptimization between SmaI to AvrII.
SARS-COV-2-7N-3 (‘7N-3’): Variation 3: deoptimization between SanDI to SmaI.
SARS-COV-2-7N-4 (‘7N-4’): Variation 4: deoptimization between SanDI to AvrII.
SARS-COV-2-7N-5 (‘7N-5’): Variation 5: deoptimization between SanDI to SmaI and AvrII to PacI.
SARS-COV-2-7N-6 (‘7N-6’): Variation 6: deoptimization between SmaI to PacI.
SARS-COV-2-7N-7 (‘7N-7’): Variation 7: deoptimization between SanDI to PacI.

However, only one candidate could be rescued using this strategy, being SARS-CoV-2-7N-1 (‘7N-1’) (SEQ ID NO:60). The remaining 6 were dead clones. Only fragment 3 of SARS-COV-2-7N-1 has been deoptimized; there being 97 codon changes.

### Summary of CD Ranges:

SARS-COV-2-77 group: minimum codon replacement: SARS-COV-2-77-3 (deoptimization in fragment 1) has 24 Ser codon replacements; maximum codon replacement: SARS-COV-2-77-7 (deoptimization in fragment 1+2+3) has 77 Ser codon replacements.
SARS-COV-2-160 group: minimum codon replacement: SARS-COV-2-160-3 (deoptimization in fragment 1) has 48 Ser codon replacements; maximum codon replacement: SARS-COV-2-160-7 (deoptimization in fragment 1+2+3) has 160 Ser codon replacements.
SARS-COV-2-4N group: minimum codon replacement: SARS-COV-2-4N-1 (deoptimization in fragment 3) has 97 codon replacements; maximum codon replacement: SARS-COV-2-4N-7 (deoptimization in fragment 1+2+3) has 496 codon replacements.
SARS-COV-2-7N group: minimum codon replacement: SARS-COV-2-7N-1 (deoptimization in fragment 3) has 97 codon replacements; maximum codon replacement: SARS-COV-2-7N-7 (deoptimization in fragment 1+2+3) has 546 codon replacements.

The clones were prepared as generally depicted in FIGS. 13 to 15. Three fragments of the SARS-COV-2 genome were ligated within a bacterial artificial chromosome (BAC) vector pCC1-4K to create a genetic construct and expressed to obtain infectious virus or vaccine candidates. A cytomegalovirus (CMV) promoter was placed at the 5′ end of SARS-CoV-2 clone and hepatitis delta virus ribozyme (HDV Rz) and simian virus 40 terminator (SV40 p(A)) were placed at the 3′ end. These elements are needed for transcription in transfected cells and to rescue infectious virus and vaccine candidates from cDNA plasmid.

The full-length infectious clone of SARS-COV-2 was assembled in a bacterial artificial chromosome as previously described for SARS-COV-1 (Enjuanes L, Zuñiga S. Castaño-Rodriguez C, Gutierrez-Alvarez J, Canton J, Sola I. Molecular Basis of Coronavirus Virulence and Vaccine Development. Adv Virus Res. 2016; 96:245-286. doi: 10.1016/bs.aivir.2016.08.003) and for Zika virus (Mutso M, Saul S, Rausalu K, et al. Reverse genetic system, genetically stable reporter viruses and packaged subgenomic replicon based on a Brazilian Zika virus isolate. J Gen Virol. 2017; 98(11):2712-2724. doi: 10.1099/jgv.0.000938). Alternatively, a split system as described by Scobey and colleagues (Scobey T. Yount B L, Sims A C, et al. Reverse genetics with a full-length infectious cDNA of the Middle East respiratory syndrome coronavirus. Proc Natl Acad Sci USA. 2013; 110(40): 16157-16162. doi: 10.1073/pnas.1311542110) was used.

### Example 7—Transformation and Purification of Second Generation SARS-COV-2 Infectious Clones

This Example describes the transformation and purification of SARS-COV-2 infectious clones from EPI300 bacterial cells (TransforMax™ EPI300™ E. coli).

### Part I: SARS-COV-2 Plasmid Transformation

Prepare 250 μl of SOC medium without antibiotic for each transformation to be performed. Maintain the medium at room temperature. Pre-chill 1.5 ml tubes on ice/at 4° C. and heat a water bath to 42° C. Thaw TransforMax EPI300 Chemically Competent E. coli cells on ice. Mix by gentle tapping. Use the cells immediately. Transfer 1-5 μl of DNA and 50 μl of cells to a pre-chilled 1.5 ml tube and incubate on ice for 30 minutes. Transfer the tubes to the water bath at 42° C. and heat shock for 30 seconds. Transfer the cells back to ice and cool for 2 minutes. Add 250 μl of SOC medium to each tube. Allow cells to recover by incubating at 37° C. for 60 minutes in a shaking incubator at 220-230 rpm. Plate the cells on LB agar plate with 12.5 μg/ml chloramphenicol and culture overnight at 37° C. Pick single E. coli colony from the plate and culture in LB medium with 12.5 μg/ml chloramphenicol overnight at 37° C. in a shaking incubator at 220-230 rpm. Preserve the transformed E. coli culture by mixing with sterile glycerol to obtain a final concentration of 20% glycerol. Store glycerol stocks at −80° C.

### Part II: Purification of SARS-COV-2 Infectious Clone

Inoculate 0.5 ml of E. coli stock in 250 ml SOY medium and culture overnight at 37° C. in a shaking incubator at 220-230 rpm. Add 750 ml of fresh SOY medium with 12.5 μg/ml chloramphenicol and 1 ml of the Copy Control Induction Solution. Incubate the culture at 37° C. in a shaking incubator with vigorous shaking for 5 hours. Collect cells by centrifugation at 2000 g for 10 min at 4° C. Resuspend the cells in 100 ml Resuspension Buffer RES-EF. Add 100 ml Lysis Buffer LYS-EF, mix carefully by inverting the tube for 4-5 times, incubate for 5 min at room temperature. Add 100 ml Neutralization Buffer NEU-EF, mix thoroughly by inverting the tube for 4-5 times. Centrifuge at 5000 g for 5 min at 4° C. to pellet cell debris. Filter the supernatant through 70 μm mesh cell strainer. Add 300 ml isopropanol to the filtered supernatant and mix well. Centrifuge at 6000 g for 15 min at 4° C. Discard the supernatant. Treat the white pellet as bacterial pellet and follow purification method according the instructions of NucleoBond Xtra Midi EF, Midi kit for endotoxin-free plasmid DNA.

Twenty-eight recombinant clones/vaccine candidates were constructed (SEQ ID NOs:39-66) and 16 recombinant clones/vaccine candidates were rescued. These were: SARS-COV-2-77-1. SARS-COV-2-77-2, SARS-COV-2-77-3, SARS-COV-2-77-4, SARS-COV-2-77-5, SARS-COV-2-77-6, SARS-COV-2-77-7 (‘Vaccine 77-7’), SARS-COV-2-160-1, SARS-COV-2-160-2, SARS-COV-2-160-3. SARS-COV-2-160-4 (‘Vaccine 160-4’), SARS-COV-2-160-5, SARS-COV-2-160-6, SARS-COV-2-160-7 (‘Vaccine 160-7’), SARS-COV-2-4N-1 (‘Vaccine 4N-1’) and SARS-COV-2-7N-1 (‘Vaccine 7N-1’).

### Example 8—Generation of Severe Acute Respiratory Syndrome Coronavirus 2 (SARS-COV-2) from Full Length DNA Infectious Second Generation Clones

The Example describes the generation of passage 0 (zero) SARS-COV-2 stocks from a full-length DNA infectious clone as described in Example 7.

### Day 1—Plating Cells

Seed BHK cells in a T25 flask(s) at ˜1.5-2×10⁶ cells in DMEM growth medium (DMEM+5% FCS) and incubate overnight at 37° C. with 5% CO₂.

### Day 2—Transfection

Measure the concentration of the DNA infectious clone plasmid on a Nanodrop spectrophotometer instrument. Approximately 10 μg of DNA is sufficient to perform the transfection. Set up tubes with 5-10 ml of F10SC disinfectant for liquid waste and pipette tip decontamination. In a sterile 1.5 ml tube dilute 20 μl Lipofectamine LTX Reagent in 500 μl Opti-MEM medium. In a separate sterile 1.5 ml tube dilute 10 μl PLUS Reagent in 500 μl Opti-MEM medium. Add 10 μg DNA into the tube with diluted PLUS reagent and mix gently. Add the diluted Lipofectamine LTX to the tube with diluted PLUS reagent/DNA and mix gently. Incubate for 20 minutes at room temperature. After incubation, remove the media from the BHK flask(s). Add the Lipofectamine LTX/PLUS reagent/DNA mixture drop-wise to the flask of BHK cells (˜1 ml). Add 4 ml DMEM transfection medium (DMEM+1% FCS) to the flask(s) and mix gently. Incubate the flask(s) at 37° C. with 5% CO₂ for 24 hours. Seed Vero E6 cells in a T25 flask(s) at ˜1.5-2×10⁶ cells in DMEM growth medium (DMEM+5% FCS) and incubate overnight at 37° C. with 5% CO₂.

### Day 3 Onwards—Propagation & Harvest of Passage 0 Virus

Discard the media from the Vero E6 cells. Flush/scrape the transfected BHK cells from the flask and directly transfer the cell/media suspension into the flask of Vero E6 cells. Incubate the Vero E6/BHK cells at 37° C. with 5% CO₂. Monitor the cells for cytopathic effect (CPE) daily. Once sufficient signs of CPE are evident (for wild type virus this is usually between 48-72 hours post-transfer of BHK cells onto Vero E6 cells) freeze the flask(s) for 1 hr-overnight. Thaw frozen cell culture flask(s) and collect the thawed cell suspension(s) into a 15 ml tube(s). Centrifuge cell suspension(s) at ˜2000 g for 5 min to pellet the cell debris. Collect clarified supernatant(s) and filter through a 0.22 μm syringe filters and aliquot into sterile 2 mL screw-cap cryo tubes. Transfer viral aliquot cryo tubes into 5 ml tubes (samples are now double contained). Store virus aliquots at −80° C. until ready to perform SARS-COV-2 plaque assay.

### Propagation of Passage 1+ Virus

Thaw an aliquot of passage 0 virus. Remove the media from a T75 flask(s) of Vero E6 cells with ˜7-8×10⁶ cells and wash once with sterile PBS. Infect Vero E6 cells with passage 0 virus at MOI 0.1-1 diluted in 3-5 ml serum free DMEM. Incubate Vero E6 flask at 37° C. with 5% CO₂ for 1 hour with periodic rocking to ensure the cells are in contact with the infection mixture. After 1 hour top up the Vero E6 flask with 3-5 ml DMEM+2% FCS and incubate at 37° C. with 5% CO₂. Monitor the cells for cytopathic effect (CPE) daily. Once sufficient signs of CPE are evident freeze the flask(s) for 1 hr-overnight. Thaw frozen cell culture flask(s) and collect the thawed cell suspension(s) into a 15 ml tube(s). Centrifuge cell suspension(s) at ˜2000 g for 5 min to pellet the cell debris. Collect clarified supernatant(s) and filter through a 0.22 μm syringe filters and aliquot into sterile 2 mL screw-cap cryo tubes. Transfer viral aliquot cryo tubes into 5 ml tubes (samples are now double contained). Store virus aliquots at −80° C. until ready to perform SARS-COV-2 plaque assay.

### Results

### Growth of Clones

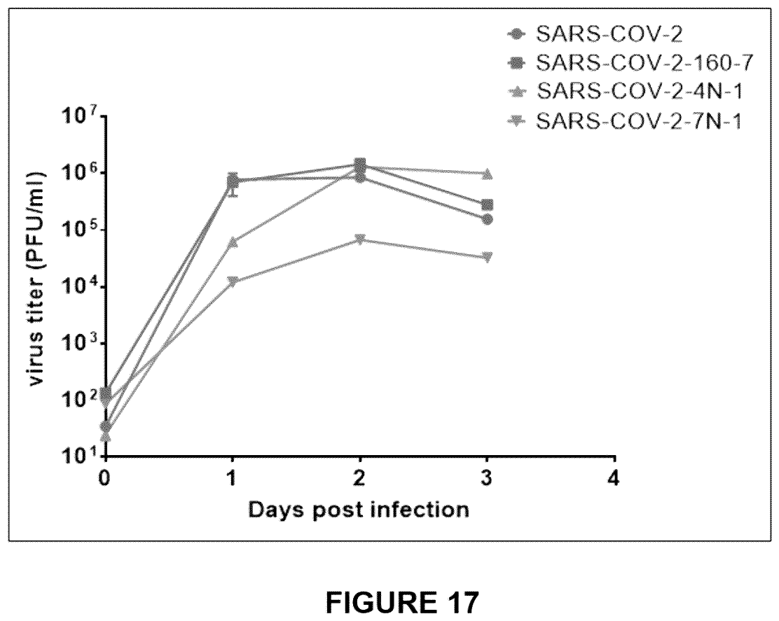

As shown in FIG. 17, SARS-COV-2 and SARS-COV-2-160-7 both grew to a similar titer (at approx. 7.5×10⁵ PFU/ml) at day 1 (24h) post infection, which were significantly higher than the titers of SARS-COV-2-4N-1 (approx. 6×10⁴ PFU/ml) and SARS-COV-2-7N-1 (approx. 1×10⁴ PFU/ml).

At day 2 (48h) post infection, SARS-COV-2, SARS-COV-2-160-7 and SARS-COV-2-4N-1 all grew to approx. 1×10⁶ PFU/ml, while SARS-COV-2-7N-1 reached approx. 6× 10⁴ PFU/ml.

At day 3 (72h) post infection, all the four strains showed reduced titer compared to day 2 post infection, which may be due to the excessive cell death.

### CPE Day 1 Post Infection

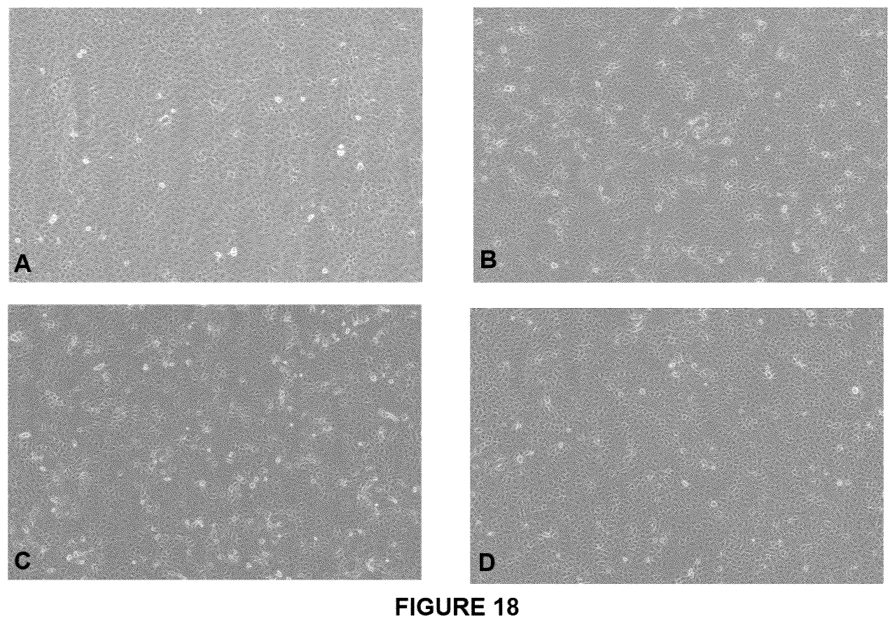

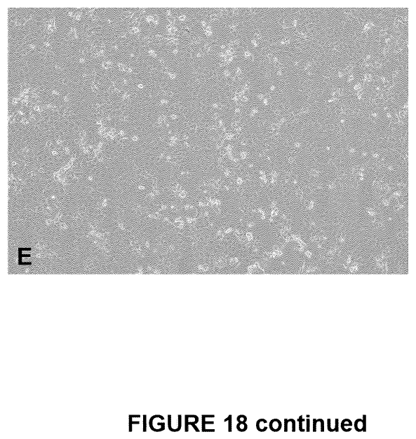

Mock: no sign of CPE (FIG. 18A); SARS-COV-2: early sign of CPE (FIG. 18B); SARS-COV-2-160-7: early sign of CPE (FIG. 18C); SARS-COV-2-4N-1: no sign of CPE (FIG. 18D); and SARS-COV-2-7N-1: no sign of CPE (FIG. 18E).

### CPE Day 2 Post Infection

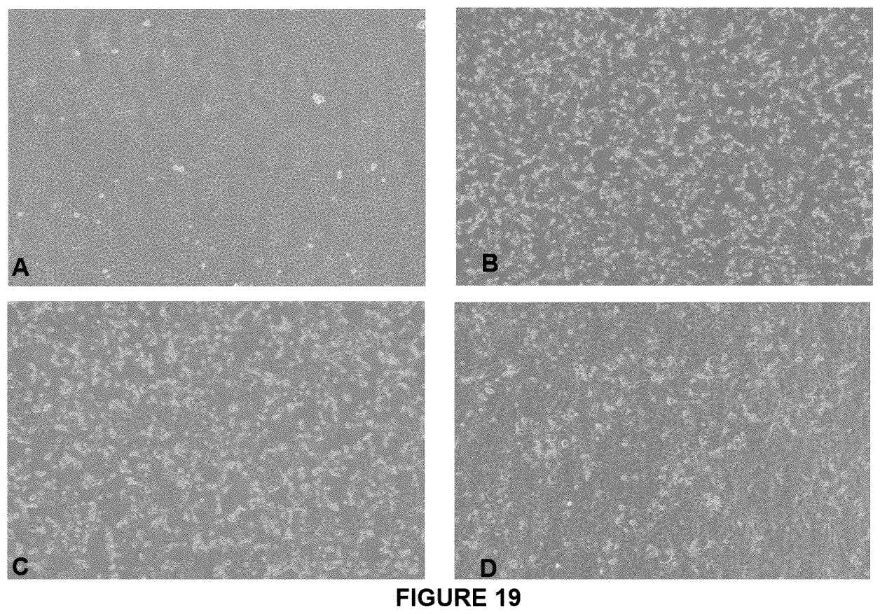

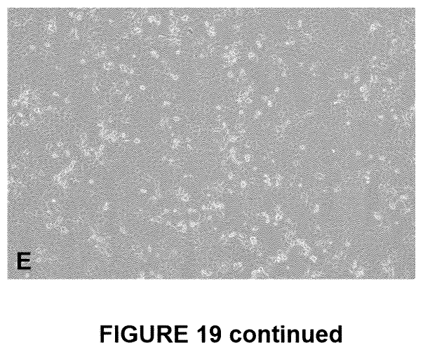

Mock: no sign of CPE (FIG. 19A); SARS-COV-2: 80-90% CPE (FIG. 19B); SARS-COV-2-160-7: 80-90% CPE (FIG. 19C); SARS-COV-2-4N-1-7 Day 2 post infection: 5-10% CPE (FIG. 19D); and SARS-COV-2-7N-1-7 Day 2 post infection: 5-10% CPE (FIG. 19E).

### CPE Day 3 Post Infection

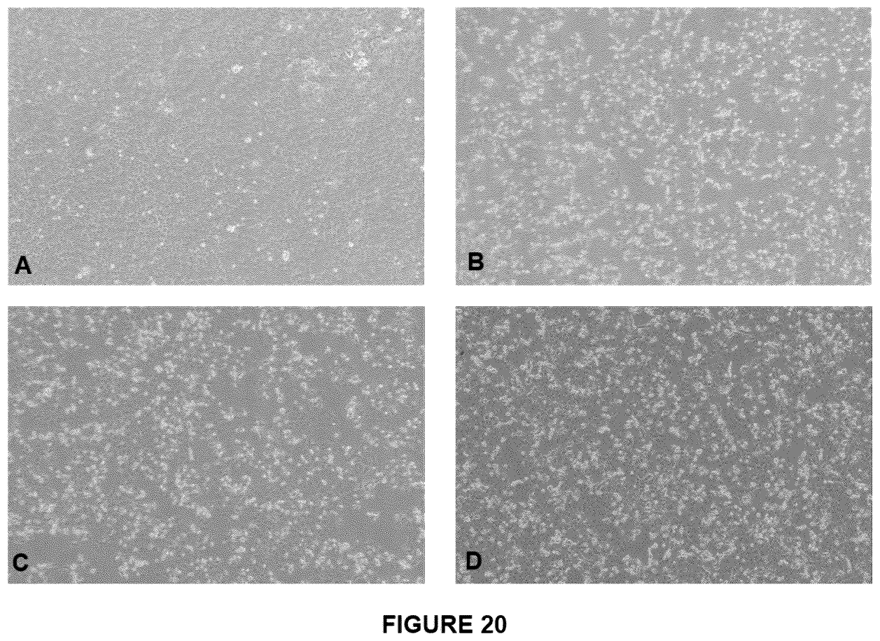

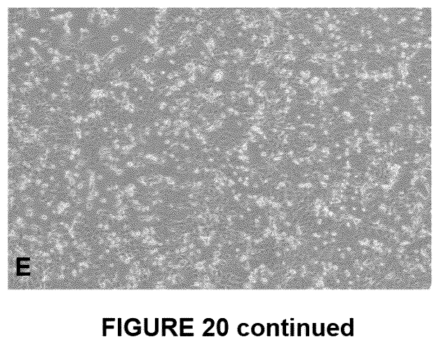

Mock: no sign of CPE (FIG. 20A); SARS-COV-2: 95% CPE (FIG. 20B); SARS-COV-2-160-7: 85-90% CPE (FIG. 20C); SARS-COV-2-4N-1: 75-85% CPE (FIG. 20D); and SARS-COV-2-7N-1: 70-80% CPE (FIG. 20E).

All clones based on SARS-COV-2-77 showed CPE in Vero E6 cells, but growth curve kinetic studies were not undertaken.

### Example 9—Severe Acute Respiratory Syndrome Coronavirus 2 (SARS-COV-2) Plaque Assay

This Example describes determining the viral titre of SARS-COV-2 samples using the plaque assay technique.

### Day 1—Plating Cells

Seed Vero E6 cells in 12-well tissue culture plate(s) at ˜2×10⁵ cells per well in DMEM growth medium (DMEM+5% FCS) and incubate overnight at 37° C. with 5% CO₂ in PC2.

### Day 2—Virus Dilution & Inoculation

Thaw virus sample(s) and make 10-fold serial dilutions of sample(s) using DMEM dilution/infection medium (DMEM serum free) in a sterile 96-well U-bottom plate(s). Prepare enough diluted virus to do each dilution in duplicate. Remove the media from the cell culture wells and wash cells once with sterile PBS. Remove the PBS from the cells and inoculate the appropriate wells with 200 μl of diluted viral solutions. Incubate the plate(s) at 37° C. 5% CO₂ for 1 hour with periodic rocking to ensure the cells are in contact with the infection mixture. During the incubation period prepare Avicel overlay by mixing 2.4% Avicel RC-581 with 2× overlay medium (2× DMEM+2% FCS) at a 1:1 ratio. Final concentration=1.2% Avicel+DMEM+1% FCS. After 1 hr incubation remove the viral inocula from the wells using a pipette then discard the liquid waste into the tube with F10SC. Overlay cells with 1 mL of 1.2% Avicel overlay mixture. Incubate cells for 72 hours at 37° C., 5% CO₂.

### Day 5—Fixing & Staining

Fix plates by working with one plate at a time, manually remove the 1.2% Avicel overlay from monolayers using a pipette then discard the waste into the tube with F10SC. Immediately rinse plates with PBS. Repeat this rinse one to two more times if a lot of residual Avicel is still present. Immediately fix and stain the cells with 0.1% Crystal Violet: 3.7% Formaldehyde (CV:FA) for at least 30 minutes at room temperature. Following staining and fixation, remove stain solution from the wells and rinse any remaining CV:FA with water. Allow plates to air dry in a BSL2 cabinet. Count the number of plaques in the wells and then calculate the number of plaque forming units per ml (pfu/ml) using the following formula:

$$pfu / ml = N / ( D \times V )$$

N=number of plaques counted
D=dilution factor (e.g. 0.0001 for 1×10⁻⁴)
V=volume of diluted virus in ml added to well (e.g. 0.2 ml)

### Results

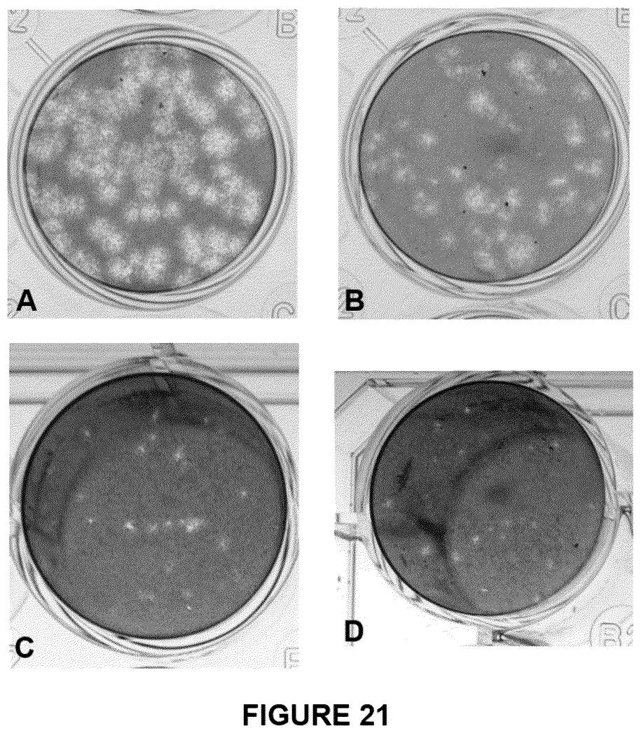

SARS-COV-2 formed plaques of a similar size with a round shape. Compared to the de-optimized strains/candidates, the plaques are bigger and have clearer boundaries (FIG. 21A).

SARS-COV-2-160-7 formed plaques of two sizes. Approximately 85% of the plaques are small plaques that have blur boundaries and irregular shapes. Approximately 15% of the plaques are bigger and similar to the WT strains (FIG. 21B).

SARS-COV-2-4N-1 had plaques of similar sizes in irregular shapes. When compared to the WT stain, the plaques are much smaller and have blurred boundaries (FIG. 21C).

SARS-COV-2-7N-1 had plaques of similar sizes in irregular shapes. When compared to the WT stain, the plaques are much smaller and have blurred boundaries and are similar in phenotypes to SARS-COV-2-4N-1 (FIG. 21D).

### Example 10—Testing Second Generation Vaccine Candidate Safety, for Cell and Tissue Damage and Reactive Inflammation in Hamsters

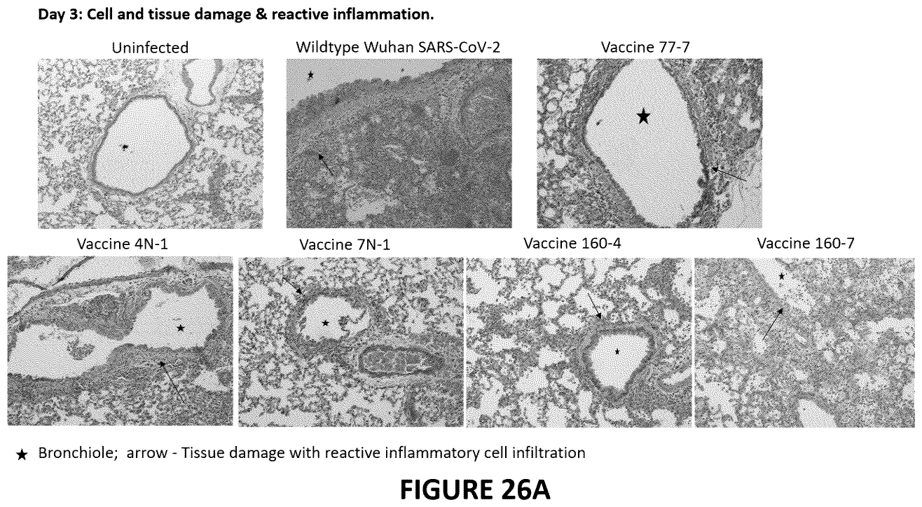

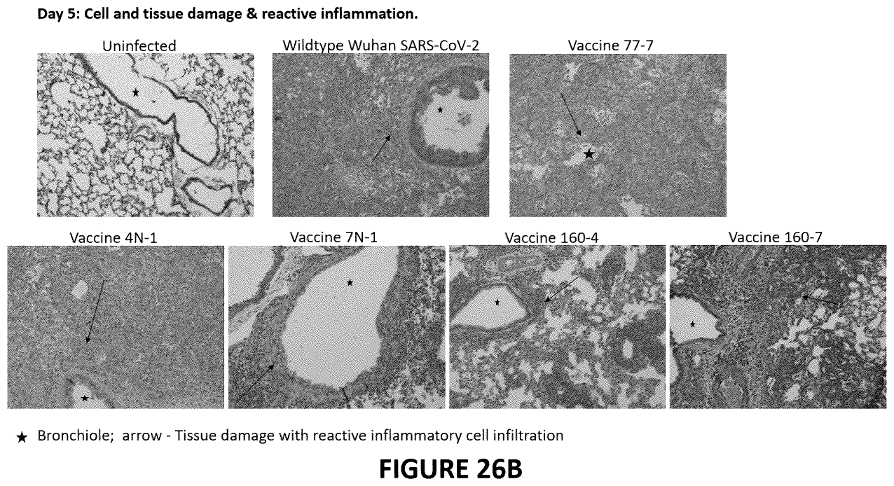

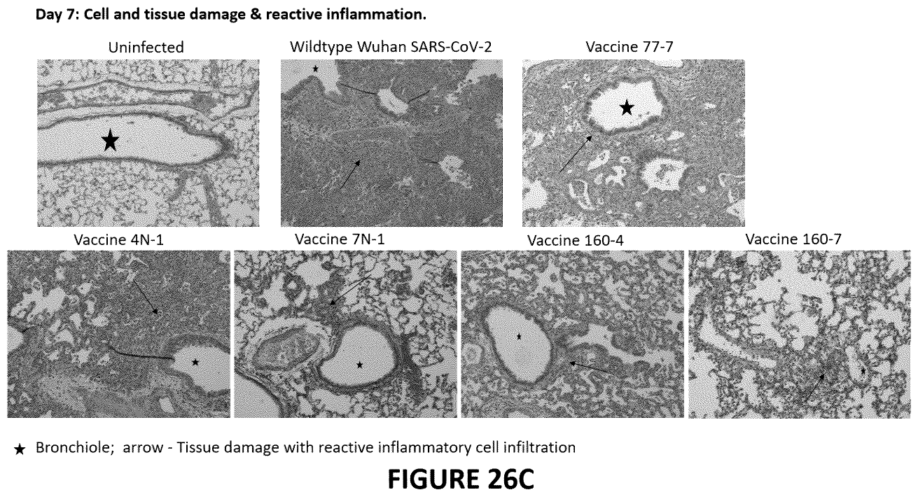

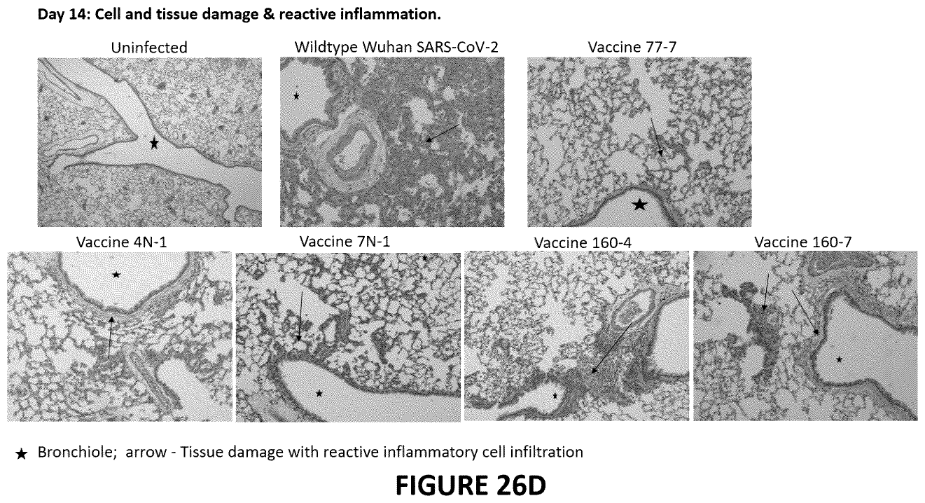

We tested the safety of the vaccine candidates in a hamster model. Hamsters were infected intranasally with 10⁵ PFU of vaccine candidates and wild-type SARS-COV-2. Lung pathology was assessed in infected hamsters, for cell and tissue damage and reactive inflammation. See FIG. 26A-D and Table 2 below. A 5-step grading system of minimum, mild, moderate, marked and severe was used to rank microscopic findings for comparison among groups.

TABLE 2

| | | Day/Incident (No. of | | | |
|---|---|---|---|---|---|
| Group | | animals showing lesions) | | | |
| Day | Group | 3 | 5 | 7 | 14 |
| Cell and tissue damage | | | | | |
| Necrosis of BEC | Wildtype | 4 | 3 | 0 | 0 |
| | 77-7 | 4 | 4 | 2 | 0 |
| | 4N-1 | 3 | 2 | 0 | 0 |
| | 7N-1 | 0 | 3 | 0 | 0 |
| | 160-4 | 0 | 1 | 0 | 0 |
| | 160-7 | 1 | 0 | 0 | 0 |
| | Uninfected | 0 | 0 | 0 | 0 |
| Cellular debris in bronchi | Wildtype | 4 | 4 | 2 | 0 |
| | 77-7 | 4 | 2 | 2 | 0 |
| | 4N-1 | 3 | 2 | 0 | 0 |
| | 7N-1 | 0 | 0 | 0 | 0 |
| | 160-4 | 0 | 0 | 1 | 0 |
| | 160-7 | 1 | 0 | 0 | 0 |
| | Uninfected | 0 | 0 | 0 | 0 |
| Diffuse alveolar damage | Wildtype | 3 | 4 | 4 | 3 |
| | 77-7 | 1 | 4 | 4 | 3 |
| | 4N-1 | 2 | 4 | 4 | 3 |
| | 7N-1 | 0 | 2 | 4 | 4 |
| | 160-4 | 1 | 2 | 3 | 4 |
| | 160-7 | 4 | 3 | 4 | 1 |
| | Uninfected | 0 | 0 | 0 | 0 |
| Necrosis of AEC | Wildtype | 3 | 4 | 0 | 1 |
| | 77-7 | 1 | 4 | 4 | 0 |
| | 4N-1 | 2 | 4 | 4 | 1 |
| | 7N-1 | 0 | 3 | 3 | 2 |
| | 160-4 | 2 | 3 | 3 | 3 |
| | 160-7 | 4 | 3 | 4 | 0 |
| | Uninfected | 0 | 0 | 0 | 0 |
| Cellular debris in alveoli | Wildtype | 3 | 4 | 4 | 2 |
| | 77-7 | 1 | 4 | 4 | 1 |
| | 4N-1 | 2 | 4 | 4 | 1 |
| | 7N-1 | 0 | 3 | 4 | 2 |
| | 160-4 | 3 | 3 | 4 | 3 |
| | 160-7 | 4 | 3 | 4 | 1 |
| | Uninfected | 0 | 0 | 0 | 0 |
| Alveolar emphysema | Wildtype | 3 | 4 | 4 | 2 |
| | 77-7 | 1 | 4 | 4 | 1 |
| | 4N-1 | 2 | 3 | 3 | 0 |
| | 7N-1 | 0 | 2 | 2 | 0 |
| | 160-4 | 1 | 1 | 0 | 0 |
| | 160-7 | 3 | 2 | 0 | 0 |
| | Uninfected | 0 | 0 | 0 | 0 |
| Reactive inflammatory patterns | | | | | |
| Necrosuppurative bronchitis | Wildtype | 0 | 1 | 0 | 0 |
| | 77-7 | 2 | 0 | 0 | 0 |
| | 4N-1 | 1 | 0 | 0 | 0 |
| | 7N-1 | 0 | 0 | 0 | 0 |
| | 160-4 | 0 | 0 | 0 | 0 |
| | 160-7 | 0 | 0 | 0 | 0 |
| | Uninfected | 0 | 0 | 0 | 0 |
| Bronchointerstitial pneumonia | Wildtype | 0 | 1 | 0 | 0 |
| | 77-7 | 0 | 0 | 0 | 0 |
| | 4N-1 | 1 | 0 | 0 | 0 |
| | 7N-1 | 0 | 0 | 0 | 0 |
| | 160-4 | 0 | 0 | 0 | 0 |
| | 160-7 | 0 | 0 | 0 | 0 |
| | Uninfected | 0 | 0 | 0 | 0 |
| Intraalveolar neutrophils and | Wildtype | 3 | 4 | 4 | 3 |
| macrophages | | | | | |
| | 77-7 | 1 | 4 | 4 | 2 |
| | 4N-1 | 3 | 4 | 4 | 1 |
| | 7N-1 | 0 | 3 | 4 | 2 |
| | 160-4 | 3 | 3 | 4 | 4 |
| | 160-7 | 4 | 3 | 4 | 1 |
| | Uninfected | 0 | 0 | 0 | 0 |
| Lymphocytes | Wildtype | 4 | 4 | 4 | 3 |
| | 77-7 | 4 | 4 | 4 | 1 |
| | 4N-1 | 4 | 4 | 4 | 0 |
| | 7N-1 | 2 | 4 | 4 | 2 |
| | 160-4 | 4 | 4 | 4 | 4 |
| | 160-7 | 4 | 4 | 4 | 2 |
| | Uninfected | 0 | 0 | 0 | 0 |
| Polymorphonuclear granulocytes | Wildtype | 4 | 4 | 4 | 2 |
| (neutrophils, heterophils) | | | | | |
| | 77-7 | 4 | 4 | 4 | 1 |
| | 4N-1 | 4 | 4 | 4 | 0 |
| | 7N-1 | 2 | 4 | 4 | 2 |
| | 160-4 | 4 | 4 | 4 | 4 |
| | 160-7 | 4 | 4 | 4 | 2 |
| | Uninfected | 0 | 0 | 0 | 0 |
| Monocytes, macrophages | Wildtype | 3 | 4 | 4 | 3 |
| | 77-7 | 4 | 4 | 4 | 4 |
| | 4N-1 | 3 | 4 | 4 | 3 |
| | 7N-1 | 0 | 3 | 4 | 4 |
| | 160-4 | 1 | 4 | 4 | 4 |
| | 160-7 | 4 | 3 | 4 | 2 |
| | Uninfected | 0 | 0 | 0 | 0 |
| Perivascular lymphocytic cuffing | Wildtype | 4 | 4 | 4 | 3 |
| | 77-7 | 4 | 4 | 4 | 0 |
| | 4N-1 | 3 | 4 | 4 | 0 |
| | 7N-1 | 0 | 4 | 4 | 2 |
| | 160-4 | 3 | 4 | 4 | 4 |
| | 160-7 | 4 | 4 | 4 | 3 |
| | Uninfected | 0 | 0 | 0 | 0 |
| Activation of mesothelial cells | Wildtype | 0 | 0 | 0 | 0 |
| | 77-7 | 0 | 1 | 0 | 0 |
| | 4N-1 | 0 | 0 | 0 | 0 |
| | 7N-1 | 0 | 0 | 0 | 0 |
| | 160-4 | 1 | 1 | 0 | 0 |
| | 160-7 | 1 | 0 | 0 | 0 |
| | Uninfected | 0 | 0 | 0 | 0 |

Key: n = 4 on each day except n = 2 for uninfected

A discussion of the results follows.

### Wild-Type SARS-COV-2

Day 3: Clear evidence of tissue damage and reactive inflammatory cells.
Day 5: Clear evidence of tissue damage and reactive inflammatory cells.
Day 7: Clear evidence of tissue damage and reactive inflammatory cells.
Day 14: Evidence of tissue damage and reactive inflammatory cells but reduced in intensity compared to earlier time points.

### Vaccine Candidates

Day 3: Clear evidence of tissue damage and reactive inflammatory cells in the lungs of hamsters infected with candidates 77-7, 4N-1, 160-4 and 160-7. Minimal damage with candidates 7N-1 but not as prominent.
Day 5: Substantial tissue damage and reactive inflammatory cell infiltration, with candidate 7N-1 significantly lower than candidates 77-7, 4N-1, 160-4 and 160-7.
Day 7: Clear evidence of tissue damage with candidates 77-7, 4N-1 and 160-4. Candidate 160-7 showed considerably reduced tissue damage and candidate 7N-1 showed negligible tissue damage.
Day 14: Tissue damage and reactive inflammatory cells were reduced in intensity compared to earlier time points. Candidate 7N-1 showed negligible tissue damage.

### Example 11—Testing Vaccine Safety: Distribution of Lesions, Bronchial and Peribronchial Distribution of Inflammatory Cells

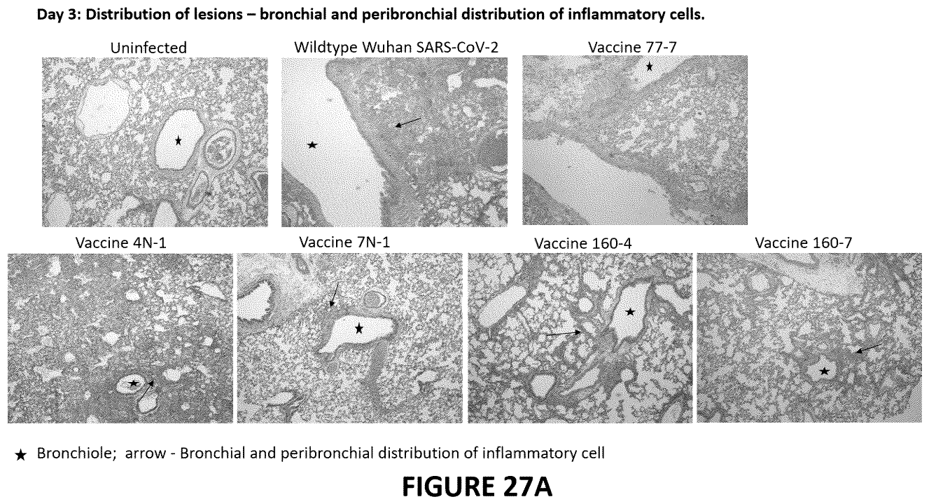

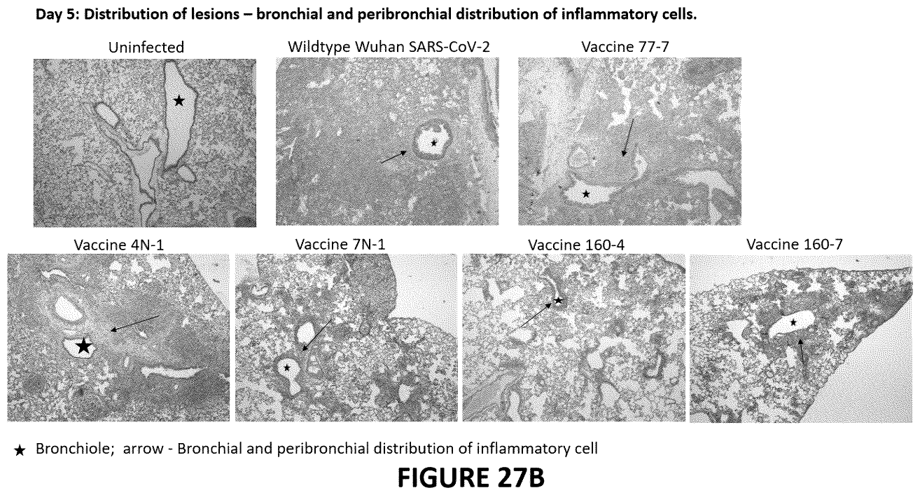

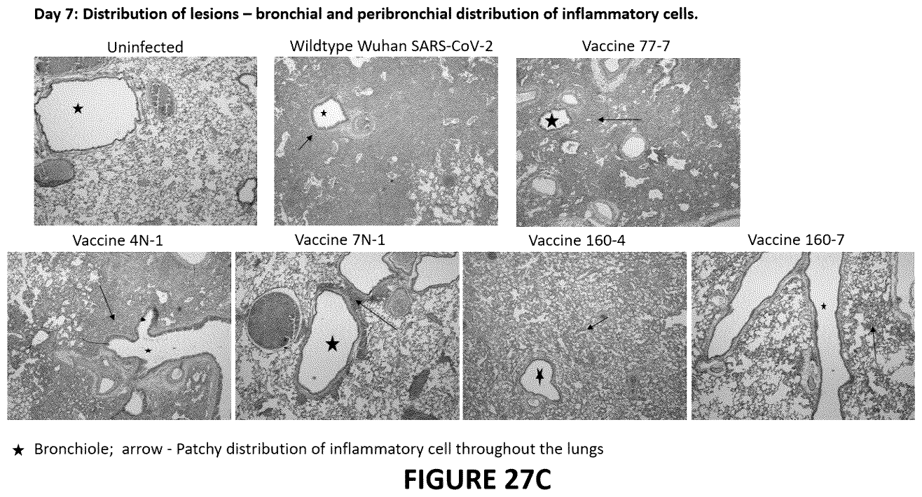

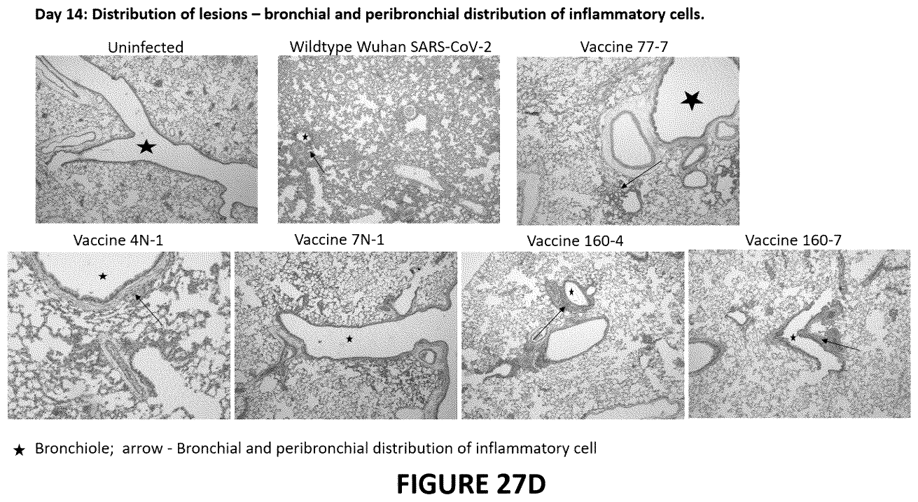

We tested the safety of the vaccine in a hamster model. Hamsters were infected intranasally with 10⁵ PFU of vaccine candidates and wild-type SARS-COV-2. Lung pathology was assessed in infected hamsters, for distribution of lesions, bronchial and peribronchial distribution of inflammatory cells. See FIG. 27A-D and Table 3 below. A 5-step grading system of minimum, mild, moderate, marked and severe was used to rank microscopic findings for comparison among groups.

TABLE 3

| | | Day/Incident (No. of | | | |
|---|---|---|---|---|---|
| Group | | animals showing lesions) | | | |
| Day | Group | 3 | 5 | 7 | 14 |
| Overall severity | | | | | |
| % of lung area affected | Wildtype | 10 | 55 | 55 | 19 |
| Mean | 77-7 | 4.50 | 28.75 | 28.75 | 4.50 |
| | 4N-1 | 15.50 | 17.50 | 31.25 | 3.00 |
| | 7N-1 | 0.50 | 3.50 | 4.75 | 3.25 |
| | 160-4 | 4.75 | 17.00 | 14.00 | 10.25 |
| | 160-7 | 14.75 | 14.00 | 21.75 | 1.25 |
| | Uninfected | 0.00 | 0.00 | 0.00 | 0.00 |
| Distribution of lesions | | | | | |
| Bronchial and peribronchial | Wildtype | 4 | 4 | 4 | 3 |
| | 77-7 | 4 | 4 | 4 | 4 |
| | 4N-1 | 4 | 4 | 2 | 3 |
| | 7N-1 | 2 | 4 | 1 | 4 |
| | 160-4 | 3 | 4 | 3 | 4 |
| | 160-7 | 4 | 4 | 2 | 1 |
| | Uninfected | 0 | 0 | 0 | 0 |
| Patchy throughout the lungs | Wildtype | 3 | 4 | 4 | 3 |
| | 77-7 | 1 | 4 | 4 | 3 |
| | 4N-1 | 2 | 4 | 4 | 1 |
| | 7N-1 | 0 | 3 | 4 | 2 |
| | 160-4 | 1 | 3 | 4 | 4 |
| | 160-7 | 4 | 3 | 4 | 1 |
| | Uninfected | 0 | 0 | 0 | 0 |

Key: n = 4 on each day except n = 2 for uninfected

A discussion of the results follows.

### Wild-Type SARS-COV-2

At days 3, 5, 7 and 14 there was marked bronchial and peribronchial distribution of inflammatory cells, with a slight reduction in the extent and intensity of inflammation at day 14 compared to the earlier time points. At days 3, 5 and 7 there was a patchy distribution of inflammatory cells throughout the lungs.

### Vaccine Candidates

Day 3: pronounced following infection with vaccine candidates 77-7 and 4N-1. Bronchial and peribronchial distribution of inflammatory cells was not as intense with vaccine candidates 7N-1. 160-4 and 160-7, with candidate 7N-1 similar to that in uninfected hamsters.
Day 5: Intense bronchial and peribronchial distribution of inflammatory cells with candidates 77-7 and 4N-1, substantially less in the lungs of hamsters infected with candidates 7N-1, 160-4 and 160-7.
Day 7: Intensity greatest for candidates 77-7 and 4N-1, with candidate 160-4 showing less intense distribution of inflammatory cells. Distribution of inflammatory cells was lowest for candidates 7N-1 and 160-7.
Day 14: Distribution of inflammatory cells was less than at earlier time points, with candidate 7N-1 showing particularly low levels of inflammatory cells.

Histopathological changes were observed in wildtype SARS-COV-2-infected groups on 3 day post infection (dpi) and time-dependent increase in severity of % lungs affected was observed on 5 dpi and 7 dpi. However, there was decrease in severity on 14 dpi. The % lungs affected was higher in wildtype SARS-COV-2-infected groups (55%) on 7 dpi which was well correlated with the distribution of lesions, cellular and tissue damage, circular changes, vascular lesions and reactive inflammatory patterns. Minimal to mild patchy lesions around bronchial and peribronchial were recorded in lungs of all SARS-COV-2-infected animals.

Histopathological changes were observed in all SARS-COV-2-infected groups on 3 dpi and time-dependent increase in severity of % lungs affected was observed on 5 dpi and 7 dpi. However, there was decrease in severity in all the groups on 14 dpi except marginal decrease in candidate 160-4. The % lungs affected was higher in candidate 4N-1 (31.25%) followed candidates 77-7 (28.75%), 160-7 (21.75%), 160-4 (14.00%) and 7N-1 (4.75%) on 7 dpi which was well correlated with the distribution of lesions, cellular and tissue damage, circular changes, vascular lesions and reactive inflammatory patterns. Minimal to mild patchy lesions around bronchial and peribronchial were recorded in lungs of all SARS-COV-2-infected animals.

### Example 12—Testing Vaccine Safety, for Circulatory and Vascular Lesions, Including Perivascular Edema, Desquamation of Endothelial Cells and Endothelialitis

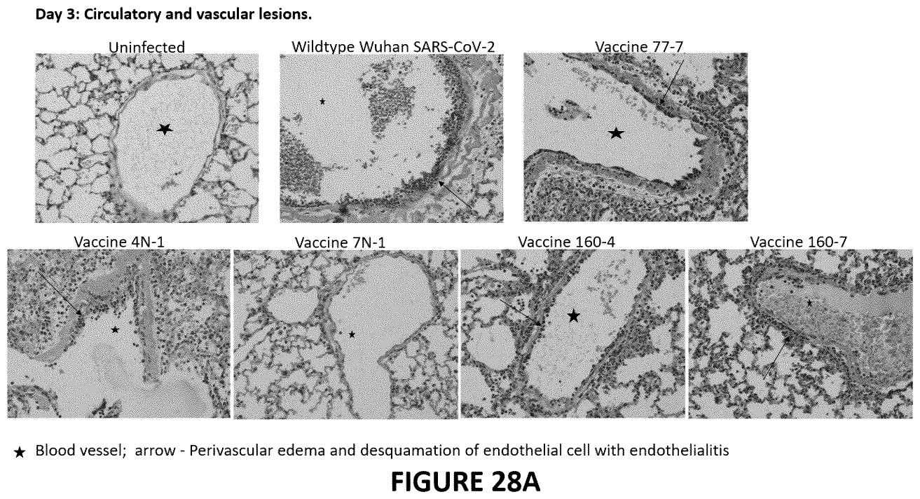

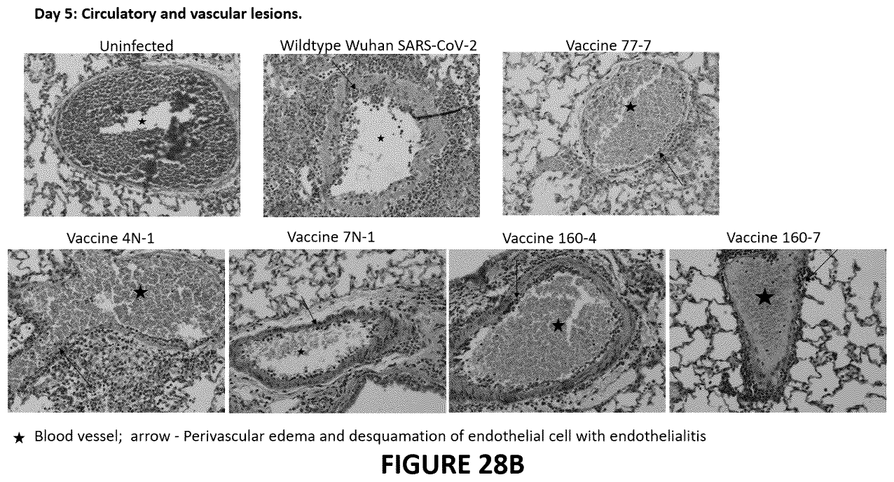

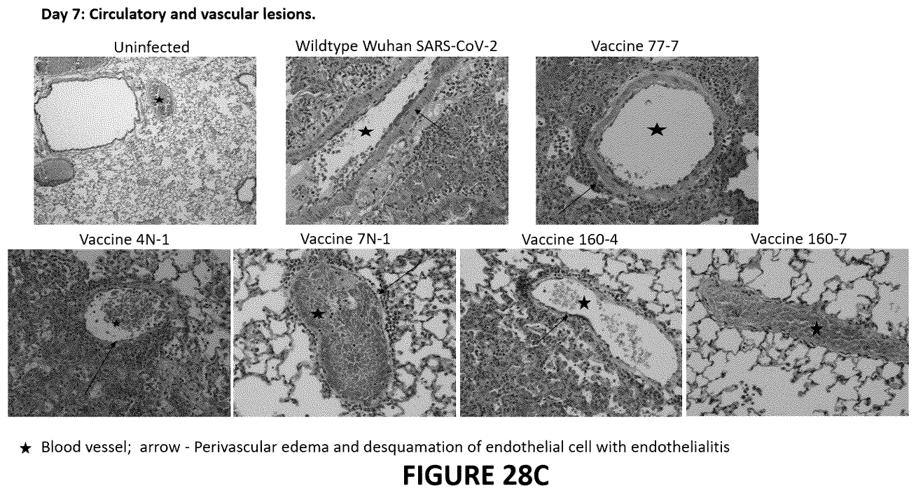

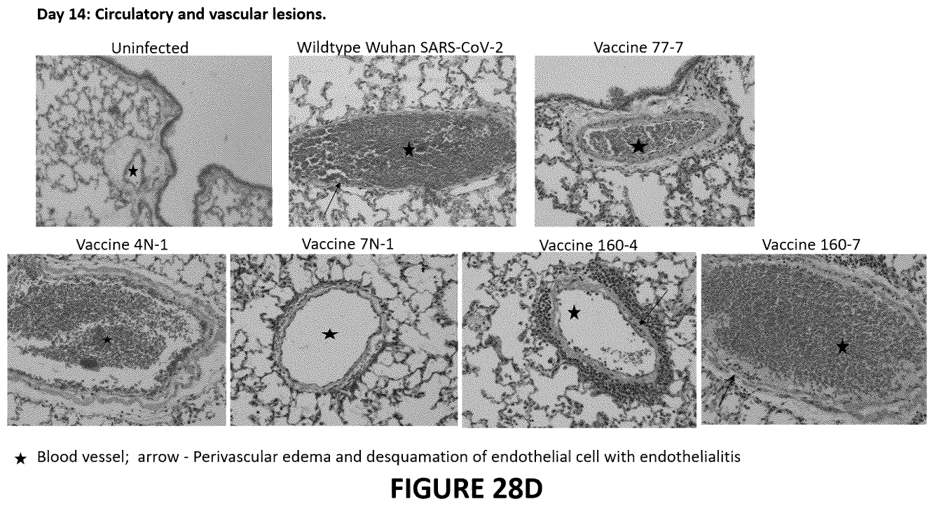

We tested the safety of the vaccine candidates in a hamster model. Hamsters were infected intranasally with 10⁵ PFU of vaccine candidates and wild-type SARS-COV-2. Lung pathology was assessed in infected hamsters, for circulatory and vascular lesions, including perivascular edema, desquamation of endothelial cells and endothelialitis. See FIG. 28A-D and Table 4 below. A 5-step grading system of minimum, mild, moderate, marked and severe was used to rank microscopic findings for comparison among groups.

TABLE 4
Group

| Day | | Day/Incident (No. of | | | |
|---|---|---|---|---|---|
| Circulatory changes and | | animals showing lesions) | | | |
| vascular lesions | Group | 3 | 5 | 7 | 14 |
| Alveolar hemorrhage | Wildtype | 3 | 4 | 4 | 2 |
| | 77-7 | 1 | 4 | 3 | 1 |
| | 4N-1 | 2 | 4 | 3 | 0 |
| | 7N-1 | 0 | 3 | 3 | 2 |
| | 160-4 | 0 | 2 | 1 | 0 |
| | 160-7 | 3 | 3 | 2 | 0 |
| | Uninfected | 0 | 0 | 0 | 0 |
| Alveolar edema | Wildtype | 3 | 4 | 4 | 2 |
| | 77-7 | 1 | 4 | 3 | 0 |
| | 4N-1 | 2 | 4 | 1 | 0 |
| | 7N-1 | 0 | 3 | 1 | 0 |
| | 160-4 | 0 | 2 | 1 | 0 |
| | 160-7 | 2 | 0 | 1 | 0 |
| | Uninfected | 0 | 0 | 0 | 0 |
| Perivascular/interstitial edema | Wildtype | 3 | 4 | 4 | 2 |
| | 77-7 | 3 | 4 | 4 | 1 |
| | 4N-1 | 2 | 3 | 4 | 0 |
| | 7N-1 | 0 | 2 | 3 | 2 |
| | 160-4 | 1 | 1 | 4 | 0 |
| | 160-7 | 4 | 1 | 4 | 0 |
| | Uninfected | 0 | 0 | 0 | 0 |
| Vascular endothelialitis | Wildtype | 4 | 4 | 0 | 0 |
| | 77-7 | 4 | 3 | 1 | 0 |
| | 4N-1 | 3 | 4 | 2 | 0 |
| | 7N-1 | 0 | 4 | 0 | 0 |
| | 160-4 | 2 | 4 | 1 | 0 |
| | 160-7 | 3 | 2 | 0 | 0 |
| | Uninfected | 0 | 0 | 0 | 0 |
| Necrosis and desquamation of | Wildtype | 4 | 2 | 0 | 0 |
| vascular endothelial cells | | | | | |
| | 77-7 | 4 | 0 | 0 | 0 |
| | 4N-1 | 2 | 1 | 0 | 0 |
| | 7N-1 | 0 | 0 | 0 | 0 |
| | 160-4 | 1 | 0 | 0 | 0 |
| | 160-7 | 1 | 0 | 0 | 0 |
| | Uninfected | 0 | 0 | 0 | 0 |

Key: n = 4 on each day except n = 2 for uninfected

A discussion of the results follows.

### Wild-Type SARS-COV-2

Vascular lesions were prominent at day 3, day 5 and day 7 but had improved markedly by day 14. Circulatory changes and vascular lesions characterized by alveolar haemorrhage, alveolar edema, perivascular/interstitial edema, vascular endothelialitis and necrosis and desquamation of vascular endothelial cells were recorded in SARS-COV-2-infected lungs of all wild-type SARS-COV-2 animals and the severity of lesions was mild to marked.

### Vaccine Candidates

Day 3: Some perivascular edema, desquamation of endothelial cells and endothelialitis were observed in hamsters infected with candidates 77-7, 4N-1, 160-4 and 160-7. These pathologies were not present in the 7N-1 group.
Day 5: Perivascular edema, desquamation of endothelial cells and endothelialitis were prominent in hamsters infected with candidates 77-7, 4N-1 and 160-4, and less intense with candidates 7N-1 and 160-7.
Day 7: Perivascular edema, desquamation of endothelial cells and endothelialitis were prominent in hamsters infected with candidates 77-7,4N- and 160-4, and less intense with candidates 7N-1 and 160-7.
Day 14: Perivascular edema, desquamation of endothelial cells and endothelialitis were reduced in all groups compared to earlier time points. Candidates 77-7, 4N-1 and 160-4 still showed some vascular lesions. Vascular lesions were not seen with candidate 7N-1 and were minimal in the 160-7 group.

Overall conclusion: Circulatory changes and vascular lesions characterized by alveolar haemorrhage, alveolar edema, perivascular/interstitial edema, vascular endothelialitis and necrosis and desquamation of vascular endothelial cells were recorded in lungs of all vaccine-infected animals. The severity of lesions was minimal to mild in candidates 77-7, 4N-1 and 160-4 and minimal in candidates 7N-1 and 160-7.

### Example 13—Testing Second Generation Vaccine Safety, for Regeneration and Repair

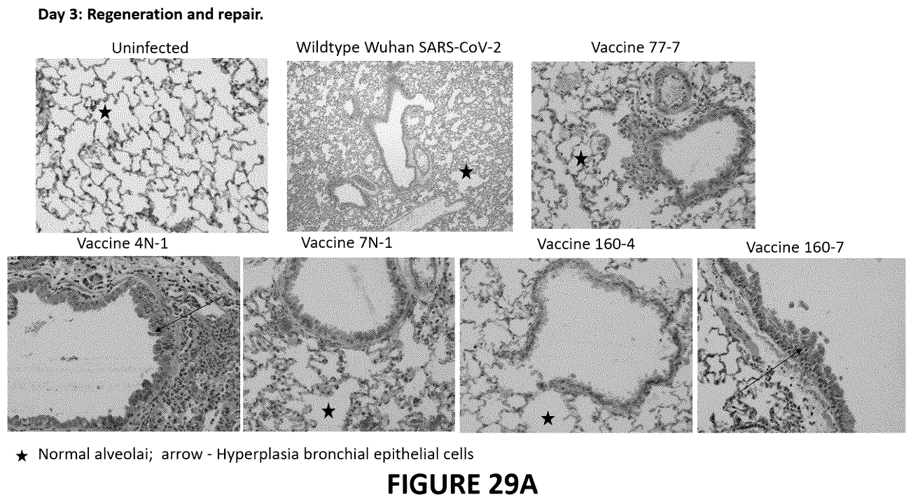

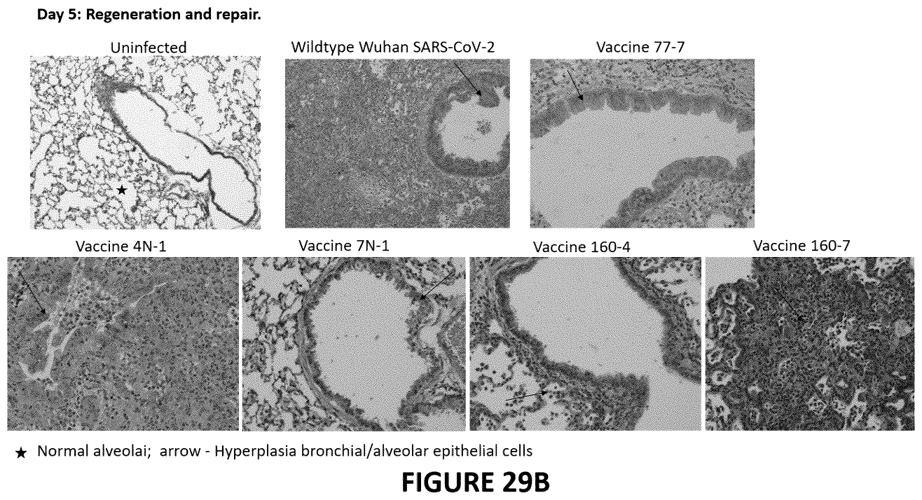

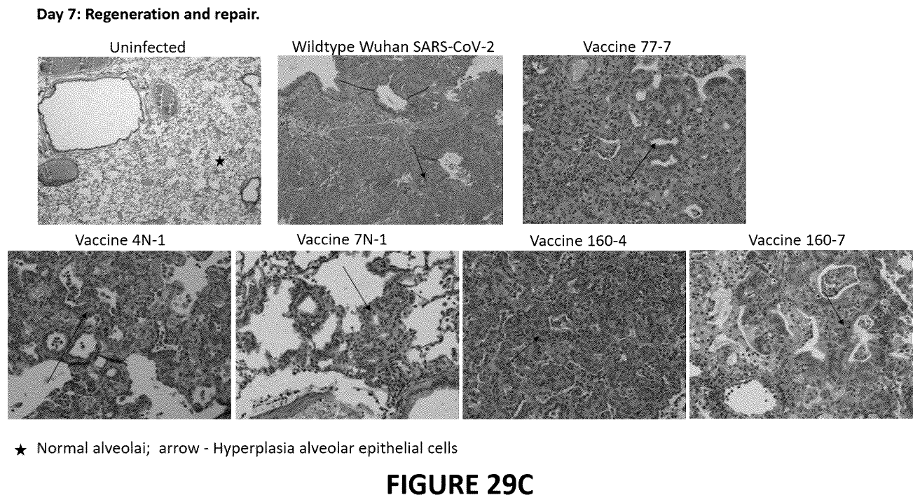

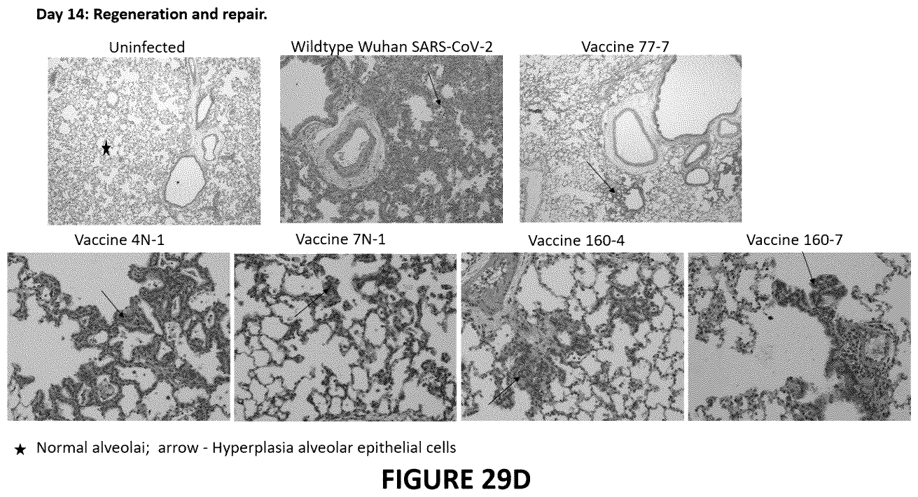

We tested the safety of the vaccine in a hamster model. Hamsters were infected intranasally with 10⁵ PFU of vaccine candidates and wild-type SARS-COV-2. Lung pathology was assessed in infected hamsters, for regeneration and repair. See FIG. 29A-D and Table 5 below. A 5-step grading system of minimum, mild, moderate, marked and severe was used to rank microscopic findings for comparison among groups.

TABLE 5

| Group | | Day/Incident (No. of | | | |
|---|---|---|---|---|---|
| Day | | animals showing lesions) | | | |
| Regeneration and repair | Group | 3 | 5 | 7 | 14 |
| Hyperplasia of BEC | Wildtype | 0 | 4 | 4 | 1 |
| | 77-7 | 0 | 3 | 0 | 0 |
| | 4N-1 | 1 | 2 | 0 | 0 |
| | 7N-1 | 0 | 0 | 0 | 0 |
| | 160-4 | 0 | 0 | 0 | 0 |
| | 160-7 | 1 | 0 | 0 | 0 |
| | Uninfected | 0 | 0 | 0 | 0 |
| Hyperplasia of AEC-II | Wildtype | 0 | 3 | 4 | 3 |
| | 77-7 | 0 | 1 | 4 | 4 |
| | 4N-1 | 0 | 2 | 4 | 3 |
| | 7N-1 | 0 | 0 | 2 | 2 |
| | 160-4 | 0 | 0 | 3 | 1 |
| | 160-7 | 1 | 1 | 1 | 1 |
| | Uninfected | 0 | 0 | 0 | 0 |
| Multinucleated or otherwise | Wildtype | 0 | 0 | 0 | 0 |
| atypical epithelial cells | | | | | |
| | 77-7 | 0 | 0 | 0 | 0 |
| | 4N-1 | 0 | 0 | 0 | 0 |
| | 7N-1 | 0 | 0 | 0 | 0 |
| | 160-4 | 0 | 0 | 0 | 0 |
| | 160-7 | 0 | 0 | 0 | 0 |
| | Uninfected | 0 | 0 | 0 | 0 |
| Pleural fibroblastic | Wildtype | 0 | 0 | 0 | 1 |
| proliferation/fibrosis | | | | | |
| | 77-7 | 0 | 0 | 0 | 0 |
| | 4N-1 | 0 | 0 | 0 | 0 |
| | 7N-1 | 0 | 0 | 0 | 0 |
| | 160-4 | 0 | 0 | 0 | 0 |
| | 160-7 | 0 | 0 | 0 | 0 |
| | Uninfected | 0 | 0 | 0 | 0 |

Key: n = 4 on each day except n = 2 for uninfected

A discussion of the results follows.

### Wild-Type SARS-COV-2

Minimal hyperplasia of alveolar epithelial cells was observed at day 3. There was significant hyperplasia of alveolar epithelial cells at day 5 and day 7, with a slight reduction of hyperplasia at day 14. Taken together, these results provide clear indication of the virulence of wild-type SARS-COV-2 in the hamster model, with massive lung pathology.

### Vaccine Candidates

Day 3: Hyperplasia of alveolar epithelial cells was prominent in candidate 4N-1, and present at reduced levels with candidates 77-7, 160-4 and 160-7. It was not present in candidate 7N-1.
Day 5: Hyperplasia of alveolar epithelial cells was prominent in candidate 4N-1, and present at reduced levels in candidate 160-7, followed by candidate 77-7 and candidate 160-4. Hyperplasia was minimal in candidate 7N-1.
Day 7: Hyperplasia of alveolar epithelial cells was prominent in 77-7, 4N-1, 160-4 and 160-7 groups and minimal in candidate 7N-1.
Day 14: Hyperplasia of alveolar epithelial cells was reduced in all groups compared to earlier time points, with 7N-1 showing best lung integrity of all groups.

Regeneration and repair characterized by hyperplasia of BEC, hyperplasia of AEC-II, multinucleated or otherwise atypical epithelial cells and pleural fibroblastic proliferation/fibrosis was recorded from day 5 in wildtype SARS-COV-2. No histopathological changes were observed in uninfected group.

Regeneration and repair characterized by hyperplasia of BEC, hyperplasia of AEC-II, multinucleated or otherwise atypical epithelial cells and pleural fibroblastic proliferation/fibrosis was recorded from day 3 dpi in candidates 4N-1 and 160-7; day 5 in 77-7 and day 7 in 7N-1 and 160-4. No histopathological changes were observed in uninfected group.

### Example 14—Challenge Experiment, Showing the Efficacy of Vaccine Candidate 7N-1 in Mice

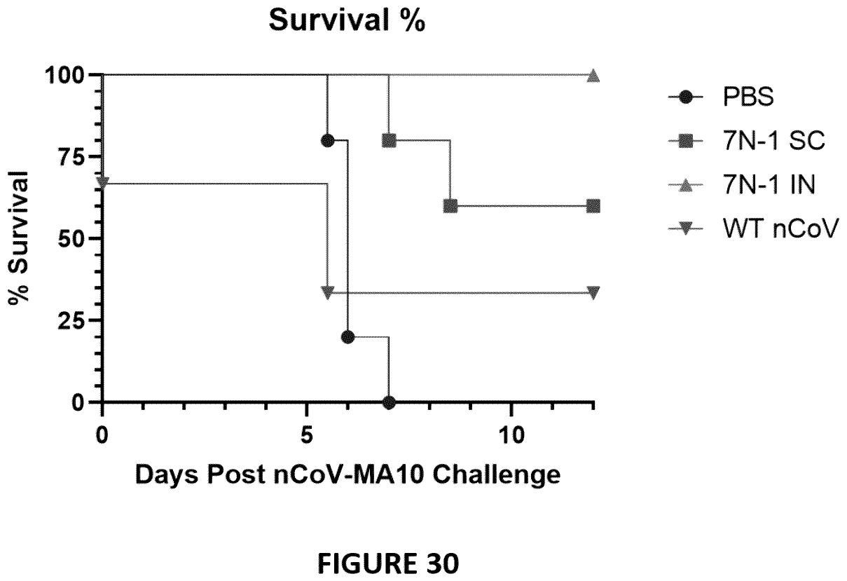

To test the efficacy of candidate 7N-1 as a COVID-19 vaccine, we established a challenge experiment using the following immunisation groups: 10³ PFU 7N-1 intranasal (7N-1 IN); 10⁵ PFU 7N-1 subcutaneous (7N-1 SC); 10³ PFU wild-type mouse-adapted SARS-COV-2 (SARS-COV-2 MA10) intranasal (WT nCOV); and unimmunised (PBS treated). HFH4-hACE2 mice were used in this study. HFH4-hACE2 mice expressed high levels of hACE2 in the lung, but varying expression levels in other tissues. See FIG. 30.

In the first 7 days post-immunisation: (i) we did not see any disease in mice given 10³ PFU 7N-1 intranasal; 10⁵ PFU 7N-1 subcutaneous; and unimmunised (PBS treated); (ii) severe disease and death was observed in 1 mouse given 10³ PFU wild-type mouse-adapted SARS-COV-2 (SARS-COV-2 MA10) intranasal.

Three weeks later, the mice were challenged with 10⁵ PFU intranasal wild-type mouse-adapted SARS-COV-2 (SARS-COV-2 MA10) and monitored over a 12-day period. 7N-1 vaccination provided strong protection from SARS-COV-2 challenge, with 100% survival in the intranasal group (green line—triangle symbol) and 65% survival in the subcutaneous group (red line—square symbol). Two mice in the 7N-1 subcutaneous vaccinated group die, although later than the unimmunised group. Death in this group is likely related to the reduced receptor expression in the periphery. All unimmunised mice died from challenge with SARS-COV-2 MA10 (blue line—PBS). Immunisation with wild-type mouse-adapted SARS-COV-2 (SARS-CoV-2 MA10—inverted triangle symbol) provided partial protection (25% survival).

### Summary

7N-1 (n=4 mice) provided full protection from rechallenge mortality when given intranasal route in HFH4-hACE2 mice.
7N-1 (n=5 mice) provided partial protection from subcutaneous route, which is probably related to the reduced receptor expression in the periphery. Death occurred later than the unvaccinated group.
Wild type virus (n=3 mice) intranasal resulted in 1 death during the primary infection, and an additional death following rechallenge.
PBS prime (n=5) showed full mortality upon wild type rechallenge.

### Example 15—Preclinical Immunogenicity Data in Animal Models

Hamsters were immunised intranasally with 10⁵ PFU with 7N-1, 4N-1, 77-7, 160-4 or 160-7 vaccine candidates. The mean neutralizing antibody titers (PRNT₁₀₀) at day 14 following immunization were determined and are shown in Table 6. Vaccine candidates 7N1, 160/7 and 160/4 were all highly immunogenic in the hamster model of infection, inducing a strong neutralizing antibody response.

TABLE 6

Average plaque count of Virus control- 138.8
Values represents the end point serum
dilution where 100% neutralization is observed

| | Animal-1 | Animal-2 | Animal-3 | Animal-4 |
|---|---|---|---|---|
| 160-7 | 640 | 640 | 1280 | 1280 |
| 160-4 | 40 | 40 | 1280 | 80 |
| 77-7 | 80 | 80 | 40 | 160 |
| 4N-1 | 40 | 40 | 40 | 40 |
| 7N-1 | 40 | 40 | 640 | 640 |

TABLE 7
Average plaque count of virus control-202.2
Values represent the end point serum dilution where 100%, 90% & 50% neutralization is observed

| | 160-7(100%) | 160-7(90%) | 160-7(50%) | 7N-1 (100%) | 7N-1 (90%) | 7N-1 (50%) |
|---|---|---|---|---|---|---|
| Animal-1 | <40 | — | — | 160 | 320 | >1280 |
| Animal-2 | 80 | 640 | >1280 | 80 | 640 | >1280 |
| Animal-3 | 40 | 640 | >1280 | <40 | — | — |
| Animal-4 | 40 | 320 | >1280 | <40 | — | — |
| Animal-5 | 40 | 320 | >1280 | 80 | 160 | >1280 |
| Animal-6 | 40 | 320 | >1280 | 80 | 320 | >1280 |
| Animal-7 | 80 | 640 | >1280 | 80 | 160 | >1280 |
| Animal-8 | 40 | 320 | >1280 | 80 | 160 | >1280 |
| Animal-9 | <40 | — | — | 80 | 160 | >1280 |
| Animal-10 | <40 | — | 640 | 40 | 160 | >1280 |
| Animal-11 | 40 | 320 | >1280 | <40 | — | 40 |
| Animal-12 | 40 | 320 | >1280 | 160 | 640 | >1280 |
| Animal-13 | 80 | 320 | >1280 | 160 | 640 | >1280 |
| Animal-14 | 40 | 320 | >1280 | 80 | 160 | >1280 |
| Animal-15 | 40 | 320 | >1280 | 40 | 160 | >1280 |
| Animal-16 | 80 | 640 | >1280 | 320 | 640 | >1280 |

The two candidates 7N-1 and 160-7 were highly immunogenic in the hamster model of infection, inducing a strong neutralizing antibody response. Hamsters were immunised subcutaneously with 10⁴ PFU 7N-1 or 160-7 vaccine candidates. At day 14 following immunization, blood was collected to determine neutralization titres. Mean PRNT₅₀ was 1280 for both vaccine candidates and mean PRNT₉₀ was 640 for 7N-1 and 640 for 160-7. Sec Table 7. Based on its high level of attenuation and safety in the mouse and hamster infection models, we selected 7N1 as our lead candidate for further analysis.

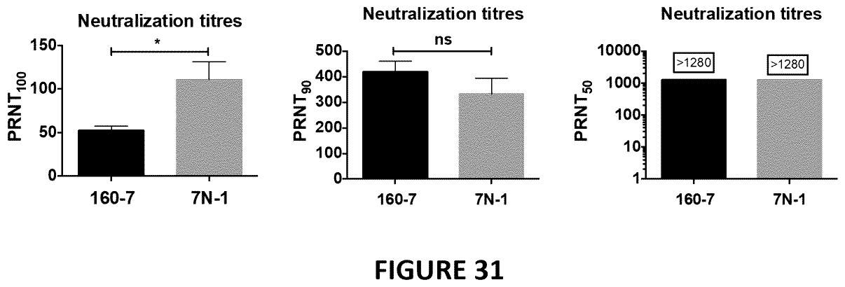

Hamsters were given single dose of 10⁴ PFU of live attenuated virus candidates 160-7 or 7N-1 subcutaneously. Neutralizing antibody titres were determined on day 14 after immunisation with live attenuated virus. The results are shown in FIG. 31. At 14 days post-vaccination, vaccinated hamsters showed very strong antibody neutralization in the blood. At 100% of neutralization, titres for 7N-1 100% were significantly higher that for 160-7. At 50% and 90% of neutralization, there was no significant difference in neutralization titres between 7N-1 and 160-7.

### Example 16—Vaccine Plaque Size after Multiple In Vitro Passage

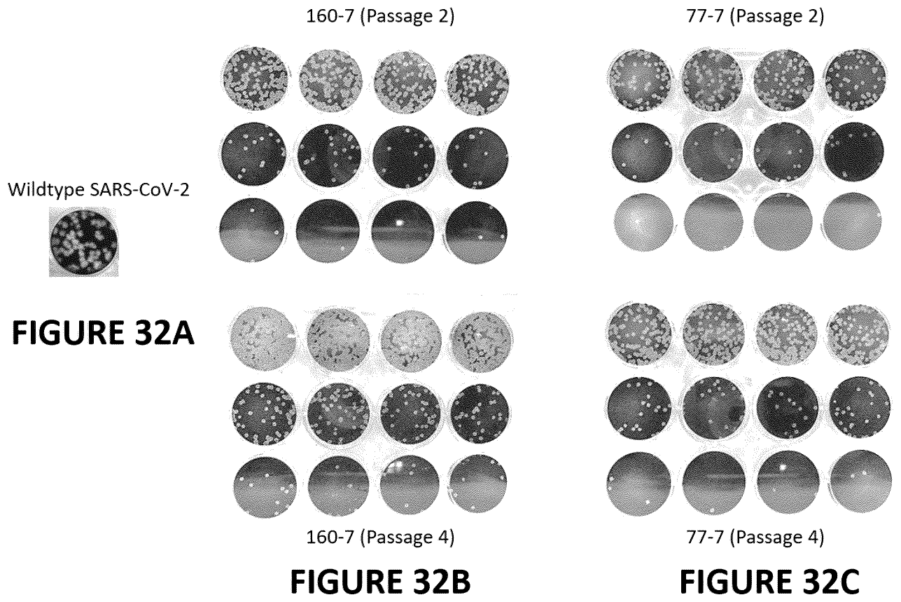

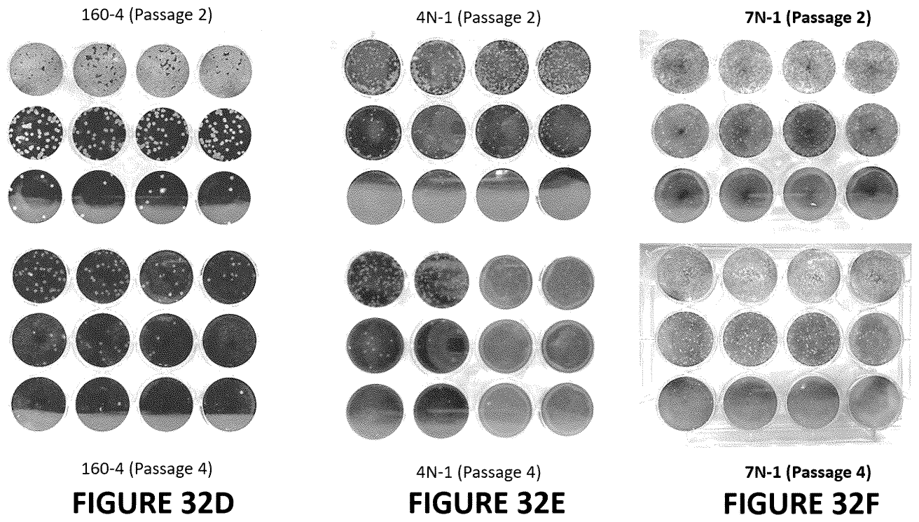

Vaccine candidates 7N-1, 77-7, 4N-1, 160-4 and 160-7 were passaged 4 times in Vero GMP cells at multiplicity of infection of 0.01 PFU/cell. Each dot represents one plaque. Vaccine candidates 4N-1 (FIG. 32E) and 7N-1 (FIG. 32F) have a small plaque phenotype compared to wildtype SARS-COV-2 (FIG. 32A) and the other vaccine candidates 77-7 (FIG. 32C), 160-4 (FIG. 32D) and 160-7 (FIG. 32B). Plaque size between wildtype SARS-COV-2 (FIG. 32A) and vaccine candidates 77-7 (FIG. 32C), 160-4 (FIG. 32D) and 160-7 (FIG. 32B) were similar.

Small plaques demonstrate a reduced ability of the vaccine to spread from the initial site of infection. This serves as useful marker of vaccine attenuation. To determine the phenotypic stability of vaccine attenuation extended in vitro passage of vaccine candidates and wildtype SARS-COV-2 was performed four times in Vero GMP cells. The plaque size of 4N-1 (FIG. 32E) and 7N-1 (FIG. 32F) remained smaller than wildtype SARS-COV-2 (FIG. 32A) after four passages. 4N-1 (FIG. 32E) and the lead vaccine candidate 7N-1 (FIG. 32F) did not revert to a wildtype plaque phenotype (FIG. 32A).

### Example 17—Vaccine Candidate 7N-1

The live attenuated SARS-COV-2 (COVID 19) vaccine described herein is based on codon de-optimization technology, which is a promising approach for achieving an enhanced safety profile (cannot revert to virulent strain), and is designed as a prophylactic, active, single dose immunization against coronavirus in humans. The vaccine should provide long-lasting protection, probably with single dose administration, and an anticipated safety profile similar to licensed vaccines for active immunization.

Live attenuated vaccines are well-known to induce a very strong immune response and to elicit both cell-mediated and humoral immune responses. To overcome the risk of reversion associated with ‘live attenuated’ vaccines we have developed the ‘live attenuated SARS-COV-2’ vaccine using codon de-optimisation technology. Using this approach, the whole virus (Wuhan isolate) is synthetically created by varying its nucleotide sequences (codons) such that all the structural proteins that generate immune response remain unaltered, while the number of non-structural proteins required for replication are altered thereby attenuating the virus. While live attenuated vaccines are well known to induce a strong immune response there is also a risk of reversion of mutation, generally this risk exists when the attenuated virus has a limited number of mutations. To address this risk, we have created a large number of mutations, thereby virtually eliminating this risk.

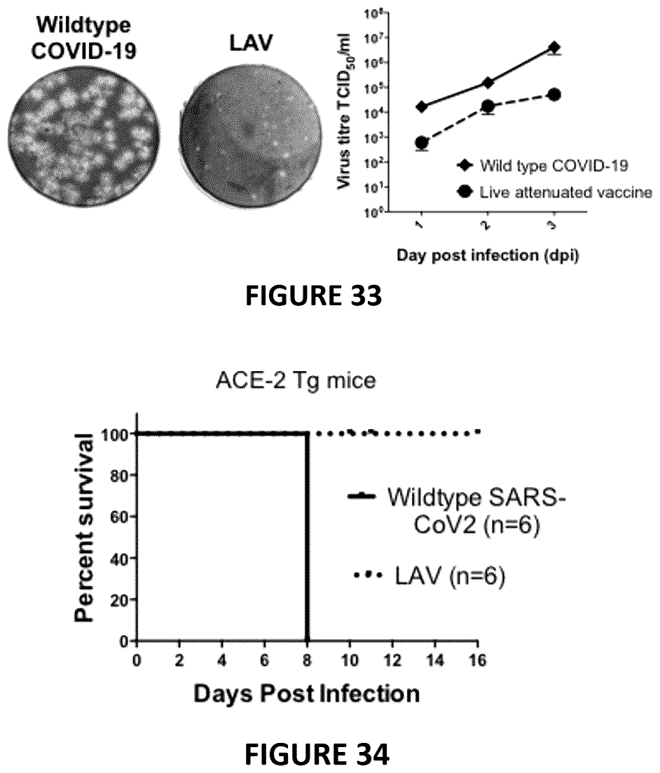

We first generated a synthetic SARS-COV-2 infectious clone based on the Wuhan strain. Next, we designed and constructed attenuated COVID-19 vaccine candidates using codon de-optimisation. For our leading vaccine candidate 7N-1, we introduced a large number of silent mutations (a total of 97 codon changes in the non-structural proteins 3 [nsP3] and 4 [nsP4]) in the replicative genes, but not in the structural proteins of the virus. There is essentially no risk of reversion to virulence due to the large number of substitutions in the gene sequences. The lead candidate, 7N-1, is highly attenuated, replicating to very low levels in mammalian cells and exhibiting a classic ‘small plaque phenotype’ indicative of a high level of attenuation (see FIGS. 33-7N-1 is labelled ‘LAV’). Small plaques demonstrate a reduced ability of the vaccine to spread from the initial site of infection. This serves as useful marker of vaccine attenuation. The attenuated ‘small plaque’ phenotype is maintained through extended tissue culture passage (see FIG. 32F), providing a strong indication that the virus is stable and will not revert to virulence.

### Safety Data in Mice

Mice infected with live attenuated vaccine 7N-1 show no signs of disease. Infection of mice with wildtype SARS-COV-2 results in substantial weight loss and disease followed by death by 7-8 days post-infection. To determine vaccine safety human ACE2 (hACE-2) transgenic mice were infected intranasally with high dose (10⁵ PFU) 7N-1 and monitored over a 10-day period. The mice exhibited no weight loss or any other disease signs. In contrast, mice infected with 10⁵ PFU wild-type SARS-COV-2 suffered substantial weight loss and died within 8 days (see FIG. 34). In contrast, mice inoculated with vaccine 7N-1 survived (see FIG. 34—labelled ‘LAV’). Thus, the vaccine is highly attenuated in mice and is safe.

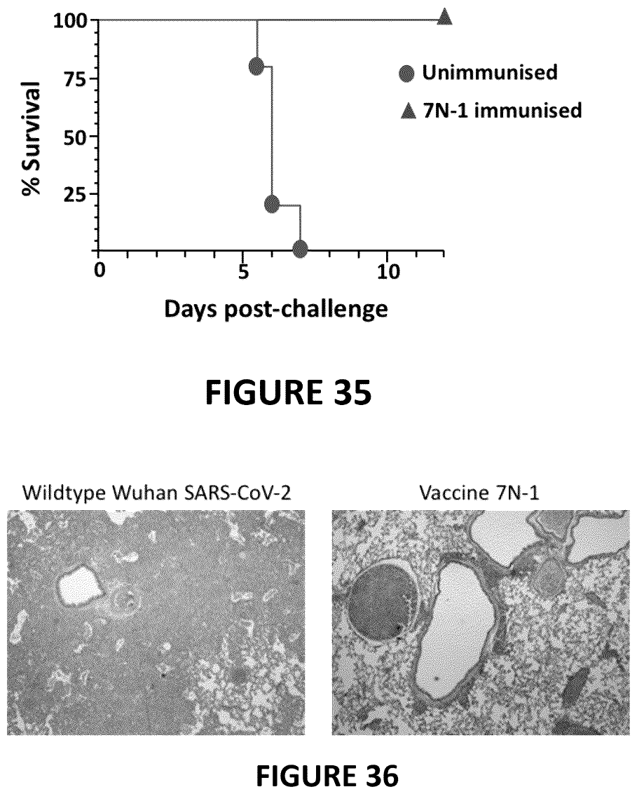

Vaccinated mice show no signs of disease after challenge with wildtype SARS-CoV-2. To test whether the vaccine protects against lethal infection with wildtype SARS-COV-2, hACE-2 transgenic mice were immunised with 10³ PFU 7N-1 intranasally followed by challenge three weeks later with 10⁵ PFU wild-type SARS-COV-2 intranasally. Infection of unimmunised mice with wild-type SARS-COV-2 resulted in 100% mortality within seven days. In contrast, immunised mice were completely protected, with no mortality (see FIG. 35). The data demonstrate the protective effects of candidate 7N-1 vaccine against a lethal challenge with wild-type SARS-COV-2.

### Safety Data in Hamsters

Hamsters infected with live attenuated vaccine show no signs of disease. To further evaluate vaccine safety, hamsters were infected intranasally with a high dose (10⁵ PFU) of vaccine candidate 7N-1 or wild-type SARS-COV-2. At days 3, 5, 7 and 14 post-infection, groups of hamsters were sacrificed for histological evaluation of lung pathology. Lung tissue sections were evaluated in a blinded fashion by experienced histopathologists at Vimta Labs Ltd (Hyderabad). Hamsters given candidate 7N-1 showed minimal lung pathology, with pathology readout scores of 3-4.5% on days 5 and 7 (days when peak inflammation is expected) while uninfected hamsters scored 0%. See Table 8 below for scores from individual animal.

TABLE 8

| Group | Vaccine 7N-1 | | | | | | | | | | | | | | | |
|---|---|---|---|---|---|---|---|---|---|---|---|---|---|---|---|---|
| Day | Day 3 | | | | Day 5 | | | | Day 7 | | | | Day 14 | | | |
| Animal No | 1 | 2 | 3 | 4 | 5 | 6 | 7 | 8 | 9 | 10 | 11 | 12 | 13 | 14 | 15 | 16 |
| Overall severity | | | | | | | | | | | | | | | | |
| % of lung area affected | 1 | 0 | 0 | 1 | 5 | 3 | 5 | 1 | 15 | 2 | 1 | 1 | 1 | 2 | 5 | 5 |
| Mean | 0.5 | | | | 3.5 | | | | 4.75 | | | | 3.25 | | | |
| Distribution of lesions | | | | | | | | | | | | | | | | |
| Bronchial and | 1 | 0 | 0 | 1 | 1 | 2 | 1 | 1 | 1 | 0 | 0 | 0 | 1 | 2 | 1 | 1 |
| peribronchial | | | | | | | | | | | | | | | | |
| Patchy throughout the | 0 | 0 | 0 | 0 | 2 | 0 | 1 | 1 | 2 | 1 | 2 | 1 | 0 | 0 | 2 | 2 |
| lungs | | | | | | | | | | | | | | | | |
| Cell and tissue damage | | | | | | | | | | | | | | | | |
| Necrosis of BEC | 0 | 0 | 0 | 0 | 0 | 1 | 1 | 1 | 0 | 0 | 0 | 0 | 0 | 0 | 0 | 0 |
| Cellular debris in bronchi | 0 | 0 | 0 | 0 | 0 | 0 | 0 | 0 | 0 | 0 | 0 | 0 | 0 | 0 | 0 | 0 |
| Diffuse alveolar damage | 0 | 0 | 0 | 0 | 1 | 0 | 1 | 0 | 2 | 1 | 1 | 1 | 1 | 2 | 2 | 2 |
| Necrosis of AEC | 0 | 0 | 0 | 0 | 1 | 0 | 1 | 1 | 1 | 0 | 1 | 1 | 0 | 0 | 1 | 1 |
| Cellular debris in alveoli | 0 | 0 | 0 | 0 | 1 | 0 | 1 | 1 | 2 | 1 | 1 | 1 | 0 | 0 | 1 | 1 |
| Alveolar emphysema | 0 | 0 | 0 | 0 | 1 | 0 | 1 | 0 | 1 | 0 | 1 | 0 | 0 | 0 | 0 | 0 |
| Circulatory changes and | | | | | | | | | | | | | | | | |
| vascular lesions | | | | | | | | | | | | | | | | |
| Alveolar hemorrhage | 0 | 0 | 0 | 0 | 1 | 0 | 1 | 1 | 1 | 1 | 1 | 0 | 0 | 0 | 1 | 1 |
| Alveolar edema | 0 | 0 | 0 | 0 | 1 | 0 | 1 | 1 | 0 | 0 | 1 | 0 | 0 | 0 | 0 | 0 |
| Perivascular/interstitial | 0 | 0 | 0 | 0 | 1 | 0 | 1 | 0 | 1 | 0 | 1 | 1 | 0 | 0 | 1 | 1 |
| edema | | | | | | | | | | | | | | | | |
| Vascular endothelialitis | 0 | 0 | 0 | 0 | 1 | 1 | 1 | 1 | 0 | 0 | 0 | 0 | 0 | 0 | 0 | 0 |
| Necrosis and | 0 | 0 | 0 | 0 | 0 | 0 | 0 | 0 | 0 | 0 | 0 | 0 | 0 | 0 | 0 | 0 |
| desquamation of vascular | | | | | | | | | | | | | | | | |
| endothelial cells | | | | | | | | | | | | | | | | |
| Reactive inflammatory | | | | | | | | | | | | | | | | |
| patterns | | | | | | | | | | | | | | | | |
| Necrosuppurative | 0 | 0 | 0 | 0 | 0 | 0 | 0 | 0 | 0 | 0 | 0 | 0 | 0 | 0 | 0 | 0 |
| bronchitis | | | | | | | | | | | | | | | | |
| Bronchointerstitial | 0 | 0 | 0 | 0 | 0 | 0 | 0 | 0 | 0 | 0 | 0 | 0 | 0 | 0 | 0 | 0 |
| pneumonia | | | | | | | | | | | | | | | | |
| Interstitial pneumonia | 0 | 0 | 0 | 0 | 1 | 0 | 0 | 0 | 1 | 0 | 1 | 1 | 0 | 0 | 0 | 0 |
| Intraalveolar neutrophils | 0 | 0 | 0 | 0 | 2 | 0 | 2 | 1 | 2 | 1 | 1 | 1 | 0 | 0 | 2 | 2 |
| and macrophages | | | | | | | | | | | | | | | | |
| Lymphocytes | 1 | 0 | 0 | 1 | 1 | 1 | 1 | 1 | 2 | 1 | 1 | 1 | 0 | 0 | 2 | 2 |
| Polymorphonuclear | 1 | 0 | 0 | 1 | 1 | 1 | 1 | 1 | 1 | 1 | 1 | 1 | 0 | 0 | 1 | 1 |
| granulocytes (neutrophils, | | | | | | | | | | | | | | | | |
| heterophils) | | | | | | | | | | | | | | | | |
| Monocytes, macrophages | 0 | 0 | 0 | 0 | 2 | 0 | 1 | 1 | 1 | 1 | 1 | 1 | 1 | 1 | 2 | 2 |
| Perivascular lymphocytic | 0 | 0 | 0 | 0 | 1 | 1 | 1 | 1 | 2 | 2 | 1 | 1 | 0 | 0 | 2 | 2 |
| cuffing | | | | | | | | | | | | | | | | |
| Activation of mesothelial | 0 | 0 | 0 | 0 | 0 | 0 | 0 | 0 | 0 | 0 | 0 | 0 | 0 | 0 | 0 | 0 |
| cells | | | | | | | | | | | | | | | | |
| Regeneration and repair | | | | | | | | | | | | | | | | |
| Hyperplasia of BEC | 0 | 0 | 0 | 0 | 0 | 0 | 0 | 0 | 0 | 0 | 0 | 0 | 0 | 0 | 0 | 0 |
| Hyperplasia of AEC-II | 0 | 0 | 0 | 0 | 0 | 0 | 0 | 0 | 0 | 1 | 1 | 0 | 1 | 1 | 0 | 0 |
| Multinucleated or | 0 | 0 | 0 | 0 | 0 | 0 | 0 | 0 | 0 | 0 | 0 | 0 | 0 | 0 | 0 | 0 |
| otherwise atypical | | | | | | | | | | | | | | | | |
| epithelial cells | | | | | | | | | | | | | | | | |
| Pleural fibroblastic | 0 | 0 | 0 | 0 | 0 | 0 | 0 | 0 | 0 | 0 | 0 | 0 | 0 | 0 | 0 | 0 |
| proliferation/fibrosis | | | | | | | | | | | | | | | | |

In contrast, hamsters infected with wild-type SARS-COV-2 had pathology scores greater than 50% at the same time points. See Table 9 below for scores from individual animals.

TABLE 9

| Group | Wildtype Wuhan SARS-CoV-2 | | | | | | | | | | | | | | | |
|---|---|---|---|---|---|---|---|---|---|---|---|---|---|---|---|---|
| Day | Day 3 | | | | Day 5 | | | | Day 7 | | | | Day 14 | | | |
| Animal No | 1 | 2 | 3 | 4 | 5 | 6 | 7 | 8 | 9 | 10 | 11 | 12 | 13 | 14 | 15 | |
| Overall severity | | | | | | | | | | | | | | | | |
| % of lung area | 1 | 52 | 5 | 10 | 50 | 40 | 80 | 50 | 80 | 50 | 50 | 40 | 20 | 1 | 35 | 0 |
| affected | | | | | | | | | | | | | | | | |
| Mean | 10 | | | | 55 | | | | 55 | | | | 19 | | | |
| Distribution of | | | | | | | | | | | | | | | | |
| lesions | | | | | | | | | | | | | | | | |
| Bronchial and | 1 | 2 | 2 | 2 | 2 | 2 | 3 | 3 | 2 | 2 | 2 | 2 | 1 | 1 | 2 | 0 |
| peribronchial | | | | | | | | | | | | | | | | |
| Patchy throughout the | 0 | 2 | 1 | 1 | 3 | 3 | 4 | 3 | 3 | 3 | 3 | 3 | 2 | 1 | 3 | 0 |
| lungs | | | | | | | | | | | | | | | | |
| Cell and tissue | | | | | | | | | | | | | | | | |
| damage | | | | | | | | | | | | | | | | |
| Necrosis of BEC | 1 | 2 | 1 | 2 | 1 | 1 | 2 | 0 | 0 | 0 | 0 | 0 | 0 | 0 | 0 | 0 |
| Cellular debris in | 1 | 1 | 1 | 1 | 1 | 1 | 2 | 1 | 1 | 0 | 1 | 0 | 0 | 0 | 0 | 0 |
| bronchi | | | | | | | | | | | | | | | | |
| Diffuse alveolar | 0 | 2 | 1 | 1 | 3 | 3 | 4 | 3 | 3 | 3 | 3 | 3 | 2 | 1 | 3 | 0 |
| damage | | | | | | | | | | | | | | | | |
| Necrosis of AEC | 0 | 2 | 1 | 1 | 1 | 1 | 2 | 2 | 0 | 0 | 0 | 0 | 2 | 0 | 0 | 0 |
| Cellular debris in | 0 | 2 | 1 | 1 | 3 | 3 | 3 | 2 | 2 | 2 | 2 | 2 | 2 | 0 | 1 | 0 |
| alveoli | | | | | | | | | | | | | | | | |
| Alveolar emphysema | 0 | 2 | 1 | 1 | 1 | 1 | 3 | 2 | 1 | 1 | 1 | 3 | 2 | 0 | 1 | 0 |
| Circulatory changes | | | | | | | | | | | | | | | | |
| and vascular lesions | | | | | | | | | | | | | | | | |
| Alveolar hemorrhage | 0 | 2 | 1 | 1 | 2 | 2 | 3 | 2 | 1 | 1 | 1 | 2 | 1 | 0 | 1 | 0 |
| Alveolar edema | 0 | 1 | 1 | 1 | 1 | 3 | 3 | 2 | 1 | 1 | 1 | 1 | 1 | 0 | 1 | 0 |
| Perivascular/interstitial | 0 | 1 | 1 | 1 | 2 | 2 | 3 | 2 | 2 | 2 | 2 | 2 | 1 | 0 | 1 | 0 |
| edema | | | | | | | | | | | | | | | | |
| Vascular | 2 | 2 | 1 | 2 | 2 | 3 | 2 | 2 | 0 | 0 | 0 | 0 | 0 | 0 | 0 | 0 |
| endothelialitis | | | | | | | | | | | | | | | | |
| Necrosis and | 1 | 1 | 1 | 1 | 1 | 1 | 0 | 0 | 0 | 0 | 0 | 0 | 0 | 0 | 0 | 0 |
| desquamation of | | | | | | | | | | | | | | | | |
| vascular endothelial | | | | | | | | | | | | | | | | |
| cells | | | | | | | | | | | | | | | | |
| Reactive inflammatory | | | | | | | | | | | | | | | | |
| patterns | | | | | | | | | | | | | | | | |
| Necrosuppurative | 0 | 0 | 0 | 0 | 0 | 0 | 3 | 0 | 0 | 0 | 0 | 0 | 0 | 0 | 0 | 0 |
| bronchitis | | | | | | | | | | | | | | | | |
| Bronchointerstitial | 0 | 0 | 0 | 0 | 0 | 0 | 2 | 0 | 0 | 0 | 0 | 0 | 0 | 0 | 0 | 0 |
| pneumonia | | | | | | | | | | | | | | | | |
| Interstitial pneumonia | 0 | 0 | 0 | 0 | 0 | 0 | 0 | 0 | 0 | 0 | 0 | 0 | 0 | 0 | 0 | 0 |
| Intraalveolar | 0 | 2 | 1 | 2 | 2 | 2 | 3 | 2 | 2 | 2 | 2 | 2 | 2 | 1 | 1 | 0 |
| neutrophils and | | | | | | | | | | | | | | | | |
| macrophages | | | | | | | | | | | | | | | | |
| Lymphocytes | 1 | 2 | 1 | 2 | 2 | 2 | 2 | 2 | 2 | 2 | 2 | 2 | 2 | 1 | 1 | 0 |
| Polymorphonuclear | 1 | 2 | 1 | 2 | 1 | 1 | 2 | 1 | 1 | 1 | 1 | 1 | 1 | 0 | 1 | 0 |
| granulocytes | | | | | | | | | | | | | | | | |
| (neutrophils, | | | | | | | | | | | | | | | | |
| heterophils) | | | | | | | | | | | | | | | | |
| Monocytes, | 0 | 2 | 1 | 2 | 3 | 3 | 3 | 3 | 3 | 3 | 3 | 3 | 2 | 1 | 1 | 0 |
| macrophages | | | | | | | | | | | | | | | | |
| Perivascular | 1 | 2 | 1 | 1 | 2 | 2 | 2 | 2 | 2 | 2 | 2 | 2 | 1 | 1 | 1 | 0 |
| lymphocytic cuffing | | | | | | | | | | | | | | | | |
| Activation of | 0 | 0 | 0 | 0 | 0 | 0 | 0 | 0 | 0 | 0 | 0 | 0 | 0 | 0 | 0 | 0 |
| mesothelial cells | | | | | | | | | | | | | | | | |
| Regeneration and | | | | | | | | | | | | | | | | |
| repair | | | | | | | | | | | | | | | | |
| Hyperplasia of BEC | 0 | 0 | 0 | 0 | 3 | 1 | 3 | 2 | 2 | 2 | 2 | 2 | 0 | 0 | 3 | 0 |
| Hyperplasia of | 0 | 0 | 0 | 0 | 1 | 0 | 1 | 1 | 3 | 3 | 3 | 3 | 2 | 2 | 3 | 0 |
| AEC0II | | | | | | | | | | | | | | | | |
| Multinucleated or | 0 | 0 | 0 | 0 | 0 | 0 | 0 | 0 | 0 | 0 | 0 | 0 | 0 | 0 | 0 | 0 |
| otherwise atypical | | | | | | | | | | | | | | | | |
| epithelial cells | | | | | | | | | | | | | | | | |
| Pleural fibroblastic | 0 | 0 | 0 | 0 | 0 | 0 | 0 | 0 | 0 | 0 | 0 | 0 | 1 | 0 | 0 | 0 |
| proliferation/fibrosis | | | | | | | | | | | | | | | | |

For all the specific pathology readouts, such as cellular infiltration, tissue damage and epithelial hyperplasia, the scores for candidate 7N-1 were dramatically lower than for wild-type virus. Histopathological changes were observed in wildtype SARS-COV-2-infected groups with increase in severity of % lungs affected was observed on 7-day post infection (see FIG. 36). The % lungs affected was lower in 7N-1 on Day 7 post infection (see FIG. 36). The results clearly demonstrate the safety of the vaccine in the hamster model. Hamster and human ACE2 are nearly identical, with only four amino acid differences, suggesting that these results are likely to provide a good model for human infection and vaccination with 7N-1.

### Candidate 7N-1 Vaccine Induces Strong Neutralizing Antibodies in Hamsters.

The route of live attenuated vaccine immunisation in humans is subcutaneous. Therefore, we determined the immunogenicity of the vaccine in hamsters when given subcutaneously. To assess immunogenicity, hamsters were immunised subcutaneously with 10⁴ PFU candidate 7N-1. Serum samples were collected two weeks later to measure PRNT₅₀ (50% of virus neutralized), PRNT₉₀ (90% of virus neutralized) and PRNT₁₀₀ (100% of virus neutralized) neutralising antibody titers (see Table 7 of an earlier Example). The neutralising antibody titers elicited by immunisation with candidate 7N-1 were very high, demonstrating the vaccine's strong immunogenicity.

### Example 18—Rational Design of Vaccine Candidates

We designed, constructed and tested various attenuated COVID-19 vaccine candidates using codon de-optimisation (CD) of the ORF1a region of the genome, from SanD1¹⁵³⁴ to PacI⁸⁵⁸⁶, which we call the deoptimised region—‘DO region’. Various combinations of CD fragments 1-3 were used to generate the candidates: Fragment 1: SanD1¹⁵³⁴ to SmaI⁴²⁵⁴; Fragment 2: SmaI⁴²⁵⁴ to AvrII⁶⁹⁸²; and, Fragment 3:AvrII⁶⁹⁸² to PacI⁸⁵⁸⁶. Fragment 3 is the shortest fragment. The numbers 1534, 8586 etc. indicate the genomic positions of the Wuhan strain. The codon changes per fragment and for the DO region are summarised in Table 10 below.

TABLE 10

| Group | Fragment 1 | Fragment 2 | Fragment 3 | Entire DO region |
|---|---|---|---|---|
| 77 | Ser: 24 | Ser: 28 | Ser: 25 | Ser: 24, Ser: 28, Ser: 25, |
| group | | | | total 77 |

77 group - Targeting most rare codon: 50% serine
(did not target Arg): 77-1 (fragment 3), 77-2
(fragment 2), 77-3 (fragment 1), 77-4 (fragments 1&2),
77-5 (fragments 1&3), 77-6 (fragments
2&3), 77-7 (fragments 1-3).
All vaccine candidates were rescued.

| | | | | |
|---|---|---|---|---|
| 160 | Ser: 48 | Ser: 59 | Ser: 53 | Ser: 48, Ser: 59, Ser: 53, |
| group | | | | total 160 |

160 group - Targeting most rare codon: 100% serine
(did not target Arg): 160-1 (fragment 3), 160-2
(fragment 2), 160-3 (fragment 1), 160-4 (fragments 1&2),
160-5 (fragments 1&3), 160-6 (fragments
2&3), 160-7 (fragments 1-3).
All vaccine candidates were rescued.

| | | | | |
|---|---|---|---|---|
| 4N | I: 39, P: 32, T: 67, | I: 24, P: 39, | I: 19, P: 10, T: 34, | I: 82, P: 81, T: 186, A: 147, |
| group | A: 62 | T: 85, A: 51 | A: 34 | total 496 |

4N group - Targeting second rare codon: IPTA (did not
target Leu, Val): 4N-1 (fragment 3) was
rescued. The following were not rescued/were dead
clones: 4N-2 (fragment 2), 4N-3 (fragment 1),
4N-4 (fragments 1&2), 4N-5 (fragments 1&3),
4N-6 (fragments 2&3), 4N-7 (fragments 1-3).

| | | | | |
|---|---|---|---|---|
| 7N | I: 40, P: 33, T: 68, | I: 4, P: 39, T: 77, | I: 0, P: 10, T: 33, | I: 44, P: 82, T: 178, A: 147, |
| group | A: 62, R: 11, G: 21, | A: 51, R: 18, | A: 34, R: 11, G: 8, | R: 40, G: 41, Q: 14, |
| | Q: 9 | G: 12, Q: 4 | Q: 1 | total 546 |

7N group - Targeting most rare codon (ARG), second
rare codon (Pro, Thr, Ala; did not target Leu,
Val) and not rare codons (Gly, Gln): 7N-1
(fragment 3) was rescued. The following were not
rescued/were dead clones: 7N-2 (fragment 2), 7N-3
(fragment 1), 7N-4 (fragments 1&2), 7N-5
(fragments 1&3), 7N-6 (fragments 2&3), 7N-7 (fragments 1-3).

For the entire SanD1¹⁵³⁴ to PacI⁸⁵⁸⁶ genomic DO region. Table 11 below covers all of the amino acids (‘Aa’) throughout the entire DO region. Note, this table is looking at the entire DO region; all the numbers are for codons, rather than nucleotides.

TABLE 11

| | | | | | | Column #7 |
|---|---|---|---|---|---|---|
| | | | | | Column #6 | Aa number |
| | | | | | Number of | of other |
| | Column #2 | | | Column #5 | rare codons | codons |
| | Rare codon | Column #3 | Column #4 | Number of | for each | (maximum |
| Column #1 | for amino | Codon | Frequency of | amino acids | amino acid | codon |
| Amino | acid (target | fraction in | codon in | in DO | in the DO | change |
| acid | codon) | humans | humans (%) | region | region | number) |
| Ser | TCG | 0.05 | 4.4 (1^{st} group) | 162 | 2 | 160 |
| Arg | CGT | 0.08 | 4.54 (1^{st} group) | 54 | 11 | 43 |
| Thr | ACG | 0.11 | 6.14 (2^{nd} group) | 193 | 6 | 187 |
| Pro | CCG | 0.11 | 6.9 (2^{nd} group) | 85 | 3 | 82 |
| Val | GTA | 0.12 | 7.1 (2^{nd} group) | 200 | 35 | 165 |
| Leu | CTA | 0.07 | 7.2 (2^{nd} group) | 224 | 25 | 199 |
| Ala | GCG | 0.11 | 7.4 (2^{nd} group) | 156 | 9 | 147 |
| Ile | ATA | 0.17 | 7.5 (2^{nd} group) | 128 | 41 | 87 |
| Cys | TGT | 0.46 | 10.6 (3rd group) | 60 | 54 | 6 |
| Gly | GGT | 0.16 | 10.8 (3rd group) | 128 | 87 | 41 |
| His | CAT | 0.42 | 10.9 (3rd group) | 37 | 24 | 13 |
| Tyr | TAT | 0.44 | 12.2 (3rd group) | 102 | 59 | 43 |
| Gln | CAA | 0.27 | 12.3 (3rd group) | 81 | 53 | 28 |
| Trp | TGG | 1 | 13.2 (3rd group) | 24 | 24 | 0 |
| Asn | AAT | 0.47 | 17 (4th group) | 134 | 96 | 38 |
| Phe | TTT | 0.46 | 17.6 (4th group) | 98 | 76 | 22 |
| Asp | GAT | 0.46 | 21.8 (4th group) | 114 | 75 | 39 |
| Met | ATG | 1 | 22 (4th group) | 44 | 44 | 0 |
| Lys | AAA | 0.43 | 24.4 (4th group) | 176 | 120 | 56 |
| Glu | GAA | 0.42 | 29 (4th group) | 153 | 112 | 41 |

| | | | | | Column #13 |
|---|---|---|---|---|---|
| | Column #9 | | Column #11 | Column #12 | Predicted |
| Column #8 | Minimum | Column #10 | Maximum | Predicted | useful |
| Minimum | Aa codon | Maximum | Aa codon | useful number | percentage |
| Aa codon | change | Aa codon | change | of Aa codon | of Aa codon |
| change | percentage | change | percentage | changes | changes |
| number in | in our | number in | in our | in the DO | in the DO |
| our design | design | our design | design | region | region |
| 24 | 15% | 160 | 100% | 0 to 160 | 0-100% |
| 11 | 26% | 40 | 93% | 0 to 43 | 0-100% |
| 33 | 18% | 186 | 99% | 0 to 187 | 0-100% |
| 10 | 12% | 82 | 100% | 0 to 82 | 0-100% |
| / | / | / | / | 0 to 165 | 0-100% |
| / | / | / | / | 0 to 199 | 0-100% |
| 34 | 23% | 147 | 100% | 0 to 147 | 0-100% |
| 0 | 0% | 82 | 94% | 0 to 87 | 0-100% |
| / | / | / | / | 0 to 6 | 0-100% |
| 8 | 20% | 41 | 100% | 0 to 41 | 0-100% |
| / | / | / | / | 0 to 13 | 0-100% |
| / | / | / | / | 0 to 43 | 0-100% |
| 1 | 4% | 14 | 50% | 0 to 28 | 0-100% |
| / | / | / | / | 0 | 0%, no need |
| / | / | / | / | 0 to 38 | 0-100% |
| / | / | / | / | 0 to 22 | 0-100% |
| / | / | / | / | 0 to 39 | 0-100% |
| / | / | / | / | 0 | 0%, no need |
| / | / | / | / | 0 to 56 | 0-100% |
| / | / | / | / | 0 to 41 | 0-100% |

Column #3: Codon fraction in certain Aa. For example, Ser has 6 synonymous codons: AGT, AGC, TCG, TCA, TCT, TCC. The number of TCG in all the 6 Ser codons is 5%. This number is a reflection of rarity of the codon within one particular Aa.
Column #4: Codon frequency in human genome. This is different from Column #3 which is the fraction of the codon. Codon frequency is not only a reflection of the rarity of the codon, but also the rarity of the Aa. We hypothesise that the codon frequency is more suitable than codon fraction, as we want to slow down viral translation.
Column #5: The Aa number in the entire DO region. It is also the total codon number for certain Aa. For instance, there are 162 Ser in our DO region (SanD1¹⁵³⁴ to PacI⁸⁵⁸⁶). There are total 162 Ser codons.
Column #6: The number of rare codons for each Aa in the DO region. For instance, TCG is the rare codon of Ser. There are 2 Ser rare codons (TCG) in our DO region, which means we cannot change these 2 codons.
Column #7: The maximum mutation number that we can make for each Aa in the DO region. 162 − 2 = 160, we can change/deoptimize at most 160 Ser codons to its rare codon (TCG).
Column #8: In all our DO region designs, there is one having the least numbers of changes of a certain Aa. In the case of Ser, 77-3 targeting sub-fragment 1 is the design having least mutations of Ser (24). In other words, in our designs targeting Ser, we used at least 24 changes. Therefore, the number could be considered the minimum number of codon changes supported by our experiment.
Column #9: The percentage of the change number (Column #8) in the maximum change we could make (Column #7). 24/160 = 15%, we changed at least 15% of Ser in our designs.
Column #10: In all our designs, there is one having the most numbers of changes of an Aa. In the case of Ser, 160-7 targeting the entire region is the design having most mutations of Ser (160). In other words, in our designs targeting Ser, we used at most 160 changes. Therefore, the number could be considered the maximum number of codon changes supported by our experiment.
Column #11: The percentage of the change number (Column #10) in the maximum change we could make (Column #7). 160/160 = 100%, we changed at most 100% of Ser in our designs.
Column #12 and #13 are our estimation of the Aa changes (by number and percentage) which should result in potentially rescuable and efficacious vaccine candidates/clones.

Although we do not have experimental data for each and every amino acid change in Table 11, it is reasonable to expect that the stated percentage and number of codon changes within the DO region will result in potentially rescuable and efficacious vaccine candidates/clones. Codon frequency can be divided into 4 groups: under 5% (1^{st} group), 5-10% (2^{nd} group), 10-15% (3^{rd} group), over 15% (4^{th} group). See the group numbering in Column #4. We have experimental data representing codon changes for the first 3 groups, and the percentage of the codon change in our designs is quite wide (see Column #9 and #11). However, we do not have experimental data for the 4^{th} group (frequency over 15%), but we believe that the number and percentage of codon changes is reasonable and should result in potentially rescuable and efficacious vaccine candidates/clones. Our reasons are: Ser and Arg are representative for the Aa that have rare codons (frequency under 5%); Thr, Pro, Ala and Ile are representative for the Aa that have less rare codons (frequency 5-10%); and, Gly and Gln are representative for the Aa that don't have rare codons (frequency 10-15%). We do not have experimental data to support the coverage of the Aa with codon frequency over 15%. Asn, Phe, Asp, Lys and Glu. We probably do not need to cover those Aa, as the frequency of these Aa is very high (we want to de-optimize, not optimize). Met and Trp have only one synonymous codon and cannot be changed.

### Example 19—Mouse Model Testing of Vaccine Candidates

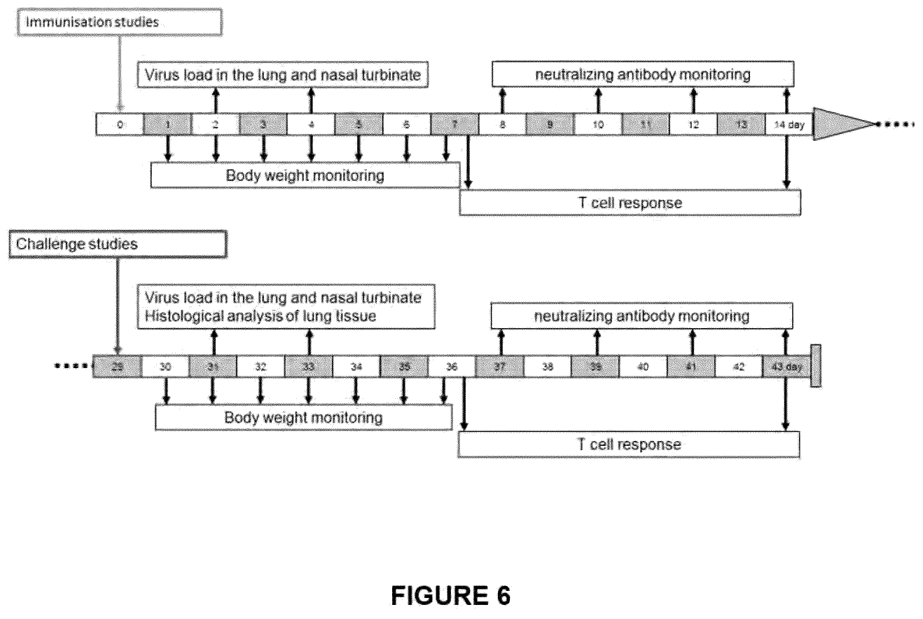

Further testing of vaccine candidates can be carried out as depicted in FIG. 6. Live attenuated SARS-COV-2 vaccine candidates can be tested in a mouse model of SARS-CoV infection using human ACE2 receptor transgenic mice. Antibody and cellular responses can be determined in immunised mice. Mice can be followed for a period of 2 weeks after immunisation. Body weight can be observed for 7 days. Virus load in the lung and nasal turbinate can be measured on day 2 and day 4 post-immunisation. Anti-SARS-COV-2 specific antibody levels (total IgG, IgM) and neutralisation titres can also be measured in the sera on various days post-immunisation. T cell immune responses can be examined on day 7 and day 14 post-immunisation. We can also evaluate vaccine efficacy by a challenge with a wild type SARS-COV-2. Immunised mice can be challenged 4 weeks after immunisation. Body weight can be observed in all groups. Challenged mice can be followed for a period of 2 weeks post-challenge. Viral titres can be measured in the lung and nasal turbinate on day 2 and day 4 post-post-challenge. Antibody titres (IgG, IgM) and neutralisation titres can be measured in the sera on specific days post-challenge. Histological analysis of lung tissue can be performed on day 2 and day 4 post-post-challenge. T cell responses can be examined at specific on day 7 and day 14 post-challenge.

### Example 20—Macaque Model Testing of Vaccine Candidates

Live attenuated SARS-COV-2 vaccine candidates can be further tested in the NHP model of SARS-COV 2 infection. Antibody and cellular responses can be determined in immunised macaque. Viremia in the sera, lung and/or nasal/oral secretions can be measured. Anti-SARS-COV-2 specific antibody levels, neutralisation titres and cellular immune response can be measured post-immunisation. We can also evaluate vaccine efficacy by a challenge with a wild type SARS-COV-2. Immunised macaque can be challenged several weeks after immunisation. Clinical symptoms can be observed in all groups. Viral titres can be measured in the sera, lung and/or nasal/oral secretions post-challenge. Antibody titres, neutralisation titres and cellular immune response can be measured post-challenge. Histological analysis of lung tissue can be performed post-challenge.

Total of five groups (each group can have five animals each). Total animals: 25.
Group 1 (N=5) Dose strength 1 Challenge on week 4 or week 9.
Group 2 (N=5) Dose strength 1, Booster on week 8, Challenge on week 9/10.
Group 3 (N=5) Dose strength 2 Challenge on Week 4 or week 9.
Group 4 (N=5) Dose strength 2, Booster on week 8, Challenge on week 9/10.
Group 5 (N=5) Challenge on week 4 or week 9.

The Nab titer (neutralizing antibodies) and the S-specific binding antibodies can be evaluated before challenge to determine vaccination efficacy. If it is too early to challenge at week 4 as the immune response after vaccination may be slow/delayed, challenge in week 9 can be undertaken.

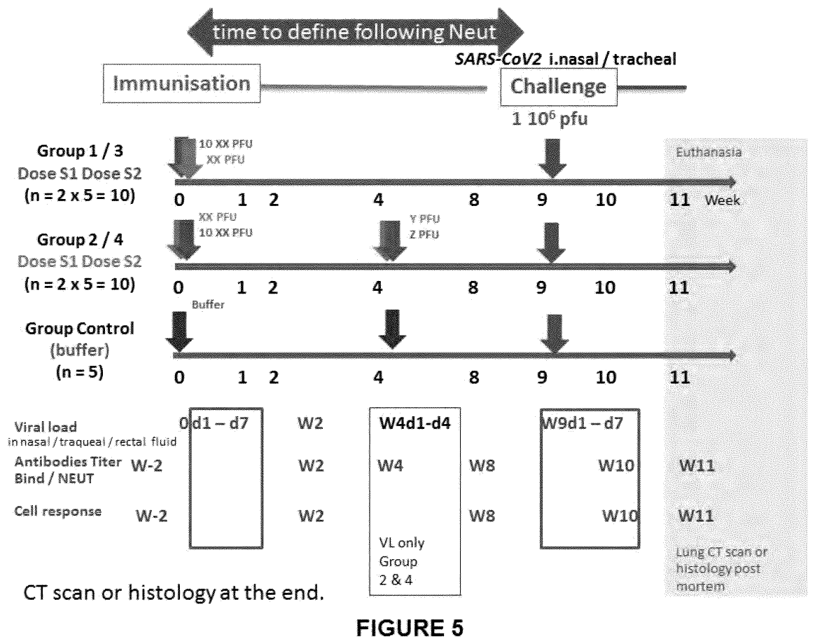

Various viral and immune parameters can be measured as described in FIG. 5.

### Example 21—Evaluation of Safety and Efficacy of Vaccine Candidate 7N-1 Against SARS-COV-2 in Cynomolgus Macaques

This Example briefly describes a study for evaluating the safety, the degree of immune response and the efficacy of vaccine candidate 7N-1 in the cynomolgus non-human primate (NHP) model after one or two immunizations.

### Protocol

The protocol is summarised in Table 12 below.

TABLE 12

| | | | | | Route, | | |
|---|---|---|---|---|---|---|---|
| | | | | Vaccine | volume, | Immunization | Challenge, |
| Groups | | n= | Species | (dose) | site | schedule | route, volume |
| 1 | Vaccinated | 5 | Cynomolgus | 7N-1 (10⁴ | s.c., 0.5 | 1 immunization | SARS-CoV-2 |
| | (prime | | macaques | PFU) | mL, | at w 0 | P1 variant |
| | only) | | | | right thigh | | i.n. (0.25 mL |
| 2 | Vaccinated | 5 | | 7N-1 (10⁴ | | 2 immunizations | per nostril) + |
| | (prime + | | | PFU) | | at w 0 and w 4 | i.t. (4.5 mL) at |
| | boost) | | | | | | week 8 post |
| 3 | Control | 5 | | DPBS | | 2 DPBS placebo | first |
| | | | | | | shot at w 0 and | immunization |
| | | | | | | w 4 | |

Experimental groups. (s.c: subcutaneous; i.n.: intranasal; i.t.: intratracheal)

As described in Table 12, fifteen cynomolgus macaques are included in this study and divided into 3 groups. Animals of groups 1 and 2 will be immunized with the 7N-1 vaccine (10⁴ PFU) by subcutaneous (s.c.) route. Animals from group 1 (n=5) will receive only one dose of the vaccine on day 0 and a placebo dose on week 4. The animals of group 2 (n=5) will be immunized both on day 0 while animals in group 2 will receive a prime and a boost on days 0 and 28 (4 weeks). Animals of control group (n=5) will receive the vehicle as placebo by s.c. route on day 0 and day 28.

All animals will then be exposed at week 8 post first immunization to SARS-COV-2 P1 variant (Brazilian strain) by intranasal (i.n.) and intratracheal (i.t.) routes simultaneously, with a 1.105 TCID50 challenge dose. Animals will be euthanized at day 14 post challenge. Samples will be collected and analyzed.

### Vaccine

The 7N-1 vaccine is a highly-purified, whole virus, SARS-COV-2 vaccine produced on Vero cells and attenuated by codon de-optimisation technology to make multiple mutations in the non-structural proteins of SARS-COV-2.

The composition of the vaccine (0.5 mL) is shown in Table 13.

TABLE 13

| | |
|---|---|
| Attenuated Wuhan | Antigen |
| SARS-CoV-2 | Units/dose): |

Active substance

10⁴ PFU

Excipients and buffer components
Dulbecco's Phosphate Buffered Saline (DPBS)/Tris Buffered Saline.
DPBS composition: 200 mg/L KCl (2.68 mM), 200 mg/L KH₂PO₄ (1.47 mM), 8000 mg/L NaCl (136.9 mM), 2160 mg/L Na₂HPO₄*7H₂O (8.06 mM); Tris buffered saline: 20 mM Tris, 100 mM NaCl, pH 7.5.

### SARS-COV-2 Virus

SARS-COV-2 P1 variant virus doses will be purchased from BEI Resources Repository (National Instituted of Health, USA).
Strain: SARS-COV-2, hCoV-19/Japan/TY7-503/2021 (P1)
Passage: 2
Sequence: Compared to the initial isolate, the following mutations are present: Spike D138Y, Spike D614G, Spike E484K, Spike H655Y, Spike K417T, Spike L18F, Spike N501Y, Spike P26S, Spike R190S, Spike T20N, Spike T1027I, Spike V1176F, N (Nucleocapsid protein) G204R, N P80R, N R203K, NSP3 (Non-structural protein 3) S253P, NSP8 (Non-structural protein 8) E92K, NSP3 K977Q, NSP3 S370L, NSP6 (Non-structural protein 6) F108del, NSP6 F184V, NSP6 G107del, NSP6 S106del, NSP12 (Non-structural protein 12) P323L, NSP13 (Non-structural protein 13) E341D, NSP6 (Non-structural protein 6) F184V. The full sequence was published and can be found online (SAMN18527803).
Titer: 4.42. 106 TCID50/mL
Volume/aliquot: 1 mL
produced on Calu-3 cells

### Preparation of the Vaccine

The vaccine will be provided in two formulations—liquid form and freeze-dried form. For the liquid form, the vaccine will be provided in vials containing 1 mL of attenuated SARS-COV-2 strain 7N-1. For the freeze-dried form, the vaccine will be provided in vials containing lyophilized powder of attenuated SARS-COV-2 strain 7N-1. The vaccine will be stored in a freezer at −80° C. SARS-Cov-2 virus strain 7N-1 (1×10⁵ PFU/ml/Vial). Freeze dried SARS-Cov-2 virus strain 7N-1 (1×10⁵ PFU/Vial).

### Example 22—Protocol for Rescue of SARS-COV-2 Virus from Full Length Constructs

This Example describes a protocol for successfully transfecting vaccine constructs, being an alternative to the method described in Example 8. We are able to rescue candidate viruses by directly transfecting Vero GMP cells without the need to use BHK cells.

### 1. Application

To rescue a recombinant SARS COV-2 virus from a full-length DNA infectious clone(s).

### 2. Materials Required

Nuclease free tips, Nuclease free tubes, Cell culture incubator, Nanodrop instrument, Microfuge, 12 well tissue culture plate, Light microscope, Sterile 1.5 mL Eppendorf tubes, SARS-COV-2 infectious clone plasmid(s), Vero-GMP cells, HEK-293 TT cells, BHK-21 cells, Vero E6 cells, Lipofectamine 2000 reagent (Thermo Fisher Scientific-11668019), Polyethyleneimine MAX (Polysciences Inc, 24765), Opti-MEM medium, HMEM, DMEM growth medium, and Fetal bovine serum.

### 3. Procedure

### Day 0—Plating Cells

Seed the cells at a recommended seed rate following Table 14 below.

TABLE 14

| Cells | Seeding density | Medium used |
|---|---|---|
| Vero-GMP | 2 × 10⁵ cells per/ml | HMEM with 10% FBS |
| Vero-E6 | 3 × 10⁵ cells per/ml | HMEM with 10% FBS |
| BHK-21 | 3 × 10⁵ cells per/ml | HMEM with 10% FBS |
| HEK-293 | 4 × 10⁵ cells per/ml | DMEM with 10% FBS |

Incubate the plates at 37° C. with 5% CO₂ overnight or until the monolayer reaches to 70-80% confluency.

### Day 1—Transfection

Prepare the transfection reagent and the DNA as provided in Table 15 below.

TABLE 15

| | Vial A | | | | |
|---|---|---|---|---|---|
| | | Volume | Vial B | | |
| | Volume of | (Lipofectamine | Volume of | Volume of | |
| Plate | OptiMEM | or PEI MAX) | OptiMEM | (DNA) | Ratio |
| 12 well | 100 μl | 6 μl | 100 μl | 2 μg | 1:3 |

Wait for 5 minutes at room temperature.

Mix the transfection reagent (Vial A) and DNA (Vial B) and incubate at room temperature for 20 minutes.

Transfer the lipofectamine/PEI MAX and DNA complex into the cells.

Gently mix and incubate the plates at 37° C. at 5% CO₂ up to 72-96 hours and observe the plates for every 12-24 hours.

After incubation, harvest the contents of the wells in to 15 ml centrifuge tube.

Freeze the tubes in −80° C. and quickly thaw the tubes in a 25° C. water bath. Repeat this step twice and clarify the sample by centrifugation at 1500 rpm for 10 minutes and store at −80° C. until further process.

### Day 5—Day 7-Passage of the Rescued Clone (Blind Passage-I)

### Day 5—Seeding Plates

Seed Vero-GMP cells at 3×10⁵ cells per/ml in a 12 well plate and incubate the plates at 37° C. at 5% CO₂.

### Day 6—Day 9—Propagation and Harvest

Infect the Vero-GMP monolayer (1 ml/well in 12 well plate) with the clarified transfected supernatant.

Incubate the plates at 37° C. at 5% CO₂ overnight (˜16 hours).

After incubation, replace the medium with fresh HMEM supplemented with 1% FBS and continue incubating the plates at 37° C. at 5% CO₂ for 60 hours.

After incubation, harvest the virus (supernatant and cells) and store at −80° C. until further process.

Propagation of the rescued virus (Blind passage-II)

If required (if the CPE is not evident), repeat the passage once again in Vero-GMP cells as described above before further scale up.

Titrate the virus following standard protocol.

Scale up the virus as required at a preferred MOI.

### Example 23—Vaccine Candidate 7N-1 Provides Full Protection from Rechallenge Mortality when Given Via Intranasal Route in HFH4-hACE2 Mice

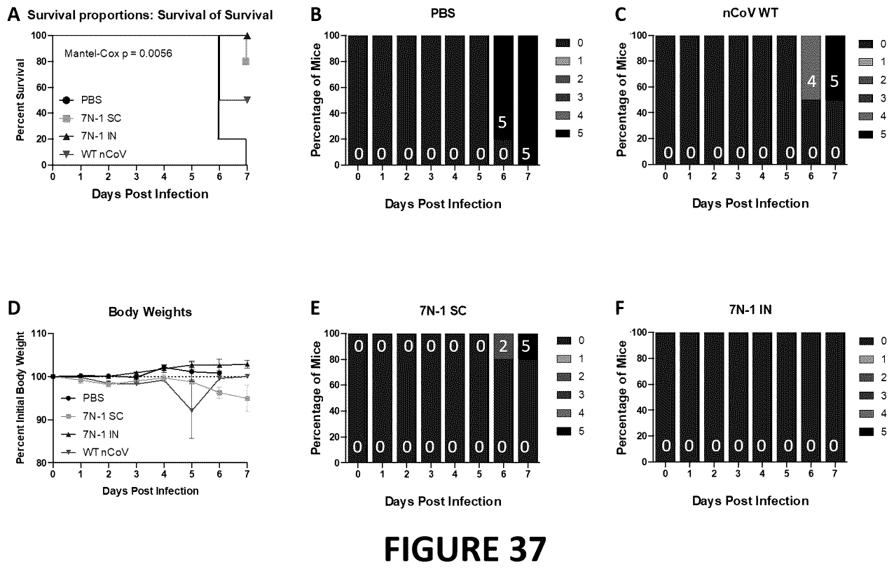

To test the efficacy of candidate 7N-1 as a COVID-19 vaccine, we established a challenge experiment using the following immunisation groups: 10³ PFU 7N-1 intranasal; 10⁵ PFU 7N-1 subcutaneous; 10³ PFU wild-type mouse-adapted SARS-COV-2 (SARS-COV-2 MA10) intranasal; and unimmunised (PBS treated). See FIG. 37A-F.

HFH4-hACE2 mice were used in this study. Three weeks later, the mice were challenged with 10⁵ PFU intranasal wild-type mouse-adapted SARS-COV-2 (SARS-COV-2 MA10) and monitored over a 7-day period. All mice in the unvaccinated group were moribund by day 7 post-infection and were euthanised. Mice given 7N-1 vaccination showed strong protection from SARS-COV-2 challenge, with 100% survival in the intranasal group and 80% survival in the subcutaneous group (death in mice given 7N-1 subcutaneous is likely related to the reduced receptor expression in the periphery). Immunisation with wild-type mouse-adapted SARS-COV-2 (SARS-COV-2 MA10) provided partial protection.

### Example 24—Form of the Vaccine and Immunisation Protocol

Live attenuated SARS-COV-2 vaccine/vaccine dose can comprise freeze-dried/lyophilized infectious virus as produced in the earlier Examples. The freeze-dried/lyophilized infectious virus can be reconstituted and administered by subcutaneous injection, inhalation or oral route. In preferred embodiments, the vaccine is administered by subcutaneous injection, intranasally or orally. The vaccine can be used for prophylactic, active, single-dose immunization against SARS-COV-2 in humans. A subject may be administered, for example, a titre of approximately 10⁴ PFU attenuated virus per vaccine dose.

There are a number of key features of the codon-deoptimized SARS-COV-2 vaccine that make it very attractive as a commercial candidate.
1) There are no licensed vaccines available for SARS-COV-2.
2) The vaccine is a live attenuated vaccine, which generally delivers more potent immunity and longer protection from infection than other vaccine formulations such as subunit vaccines. It has also no adverse effect due to anti-vector immunity that will likely be problem for adenovirus-based vaccines, especially if annual re-vaccination is needed.
2) The vaccine is expected to replicate to very low levels in mammalian cells but still induces potent immunity.
3) We have achieved attenuation of the vaccine through targeted mutation of the region encoding for non-structural proteins (ORF1a region). We have introduced a number of silent mutations, which are expected to interfere with replication of the vaccine construct but have no effect on the structure of encoded proteins. By engineering multiple mutations into the vaccine, there is essentially no chance that the vaccine will revert to virulence. This is a crucial aspect of our vaccine design since safety of live attenuated vaccines is the major concern in bringing these types of vaccine to market. In contrast, vaccines based on a single attenuating mutation are highly susceptible to reversion and have considerable safety issues.
4) The vaccine is expected to provide cross-protection against other coronaviruses e.g. SARS-COV-1 and MERS-COV.
5) Other vaccine formulations are currently under development in other labs around the world (e.g. subunit vaccine, inactivate whole virus vaccine, recombinant virus vaccine, various RNA and DNA vaccines). Our approach involves the application of codon deoptimization technology to ORF1a to develop an attenuated vaccine.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention.

In the present specification and claims (if any), the word ‘comprising’ and its derivatives including ‘comprises’ and ‘comprise’ include each of the stated integers but does not exclude the inclusion of one or more further integers.

The reference to any prior art in this specification is not, and should not be taken as an acknowledgement or any form of suggestion that the prior art forms part of the common general knowledge.

1. A live attenuated-severe acute respiratory syndrome coronavirus 2 (SARS-CoV-2), SARS-COV-2, SARS-COV-2 particle or SARS-COV-2 nucleic acid comprising a partly codon deoptimized SARS-COV-2 genome.

2. A recombinant, isolated or substantially purified nucleic acid comprising a partly codon deoptimized SARS-COV-2 genome or partly codon deoptimized region thereof.

3. A vector, plasmid or genetic construct comprising the nucleic acid of claim 2.

4. A cell or isolate containing the live attenuated SARS-COV-2, SARS-COV-2, SARS-COV-2 particle, or SARS-COV-2 nucleic acid of claim 1.

5. An immunogenic composition, pharmaceutical preparation or vaccine comprising the live attenuated SARS-COV-2, SARS-COV-2, SARS-COV-2 particle or SARS-COV-2 nucleic acid of claim 1.

6. (canceled)

7. (canceled)

8. A method of: (1) vaccinating a subject; (2) prophylactically immunizing a subject against SARS-COV-2 or SARS-COV-2-like virus; (3) preventing a subject from contracting a SARS-COV-2 infection naturally or a SARS-COV-2-like infection naturally; (4) reducing the severity of a natural SARS-COV-2 disease or natural SARS-COV-2-like disease in a subject; (5) treating a subject having a natural SARS-CoV-2 infection or natural SARS-COV-2-like infection; or (6) eliciting an immune response in a subject, said method comprising the step of administering to the subject: a live attenuated SARS-COV-2, SARS-COV-2, SARS-COV-2 particle or SARS-CoV-2 nucleic acid comprising a partly codon deoptimized SARS-COV-2 genome; a recombinant, isolated or substantially purified nucleic acid comprising a partly codon deoptimized SARS-COV-2 genome or partly codon deoptimized region thereof; a vector, plasmid or genetic construct comprising the recombinant, isolated or substantially purified nucleic acid; a cell or isolate containing the live attenuated SARS-COV-2, SARS-COV-2, SARS-COV-2 particle or SARS-COV-2 nucleic acid; or the immunogenic composition, pharmaceutical preparation or vaccine of claim 5.

9. (canceled)

10. (canceled)

11. A method of generating a live attenuated SARS-COV-2 vaccine, SARS-CoV-2, SARS-COV-2 particle or SARS-COV-2 nucleic acid, or recombinant, isolated or substantially purified nucleic acid comprising a partly codon deoptimized SARS-COV-2 genome or partly codon deoptimized region thereof, comprising the step of partly codon deoptimizing a SARS-COV-2 genome.

12. The method of claim 11, further comprising the step of enabling the partly codon deoptimized live attenuated SARS-COV-2 to replicate.

13. The method of claim 12, further comprising the step of preparing a vaccine dose containing the replicated SARS-COV-2.

14. The live attenuated SARS-COV-2, SARS-COV-2, SARS-COV-2 particle or SARS-COV-2 nucleic acid of claim 1,
wherein the partly codon deoptimized SARS-COV-2 genome comprises or consists of codon changes within the ORF1a region of SARS-COV-2.

15. The live attenuated SARS-COV-2, SARS-COV-2, SARS-COV-2 particle or SARS-COV-2 nucleic acid of claim 14,
wherein the partly codon deoptimized SARS-COV-2 genome comprises or consists of codon changes within the ORF1a region of SARS-COV-2, excluding the 5′ region of ORF1 and/or excluding the 3′ region of ORF1a corresponding to the ribosomal frameshift region; or
wherein the partly codon deoptimized SARS-COV-2 genome comprises or consists of codon changes within the ORF1a region of SARS-COV-2 corresponding to between about nucleotide position 1534 and about nucleotide position 8586 of the wild-type Wuhan SARS-COV-2 genome.

16. The live attenuated SARS-COV-2, SARS-COV-2, SARS-COV-2 particle or SARS-COV-2 nucleic acid of claim 14,
wherein the partly codon deoptimized SARS-COV-2 genome comprises or consists of codon changes within the ORF1a region of SARS-COV-2 corresponding to:
(1) between about nucleotide position 1534 and about nucleotide position 4254 of the wild-type Wuhan SARS-COV-2 genome;
(2) between about nucleotide position 4254 and about nucleotide position 6982 of the wild-type Wuhan SARS-COV-2 genome;
(3) between about nucleotide position 6982 and about nucleotide position 8586 of the wild-type Wuhan SARS-COV-2 genome;
(4) between about nucleotide position 8586 and about nucleotide position 11165 of the wild-type Wuhan SARS-COV-2 genome;
(5) between about nucleotide position 11165 and about nucleotide position 12718 of the wild-type Wuhan SARS-COV-2 genome; or
(6) any combination of (1) to (5).

17. The live attenuated SARS-COV-2, SARS-COV-2, SARS-COV-2 particle or SARS-COV-2 nucleic acid of claim 14,
wherein the partly codon deoptimized SARS-COV-2 genome comprises or consists of between about 10 and about 1850 codon changes within the ORF1a region; or
wherein the partly codon deoptimized SARS-COV-2 genome comprises or consists of between about 24 and about 546 codon changes within the ORF1a region.

18. The live attenuated SARS-COV-2, SARS-COV-2, SARS-COV-2 particle or SARS-COV-2 nucleic acid of claim 14,
wherein the partly codon deoptimized SARS-COV-2 genome comprises or consists of codon changes to synonymous codons that are used less frequently, moderately, less rarely, and/or rarely in the genome of Homo sapiens; or
wherein the partly codon deoptimized SARS-COV-2 genome comprises or consists of
codon changes to one or more rare codons, one or more less rare codons, one or more moderate codons, one or more codons containing CG (CpG) dinucleotides, one or more codons containing UA (UpA) dinucleotides, or any combination thereof.

19. The live attenuated SARS-COV-2, SARS-COV-2, SARS-COV-2 particle or SARS-COV-2 nucleic acid of claim 14,
wherein the partly codon deoptimized SARS-COV-2 genome comprises or consists of synonymous codon changes whereby one or more Ser codons are changed, one or more Arg codons are changed, one or more Thr codons are changed, one or more Pro codons are changed, one or more Val codons are changed, one or more Leu codons are changed, one or more Ala codons are changed, one or more Ile codons are changed, one or more Cys codons are changed, one or more Gly codons are changed, one or more His codons are changed, one or more Gln codons are changed, one or more Trp codons are changed, one or more Asn codons are changed, one or more Phe codons are changed, one or more Asp codons are changed, one or more Phe codons are changed, one or more Lys codons are changed, one or more Glu codons are changed, or any combination thereof.

20. The live attenuated SARS-COV-2, SARS-COV-2, SARS-COV-2 particle or SARS-COV-2 nucleic acid of claim 14,
wherein the partly codon deoptimized SARS-COV-2 genome comprises or consists of synonymous codon changes whereby:
one or more Ser codons are changed to the rare TCG codon;
one or more proline codons are changed to the less rare CCG codon;
one or more threonine codons are changed to the less rare ACG codon;
one or more isoleucine codons are changed to the less rare ATA codon;
one or more alanine codons are changed to the less rare GCG codon; and/or
one or more arginine codons are changed to the rare CGT codon or less rare CGA codon.

21. The live attenuated SARS-COV-2, SARS-COV-2, SARS-COV-2 particle or SARS-COV-2 nucleic acid of claim 14,
wherein the partly codon deoptimized SARS-COV-2 genome comprises or consists of codon changes within the ORF1a region of SARS-COV-2 comprising:
a deoptimized nucleotide sequence as shown or substantially as shown in any one of SEQ ID NO:33-37;
a deoptimized nucleotide sequence as shown or substantially as shown in any one of SEQ ID NO:39-68;
a deoptimized nucleotide sequence as shown or substantially as shown in any one of
FIGS. 22 to 25;
a deoptimized nucleotide sequence as shown in any one of SEQ ID NO:33-37 but with up to 10% fewer or up to 10% more codon changes than shown;
a deoptimized nucleotide sequence as shown in any one of SEQ ID NO:39-68 but with up to 10% fewer or up to 10% more codon changes than shown; or
a deoptimized nucleotide sequence as shown in any one of FIGS. 22 to 25 but with up to 10% fewer or up to 10% more codon changes than shown.

22. The live attenuated SARS-COV-2, SARS-COV-2, SARS-COV-2 particle or SARS-COV-2 nucleic acid of claim 14,
wherein the partly codon deoptimized SARS-COV-2 genome comprises or consists of codon changes as listed in Table 1b, either individually or in combination with each other, or in Table 11, either individually or in combination with each other.

23. The live attenuated SARS-COV-2, SARS-COV-2, SARS-COV-2 particle or SARS-COV-2 nucleic acid of claim 14,
wherein the partly codon deoptimized SARS-COV-2 genome comprises the nucleotide sequence or substantially the same nucleotide sequence as clone SARS-COV-2-77-1, SARS-COV-2-77-2, SARS-COV-2-77-3, SARS-COV-2-77-4, SARS-COV-2-77-5, SARS-COV-2-77-6, SARS-COV-2-77-7, SARS-COV-2-160-1, SARS-COV-2-160-2, SARS-COV-2-160-3, SARS-COV-2-160-4, SARS-COV-2-160-5, SARS-COV-2-160-6, SARS-COV-2-160-7, SARS-COV-2-4N-1 or SARS-COV-2-7N-1, or any variant thereof such as SARS-COV-2-4N-1-Alpha (B1.1.7), SARS-COV-2-4N-1-Beta (B1.351), SARS-CoV-2-4N-1-Gamma (P1), SARS-COV-2-4N-1-Delta, SARS-COV-2-7N-1-Alpha (B1.1.7), SARS-COV-2-7N-1-Beta (B1.351), SARS-COV-2-7N-1-Gamma (P1), or SARS-COV-2-7N-1-Delta.

24. The live attenuated SARS-COV-2, SARS-COV-2, SARS-COV-2 particle or SARS-COV-2 nucleic acid of claim 14,
wherein the partly codon deoptimized SARS-COV-2 genome comprises the nucleotide sequence of clone SARS-COV-2-7N-1, or substantially the same nucleotide sequence as clone SARS-COV-2-7N-1, or any variant thereof such as SARS-COV-2-7N-1-Alpha (B1.1.7), SARS-COV-2-7N-1-Beta (B1.351), SARS-COV-2-7N-1-Gamma (P1), or SARS-COV-2-7N-1-Delta.

25. A vaccine comprising live attenuated SARS-COV-2, SARS-COV-2, SARS-CoV-2 particle or SARS-COV-2 nucleic acid comprising a partly codon deoptimized SARS-COV-2 genome, wherein the partly codon deoptimized SARS-COV-2 genome comprises the nucleotide sequence of clone SARS-COV-2-7N-1 or the sequence of SEQ ID NO:60, or substantially the same nucleotide sequence as clone SARS-COV-2-7N-1 or the sequence of SEQ ID NO:60, or any variant thereof such as SARS-COV-2-7N-1-Alpha (B1.1.7), SARS-COV-2-7N-1-Beta (B1.351), SARS-COV-2-7N-1-Gamma (P1), or SARS-COV-2-7N-1-Delta.
